SEQUENCE LISTING (177 sequences)

SEQ ID NO: 1 - protein, 243 aa, Chlamydia trachomatis
MLRVLAYRQVSFSKFPRLFKSFVSLLSSLKQHYSFMLPGDPLPKKKALMLTFDHASVDFYTHVFPLLQNLQIPAVIGVAWRYVADLEGEDLPIDVRIAPSDFLAFQDEIFSYHQPFCSVRELCHMAASPLVRFASSGFAIRNLKYAPPYLHTEILLSKILLENAIQSPVESFFFPLGKSDVVSQHFVQETYRYSFVLGNTASFSYSTQSLHGIPRIDMPLDSQRVPSLYQLSYRHLKQFLVLR

SEQ ID NO: 2 - protein, 53 aa, Chlamydia trachomatis
MHSSSPISEICSMETAERVLEEKEHTPTIISGQIVIYPEDPFAIPSVDPCFES

SEQ ID NO: 3 - protein, 819 aa, Chlamydia trachomatis
MNSTNNTDSQNLDPNASEVEKLLDESAEAEEKVDDHTPPSELFILPLNKRPFFPGMAAPLLIEAGPHYEVLTLLAKSSQKHIGLVLTKKEDANTLKVGFNQLHRVGVSARILRIMPIEGGSAQVLLSIEDRIRIVKPIQDKYLKAKVSYHKENKELTEELKAYSISIVSIIKDLLKLNPLFKEELQIFLGHSDFTEPGKLADFSVALTTATREELQEILETTDMHDRIDKALVLLKKELDLSRLQSSINQKIEATITKSQKEFFLKEQLKTIKKELGLEKDDHAVDLEKFMERFNKRDVPQYAMDVIQDEMDKLQTLETSSAEYAVCRNYLDWLTIVPWGIQTKEYHDLKKAESILNKDHYGLEDIKQRILELISVGKLANGMKGSIICLVGPPGVGKTSIGRSIAKVLHRKFFRFSVGGMRDEAEIKGHRRTYIGAMPGKLVQALKQSQIMNPVIMIDEVDKIGSSYHGDPASALLEVLDPEQNKDFLDHYLDVRVDLSNVLFILTANVLDSIPDPLLDRMEVLRLSGYILEEKLQIATKYLVPRARKEMGLSAQNVTFQPEALKHMINNYAREAGVRTLNENIKKVLRKVALKIVQNQEKNLSKKSRFTITPKNLQDYLGKPVFSSDRFYEKTPVGVATGLAWTSLGGATLYIESVQVPSSSGKADMHLTGQAGDVMKESSQIAWTYLHSALERYAPGQPFFEKSQVHIHIPEGATPKDGPSAGITMVTSLLSLLLDVPVLNNLGMTGELTLTGRVLGIGGIREKLIAARRSKLNILIFPEDNRRDYDELPAYLKKGLKVHFVTHYDDVFKIAFPGV

SEQ ID NO: 4 - protein, 275 aa, Chlamydia trachomatis
MMDYELLDSGDGKKLERFKDVCLIRSSATAIWPKSSPSLWGQYSAEFVRMGEQGQWRYRNRNLKEWWITIDSVSCLLKLTPFGHVGIFPEHAGFWKDLQPSVAKPSCRVLNLFAYTGASSIFCAQQGATVYHVDASKAAVKWAQKNVEGNAFQDKRIFWIIEDVFSFLKKEIRRGKTYDVILLDPPTYGRGPDGETFKIDRDFFPLLELCSQLLSSSFSHMLITSHTPGHTPEFLHCLARRALLMLPLQGWRLGENFCGEGNQRLPSGVFAQWSL

SEQ ID NO: 5 - protein, 199 aa, Chlamydia trachomatis
MFSGIIQEVARVDLIHHLRDSMEIGVFARKLIDVVPGSSFSVDGICLTLVKRQYELLFFDVTEETMAWTTIKDYTVGTMVNLERSVRLGDEIGGHFVSGHVCGIGTIIAIEKSYMFFKAPANLVPYILEKGFIAIDGISLTIARVKGDIFSVSLIPETRARTSLGYKQVGAHVNMEPDMMTKMQVDTIMRFHAEKEISK

SEQ ID NO: 6 - protein, 639 aa, Chlamydia trachomatis
MNVRTYSVQRGGVKTISASAVPPTAAVLSRKKRAIEEKKEEASSGKIENLDASKYDLTPKNIEEKLGITPEQKSTVKDLLNKLKKVISAYNSMPDKNSEAGQNSLIQQGKYVDAIQKKLPASSQAQPKQAKAKEQKAEEKPKTTPIEGVLETIKTEFKGHRVPVEKIIHGIWIAGAPPDGIEDYMRVFLDTYEGFDFYFWVDENAYAAAKFSSILKKVAFDAAIQDLRSATDESTKAFVKDYDELKQKYEKKVAETTSQAEKDQYLKDLKDLLEKFTKISDEIRGKFDRLFLKNVIVAQNGFFNFCLLKGLGNINDETRAEYLEKELKLPTEEIEQYKKLKETNKEKIAAIVKQLNEKLGSDRVKIKDIKELQSMKQARNVYNYEQEMFLRWNYAAATDQIRMYMLEELGGLYTDLDMMPSYSQEVLELIKKHSDGNRMFEDMSSRRAISDAVLKMAVGKATTVSMEEVAKDIDVSRLTEEDKTKLNALFKDLEPFAKPDSKGAEAEGGEGAKGMKKSFFQPIDLNIVRNTMPILRRYHHYPELGWFIRGLNGLMVSHKGSTAVSAVIVGQQAAYQELAALRQDVLSGEFFHSLENLTHRNHKERIGNHLVANYLAKSLFFDYCQDSVMPEAVSTLGIR

SEQ ID NO: 7 - protein, 264 aa, Chlamydia trachomatis
MRCSAYCTASSYHLHVLFHLLKVNYPSVLSREYVLISSEELDESDKAAVFFPFGVCVFWGWEETEELQVIRTITPSAVNPLPNPEIDSYDFHYGEKLQIRRDRLVLTNSNLNTKLAISFGLAQSIKLTVFEETIYKTVENSKSLPQELASKGKISLSRKTIAKKIGELFLDKASVNLHSDILDEPDFFWEHPETQPFYIDVLTCLDVNARVNVLNHRLAILGDVLEILNDQLNHQHSSALEWTVIWLIALEVLVTLLKDVFNII

SEQ ID NO: 8 - protein, 975 aa, Chlamydia trachomatis
MNRVIEIHAHYDQRQLSQSPNTNFLVHHPYLTLIPKFLLGALIVYAPYSFAEMELAISGHKQGKDRDTFTMISSCPEGTNYIINRKLILSDFSLLNKVSSGGAFRNLAGKISFLGKNSSASIHFKHININGFGAGVFSESSIEFTDLRKLVAFGSESTGGIFTAKEDISFKNNHHIAFRNNITKGNGGVIQLQGDMKGSVSFVDQRGAIIFTNNQAVTSSSMKHSGRGGAISGDFAGSRILFLNNQQITFEGNSAVHGGAIYNKNGLVEFLGNAGPLAFKENTTIANGGAIYTSNFKANQQTSPILFSQNHANKKGGAIYAQYVNLEQNQDTIRFEKNTAKEGGGAITSSQCSITAHNTIIFSDNAAGDLGGGAILLEGKKPSLTLIAHSGNIAFSGNTMLHITKKASLDRHNSILIKEAPYKIQLAANKNHSIHFFDPVMALSASSSPIQINAPEYETPFFSPKGMIVFSGANLLDDAREDVANRTSIFNQPVHLYNGTLSIENGAHLIVQSFKQTGGRISLSPGSSLALYTMNSFFHGNISSKEPLEINGLSFGVDISPSNLQAEIRAGNAPLRLSGSPSIHDPEGLFYENRDTAASPYQMEILLTSDKIVDISKFTTDSLVTNKQSGFQGAWHFSWQPNTINNTKQKILRASWLPTGEYVLESNRVGRAVPNSLWSTFLLLQTASHNLGDHLCNNRSLIPTSYFGVLIGGTGAEMSTHSSEEESFISRLGATGTSIIRLTPSLTLSGGGSHMFGDSFVADLPEHITSEGIVQNVGLTHVWGPLTVNSTLCAALDHNAMVRICSKKDHTYGKWDTFGMRGTLGASYTFLEYDQTMRVFSFANIEATNILQRAFTETGYNPRSFSKTKLLNIAIPIGIGYEFCLGNSSFALLGKGSIGYSRDIKRENPSTLAHLAMNDFAWTTNGCSVPTSAHTLANQLILRYKACSLYITAYTINREGKNLSNSLSCGGYVGF

SEQ ID NO: 9 - protein, 804 aa, Chlamydia trachomatis
MDAQEKKYDASAITVLEGLQAVRERPGMYIGDTGVTGLHHLVYEVVDNSIDEAMAGFCTEVVVRILEDGGISISDNGRGIPIQIHEKESAKQGREISALEVVLTVLHAGGKFDKDSYKVSGGLHGVGVSCVNALSEKFIAKVFKDGQAYSMEFSRGAPLTTLQVLGPTDKRGTEVLFYPDPAIFSTCVFDRAILMKRLRELAFLNRGVTIVFEDDRDTGFDKVVFFYEGGIQSFVSYLNQNKEILFPNPIYMQGSRPGDDGDIEFEAALQWNSGYSELIYSYANNIPTRQGGTHLTGFSTALTRAVNSYIKAHNLSKSDKLSLTGEDIKEGLVAIVSVKVPNPQFEGQTKQKLGNSDVGSVSQQISGEVLTTFFEENPQIAKTIVDKVFVAAQAREAAKRARELTLRKSALDSARLPGKLIDCLEKDPEKCEMYIVEGDSAGGSAKQGRDRRFQAILPIRGKILNVEKARLQKVFQNQEIGSIIAALGCGIGKDNFNLSKLRYKRIIIMTDADVDGSHIRTLLLTFFYRHMSALIENECVYIAQPPLYRVSKKKDFRYILSEKEMDGYLLNLGTKESQIVFDDTLRDLRGEALETFVNLILEVESFIVSLEKKAIPFSEFLDMFRDGAYPLYYYPPESGKQGGSYLYSHEEKEAAIAANEESASRILELYKGSVLEELQRDLGDYGYDIRNYLHPKGSGITVSTEDPKISPYVCYTLKEVIDYLKGLGRKGIEIQRYKGLGEMNADQLWDTTMNPEQRTLVRVSLKDAVEADHIFTMLMGEEVPPRREFIESHALSIRMNNLDI

SEQ ID NO: 10 - protein, 792 aa, Chlamydia trachomatis
MLGIRKKTILQLAVLLLLTFSRSSFCSTSEGRMVVESITITTQGENTQNKRAIPKIKTKQGTLFSQADFDEDLRTLSKDFDRVEPIVEFRNGQAVISLILTAKPVIREINISGNEAIPTHKILKTLELYKNDLFDRELFFKNFDALRTLYLKRGYYDSQLSYSHNHNEKEGFIDISIEIKEGRHGRIKKLTISGITRTEASDLGDIVLTKQYSTTTSWFTGAGVYHPDMVEQDLFAITNYFQNKGYADAKVSKEVSTDAKGNITLLIVVDKGPLYTLGHVHIEGFTALSKRLLDKQLLVGPNSLYCPDKIWTGAQKIRSAYARYGYVNTNVDVSFSAHPTLPVYDVTYRVSEGSPYKIGLIKIKGNTHTKHDVILHETSLFPGDTFDRLKLEGTETRLRNTGYFKSVSVYTVRSQLDPLDSNDLYRDVFIEVKETETGNLGLFLGFSSIDHLFGGAEIAESNFDLFGARNFLKKGFKSLRGGGEYLFLKANLGDKVTDYTVKWTKPHFLNTPWILGVELDKSINKALSKDYSVDTYGGNISTTYILNDKLKYGMYYRGSQTSLSLRKKTSSSNRPGPDLDSNKGFVSAAGLNVLYDSIDNPRKPTMGIRSSLNFELSGLGGTYQFTKLTASGSIYRLLTKKGVLKVRAEAKFIKPFGTTTAQGIPVSERFFLGGETTVRGYKPFIIGPKFSPTEPQGGLSSLLLTEEFQYPLISQPCINAFVFLDSGFIGIEEYTIRLKDLCSSAGFGLRFDMMNNVPIMLGWGWPFRPTEILNNEKIDVSQRFFFALGGVF

SEQ ID NO: 11 - protein, 490 aa, Chlamydia trachomatis
MNKKIIKERVSSSVPSTPSDQPADPTNPVTKMLFECKLSQNQGDASADVDAMVTTAHVKLDAKNQQVRQVAKGEEDQNVIVLSQLEGYLQKGSRSIAGLFLPTSGGPMVGDINMGTSPGNTIKGLPSSWGSAMFLGNEYAASVGIVQDVVGEHGARLDDLIERITKYSGSGEGSMTKLLEDLGSPKESQSGQTPSVTLEKPTDAKWLLLSAKNAMTGTLRFEKKQGALPTTPDPTITNLKARGLGTIGTATPTDKLQNIVSVADLTTILKDLQENAPSEGTGSDTGSDVGSGSGSGTDSGSGGVAGVSVSAGTTSTFPEWTGQEVPYLVEQSPDSGSGSPSVQNPLKSSNADNYIKKDNGTYSLLRRGLYLITFAYEFSSPTQNGAVTCTGTLRKTTTPESGDVGAQPTSGDATAPTDPEPSPAFSVVGSSDGKTVVGSAYILVPGDKALSEHTLTLALTTNENTEVNPTSVTDPASKSYVAISYMGAGY

SEQ ID NO: 12 - protein, 486 aa, Chlamydia trachomatis
MCFIGIGSLLLPTALRATERMRKEPIPLLDKQQSFWNVDPYCLESICACFVAHRDPLSAKQLMYLFPQLSEEDVSVFARCILSSKRPEYLFSKSEEELFAKLILPRVSLGVHRDDDLARVLVLAEPSAEEQKARYYSLYLDVLALRAYVERERLASAAHGDPERIDLATIEAINTILFQEEGWRYPSKQEMFENRFSELAAVTDSKFGVCLGTVVLYQAVAQRLDLSLDPVTPPGHIYLRYKDKVNIETTSGGRHLPTERYCECIKESQLKVRSQMELIGLTFMNRGAFFLQKGEFLQASLAYEQAQSYLSDEQISDLLGITYVLLGKKAAGEALLKKSAEKTRRGSSIYDYFQGYISPEILGVLFADSGVTYQETLEYRKKLVMLSKKYPKSGSLRLRLATTALELGLVKEGVQLLEESVKDAPEDLSLRLQFCKILCNRHDYVRAKYHFDQAQALLIKEGLFSEKTSYTLLKTIGKKLSLFAPS

SEQ ID NO: 13 - protein, 311 aa, Chlamydia trachomatis
MTDSESPIPKKSIPARFPKWLRQKLPLGRVFAQTDNTIKNKGLPTVCEEASCPNRTHCWSRHTATYLALGDACTRRCGFCDIDFTRNPLPPDPEEGAKIAESAKALGLKHIVITMVSRDDLEDGGASALVHIIETLHTELPTATIEVLASDFEGNIAALHHLLDAHIAIYNHNVETVERLTPFVRHKATYRRSLMMLENAAKYLPNLMTKSGIMVGLGEQESEVKQTLKDLADHGVKIVTIGQYLRPSRRHIPVKSYVSPETFDYYRSVGESLGLFIYAGPFVRSSFNADSVFEAMRQRETSTSALLPNKD

SEQ ID NO: 14 - protein, 253 aa, Chlamydia trachomatis
MKIGFWRRLYEVCYTSLIGCALKLRYRVLVEGIESINQNSQKGALFLSNHVAEIDPVILEHVFWLKFHVRPIAVDYLFNNPVVKWFLNSVRAIPVPSVVPGRDDKRLLERMERFYVCVTQALDRKESLLLYPSGRLSRNGKEEIVNQQAAYTILHRAKECDVFLVKITGLWGSSFSRYRTGSTPKLGKVFKEAVKALLRCGIFFMPKREVRVSVCPADYLVLKQFPTKQEFNTFLSDWFNQEGGETPLEVPYA

SEQ ID NO: 15 - protein, 468 aa, Chlamydia trachomatis
MHHRKFLAVSIAFVSLAFGLTSCYHKKEEPKDVLRIAICHDPMSLDPRQVFLSKDVSIVKALYEGLVREKEAAFQLALAERYHQSDDGCVYTFFLKNTFWSNGDVVTAYDFEESIKQIYFREIDNPSLRSLALIKNSHAVLTGALPVEDLGVRALNAKTLEIVLENPFPYFLEILAHPVFQIVPDIHTAMQLFQKNHIDLVGLPWSSSFSLEEQRNLPREKLFDYPVLSCSVLFCNIHQTPLNNPSLRTALSLAINRETLLKLAGKGCSATSFVHPQLSQIPATTLSQDERIALAKGYLTEALKTLSQEDLEKITLIYPIESVCLRAVVQEIRQQLFDVLGFKISTLGLEYHCFLDKRSRGEFSLATGNWIADYHQASAFLSVLGNGTRYKDFQLINWQNQKYTNIVAQLLIQESSDLQLMAEQLLLKESPLIPLYHLDYVYAKQPRVSDLQTSSRGEIDLKRVSLAE

SEQ ID NO: 16 - protein, 313 aa, Chlamydia trachomatis
MLAGSKRKHKTPEDTSSSSSKRARSSSSQVVPRLLQHHELIQLYSAHQQRNNEPVKMICETILQAKRSVLLKIFNIGSPRILAALAEASNRAPVSVHYQMGPFSKHCTEGNVQFRPRRGCSLLHRKTLLIDNNIVVTGTANYTEASLEKDVNLTAKIFSEHLYRWAFRHDRGEVRVGSQQVSYYSLSQIRRDLCVKAILEANGIVLRERTCEGILHTKVCCIDSSTLIIGSVNWSRGGLTLNLEEFLIINPLTETQLECYNELWAHIETNSRLMTKELIQLHEKRKKSITDPKQISSSTQDEENASTSAEQQF

SEQ ID NO: 17 - protein, 790 aa, Chlamydia trachomatis
MLVPLSLLQKFFSSPLSIEEILQACDRIGIEAECSNVFPDSLNTVVTGKILSASPHPDAERLTVAIVFDGKGERQIICGAPNCRAGIIVPIALPGAKLRNASGEITTIKKAKIRGLESQGMCCGADELGFPHLQKAERGIFEFPADTPLGESACMLLAGAPLECSLTPNLGHCASLLGLAREISFLSPVSLNIPEEFSFASLPQETSICDMHDAGACPVFYSVKISGLSCRRSPEYLQAALTALGQKPLNAIVDITNYVMLSLGQPLHAYDSQAVEQKSLHAATLQSAQPLTLLNQETYTLPAGSLVVADQHNILGLAGVMGSAASSCSENTTEIILEAAYFQPQAVRKYQRTIQLHTEAAYRFTRGVDPQGVLPVLHAAIHMIQSLFPDAQISPIQKIGDDSFSPLSLSVRPKTIKRLLDIELSTAEIVAKLSSLGFQTAVEEQAVRVEVPSYRHDIQEETDLVEEICRTTPFVQKTQKILPTYTPIYSLKRELTAFLANGGLQQFFTYSLLDTEVSSLSLQESSLIPVQNSSWKLRDSLLPGMLKSAATNLHRQAPYVYAFEIGNVYSKEQNRYQEEERVAILLSRQVMDDSWQGKTPLSFYTIKGWVEKLLCQSGASIEDFSLQPSQHPNFHPYQQAALYQKKHLLGIFGTLHPQLCRKAQIKHDVVFAELSLNVLLSLKKKSGPHYVPYPIYPASSRDITITIDRDLPADLVRRELLSFESKWLESVHIVSVYQGRDSASQSKNVSLRMVFRDHERTLSGQEIEEEYERLTALLDKKLANIGQGNS

SEQ ID NO: 18 - protein, 800 aa, Chlamydia trachomatis
MFEKFTNRAKQVIKLAKKEAQRLNHNYLGTEHILLGLLKLGQGVAVNVLRTLGVDFDTAKHEVERLIGYGPEIQVCGDPALTGRVKKSFESANEEAALLEHNYVGTEHLLLGILNQSDGVALQVLENLHVDPKEIRKEILKELETFNLQLPPSSSITPRNTNSSSSSKSSSPLGGHTLGGDKPEKLSALKAYGYDLTEMFKESRLDPVIGRSAEVERLILILCRRRKNNPVLVGEAGVGKTAIVEGLAQKIVSGEVPEALRKKRLITLDLALMIAGTKYRGQFEERIKAVMDEVRKHGNILLFIDELHTIVGAGAAEGAIDASHILKPALARGEIQCIGATTLDEYRKHIEKDAALERRFQKIVVQPPSVDETVEILRGLKKKYEEHHNVFITDEALVAAAKLSDQYVHGRFLPDKAIDLLDEAGARVRVNTMGQPSDLVRLEAEIEKTKQAKEQAIGTQEYEKAASLRDEEKKLREKLGNMKQQWESNKEEHQVPVDEEAVAQVVSVQTGIPAARLTEAESEKLLTLETTLQKKVIGQSQAVASICRAIRRSRTGIKDPNRPMGSFLFLGPTGVGKTLLAQQIAIEMFGGEDSLIQVDMSEYMEKFAATKMMGSPPGYVGHEEGGHLTEQVRRRPYCVVLFDEIEKAHPDIMDLMLQILEQGRLTDSFGRKIDFRNTIIIMTSNLGADLIRKSGEIGFGLRSHMDYAVIKEKIDAAVKKHLKPEFINRLDESVIFKPLEKEALSEIIHLEINKLGSRLQNYQMDLNIPDSVISFLVTKGHSPEMGARPLRRVVEQYLED

SEQ ID NO: 19 - protein, 426 aa, Chlamydia trachomatis
MYVRSIFFSIIAFLTVGCSFSPPESGLIIAIHDDPRSLSPEKGENAFHFSLSKALFATLFREELSGLTPALVSSYQVSEDGRFYRFCIRKDAKWSDGSLLLAEDVIAAWEHTKQAGRYSLLFEKLSFRASSSSEILIELKEPEPQLLAILASPFFAVYRPENPFLSSGPFMPKTYVQGQTLVLQKNPYYYDHAHVELHSIDFRIIPNIYTALHLLRRGDVDWVGQPWHQGIPFELRTTSALYTHYSVDGTFWLILNPKDPVLSSLSNRQRLIAAVQKEKLVKQALGTQYRVAESSPSPEGIIAHQEASTPFPGKITLIYPNNITRCQRLAEVLQEQCRDAGIQLTLEGLEYHVFVQKRATQDFSVSTATSIAFHPLAKSKFDQTALDNFTCLPLYHIEYDYILSRPLDQIVHYPSGSVDLTYAHFH

SEQ ID NO: 20 - protein, 223 aa, Chlamydia trachomatis
MKFFSLIYKDQEVVPNKKVLSPDAYTAVLTAQELLEKTQEDCEAYTQNTHEECAKLREEAKNQGFQEGSKAWSKQLAFLITETQAMREQIKASLVPLAIASIKKIIGKELETKPETVVSIISESLKDLTQNKRIVIHINPQDLAIVEQHRPELKKLVEYADVLLLSPKASVSPGGCIIETETGIVNAQLDVQLAALEQAFSAILKHKKPADASTIDQPQSKKD

SEQ ID NO: 21 - protein, 605 aa, Chlamydia trachomatis
MRKKTAYSESSIISLASLDHIRLRAGMYIGRLGDGSQAEDGIYTLFKEVVDNAIDEFVMGYGHTIHITGDAHELSIRDEGRGIPLGKVIDCVSKINTGAKYTQDVFHFSVGLNGVGLKAVNALSQHFSVRSVRNKKFLKASFSKGILLHTEQGATQDPDGTEVVFSPDHELFENFSFQVEFLKKKIRQYTYLHPGLTIIYNGERIVSTRGLLDLFEEEVQTPLLYSPITFQHSDLAFLFSHTETSSEQYFSFVNGQETTDGGTHLVAFKEGIVKGVNEFFGKNFSSQDIREGLAGCIAIKIASPIFESQTKNKLGNTNIRAELAKRVKEAVLSSLKKNPSSAERIQEKIKLNEKTRKNAQFLKQELKDKQKKLHYKIPKLRDCKFHLTDNSLYGKNSSIFITEGESASASILASRNPLTQAVFSLRGKPMNVFSSKEETIYKNDELFYLTTALGLHKDSLQNLRYNQVILATDADVDGMHIRNLMITFFLKTFLPLVASNHLFILETPLFKVRHKDATFYCYSEEEKLSTIEHIGKKESSLEITRFKGLGEISPKEFKSFIGADMRLTPVSLPDTETLDTLLQFYMGKNTKERKLFIIENLVTNL

SEQ ID NO: 22 - protein, 342 aa, Chlamydia trachomatis
MFSLLNDCQKAAETVVMQAMLSLMRYRKAHKFIPFWKKPDQTYVTPADYAIQYYFYQKLTSLFPHIPLVGEETLNPATDHPRIPQILQFAQQLDPKVSCQDLYQALSPESSHSSLFWLTDPIDGTSGFIKQRCFAIALSLFYEHTPVLSVIACPSSENNSFKIYSAAKGEGLTICNPTHSFPFSLHEDFQPTCKFCEASLSARNHQHLTTHILSKHLPWNPQPIRADSQCKYAWVADNTVDFFIRIPYSPPRAHYRDHAPGVFLIEEAGGLVTDISGNPLPFSNPNLYLDRHPLILASANEQMHSTILETLYKLRHQATQNMLPLATHISATKHKQLSALQL

SEQ ID NO: 23 - protein, 284 aa, Chlamydia trachomatis
VFKFFGNKLRSLFKKTLSSDLLEYAEVLLYEGDFGPKLTEAFCEELRRCKNPDERAVKELIRSFLSKIISKLPQREPLSVRPFSTLVLGTNGSGKTTTVAKLAHYYLSQNQKVLIVATDTFRSAGMDQMRCWAETLNCGFISGKPGGDAAAIAFDGISAAVARDYDHVIIDTSGRLHTHTNLLKELQKIATVCNKAFPGAPHETLMTIDATLGSNTLSQVQLFHEAVPINGLIFTKVEGSAKGGSLFRIADELKIPTRFVGYGETIHDFEPFAIDRFLDKLLDV

SEQ ID NO: 24 - protein, 420 aa, Chlamydia trachomatis
MLKFQLCLLFLFGYLTIVFEHIVRVNKSAVSLAMGGLMWLVCFSHIPHIDHVMMFEEIADMAQVIFFLFAAMAIVELIDAHRGFSIVVRCCNVESRSVLLWVLLTLSFFLSAALDNLTSIIIIISILKRLVKAREDRLLLGALCVIGVNAGGAWTPLGDVTTTMLWINDKISTSGIITTLFLPSVVCVVIAGICGQLLLKKRRCSGLSEDLDREPALPKSNLIACVGFGSLLMVPMWKAVLGVPPFMGALLGLGLVWLVSDWIHSPHGEGRNHLRMPHILTRIDISSVTFFIGILLAVNALTYSHVLRDLSVSMDALFSRNTLAVLLGLVSSVLDNVPLVAATIGMYDLPMNDPLWKLIAYTAGTGGSILIIGSAAGVAYMGMEKVSFGWYVKHASWIALASYFGGLAVYFLMENCVSLF

SEQ ID NO: 25 - protein, 100 aa, Chlamydia trachomatis
MARKDRLTNERLNKLFDSPFSLVNYVIKQAKNKIARGDVRSSNVAIEALNFLDLYGIQSEYAERDDRERHLSATGERRREQGFGTSRRKDPSLYNWSDVK

SEQ ID NO: 26 - protein, 548 aa, Chlamydia trachomatis
SSRILITSALPYANGPLHFGHITGAYLPADVYARFQRLQGKEVLYICGSDEYGIAITLNAELAGMGYQEYVDMYHKLHKDTFKKLGISVDFFSRTTNTYHPAIVQDFYRNLQERGLVENQVTEQLYSEEEGKFLADRYVVGTCPKCGFDRARGDECQQCGADYEARDLKEPRSKLTGAALSLRDTEHAYLHLERMKEDLLAFVQGIYLRPHMRNFVTDYIEHLRPRAVTRDLSWGIPVPDLENKVFYVWFDAPIGYISGTMDWAASIGDPEAWKKFWLDDTVTYAQFIGKDNTSFHAAIFPAMEIGQSLPYKKVDALVTSEFLLLEGFQFSKSDGNFIDMDAFLETYSLDKLRYVLAAIAPETSDSEFSFQEFKTRCNSELVGKYGNFVNRVLAFAVKNGCTELSSPQLEQKDLDFISKSQKLAKDAAEHYAQYSLRKACSTIMELAALGNGYFNDEAPWKLAKEGNWNRVRAILFCACYCQKLLALISYPIMPETALKILEMIAPHSLDLGSQDPDRLQSLWTDSFFDYSEEKFSLKEPELLFTMVE

SEQ ID NO: 27 - protein, 365 aa, Chlamydia trachomatis
MRVLSLFLKDFRNYTDLRLELGPEMNSIFGLNAQGKTNLLEALYILSLGRSFRTSRLTDAIRFGASHFFIEAVFSHKEVFHTLSIQVDKKGKKILFDGAPITKLSELVGLFPVILFSIKDIAIIEGSPSERRRFLDLLLAQASDKYTEHISLYHKALDQRNASIKAQNQKAISAWNSPLIAYGSLVAFLRNECTKKLNTIFQTLWDNTLKETLSLRYESSLITEESPTLNDIASNYYEQLRIANTKDLDLGYTMVGPHRDELLLTINDLPVAKFSSEGQKHSLLAVLRFAECVYLQEEFCIHPLLCMDDIHACLDQQRLDQLLQLSNSLGQVVTTSTICPDHRSTTSCIFHVTQAQVSLVAPQSL

SEQ ID NO: 28 - protein, 425 aa, Chlamydia trachomatis
PEQKSTVKDLLNKLKKVISAYNSMPDKNSEAGQNSLIQQGKYVDAIQKKLPASSQAQPKQAKAKEQKAEEKPKTTPIEGVLETIKTEFKGHRVPVEKIIHGIWIAGAPPDGIEDYMRVFLDTYEGFDFYFWVDENAYAAAKFSSILKKVAFDAAIQDLRSATDESTKAFVKDYDELKQKYEKKVAETTSQAEKDQYLKDLKDLLEKFTKISDEIRGKFDRLFLKNVIVAQNGFFNFCLLKGLGNINDETRAEYLEKELKLPTEEIEQYKKLKETNKEKIAAIVKQLNEKLGSDRVKIKDIKELQSMKQARNVYNYEQEMFLRWNYAAATDQIRMYMLEELGGLYTDLDMMPSYSQEVLELIKKHSDGNRMFEDMSSRRAISDAVLKMAVGKATTVSMEEVAKDIDVSRLTEEDKTKLNALFKDLE

SEQ ID NO: 29 - protein, 95 aa, Chlamydia trachomatis
MLIFFDKSQSGALPDRLERAGNLLRFAVNRGMASQIKVTSAQSGHIFFSEKMISVCKRIACIVLCIVLAPFCLLGALIGTIAYKLSNSYQNALYL

SEQ ID NO: 30 - protein, 49 aa, Chlamydia trachomatis
MYQEQVDAVQAYHSLEQDALFVNSRQHSHFVQVISLVSSLMHSLSPTSE

SEQ ID NO: 31 - protein, 123 aa, Chlamydia trachomatis
KKSFFQPIDLNIVRNTMPILRRYHHYPELGWFIRGLNGLMVSHKGSTAVSAVIVGQQAAYQELAALRQDVLSGEFFHSLENLTHRNHKERIGNHLVANYLAKSLFFDYCQDSVMPEAVSTLGI

SEQ ID NO: 32 - protein, 194 aa, Chlamydia trachomatis
PEDTSSSSSKRARSSSSQVVPRLLQHHELIQLYSAHQQRNNEPVKMICETILQAKRSVLLKIFNIGSPRILAALAEASNRAPVSVHYQMGPFSKHCTEGNVQFRPRRGCSLLHRKTLLIDNNIVVTGTANYTEASLEKDVNLTAKIFSEHLYRWAFRHDRGEVRVGSQQVSYYSLSQIRRDLCVKAILEANGIV

SEQ ID NO: 33 - protein, 634 aa, Chlamydia trachomatis
MLTSELNAAQVTAVTAPLQPVLVLAGAGAGKTRVVTHRILYLIEESRLDPKQILAITFTNKAANELKERVQSQCRELGYSDVPMVSTFHSLSVYILRRSIHLLDRQSNFVIYDQSDSEKLIKQCLRKLNLDKKLCNAMQFTISQAKNRLQSPEDLDSKEYPDPTRTVYAEYQEQLRAANALDFDDLLFLTEKLLRTPEVQQEYANHWKALLIDEYQDTNHAQYLIAKRLAAAHNNIFVVGDPDQSIYSWRGANISNILNFEQDYSQALVVRLEENYRSCGTILEAANALIQNNSARLEKTLRSVKGPGDKIFCFTGKNDRDEAEQVLEEISNLHSYKDIPLSDICILYRTNFQSQSFEAALLKRGYPYEIIGGISFYKRREIQDILAFLRLFSNNYDMAAFERTISLKKCGIGATTLAALMNYAKTTDLPILQACWDVLEKKSIRLTKKQQQGLFSYLTHFHQMEQLYGNCDLHEFINETIRITDYLSILKEDPETYEDRKNNLEQLLAETQTWGKSSENLPGFLEDLALKSSADETASSHDRLKLMTIHNSKGLEFPVVFLVGLEENLLPHANSKGMHENIEEERRLCYVGITRAQEYLYLSRAKTRFLWGTERTMVPSRFISELPRALLKFV

SEQ ID NO: 34 - protein, 649 aa, Chlamydia trachomatis
MRVDVDKYLFIGREKSEFFSACREIGAVEFLSKSKLKDSEKVRKLSEGLKVLNLLTKSCSPADLVSTKSGYLVTEQLLQEIFDLNQEITTLTESLKALGKEIVRVKPLGDFSSEEIRELTLKTGLAVRFLYKRHIEGAPLEVEEENVFYLATAYNYDYYAVIGIVSLSKDIFTEIEAPRSVNELREEEGHLQALLRKKKARVCELYAYREDLLEALCEQCNEQSLQHAEVSAEDLFDDKVFSALGWVIVDRLDEVKKLCDSLGIYLERVQPDPDEVIPTYLENHGLGALGESLVNIYDTPASTDKDPSLWVFFSFFVFFSMIINDAGYGLVFLATSLFLSFKARKQIKRSIALKRFLQMFMILGLGCVCWGGATTSFFGVSVSYTSPFREYSLTHFLALKKAEYYLKERPKGYKELVHDYPILKEKKTPKEFLLAQSTSSGDSVYKAVVYDKFIDNILMEIALLVGVVHLSLGMLRYCRQRYSSIGWVIFMCGAYMYLPIYLQAVSLIHYALHIPYELGGLVGYYVAFIGLGVAILGGVIQRGLRGLDEITAVIQVFSDVLSYLRLYALSLAGAMVGNTVMVMSERFSPAVGILIIIFGHTVNIALSIMGGVIHGLRLNFIEWYHYSFDGGGKLLHPLKKVICQKSQNL

SEQ ID NO: 35 - protein, 291 aa, Chlamydia trachomatis
MRMQIPRSVGTHDGSFHADEVTACALLIMFDLVDENKIVRTRDPQKLAQCEYVCDVGGRYSTEHKRFDHHQVSYTGSWSSAGMVLDYLHGLGFLSHDEYEYLNNTLVHGVDEQDNGRFFSKEGFCSFSDIIKIYNPLEEGGNTDKEFFFALRFAIDLLTRLREKFCYDRVCRDIVKQVMEKESVCLRFDRPLAWQENFFSLGGESHPAAFVSFPCSDQWILRGIPPTLDRRMEVRIPFPEEWAGLLGDQLVQATGIPGAIFCHKGLFLSVWDSQESCEEALNLVLKQQRLV

SEQ ID NO: 36 - protein, 223 aa, Chlamydia trachomatis
MPLYLGLNPSSAKQYSAVFQPIIRIVPFSKHVPQIRHARKFLVSTSHILLTSPSSTACFFSSIRKRIPLSLFRNKSFVTIGEATSSRVKAFLPKANIVQSAFPQAEAFLPILSTLPSSSSLLYPHSVLARPILRSLLQTLPFSSFSYPHYTVKPITLSVSTFLPHKTIILTSPSIVRAYAQLFPCLPDKEHWCLGEISAAEFKQIFHTPPSKILFSTKEPTGC

SEQ ID NO: 37 - protein, 176 aa, Chlamydia trachomatis
MTEIPSSFVLPDPEWIYRVGIGQDSHRFLPDEDPKPCILGGIIFENTPGFEANSDGDVVFHAICNAFSSVTHKGILGGLADELLKTKGITDSVVYLQEAVASLKPTQRVSHLAITIEGKRPKLLPQLPSMRKRIAEVLHIPLDSINITATSGEGLTAMGQGYGVQCFCVLTIMEYC

SEQ ID NO: 38 - protein, 790 aa, Chlamydia trachomatis; identical to SEQ ID NO: 17

SEQ ID NO: 39 - protein, 294 aa, Chlamydia trachomatis
KKVAETTSQAEKDQYLKDLKDLLEKFTKISDEIRGKFDRLFLKNVIVAQNGFFNFCLLKGLGNINDETRAEYLEKELKLPTEEIEQYKKLKETNKEKIAAIVKQLNEKLGSDRVKIKDIKELQSMKQARNVYNYEQEMFLRWNYAAATDQIRMYMLEELGGLYTDLDMMPSYSQEVLELIKKHSDGNRMFEDMSSRRAISDAVLKMAVGKATTVSMEEVAKDIDVSRLTEEDKTKLNALFKDLEPFAKPDSKGAEAEGGEGAKGMKKSFFQPIDLNIVRNTMPILRRYHHYPEL

SEQ ID NO: 40 - protein, 62 aa, Chlamydia trachomatis
VDENAYAAAKFSSILKKVAFDAAIQDLRSATDESTKAFVKDYDELKQKYEKKVAETTSQAEK

SEQ ID NO: 41 - protein, 621 aa, Chlamydia trachomatis
MRRSVCYVTPSVARAGQISTWRFEYSSANFLPEGTLLKFDLGIDGRPIDWEIPSTDLSQPCNTIYLETPSEDIVAAKAVYAPGGYIPTFEFTLPCDVEAGDTFSIILGSSPNFPQEDSSGNGAQLFTQRRKPFSLYVDPSGKGSFEDPDIFTMDIRGNVLKNIRIFAPSYVIKNKRFDITVRFEDEFGNLTNFSPEETHIELSYEHLRENLNWQLFIPETGFVILPNLYFNEPGIYRIQLRNQATKEVFTSAPIKCFAETSSHLLWGLLHGESERVDSEGNIESCLRYFRDDCALNFFATSSFEIQDGLTPETIKTINQTVADFNEEDRFIALSGAQYLSEEPGEGIREVLLMKEPKSPGKHKECKLFPLSKLYKQSTSHELISIPSFTASKKFGYNFNNFHPEFERVVEIYNAWGCSERTEAEGNPFPIKGSIDSENPEGTVLSALKRNLRFGFVAGGLDDRNLYNHFFDSDQQQYSPGLTAVICNKYSRDSLLEALYQRQCYATTGQRIIVNFQITSAPMGSELSTAIKPGLVINRHISGYVAGTAKIASIEIIRNEDILHTFHPDGNNFEYEYDDLSPFAQVTLKDPQNGAPFAFYYLRVTQENGAMAWSSPIWIDLN

SEQ ID NO: 42 - protein, 306 aa, Chlamydia trachomatis
MRLIVRIFFFLCILSAPYSFASTCSAAQGASSCLPCATAPQQELPASPEKTEKPFRCPSYRPTVEAQHLLPPQEDLSSGAFSETYPDLTTQAVILLFLALSPFLVMLLTSYLKIIITLVLLRNALGVQQTPPSQVLNGIALILSIYVMFPTGVAMYNDAKKGIESSAVPRDLFSAEGAETVFVALNKSKEPLRSFLIKNTPKPQIQSFYKISQKTFPPELRQQLTPSDFMIIIPAFIMGQIKNAFEIGVLIYLPFFVIDLVTANVLVAMQMMMLSPLSISLPLKLLLVVMVDGWTLLLEGLMISFK

SEQ ID NO: 43 - protein, 346 aa, Chlamydia trachomatis
MQADILDGKQKRVNLNSKRLVNCNQVDVNQLVPIKYKWAWEHYLNGCANNWLPTEIPMGKDIELWKSDRLSEDERRVILLNLGFFSTAESLVGNNIVLAIFKHVTNPEARQYLLRQAFEEAVHTHTFLYICESLGLDEKEIFNAYNERAAIKAKDDFQMEITGKVLDPNFRTDSVEGLQEFVKNLVGYYIIMEGIFFYSGFVMILSFHRQNKMIGIGEQYQYILRDETIHLNFGIDLINGIKEENPEIWTPELQQEIVELIKRAVDLEIEYAQDCLPRGILGLRASMFIDYVQHIADRRLERIGLKPIYHTKNPFPWMSETIDLNKEKNFFETRVIEYQHAASLTW

SEQ ID NO: 44 - protein, 243 aa, Chlamydia muridarum
MLRVLAYRQVAFSKFPYALRSFLDFLHSLKQHYSFILPGDPLPNRKAIILTFDYASVDFYKHVFPFLQKFQIPAVVGVAWRYVSRLESENLPIDMRISPSDFLAFQDEIFSYYQPFCSVKELCHMAQSSIIRFASSGFAIRNLKNSPPYLHTEISLSKILLEEAIQAPVEVFFYPFGKSDLVSQHFVQEVYRYSFVLGDAASFFYSMQSQHSIPRIDMALDSRGIPSLYQLSFRQLKRFLILH

SEQ ID NO: 45 - protein, 53 aa, Chlamydia muridarum
MPISLPTSEVCSTETAELTPEGKERNLTIISGQVVIYPEDPFAIPSVDPCSAS

SEQ ID NO: 46 - protein, 240 aa, Chlamydia muridarum
MTEPLTDQQIILGTSTTPVAAKITASEGISLTITNNAQANSSVNIGLDAEKAYQLILDKLGDQIFDGITGSIVESAVQDIIDKITSDPSLGLLKAFYNFQITGKIQCNGLFTSSNVTTLLGGTEIGRFTVTPRSSGSMFLVSADIIASRMEGGVVLALVKEGDTQPCAISYGYSSGVPNLCSLKTCVTNSGSTPTTYSLRIGGLESGVVWVNALSNGNDILGITNTSNVSFLEVIPQKNT

SEQ ID NO: 47 - protein, 102 aa, Chlamydia muridarum
MQNKRKLRNDFIKIVKDVEKDFPELDLKIRVNKERVTFLNSPLELYHKSISLILGLLQQIEKSLELFPDSPVLEKLEDNSLKLKKALIMLILSRKDMFSKAE

SEQ ID NO: 48 - protein, 275 aa, Chlamydia muridarum
MMDYELLDSGDGKKLERFKDVVLIRPSATAIWPKTSPALWKQYSAEFVRVGEKGEWKYRNSHLKEWWITIDSVSCLLKLTPFGHVGIFPEHATFWQDLSPSLSKPSCKVLNLFAYTGASSIFCAQQGAAVYHVDASKAAVKWAQKNVEGNSFSEKRIFWIIEDVFSFLKKEIRRGKTYDVILLDPPTYGRGPDGETFKIDRDFFPLLEACSQLLSSSFSHMLITSHTPGHTPEFLHCLAKRALPMLSSQKWRLGENFCGEGDQRLPSGVFAQWSL

SEQ ID NO: 49 - protein, 198 aa, Chlamydia muridarum
MFSGIIQEVARVDLIHHYGDSMEIGIFARNLVDGVPGSSIAVDGICLTLVKREFELLFFDVTEETMACTTIKNYTVGSMVNLERSVRLGDEIGGHFVSGHVCGVGTIIAVEKSYMFFKAPTNLVPYVLEKGFIAIDGISLTIAQVRGDIFSVSVIPETRARTSLGYKQVGSHVNMEPDMMTKMQVDTVMRFQAEKIGK

SEQ ID NO: 50 - protein, 3225 aa, Chlamydia muridarum
MEQALCTMTLSNTASSTVSPKSTQLNHTLSNQTHPVAQSNRNIDFQIYDEQIHNNHSTEDVVAIGRRIQQEYANLTTSKQVNFSASPSNHTGNWKISLLYNLSMLVANLFPTTIQPVQPQKVIFKNQDSSKETQLTKSSKVVSSRSSLKEIFSKKSPPLRKSFSSSKISRAIPRRKKRSNDDPVQATNTYDLTAENILEKLSLTQEQQIKHDNLISNLKEAINRYSDLNRKNSRKGQSLLVRQAKILDEILSQTKSTEERASNSVMTTIKKEFTSHRVPVEKNIHGIWIAGSPPEGTDEYIKLFLHTYPEFSFLFWVDKTAYGAAKFSSTLKRIAFDAAVNSLREATPEPVKQFVQRYDKLKKSYDTSRDFDEKQRLSEQLVELYDNYNKFSKEIQSNFDVLLLHEMITIQDSFFNYCQLKGVGAITDETRIEYLEKVLKVKEEDLSHYKETIKRNKESIEKLVKEINDSTGRERVVIKDIRDLKSLQDLTNSYNYETEMLLRWNYAAATDQLRMYMLKEYGGIYTDLDIMPQYSQDVLQKIMDVGGSRFFEHDKLRRTLSFAALKLGSGKQTTVSFEEAKKAMTLPTFTLQDKSQISEIFKYLETETQAKKSLFQPMDVTVVRDFMPILQRYHKWQTGWNVRGLNGLMMAHKDSAVVDAVIARQRAAYDEMRALRQNVVSGEFFRSLGDLEHVNREKNIGGYLAKNYLGGSLFFDFRQDSVIPGAISTLGISGPDIIMDTMSDYFTNLGPVGEDFLYEGKLGKAAFLGAYQAQKTPKGELTYDWLHPLSIGANDVTPADASTWCETRQHCAAELLLSDSISSDEHPKGIRRERVNPNDFSKLWSKEAQGILSSDFADLLPRFNLLIESSALDIHTLSALDRDIQHLFTKVQKDPVASVAVFSLQLQLAEMIRAIPFPIRNQVHILPEAQAHFEADWKKAIQLYLHSHPQTEVVIWYSSTHTQIVFGKDLLAVAERVAAAKSLMSDHDPSLITSYLKYKTQSHLGVLTEFDQEDFFELMVDIAEEPELHKQLLKIEEQVNSGLYSHVEHSLGEWLKLSKEERKSKFLKILKETFQEEEREDSQQQHKTWFEELYEKRHQERVKDPAKKIQELITVFQESQRVQAQDIDTYFAHKPFYQDLMKDGYAFEDISVITKYLLASDGVSGIITTDPIFPPPSKQLIDAMKQSLGEDFGELHYTLQMVYDWLSKETNSVTSEQAKQKLPQKLHEKLEGYTTHDLLIPPIDGSVSALGLRFSTEEGKVSDRVLTSIAPGVPNSASYAMTSYLYGLFLITKDIQSGRLTHEILKERLQTYGGAYFINESKIDVLLALSRKKAQISLIDAHKALTGFSSFSEASLALLTGRMPGTSRVLSREVEFGRPSAIVMEGATAIRAQSYDAVGLRKDFFLLPHTVPSIQSIVEQAKYTVLSWPEFYENHADKWNDLANRFGAEDLSVHPQTFLYDTEGRCMGLALLYMLADDSVSYRLLQQNLMTLASLFDEQNRRNIPLTPADQKFLNKGLSLIEWLQFKGNQQLQTEGFFHTLDWDIPQLMKHFASSTVKSWLITTPAHSLVLSLMGNFFRVTDPNYGHTDFPSLEAAITFLERMVQVSPAVLERYGFDKEKSVTSQLKVHSLETSELQNAVFASSDLGFTSRYFTTTLEEMTVRGPITMNQRHTDWATLYKIGGTVQGKRIDSRTRESDLNFLKINGDILEEFLTRTVLDSDLVELIQSLLKTHGLEPGTTLISPSSIVETAIDHVSLLQAVKTKTSRMHTILQSLGERIFKLFKNSGVQDSDKISIDRVQLVDESDSATIDFTVIKDKQRSQKKSITVGIESLAGSFRKFSASMHEVIGTGVLDLDLGMTVVSLVQYVRLVEAGQGKDALAVANLVMNLKIALEVSIGNVIQALGKKLLTQEGLNTFRLETELARQLHKVGARVGGTVGKTLTRVAHVLELPILESAIGTWNLYNSVNELLHADSWSDQVAARVQVAFDSISLGITIASVVSPALMMAAGPIAAIGMGAASIARNVARKEERHSQWLKYKSFLDNGSKHTVAAFPHKGLIDLSENLVLGNIVLNLRVYPPLLTGDRSYNANRWFGNKPGWSDWQVRERLGYAYRSSPSYALARGHANSFWPLSMPSIDKGVYRTVILGYGIQYKAVTEVVYLSNEMVWREAVMQFDSRYYVEPLTAEKECATVLAGDTPLSVIPVRLLEEESLEREKNAASYKNYQIIIEGGKGGLTVQIGGAGYYKLTAQPGKGNILSFRAIPGYLSVTFDLSRLEQEVPLTKQNGTALKILKVRQTGFDTIVGSSGGSDNLTGNHNTKFYLSTGGGHVTSGSGKNWYNIPSLTRSLGFDFTSNAIEHKASVEMLLADFSPANNLSLIVERFANVSIHVFNYSNKTAPYTVNLKDGVSLQASKQGFSGSLLEVASFDQTLWQKSYPEDRGFVEDILSWLLKLQWSLASRVRIFLQGGTAQYETTQKSLIYRPDPHSSISIQATDSYNTQVYGNVGCSYILFSSPGVTAKTLDILLFEDSGLPQIIDLSTLVPTSIQGFLYPGGFINFQVSSARYAFPFSVSWRRHHYTFPAQTIIQVLPRLRFELGDWFTILQKSVGKWVTLYQHDMIIPERIEGVLSLNNTATLMSHHNQTTHMLGVENRGDLSLKVLGILQSGQIKGSKSENSASGHFSKLLSVPAHTIKNLSFKEEEGTKSKNILFYSVLEETFLKATDKPLTIIKRDKWSLYDEIQVFATTLNLQNFLRYHISEETPMLSRLLMYAQKRVSIQNRDLALKFFYVRERSGIGAIRLVFKNFFEESLQGILHGTLEREVKPMLAENPNTLIHSSYRNHLELILGEETLDLAIIVQEFSSSKNIVELQKDLATHRLIYPQGKEALSLAIYTHTFSKESLVTSKQRSEGLKFIDPSMQEYRLPETTILENSYYLDPSTGDLYLTKIIAQPSQSRAFLIKFKKYKRHWLDFQKLVLSGSHLELIASTGTALTFVGPELRHLEIDFPGALNGTIAKEHISSRASIVIPTNNQVTHYDPRIAKQHYSSINYMLWDLRDRATLSKRAKTLDSYLLEVCMNLDSSNPTWNIPNDVLNYAVGYYRTILPSWVKNKIRVGSLIKVEADTAKTLSFSLITTQNDLFQPVVDKGFYIYYSVSKLANHVKLRNVTGDMLLDIDKETTFIVRGVDESDYDKKQIYVVLDLTTEEERKLRTDKNIIIIPGGERLNR

SEQ ID NO: 51 - protein, 264 aa, Chlamydia muridarum
MRCSAYCSASSYHLHVLFHLLKARYPSVLSREYVLISSEELNESDKAAVFFPFGVSVFWGWEEAEELQVIQAITSASVDPLPNPEIDSYDFHYGEKLQIRRDRLVLTNSNLNTKLAISFGLAQSIKLTVFEETIYKTVENSKSLPQELASKGKISLSRKTIAKKIGELFLDKASVNLHSDILDEPDFFWEHPETQPFYIDVLTCLDVNARVNVLNHRLAILGDVLEILNDQLNHQHSSALEWTVIWLIALEVLVTLLKDVFNII

SEQ ID NO: 52 - protein, 986 aa, Chlamydia muridarum
MPQALIIKELMNQVIKTIALCYQKYISRASNKTFSIHNTLSLSLLPKCLLGSLIIYTSHAFGEMELAISGHKYGKDRDAFTMISSCPEGTNYMINRKLILSEFSSLDKFSSGGAFKNLAGKIAFLGKHSSSSIHFKHLNINGFGSGIFSESAIEFSDLRKLVAFGSESTGGIFTARDDISFKNNHYIAFRNNIAKGNGGVILLQGDERGTVSFTDQQGAIIFANNQALVSPSIKHSGRGGAISGDFAGSRIIFLNNQQITFEENSAVHGGAIYNKNGVVEFLGNGGTLSFKENSTRANGGAIYTGKFKANQQTAPIIFSQNNANQKGGAIYAQYVNLEQNQDAIRFENNSAKEGGGAISSSQCAITAHNSITFSNNFAGDLGGGAILLGGKQPSLSLVAHNGNIAFIGNTMLPATKKASLPRNNSILVKESPYKIQFAANKNRSIIFFDPVIALSPSTSPVEINSPEHETPFFSPKGTIVFSGANLIDDAKEDIANRTSIFNQPVLLHNGTLSIESGAHLVVQSFKQTGGRISLSPGSSLALYTTNTLFHGNVSSTDPLEINGLSLGVDTSPSNLYSEIRAGSAPLKLSGSPDIHDPERLFYENRDSAASPYQMEILLSSDKIIDVSNFTIDAPVPNKEAGFQGSWHFSWQPNTVNNTKHKVLRASWIPTGEYILEPSRVGNAIPNSLWSTFLLLQTASHNLGDHLCNNSDLVPTSYLGLLIGGIGAEMRTYSAEKESFISRSGTTGTTIIRLTPTLTLSGGATHMFGDSFVTKLPEFIASEGMVQNVGLTQILGPLTVKSTLCAALDHNAMIRLSAQKNHTRAKWDTFGIRGTLGASYSLLDYENMVRIFTFANVEATNVLQKSFTETGYNPRSFARTRLTNIAVPVGIGYEFCLSNHSFALLGKGHIGYSRDIKRKNPVTFAQLAMNNFSWTANGCQVPTSAHTIANQLILRYKACSLYVNAVATKLESTYLSSSLSCGGYVGF

SEQ ID NO: 53 - protein, 804 aa, Chlamydia muridarum
MDAQEKKYDASAITVLEGLQAVRERPGMYIGDTGVTGLHHLVYEVVDNSIDEAMAGFCTEVVVRILEDGGISVSDNGRGIPVQIHEKESAKQGREISALEVVLTVLHAGGKFDKDSYKVSGGLHGVGVSCVNALSEKFIANVFKDGQAYSMEFSKGAPLTALQVLGSTDKRGTEILFYPDPTVFSTCVFDRAILMKRLRELAFLNRGITIIFEDDRDAGFDKVTFFYEGGIQSFVSYLNQNKESLFPTPIYIQGSKRGDDGDIEFEAALQWNSGYSELIYSYANNIPTRQGGTHLTGFSTALTRAVNSYIKSHNLAKSDKLSLTGEDIREGLVAIVSVKVPNPQFEGQTKQKLGNSDVGSVAQQISGEVLTTFFEENPQIAKTIVDKVFVAAQAREAAKRARELTIRKSALDSARLPGKLVDCLEKDPEKCEMYIVEGDSAGGSAKQGRDRRFQAILPIRGKILNVEKARLQKIFQNQEIGSIIAALGCGIGTDNFNLSKLRYKRVIIMTDADVDGSHIRTLLLTFFYRHMSALIENGCVYIAQPPLYRVSKKKDFRYILSEKEMDGYLLNLGTRESRVVFDDTARELEGTALESFVNLILEVESFIIALEKRAIPFSEFLDMFRDGAYPLYYYPPESGKQGGNYLYSNEEKEAAVAANEEAAARIIELYKASAFEEIQHNLNEYGYDIRHYLHPKGTGITVSTEDSKTPNYTCYTLKEVIDYLKGLGRKGIEIQRYKGLGEMNADQLWDTTMNPEQRTLVRVSLKDAVEADHIFTMLMGEEVPPRREFIESHALSIRMSNLDI

SEQ ID NO: 54 - protein, 740 aa, Chlamydia muridarum
MLGIRKKTILQLAILLLLPFSQNAFGTSPEGRMVVQSITIETRGENAQNKRAIPKIKTKQGTLFSQTDFDEDLRTLSKDFDRIDPIVEFRNGQAVISLILTAKPVIRHINISGNEAIPTHKILKTLELYKNDLFDREVFFKNFDALRTLYLKRGYYDSQLSYSHDHNEQEGYIDISIQIKEGPYGRIKKLTISGITRTEASDLGDIVLTKQYSTTTSWFTGAGVYHPDMVDQDLFAITNYFQNKGYADAKVTKEVSTDAKGNLSLVINVDKGPLYTLGHVHIEGFTALSKRLLDKQLLVGPNSLYCPEKVWAGAQKIRNAYARYGYVNTNVDVSFAVHPTLPVYDVTYRVSEGAPYKIGLIKIKGNTHTKHDVILHETSLFPGDTFDRLKLEDTETRLRNTGYFKSVSVYTVRSQLDPLDSNNLYRDVFIEVKETETGNLGLFLGFSSIDHLFGGAEISESNFDLFGARHLFKKGFKSLRGGGEYLFLKANLGDKVTDYTVKWTKPHFLNTPWILGVELDKSINKALSKDYSVDTYGGNISTTYILNDKLKYGMYYRGSQTSLSLRKKTENPNKPGPDLDSNKGFVSAAGLNVLYDSIDNPRKPTMGIRSSLNFELSGLGGTYQFTKLTASGSIYRLLTKKGVLKIRGEAKFIKPFGTTTAQGIPVSERFFLGGESTVRGYKPFIIGPKFSPTEPQGGLSSLLLTEEFQYPLISQPSINAFVFLDSGFIGTEEYTIRLKN

SEQ ID NO: 55 - protein, 434 aa, Chlamydia muridarum
MNDKTIIHKESVAFAAIPPTQPAAPVTKMLLECTQTGGNTGVDGMVTTAYVNLDAKNQQVRQVGAGTEDQDVVVLSQLDGYLQKGAKSIAGLFLSTSGGMMVGDINMGTNQQNTIRGLPSSWGEASFLGNDYAASVGIVNDVLGNYEDRLNDLIAKINMYAGTGEGSMAKLITDLGSPKETTSGSTTTITFDKPEEAKWLLLSAKNAMTGTLRFEQQQGALPTAAAPTIKNLKTGDIGVVGTATPKEKLQKIVTVSDLTTILNDLQNNSSGGSGSGTGSTTTPTFPNWTGQEVPYFVEQNPNGTSGSGTQSPIKSTNADKYIKKENSTYSLLRRGVYLVTFAYEFTSSQNQAVTCTGTLTEKTTGGTGSNPETLFSVVGSSDGLTVVGSFYILVPGEKAFSEHTLELKLTTGTAGTAATASKSYVAISYMGAGY

SEQ ID NO: 56 - protein, 491 aa, Chlamydia muridarum
MWKHWIRIIGIGSLLSPTVLLALGEKTPAFFLERNRNFWNIDPYCLESICACFISNQDPLSAERLEVLFPQLSQEDLLVFAKCILSSKNAEYLFSDAEEKIFAKLALPRVSLGCNRADDLAKVLVLAEAGSTAKEQKARYYSLYLDVLALRAYVERARLVNATCEVASQIDLASIEAINTILFEKEGVRYPSKKEMFESRFSELAAVTDSKFGVCLGSVVLYQAVAQRLNLSLEAVTPPGHIYLRYKDVVNIETTSGGRHIPTEKYCECIPESQLRVRSQEELIGLTFMNRGAFFLQKGEFLQASIAYEQAKKYLADEQISDLLGITYILLGKKKEGEDILKKSSERTRKSSAIHDYFERNISPEILAVLFADSGVTYQETLDYHKKLLVLVEKFPRSGALRLRLAATALELGLVKKGEQLLEKSVEDAPDDLFLRLQLCKILCNRHDYTRAKQHFDQAKKILAEEGMLSEKSSYTLLKILEKKLALVSPH

SEQ ID NO: 57 - protein, 308 aa, Chlamydia muridarum
MSNSPESSTPKQSIPARFPKWLRQKLPLGKVFSRTDGTIKNKGLPTVCEEASCPNRTHCWSRHTATYLALGDACTRRCGFCDIDFTKKPLPPDPQEGEKIAASAKALGLKHIVITMVSRDDLEDGGADALARIITTLHIELPEATIEVLASDFEGNIDALHHLLDARIAIYNHNVETVERLSPLVRHKATYRRSLMMLEQAAQYLPDLMIKSGIMVGLGEQESEIKQTLKDLADHGVKIVTIGQYLRPSRRHIPVKSYVSPETFDYYRSVGEALGLFIYAGPFVRSSFNADAVFEAMSQRERLSASIQ

SEQ ID NO: 58 - protein, 253 aa, Chlamydia muridarum
MKMGGWRKLYEVVYSSLIGCALKLRYRVSVEGLEAISPNSQKGALFLSNHVAEIDPVILEHVFWLKFHVRPIAVDYLFNSPVVKWFLDSVRAIPVPSVVPGRDDKRLLERMERFYACATQALDNKESLLLYPSGRLSRDGTEEIVNQHSAYTILHRAKECDVFLVKITGLWGSSFSRYKTGSTPKLGKVFKEAAKALLCRGIFFMPKREVKVSVCPADYSVLKQFPTKQEFNTFLSDWFNQKGGETPLEVPYT

SEQ ID NO: 59 - protein, 527 aa, Chlamydia muridarum
MPKKKFLYILIPFLSLTFGLTSCHQKEENLRNILRVAICHDPMSLDPRQVFLIKDVCIAKALYEGLVRENDGSLHLALAERYSLSQDRCVYTFFLKKTFWHNGDLVTAYDFEESIKQFYLHEVDNVALRLLALIKNSHAVLKGDIPVENLGVRALDEHTLEITLEHPSSHFLETLTHPVFYPVHASLREYYRNRSKRSLPIISNGPFIIRCYEPQNFLLLDKNPFYHDQKNVSLDAVRLQIVPDIHTAVQLFQKKYVDLVGLPWSSSFPLEEQKNLSQDFLYDYPVLNCTVLFCNVNHKPLDNPSLRAALSLAIDRETLLKLAGKGSIATSFVHPSLSKMPLDVLSQKERISLAKNYLAEALKTVPQEELKKITLIYPIESIVLRAVVQEIRQQLFDVLGFKISTLGLEYHSFLDKRSKGEFSLSTGNWVADYQQAKAFLSILGNGTKYKDYQVIDWQNQEYTDIVSRLLVEDSTDLQILAEQLLLKESPLIPLYHLDYAYAKHPKVSNLQTSSLGEIDLKRVSLVE

SEQ ID NO: 60 - protein, 358 aa, Chlamydia muridarum
MTSPLSTAASSYLRTLQRAFPLGGGGYPTNPNSAQTALRVQTAATSLAIVPYPRPLSSEPVSFFSKHCKNDAQSIIENAILSASSSVFLKIFSLSSEPIVQDLICKSEESIPVTIHYQHIPEPIVNKLEEAGAELIRCHRNRRSLLHRKTMILDEKQVITGSANFTRNSINNDVNLLARVNNVHIANLMQKNQKGKAIIENSTKGKQTVCYLPINHKKCGNERQIVKAINNATSSIQIGMCILTHRGILQALNEAATQRSVLVTIIIDSLESQQTIDILKALGSKLRVRVGTGDRIHCKACILDHNTAIIGSANWAASGLKANKEDIIIVNPLTERQREDLSIWWRYLCDNSAILPED

SEQ ID NO: 61 - protein, 740 aa, Chlamydia muridarum
MLTPIPLLQRFFSSPLSIKEILQACDRIGIEAECSNVFPDSLNTVVTGKVLSTSPHPDAERLSVAIVFDGKEERQIICGAPNCRAGIIVPVALPGAKIRNALGEVTTIKKSKIRGLESLGMCCGADELGFPHLQTGERGIFEFPENTPLGESACLLLAGASLECSLTPNLGHCASLLGLAREISFLSDTSLTIPEEFSFSPLPQESHSCDTHNLEASPVFYSVKISGLSWKQSPEDLQASLIALGQKPLNAIVDITNYVMLSLGQPLHAYDSQAVDQKSLHATTVRSPQQLTLLNKETYTLPEGALVVADQHNILGLAGVMGSAASAYSESTTEIILEAAYFLPQAVRKYQRTIQLHTEAAYRFTRGIDPQGVLPALYAAIHMIRALFPEAQISPIQKIGEYKSDPLSLNIRPKTLKRLLDLSLSPSEIAEKLSPLGFQTIAEESSVRVEVPLYRHDIQEETDLVEEICRTTPFVQKTQKILPTYTPIYALKRSLSTFLADSGLQQFFTCSLLDSEIAALSLQESSLIPVQNSSLKLRDSLLSGMLKSAATNLNRQAPYVYAFEIGNVYSKEHNQYREEEHIAILLTRQVMDDSWHVKTPLSFYTIKSWVEKLLCHAGVSIEDFVLQASQHPNFHPYRQAALYHKKQLLGVFGTLHPQLCKKSQIKHDVVFAELSLNVLLSLKKKTEPHYVPYPIYPSSSRDITITIDKDLPADLVRRELLSFESKWLESVHIVSVYQGK

SEQ ID NO: 62 - protein, 870 aa, Chlamydia muridarum
MSYMLKSIPPRHIQVFMFEKFTNRAKQVIKLAKKEAQRLNHNYLGTEHILLGLLKLGQGVAVNVLRTLGVDFDTAKNEVERLIGYGPEIQVYGDPALTGRVKKSFESANEEASILEHNYVGTEHLLLGILNQADGVALQVLENLHIDPKEIRKEILKELETFNLQLPPSSSITPRNTNSSSSSSSKSSSLGGHSLGGDKPEKLSALKAYGYDLTEMFKEARLDPVIGRSAEVERLILILCRRRKNNPVLIGEAGVGKTAIVEGLAQKIVSGEVPEALRKKRLITLDLALMIAGTKYRGQFEERIKAVMDEVRKHGNILLFIDELHTIVGAGAAEGAIDASHILKPALARGEIQCIGATTLDEYRKHIEKDAALERRFQKIVVQPPSVDETVEILRGLKKKYEEHHNVFITDEALVAAAKLSDQYVHGRFLPDKAIDLLDEAGARVRVNTMGQPSELLRLEAEIESTKQAKEQAIGTQEYEKAASLRDEEKKLREKLSNMKQQWESNKEEHQVPVDEEAVAQVVSVQTGIPAARLTEAESEKLLMLENTLQKKVIGQDQAVASICRAIRRSRTGIKDPNRPMGSFLFLGPTGVGKTLLAQQIAVEMFGGEDSLIQVDMSEYMEKFAATKMMGSPPGYVGHEEGGHLTEQVRRRPYCVVLFDEIEKAHPDIMDLMLQILEQGRLTDSFGRKIDFRNTIIIMTSNLGADLIRKTGEIGFGLRSHMDYGVIKEKIDAAVKKHLKPEFINRLDESVIFKPLEKEALSEIIHLEINKLGSRLQNHQMALNIPDSVVSFLVDKGHSPEMGARPLRRVVEQYLEDPLAEMLLKESCRQEARKLCARLIEERVIFEREDEISALAVEGDGPASVTADES

SEQ ID NO: 63 - protein, 428 aa, Chlamydia muridarum
MSFRCIFLVIVIFLITGCSYSLPKHTLTIAIHDDPHSLSPEQGENAFNFSLSRILFATLFREEPSGLIPALASSYQPSENGLCYRFFIRKDAKWSDGSLLLAEDVVAAWTHTKKTGRYPELFDNLTFYAPSSSEVVVELKDPSSQLLAILASPFFSVYRPESPLLSSGPFMVHTYLQGETLVLHKNPHYYDDALVELQSIDFRIIPNIYTALHLLRRGDVDWVGQPWHQGIPFELRSASPFYAHYPVDGTFWLILNSKDPILSSLANRKRLVAAIRKEELVRYALGDQYKVVESPSPNEDERFCCYEDSSAQFPGKITLIYPNNITRCQRLAEVLQEQCRNAGINLVLQGLEYHVFVQKRAMQDFSISTATSITFHPLTCAQFDHTALNDFTCLPLYHMEYDYILGKPLDHVIHHPSGSVDLIYARFH

SEQ ID NO: 64 - protein, 235 aa, Chlamydia muridarum
MALPKDPHLVKRMKFFSLIFKDQEVVPNKKVLSPDAYTTVLNAQELLEKTQEDCDAYTQHTHEECANLRAEAKNQGFQEGSEAWSKQLAFLITETQAMRDQIKSSLVPLAIASVKKIIGKELETKPETVVSIISESLKELTQNKRIVIHINPQDLAIVEQHRPELKKLVEYADVLLLSPKASVSPGGCIIETETGIVNAQLDVQLAALEQAFSAALKQKQPTETFSTDQTQSKEG

SEQ ID NO: 65 - protein, 591 aa, Chlamydia muridarum
MRKKTAYSEASIISLASLDHIRLRAGMYIGRLGDGSQSEDGVYTLFKEVVDNAIDEFVMGHGHTIFITGDSQKLSIRDEGRGIPLGKVIDCVSKINTGAKYNQDVFHFSVGLNGVGLKAVNALSQHFSVRSVRNKKFLKASFSKGILLHTEQGDTQDADGTEISFSPDKELFENFSFHDEFLKKKIRQYTYLHPGLTILYNGETFVSKLGLADLFEEEIQTPLLYPPIAFQHPDLSFLFSHTETSSEQYFSFVNGQETTDGGTHLIAFKEGVVKGINEFFKKNFSAQDIRDGIAGCIAIKIASPIFESQTKNKLGNTNIRADLAKRVKEAVILTLKKHPSSADRIQEKIKLNEKTRKNAHYKIPKLRDCKFHLTDNSLYGNNSSIFITEGESASASILASRNPLTQAVFSLRGKPMNVFSSKEETIYKNDELFYLATALGLHKNSLQNLRYNHIILATDADVDGMHIRNLMITFFLKTFLPVVEQNHLFILETPLFKVRYKDETFYCYSEEEKLSTIERLGKKESSLEITRFKGLGEISPKEFKSFIGPDMRLTPVSPPNADTLDALLQFYMGKNTKERKHFIIENLVTNL

SEQ ID NO: 66 - protein, 342 aa, Chlamydia muridarum
MFPLLIDCQKAAETVVMQAMLALMRYRQSRTFIPSWEKPDQTHVTPADYAIQYYFHQTLTSLFPHIPLVGEETLNPDTDHHKIPQILNFAKQLDPQVSCQNLYKALSSQNSHSSLFWLTDPIDGTSGFIKQRYFAVALSLFYEHSPILSVIANPTSNNASFKMYSAAKGEGLTIYTPTRSILFSLQEDFQLTHQFCEASLAARNHQHLTTHILSQHLPWHPRPVRADSQSKYAWVADNTVDFFIRIPYSPPRAHYRDHAPGVFLIEEAGGLVTDISGSPLTFSNPNLYLEKHPLILASANERIHNTILETLYRLRHRSTLNRLSPEEQSSFTKNKQLSALQL

SEQ ID NO: 67 - protein, 256 aa, Chlamydia muridarum
MFKFFGNKLRSLFKKTLPSDGDFGPKLTEAFCNELRRHKHPDERLIKELIRSFLSDIIAKLPQKKSSSAHPHVTLILGTNGSGKTTTVAKLAHYCLSQNQTVLIVATDTFRSAGMEQMRCWADSLGCGFISGKPRNYDQVIIDTSGRLHTHTNLLKELQKIATVCNKAFPGAPHETLMTVDATLGSNTLSQVKLFHEAVPIDGLIFTKIEGSAKGGSLFRIADELKIPTRFVGYGETVYDFEPFSIDRFLDKLLDV

SEQ ID NO: 68 - protein, 363 aa, Chlamydia muridarum
MLKFQLCALFLFGYLAIVFEHIVRVNKSAVSLAMGGLMWLVCFSHMPHIDHVIMAEEIADMAQVIFFLFAAMAIVELIDAHKGFSIVVRCCNVESRDNLTSIIIIVSILKRLVKAREDRLLLGALCVIGVNAGGAWTPLGDVTTTMLWINDKISTAGIITSLFLPSQLLLKKRRCSGLLEDLDSEPALPKSNLIAFVGFGSLLMVPMWKAVLGVPPFIGASDWIHSPHGEGRNHLRMPHILTRIDISSVTFFIGILLAVNALAYSHVLQDLSVSMDAVFSRNTLAILLGLVSSVLDNVPLVAATIGMYDLPMNDPLWKLIAYYMGMEKVSFGWYVKHASWIALISYFGGLAVYFLMENFMGLF

SEQ ID NO: 69 - protein, 100 aa, Chlamydia muridarum
MARKERLTNEKLNKLFDSPFSLVNYVIKQTKNRIARGDVRSSNVAIEALNFLDLYGIQSECLERDDREQYASGAGEKRKEQSSGNSRRKDPSLYNWSDVK

SEQ ID NO: 70 - protein, 548 aa, Chlamydia muridarum
SSRILITSALPYANGPLHFGHITGAYLPADVYARFQRLLGKEVLYICGSDEYGIAITLNAELAGMGYQEYVDMYHKLHKDTLKKLGISVDFFSRTTNPHHPAIVQDFYRNLQDKGLMENLVTDQLYSEEEEKFLADRYVVGTCPKCGFDRARGDECQQCGADYEARDLKDPRSKLTGKALSLRETEHAYFHLERMKEELLTFVEGIYLRPHMRNFVIDYIKNLRPRAVTRDLSWGVPVPGLENKVFYVWFDAPIGYISGTMDWAASIGDPDAWKKFWLDDKVTYTQFVGKDNTSFHSVIFPAMEMGQSLPYKKVDALVTSEFLLLEGFQFSKSEGNIIDMDAFLETYSLDKLRYVLAAIAPETSDSEFAFQEFKTRCNSDLVGKFGNFVNRVVAFAAKNGCTELSHPQLEQQDLEFIAEAQKLAKEAADHYQQYSLRKACSTIMELAALGNGYFNDQAPWKLAKEDLWNRVRSILFCACYCQKLLALISYPIMPETAWTIWGMIAPGSLDLRSQDPERLQSIWTEAFFDYSEESFSLKEPELLFTVVE

SEQ ID NO: 71 - protein, 365 aa, Chlamydia muridarum
MRVHSLFLKDFRNYSELRLELGPEMNSIFGLNAQGKTNILEALYILSLGRSFRTSRLTEAIRFGSSHFFIEAVFSQNQVFHTLSIQVDKRGKKILFDGAPITKLSALVGLFPVILFSVKDTTIIEGSPAERRRFLDLLLAQASEKYTGQIALYHKALDQRNAAIKTQDYKTIAAWNSPLIAYGSLVALLRYECAKKLHKIFQNLWDNTLKETLSLRYESSLITTESPTLNDIASNYYEQLRLANTKDFELGYTTVGPHRDELIITLNDLPVSKFSSEGQKHSLLAVLRFAECVYLQEEFLIHPLLCMDDIHACLDQNRLDQLFQLSTSLGQTVTTSTICPNHLDSNSSIFHVTQAQVSLVTSNFL

SEQ ID NO: 72 - protein, 413 aa, Chlamydia muridarum
PVQATNTYDLTAENILEKLSLTQEQQIKHDNLISNLKEAINRYSDLNRKNSRKGQSLLVRQAKILDEILSQTKSTEERASNSVMTTIKKEFTSHRVPVEKNIHGIWIAGSPPEGTDEYIKLFLHTYPEFSFLFWVDKTAYGAAKFSSTLKRIAFDAAVNSLREATPEPVKQFVQRYDKLKKSYDTSRDFDEKQRLSEQLVELYDNYNKFSKEIQSNFDVLLLHEMITIQDSFFNYCQLKGVGAITDETRIEYDLSHYKETIKRNKESIEKLVKEINDSTGRERVVIKDIRDLKSLQDLTNSYNYETEMLLRWNYAAATDQLRMYMLKEYGGIYTDLDIMPQYSQDVLQKIMDVGGSRFFEHDKLRRTLSFAALKLGSGKQTTVSFEEAKKAMTLPTFTLQDKSQISEIFKYLE

SEQ ID NO: 73 - protein, 101 aa, Chlamydia muridarum
MFISFDKTLSRHSTDLAEAFGNKLLYPVTQGFGTRISVKPVLVIDDMTIHGFSVKQESLCKRICCVIACILLAPIVLLMTLIGVIAYNCSATYKRVQDLYL

SEQ ID NO: 74 - protein, 48 aa, Chlamydia muridarum
MGANKIHAITQQIRLQRDSCFTEKIQNSSLQPLNVIAGLTETLNPNAE

SEQ ID NO: 75 - protein, 527 aa, Chlamydia muridarum; identical to SEQ ID NO: 59

SEQ ID NO: 76 - protein, 206 aa, Chlamydia muridarum
PNSAQTALRVQTAATSLAIVPYPRPLSSEPVSFFSKHCKNDAQSIIENAILSASSSVFLKIFSLSSEPIVQDLICKSEESIPVTIHYQHIPEPIVNKLEEAGAELIRCHRNRRSLLHRKTMILDEKQVITGSANFTRNSINNDVNLLARVNNVHIANLMQKNQKGKAIIENSTKGKQTVCYLPINHKKCGNERQIVKAINNATSSI

SEQ ID NO: 77 - protein, 890 aa, Chlamydia muridarum
MWVSRSGVISQLVLVSLVFYFVCAIIYIYSFFLVKRSCLKLSQQLKRIDIPTILHHGFCGKVIYAKQEKRVTDYYVAYYQRVRTFCRRFRYFGISSNPILLKFIQDFESITTLIKKHNSEIISYLLDLYKDFFDRVLHYPLDEQQRRSIISEEENCLVISSAGSGKTSSILGKVRYLIEIKGIDPRRIALISYTNKASSELTKRLATDGLRGYTFHKLAVDLIGSITGVKPSICSNPEELIEEICQKLLKTKDFLKNVLKYFVFYDDCESERKIRDQLSEQKKPFLKAVFPDMDGKTIYVRSKQEQTTCFILSSLGISFRYEEPYEFQVADETHSQYRPDFSLYVTAQNGSLRRIYLELFAINKQGRVPPWFVEGTNKTYKEVNQIYLDGIAWKKQTHKKFKTELLTLTSEDFYNSNIRENIWDTLRKLLERAGIPIQEKKEEDLYNALLPKKSVQEKALIKLIVRFISLMKSNHKSIKKVLYCAKIAKDIPSSWLIEKVFKPVYERYVDTLAKRGQIDFTDAILQATEICRHSSSGKYDYIIVDEFQDISIDCCRFLQELRKGNPPAKMYCVGDDWQSIYRFAGSNITVFNQFSDYFGPQEIHRLETTYRLGEPLVSLSSSFVQRNNFQIRKDCRSFNPDIRTELVFRSYYHRSEKDYCSCLELEISKIPEDKSIFLLGRYSFDDYYLSKKYQLVKTGSNFFYIIGNRKIEFLTVHKSKGLEADYVILLQCNADRYGFPSLVTDNKVFNYVLSESDSFPFSEERRLFYVAITRAKIKTTVLYDKKFPSEFVKEFIKVDEKRAQVPRTPVNAYKIWTRSEESLLLQLCKGMSVKDIATQTGRSPTAITARLRKLQKVGLSKRPQFVKSHNSFIRKKRFCRPEWKQSSRRY

SEQ ID NO: 78 - protein, 589 aa, Chlamydia muridarum
MRINVDKYLFIGRKKSEFFSACRELGAVEFLAKNKLKDSENVRRISEGLKTLNLLTNKYSPSDLVLVKSGYLTTEQLLQEIFDLNHEITTITDSLKALSKEIFRVKPLGNFSSEEIRELTLKTGLSVRFFYKKHIEGAPLEVEEENVFYLATAYNYDYYVVIGVVSLSKDIFTEIEAPRSVGELREEEEHLQTLLRKKKARVQCNEQTLQHAEASTEDLFDDKVFSALGWVIVDRLTEVEKLCNSLGVYLERVQPDPDEVIPTYLENHGLGALGESLVNIYDTPASTDKDPMIINDAGYGLIFLATSLFLSFKARKQVKHSLALKRFLKMFMILGGGCVCWGGATTSFFGVSVSYTSPFREYSLTHFLAMKKAEYYLKERPKGYKELVHDYPVLKDKKTPKEFLLAQGTSSGDSVYKAVVYDKFTDNILMEIALLVGVVHLSLGMLRYCRQRYSSIGWVVFMCGAYMYLPIYLQAVSLIHYALHVPYELGGQVGYYVTFDEVTAVIQVFSDVGAMVGNTVMVMSERFSPAVGVLIIIFGHTVNIALSIMGGVIHGLRLNFIEWYHYSFDGGGRLLHPLKRVICQKSQNI

SEQ ID NO: 79 - protein, 291 aa, Chlamydia muridarum
MRMQIPRSVGTHDGSFHADEVTACALLIMFDLVDKDKIIRTRDSEKLAQCEYVCDVGGRYSIADKRFDHHQVSYTGSWSSAGMVLDYLHHLGSLPHEEYEYLNSTLVHGVDEQDNGRFFSKEGFCSFSDIIKIYNPLEEGGNTDKEFFFALHFAIDLLTRLREKFRYDRICRDVVKQVMEKEDVCLRFDRPLAWQENFFSLGGENHPAAFVSFPCSDQWILRGIPPTLDRRMEVRIPFPEDWAGLLGDQLVKATGIPGAIFCHKGLFLSVWDSRESCEEALNLVLKQQGVV

SEQ ID NO: 80 - protein, 223 aa, Chlamydia muridarum
MALYLGLNPSFAKQYSAIFQPIIRIIPFSKHAPQMRQARQFLVSASHVLLTSPSSTHCFFSSIKKTVSPSALMSKCFVTIGEATSSRVRAHLPKANIVQVPFPQSEAFQPILSSLPSNTSILYPHSSLARPIIRSLLNKLQYPFFSYSHYTVKPIKLSLNIFSPHKIIILTSPSIVRAYAKLFPSLPSKEHWCLGEISAEEFKKIFHCPPSKILFPIKESQGC

SEQ ID NO: 81 - protein, 176 aa, Chlamydia muridarum
MSEPSSCFVLPDPEWIYRVGIGQDSHRFLPSDNPKPCVLGGIIFENTPGFEANSDGDVVFHAICNAFSSVTHQVILGALADELLKAKGISDSAVYLKEAVASLKPTQKISHLAITIEGQRPKLLPKISAMREKIAEILRISLDSVNITATSGEGLTSMGQGHGVQCFCVLTVMEFC

SEQ ID NO: 82 - protein, 766 aa, Chlamydia muridarum
MLTPIPLLQRFFSSPLSIKEILQACDRIGIEAECSNVFPDSLNTVVTGKVLSTSPHPDAERLSVAIVFDGKEERQIICGAPNCRAGIIVPVALPGAKIRNALGEVTTIKKSKIRGLESLGMCCGADELGFPHLQTGERGIFEFPENTPLGESACLLLAGASLECSLTPNLGHCASLLGLAREISFLSDTSLTIPEEFSFSPLPQESHSCDTHNLEASPVFYSVKISGLSWKQSPEDLQASLIALGQKPLNAIVDITNYVMLSLGQPLHAYDSQAVDQKSLHATTVRSPQQLTLLNKETYTLPEGALVVADQHNILGLAGVMGIILEAAYFLPQAVRKYQRTIQLHTEAAYRFTRGIDPQGVLPALYAAIHMIRALFPEAQISPIQKIGEYKSDPLSLNIRPKTLKRLLDLSLSPSEIAEKLSPLGFQTIAEESSVRVEVPLYRHDIQEETDLVEEICRTTPFVQKTQKILPTYTPIYALKRSLSTFLADSGLQQFFTCSLLDSEIAALSLQESSLIPVQNSSLKLRDSLLSGMLKSAATNLNRQAPYVYAFEIGNVYSKEHNQYREEEHIAILLTRQVMDDSWHVKTPLSFYTIKSWVEKLLCHAGVSIEDFVLQASQHPNFHPYRQAALYHKKQLLGVFGTLHPQLCKKSQIKHDVVFAETEPHYVPYPIYPSSSRDITITIDKDLPADLVRRELLSFESKWLESVHIVSVYQGKDVTSQSKNISLRLVFRDHERTLSGQEIEKEYERLTTLLDKNLANIGKGNS

SEQ ID NO: 83 - protein, 646 aa, Chlamydia muridarum
MVNHAESKSLHEKDFPVTQTYILVAMRRSVCYVTPSVARAGQISTWRFEYSSANFLPEGTLLKFDLGIDGRPIDWEIPSTDLSQPCNTIYLETPSESIVTAKAVYAPGSYIPTFEFILPCEIEAGDTFSIILGSSPNFPQEDASGNGAQLFTQRRKPFSLYVDPTGKGNFEDPDIFTIDIRGNVLKNIRIFAPSYVVKNKRFDITVRFEDEFGNLTNFSPEETQIELSYEHLRENLSWQLFIPETGFVILPNLYFNEPGIYRIQLRNQATKEIFTSAPIKCFTETSPHLLWGLLHGESERVDSEGNIESCLRYFRDDCALNFFATSSFEIQDGLTPETIKSINQTVSDFNEEDRFIALSGAQYVSEEPGEGIREVLLIKEPKSPGKHKECKLFPLSKLYKQSTSHELISIPSFTASKKFGCDFQNFHAEFERVVEIYNAWGCSERTEAEGNPFPIKGSIDSENPEGTILSALKRNLRFGFVAGGLDDRNLYSNFFDSDQQQYSPGLTAVICNKYSRDSLLEALYQRQCYATTGQRIIVSFQITSAPMGSELSTAIKPGLMINRHISGYVAGTAKISTIEIIRNGDTLHTFYPDGNNFEYEYDDLTPFAQATLADPKNGAPFAFYYLRVTQENGAMAWSSPIWIDLN

SEQ ID NO: 84 - protein, 311 aa, Chlamydia muridarum
MRLICRIFFFLCVLSAPQGFSNVCSDASCSKVQGSSSCRPCGATPQQEIQDSPGTPPPPFRCPSYRQPVEAQDLLASQEDLSSGAFSDTYPDLTTQAVIILFLALSPFLVMLLTSYLKIIITLVLLRNALGVQQTPPSQVLNGIALILSIYVMFPTGMAMYNDAKKGIESDAVPRDLFSAEGAETVFVALNKSKEPLRSFLINNTPKPQIQSFYKISQKTFPPELRQQLTPSDFVVVIPAFIMGQIKNAFEIGVLIYLPFFVIDLVTANVLVAMQMMMLSPLSISLPLKLLLVVMVDGWTLLLEGLMISFK

SEQ ID NO: 85 - protein, 346 aa, Chlamydia muridarum
MQADILDGKQKRVNLNSKRLVNCNQVDVNQLVPIKYKWAWEHYLNGCANNWLPTEISMGKDIELWKSNVLSEDERRVILLNLGFFSTAESLVGNNIVLAIFKHVTNPEARQYLLRQAFEEAVHTHTFLYICESLGLDEKEIFNAYNERASIKAKDDFQMEITGKVLDPNFRTDSVEGLQEFIKNLVGYYIIMEGIFFYSGFVMILSFHRQNKMVGIGEQYQYILRDETIHLNFGVDLINGIKEENPEIWTTELQQEIIEMIQRAVDLEIDYARDCLPRGILGLRASMFIDYVQHIADRRLERIGLKPIYHTKNPFPWMSETIDLNKEKNFFETRVTEYQHAASLTW

SEQ ID NO: 86 - DNA, 732 bp, Chlamydia trachomatis
atgcttcgagtcttagcttatcgacaggtttctttttctaagtttcctcgcttattcaaatcgtttgtgagccttttgtcctctcttaagcaacactattctttcatgttgcctggagaccctctccctaagaaaaaggcccttatgcttacattcgatcacgcttctgttgatttctacacacatgtctttcctcttcttcagaacttgcagatacctgcagttattggagttgcctggagatatgtggctgatttagaaggagaggatcttcccattgatgtgcgaattgctccctctgattttttagcttttcaggacgaaatattttcgtatcatcagcctttttgttcagtaagagagctgtgtcatatggctgcaagccctttagtgcgatttgcttcttcagggtttgcgattagaaatcttaaatatgctcctccttatctacacacagagattttattatctaaaattttattggagaatgccattcaaagcccagtagaaagctttttctttccattggggaaaagtgatgtagtaagtcagcattttgttcaggagacttatcggtattcttttgtgttggggaatacggctagtttttcctattccacacaatcattgcatgggattccgcgtatcgatatgcccttagatagtcaaagggttccatccctttatcaactttcttatcgccacttgaaacagtttttagttttgcgctag

SEQ ID NO: 87 - DNA, 162 bp, Chlamydia trachomatis
atgcatagctcctcccccatttctgaaatctgttctatggaaacagcagagcgagttcttgaagaaaaagagcatacccctactattatttcaggccagatagtgatttacccagaagatccttttgcaattccctcagttgatccatgcttcgagtcttag

SEQ ID NO: 88 - DNA, 938 bp, Chlamydia trachomatis
gtgaactcgacgaataatacagactctcaaaatctagatccaaatgcttcagaggtcgaaaaacttctcgacgaatccgcagaggcagaagagaaagtagatgatcatacgcccccttcagaactttttattctccctctgaataagcgtccttttttccctggtatggcagcccctctccttatagaggcaggcccccattatgaagtacttacactattagcaaaatcatcccagaaacacatcggtcttgtcttaacaaaaaaagaagatgccaacactctaaaagttggttttaatcagcttcatcgagtgggcgtatccgcgcgcattttacgcataatgcctattgaaggcggtagcgctcaagtgttattgagtatagaagaccgtattcgcatcgttaagccaattcaagataagtatctaaaagccaaagtctcctatcacaaagagaacaaagagctgactgaagagctaaaagcttactcaattagcatcgtatcgattatcaaagatcttctaaaactaaaccctctgttcaaagaagagctacaaattttccttggccactctgatttcactgagccagggaaactcgcagatttctccgttgctttaactacagcaacacgagaggaacttcaggaaattttagaaaccacagacatgcatgatcgcatagacaaagcattggtcttactcaaaaaagagttagatctaagccgtctgcaaagtagtatcaatcaaaaaattgaagcgaccatcacaaaaagtcagaaagaatttttcctaaaagaacaacttaaaacaataaagaaagagttagggctagagaaagacgatcacgctgttgatcttgagaagtttatggaacgcttcaataaacgagacgttcctcaatacgccatggacgttatccaagatgaaatggataa

SEQ ID NO: 89 - DNA, 828 bp, Chlamydia trachomatis
atgatggattacgagttgttagatagtggggacggtaagaaactagagcgatttaaggatgtttgcttaattcgttcttctgccacggctatttggccaaagagctctccctccttatgggggcagtattctgctgagtttgttcgtatgggagaacaagggcaatggcggtatcgcaacagaaatcttaaagaatggtggataaccatcgattctgtttcgtgtttattgaagctgactccttttggtcatgtaggtatttttcccgagcatgctgggttctggaaagatttacaaccttctgtagccaagccttcatgtagagttttgaatctctttgcttatactggagcttcttctattttctgtgctcaacaaggagcaacagtctatcatgtggatgcttctaaagcagctgttaagtgggcccaaaaaaacgtggaagggaacgcttttcaagataaaaggattttttggatcatagaagatgtgttttctttcttgaagaaggaaattcgtcgaggaaagacttatgatgtcattcttttggatccacctacctatgggcgaggtcctgatggagaaacattcaaaatagatcgcgattttttccctttattagagttgtgttcacagctcctctctagttctttctcgcatatgctgattacttcacatacaccgggacatactcccgagtttttgcattgtttagctagaagagcgcttcttatgcttcctctccaagggtggcgtttaggagaaaatttttgtggtgaggggaatcaacggttgccttcaggggtgtttgcgcaatggagtttatag

SEQ ID NO: 90 - DNA, 606 bp, Chlamydia trachomatis
ttgtctatgttttcaggcattattcaagaagtcgcacgggtagatcttattcaccatctcagggattccatggagattggagtttttgctcgcaagttgatcgatgtggttccggggagtagcttctctgtcgatggcatatgtttgactctggtcaaacgacagtacgaattactcttttttgatgtgactgaagaaaccatggcttggactaccatcaaagattatacggtgggaaccatggtaaatttagaacgctcggttcgattaggagatgaaataggaggacattttgtctctgggcatgtctgtgggataggcactattattgctatagagaaatcctatatgttttttaaggctccagctaatttagtgccttatattttagagaaaggcttcattgctattgatggcatcagtttgacaattgcacgagttaaaggggacatcttttcagttagtttgattccggagactcgagcgcgcacctcattgggttataaacaggtgggtgctcacgtgaatatggagcctgatatgatgacaaaaatgcaggtggacacaattatgcgtttccatgccgaaaaagagatcagcaaatga

SEQ ID NO: 91 - DNA, 1920 bp, Chlamydia trachomatis
gtgaacgttcgtacgtactctgttcagagggggggggtaaaaacgatttctgctagtgcagttcctcctacagcagctgttttatcgagaaaaaagcgtgctatagaagagaagaaggaggaagcttcttctggaaagatagaaaatcttgatgctagcaaatacgatcttactcccaagaacatagaagaaaaactaggaattactcctgaacagaaatctactgttaaagacctattaaataaactgaaaaaggtcattagtgcttacaactctatgccagataaaaattcggaagcgggacagaattccttgattcaacaaggaaaatacgtcgatgccattcagaagaagcttccagcatcatcgcaggctcagcctaaacaggcaaaagctaaggaacagaaagccgaagaaaaacctaagacgactccgattgaaggtgttcttgaaaccatcaaaacagaatttaaaggccatcgtgtacctgttgagaaaatcatccatggaatatggatcgcaggagcgcctccggatggtatcgaagattatatgcgagtctttttagatacttatgaaggttttgacttctacttctgggtagatgagaatgcttatgcagcagctaaattttctagcattttgaagaaggtcgctttcgatgcggctattcaagatctacgatctgccacagatgagtctacgaaggcctttgttaaagactacgatgaattaaaacagaaatatgaaaagaaagttgcggagacgacttctcaagcagaaaaagaccaatatctcaaagatctaaaggatcttttagagaaatttacaaaaatcagtgatgagattcgtggaaaatttgatcggctgtttcttaagaatgtgattgttgctcagaacggattctttaatttctgcttgctgaaaggcctcggcaatatcaatgacgaaacgcgtgcagagtatttagagaaagaactcaaacttcctactgaggagatcgaacagtataaaaagcttaaagagacgaacaaagagaagatagccgctattgtaaaacaactaaacgagaaacttggatcggatcgggtaaaaatcaaagacattaaagagctgcaatctatgaagcaagctcgaaatgtctacaattatgaacaggaaatgtttctgcgctggaactatgcagccgcaacagatcagattcgtatgtatatgttggaggaacttggaggtctttatactgatctggatatgatgccttcatactctcaggaagtattggagcttatcaaaaagcacagtgatggaaaccgaatgtttgaggatatgagctctagacgggcgatttctgatgcggttttaaagatggctgtaggtaaggcgacaacagtttccatggaagaggtagcaaaggatatcgatgtttctcgcttaacagaagaggataagacaaaattaaatgctctatttaaggatctagagccatttgcaaaaccggattctaaaggagctgaagcagaagggggtgaaggagcaaaaggtatgaaaaagagctttttccagcccatagatctgaatattgtcagaaataccatgcctatcttgagacgctatcatcactatcctgagttaggatggtttattcgaggattgaacggattgatggtctctcataagggaagcactgcggtttctgctgtcattgtagggcaacaggctgcctaccaggaactagcagcacttagacaagatgtcctttcaggggagtttttccattctttagaaaatttgacacatagaaaccataaggagcgtattggaaatcatctcgtcgctaattatttggctaaaagtctcttttttgattactgccaagattcagtgatgccggaggctgtaagtaccttaggtattagatga

SEQ ID NO: 92 - DNA, 795 bp, Chlamydia trachomatis
atgcgctgttcagcttattgcactgcttcatcctatcacttgcacgtgctctttcatcttctaaaagtaaactacccctcagttctatcccgagaatacgtgctgatttcctcagaggaactagatgaaagcgacaaagctgctgttttcttcccctttggcgtttgcgtgttctggggctgggaagaaactgaagaattacaagtgatccgtactatcaccccatctgcagtcaatcctcttcccaacccagaaatcgatagttacgatttccattacggagagaaactccaaatccgtcgcgataggctggttctgactaactcgaacctaaatacaaaacttgctatttcctttggattagctcagtctatcaagcttacagttttcgaagaaacgatttacaaaacggttgaaaactcgaaatctcttccccaagaactagcttccaaagggaaaatttctctatcccgtaaaacgattgcaaaaaaaatcggggaactgtttctagataaagcttctgtgaacctacactccgatatccttgatgaaccagatttcttctgggaacatccagaaactcaacctttttacatcgatgttttaacctgcctagacgtcaatgctcgcgtaaacgtgctgaatcatagacttgctatcttaggggacgttcttgaaattcttaatgaccaactaaaccatcaacactcctccgctctagaatggaccgtaatctggttaatcgctctagaagtgttggtcacactactgaaagacgtgtttaacatcatataa

SEQ ID NO: 93 - DNA, 2928 bp, Chlamydia trachomatis
atgaatcgagttatagaaatccatgctcactacgatcaaagacaactttctcaatctccaaatacaaacttcttagtacatcatccttatcttactcttattcccaagtttctactaggagctctaatcgtctatgctccttattcgtttgcagaaatggaattagctatttctggacataaacaaggtaaagatcgagatacctttaccatgatctcttcctgtcctgaaggcactaattacatcatcaatcgcaaactcatactcagtgatttctcgttactaaataaagtttcatcagggggagcctttcggaatctagcagggaaaatttccttcttaggaaaaaattcttctgcgtccattcattttaaacacattaatatcaatggttttggagccggagtcttttctgaatcctctattgaatttactgatttacgaaaacttgttgcttttggatctgaaagcacaggaggaatttttactgcgaaagaggacatctcttttaaaaacaaccaccacattgccttccgcaataatatcaccaaagggaatggtggcgttatccagctccaaggagatatgaaaggaagcgtatcctttgtagatcaacgtggagctatcatctttaccaataaccaagctgtaacttcttcatcaatgaaacatagtggtcgtggaggagcaattagcggtgacttcgcaggatccagaattctttttcttaataaccaacaaattactttcgaaggcaatagcgctgtgcatggaggtgctatctacaataagaatggccttgtcgagttcttaggaaatgcaggacctcttgcctttaaagagaacacaacaatagctaacgggggagctatatacacaagtaatttcaaagcgaatcaacaaacatcccccattctattctctcaaaatcatgcgaataagaaaggcggagcgatttacgcgcaatatgtgaacttagaacagaatcaagatactattcgctttgaaaaaaataccgctaaagaaggcggtggagccatcacctcttctcaatgctcaattactgctcataataccatcattttttccgataatgctgccggagatcttggaggaggagcaattcttctagaagggaaaaaaccttctctaaccttgattgctcatagtggtaatattgcatttagcggcaataccatgcttcatatcaccaaaaaagcttccctagatcgacacaattctatcttaatcaaagaagctccctataaaatccaacttgcagcgaacaaaaaccattctattcatttctttgatcctgtcatggcattgtcagcatcatcttcccctatacaaatcaatgctcctgagtatgaaactcccttcttctcacctaagggtatgatcgttttctcgggtgcgaatcttttagatgatgctagggaagatgttgcaaatagaacatcgatttttaaccaacccgttcatctatataatggcaccctatctatcgaaaatggagcccatctgattgtccaaagcttcaaacagaccggaggacgtatcagtttatctccaggatcctccttggctctatacacgatgaactcgttcttccatggcaacatatccagcaaagaacccctagaaattaatggtttaagctttggagtagatatctctccttctaatcttcaagcagagatccgtgccggcaacgctcctttacgattatccggatccccatctatccatgatcctgaaggattattctacgaaaatcgcgatactgcagcatcaccataccaaatggaaatcttgctcacctctgataaaattgtagatatctccaaatttactactgattctctagttacgaacaaacaatcaggattccaaggagcctggcattttagctggcagccaaatactataaacaatactaaacaaaaaatattaagagcttcttggctcccaacaggagaatatgtccttgaatccaatcgagtggggcgtgccgttcctaattccttatggagcacatttttacttttacagacagcctctcataacttaggcgatcatctatgtaataatcgatctcttattcctacttcatacttcggagttttaattggaggaactggagcagaaatgtctacccactcctcagaagaagaaagctttatatctcgtttaggagctacaggaacctctatcatacgcttaactccctccctgacactctctggaggaggctcacatatgttcggagattcgttcgttgcagacttaccagaacacatcacttcagaaggaattgttcagaatgtcggtttaacccatgtctggggaccccttactgtcaattctacattatgtgcagccttagatcacaacgcgatggtccgcatatgctccaaaaaagatcacacctatgggaaatgggatacattcggtatgcgaggaacattaggagcctcttatacattcctagaatatgatcaaactatgcgcgtattctcattcgccaacatcgaagccacaaatatcttgcaaagagcttttactgaaacaggctataacccaagaagtttttccaagacaaaacttctaaacatcgccatccccatagggattggttatgaattctgcttagggaatagctcttttgctctactaggtaagggatccatcggttactctcgagatattaaacgagaaaacccatccactcttgctcacctggctatgaatgattttgcttggactaccaatggctgttcagttccaacctctgcacacacattggcaaatcaattgattcttcgctataaagcatgttccttatacatcacggcatatactatcaaccgtgaagggaagaacctctccaatagcttatcctgcggaggctatgttggcttctaa

SEQ ID NO: 94 - DNA, 2415 bp, Chlamydia trachomatis
atggacgcacaagaaaagaaatacgacgcatcagccatcaccgttttagaaggattgcaagctgttcgtgagcgtcctggaatgtacattggtgatacaggagttaccggattgcatcacttggtttatgaagtggtggataacagtatcgatgaggcaatggcgggtttttgtaccgaggtcgttgttcgcatattggaagacggaggtatttctatttcggataacggtcgaggaattcctattcagattcacgaaaaagaatctgccaagcaaggacgcgaaatttccgctttagaggtggttcttaccgtactacatgcgggaggtaaatttgataaagacagttataaggtttctggaggcttgcatggagtaggagtttcgtgcgttaacgctctgtccgaaaaattcattgcaaaagtatttaaagatggacaagcgtatagcatggagttttcaagaggcgctccgttaacgactttacaggtgcttggtcctacagacaaacgaggtacagaagttctcttttatccagatccagctatattctctacgtgcgtctttgatcgcgcgattttaatgaagcgtttgcgagaattagcatttttaaatcgtggagtcacgattgtttttgaagatgaccgtgatacgggatttgataaagtcgttttcttctatgaaggcggaattcaatcattcgtcagttacctcaaccaaaataaagaaattcttttcccaaatcctatttatatgcagggatcgagacctggagatgatggggatatagaatttgaggccgctttacagtggaattctggttactccgaattaatctattcttacgctaataatattcctacaagacaaggcggaactcatttaacaggattttctacggctcttactcgggcagtaaactcctatattaaagctcataatctatctaaaagtgacaagttgtctttgactggagaagacatcaaagaggggctagtagcaattgtttccgttaaagtgcccaatcctcagtttgaagggcaaacaaaacaaaaacttggaaatagtgatgtcggatcggtttctcagcaaattagtggtgaggtgttaaccacattttttgaagaaaatcctcaaattgctaaaacgattgtagacaaggtgttcgttgcagcccaagctagagaagctgctaagagagctagagagctaactcttcggaaaagtgctttagatagcgctcgcttaccaggtaagttaattgactgcttggaaaaagatcctgaaaaatgtgaaatgtatattgtggaaggagactctgcgggaggttcagctaagcagggacgagatcgtcgttttcaagcaattcttcctattcgtggtaagatcctgaatgttgagaaagctcgtttacaaaaagttttccaaaaccaggaaattggaagcattatagcagctttaggatgtggaattgggaaggataatttcaacctcagcaaattgcgttataaacgtattattattatgacagatgctgacgtggacggttctcatattcgaaccttgttgctaacattcttctatagacatatgtccgctttgatagaaaatgaatgcgtctatattgcgcaacctcctttatatcgagtgagcaagaaaaaagatttccgttacattctctcagaaaaagagatggatggctatttgctgaacttaggaacaaaagaaagccagatagtttttgatgacaccttaagggatctgagaggggaagctttagaaacgttcgttaatcttattctagaggtcgaaagttttattgtttctttagagaaaaaagcgattccattttctgaattcttggatatgttccgagacggagcttatcctttgtattactatcctccagaaagtgggaagcagggaggcagttatctctattctcatgaagagaaggaagcagctattgctgccaatgaagaatctgcttcgcggattttagaattatataaaggttctgttctcgaagaacttcagcgcgatttaggtgattacggttacgatatccggaattacctccatccaaaaggatctggtattacagtttctacagaagatccgaaaatatctccgtatgtttgttacaccttgaaagaggtaatagactacttaaaaggccttggaagaaaaggaattgagattcaacggtataaaggtcttggagagatgaatgcagatcagttgtgggatacaacaatgaatcctgagcaaaggactctcgttagagtctctttaaaggatgcggtcgaagcagaccatatctttacaatgttaatgggagaagaggtacctccaagacgtgagttcattgagagtcacgctttatccattcggatgaataatttagatatttag

SEQ ID NO: 95 - DNA, 2379 bp, Chlamydia trachomatis
atgcttggaatacgcaaaaaaacgattctgcaactcgctgttttactgttgctcaccttttcacgaagttctttctgttcaacttcagaaggacgtatggtcgtagagtctatcaccattacgactcaaggagagaatactcaaaataaacgagctattcctaaaataaaaacaaagcaggggacgttgttctctcaagcagattttgatgaagatctaagaacactttcgaaagattttgatcgagtagagcctatcgtagagtttcgtaatggacaagctgtgatctctctgattctgacggcaaaacctgttatcagagagatcaatatttcaggaaatgaagctatccccactcataaaattctgaaaactttagagctttataaaaatgatctttttgatcgggaattattctttaaaaattttgatgcgctaagaactctttatttgaaacgagggtactacgattctcaactctcctattctcataatcataatgagaaagagggctttatcgatatttccatcgagattaaagaaggacgtcacggtcgcataaaaaaattaacgatttcgggaattacgcgaacagaagcatcagacttaggtgacattgttttaactaaacaatactccacaacaacgagctggttcactggtgccggagtgtatcatccggacatggtagagcaagacttatttgctatcacaaattacttccaaaataaaggatatgctgatgctaaagtaagcaaagaggtctctacagatgctaaaggaaacattactttgcttatcgttgtagacaaaggacctttatacacattaggtcacgtacatatagaaggattcacagcgttatccaaaagactgctcgataaacaactattggttggacctaactccttatattgcccagataaaatttggactggagcacaaaagattcgtagcgcatacgctagatatggctacgtgaacactaacgttgatgtctccttctcagcgcaccccactctacctgtttacgatgttacctatcgagtgagtgaaggatctccctacaaaatcgggttaattaaaatcaaagggaacactcatactaagcatgatgtgattttgcatgagactagtcttttccctggagacacttttgatagattaaaactggaaggtacagagactcgtttacgcaacaccggctactttaaaagtgtaagtgtctatacggttcgttcccaattagatcctcttgattctaacgacctttatcgagatgtttttattgaagtcaaagagactgaaacaggaaatcttgggctattcttaggattcagctccattgaccatttatttggaggggcagaaattgcagaaagcaactttgatttatttggagcccgaaactttctcaaaaaaggattcaaatctttaagaggtggtggagaatacctcttcctaaaagctaatttaggagataaggtcaccgattacactgttaaatggacgaaaccacacttcttaaataccccttggattcttggagtagaattagataaatcaattaataaagctttatcaaaagactactctgtggatacctatggagggaatatcagtaccacctacattcttaacgataagttaaaatatgggatgtattaccgtggtagccaaacaagcttaagtttgcgcaaaaaaacgtccagctctaatagacctggaccagatttagatagtaataaaggatttgtttccgcagcgggactcaatgttctctatgattctattgataatcctagaaaacctactatgggaatccgcagctccttaaactttgaattatctggtttaggcggaacttaccaatttactaaactaacagctagtggttctatctatcgcttattaactaaaaaaggtgttttgaaagtccgtgcagaagctaagtttatcaaacctttcggaacaacaactgcacaaggcattcctgtcagcgaacggttcttcttaggaggtgaaaccactgttcgcggttacaaaccttttattattggaccgaaattttctcctactgaaccacaaggaggcttgtcttccctactattaacagaagaatttcaatatcctttgatttctcaaccttgcattaatgcctttgtatttctagattccggattcattgggatagaagagtacactattcgcctgaaagacctttgcagtagcgccggatttggtctacgctttgatatgatgaataatgtgccaattatgctaggctggggttggccgttccgcccaacagaaatcctcaataatgaaaaaattgatgtatctcaaagattcttttttgccttgggaggagtattctag

SEQ ID NO: 96 - DNA, 1473 bp, Chlamydia trachomatis
atgaacaaaaaaataattaaagaacgcgtttcttcgtctgttccgtcgacaccttctgatcagcctgccgatcccacgaatcctgttactaaaatgctatttgaatgcaagctatctcagaatcaaggggatgcttctgccgatgttgatgcgatggtcaccacagctcatgtcaagctagacgctaagaatcaacaagttcgtcaggtagctaaaggggaagaagaccaaaatgtgattgtactctctcagcttgaaggatacttacagaaaggatctcgatcgatcgcgggattgttcctaccgacttctggagggccgatggttggagacattaatatggggacaagcccagggaataccatcaaagggcttccttcttcttggggatccgctatgttcctcgggaatgaatatgcagcttctgtagggattgtacaagatgttgtcggagagcatggagctcggttggatgatttgattgagaggattaccaagtattctggttcaggagagggtagtatgactaaacttcttgaagatctcggtagccctaaggaatctcagtctggacagacgccttctgtgactctagagaagcctacagatgctaagtggttattgttaagtgctaagaacgctatgacagggaccttgcgttttgagaaaaaacaaggagctttgccaacaactccagatcccacgataacaaatctgaaagcaagaggattaggaactattggaacagctactccgacagataagttacagaatatagtttctgttgccgacctcacaaccatactgaaagatttgcaggaaaatgctccttcggaaggaacaggttcagatacaggttcagacgtgggatctggatctggttcgggaacggactcaggatcagggggtgtagcaggtgtaagtgtaagcgcaggaaccacatcgaccttccccgagtggacgggacaagaggtgccttatttggtagagcaatccccggattctggttcaggttcaccaagtgtacagaatcctcttaagtctagtaatgcagacaattatatcaaaaaggataacgggacttattcgcttttgcgaagaggtctttatcttatcacctttgcgtacgagtttagttctccaacacaaaatggagctgttacctgtacagggactctcagaaagacaaccactccagaatcaggagatgtaggagctcagcctacgagtggggatgctacagccccaactgatccagagccttcaccagcattttccgtggtaggctcttcggatgggaaaactgtagttgggagtgcatacattttagtcccaggggacaaagcgctttcagaacatacattaactttggcgttgactactaatgaaaatacagaagtaaatcccacttcagttacagatccggcttctaagagttatgtagctatctcgtatatgggtgctggttattaa

SEQ ID NO: 97 - DNA, 1461 bp, Chlamydia trachomatis
atgtgctttattgggataggcagtcttctgttaccgaccgctctgcgagcgactgaacggatgagaaaggagcctatcccgctcctagataagcaacaaagcttttggaatgtagatccttattgtctggaatctatatgcgcttgttttgtagcgcatcgagatcctttgagtgcaaaacagttaatgtatctgtttcctcagctctcagaagaggatgtatctgtttttgctcgatgcattttgtcttcaaagcgtccagaatacctcttttcaaaatcggaggaagagctctttgcaaaattgattttgccaagggtttctctaggtgttcatcgggacgatgatttagcgagagtgttggtgttagcggagccttctgcagaagagcagaaggctcgatactattcattgtatctggatgttttagctttgcgtgcatacgttgaaagagagcgtttggcgagtgctgcacacggagatcctgagcggatagatttggcaaccatagaagctattaataccatcctttttcaggaagaaggatggaggtatccttcaaaacaagagatgtttgaaaacaggttttctgagttagctgctgttacagatagtaagtttggagtttgcttgggaactgtagtgctttatcaagctgtcgcccagcggcttgatttgtctctggaccctgtcacccctcctggacatatttacttacgctataaggacaaggtgaatattgaaaccacttctggaggaaggcatcttcctactgaaaggtattgtgaatgcataaaagagtcgcagttaaaggtgcgttcgcagatggagcttatagggttaacttttatgaatagaggagctttctttttgcaaaaaggagagtttcttcaggcgtccttagcttatgagcaagctcaatcatatttatcagacgagcagatttctgatttgttagggattacttatgttcttttaggaaagaaggcggcgggagaggctcttttaaagaaatctgcagaaaagactcggcgagggtcatctatctatgactatttccaaggatatatttcccccgaaatcctaggggtgttgtttgccgattcaggggtgacctatcaagaaactttggagtatcgaaaaaaactagtgatgctttccaagaagtatccaaaaagtggatctcttaggttgaggttggcgacaacagcattggagctagggctggtcaaggagggggtgcagttgttagaagagagtgttaaggatgccccagaggacctctctttacgtctgcagttttgtaaaattctttgcaatcgacatgattatgtccgagcaaaatatcattttgatcaagcgcaagctcttctcattaaagaagggttgttttccgaaaaaacttcctatactctcttaaaaactatcgggaaaaagctatctctttttgctccgagttaa

SEQ ID NO: 98 - DNA, 936 bp, Chlamydia trachomatis
atgaccgattcagaatctcctattcctaaaaaatctatacccgccagattccctaagtggctacgccagaaactccctttagggcgggtatttgctcaaactgataatactatcaaaaataaagggcttcctacagtctgtgaggaagcctcttgtccgaatcgcacccattgttggtctagacatacagctacctatctagccttaggtgatgcctgtacacgtcgctgcggcttttgtgatattgacttcactagaaatcccttacccccagatcctgaagaaggagctaaaattgctgagtccgcaaaagctttgggactcaagcacattgtcatcactatggtttcccgtgatgatttagaggatggaggagccagcgctcttgtccacattattgaaacgttgcacacagaattaccaacagcaactattgaagtcctagcctctgattttgaaggtaacattgctgccttacatcacttactggatgcgcacattgctatctataatcataatgtagaaacagtagaacgactcactccatttgtccgtcataaagctacctaccgaagatcgcttatgatgttggagaatgcagccaaatacctccctaacctgatgactaaatcaggtatcatggtaggattaggagagcaagaaagcgaggtcaagcaaaccttaaaagatcttgctgatcatggcgttaagatcgtaactataggccagtatcttcgtccttcacgcagacatattcctgtgaaaagctatgtgtctcccgagactttcgattattatcgctcggtaggagagtcgttaggactctttatttatgctggcccattcgttcgttctagctttaatgcggattctgtttttgaagccatgcgccagcgggaaacctcaacctctgcgctacttccgaataaagattag

SEQ ID NO: 99 - DNA, 762 bp, Chlamydia trachomatis
atgaagatagggttttggcgtagactgtatgaggtgtgttatacttctcttatagggtgcgctttgaaacttcgctatcgtgtattagtcgaagggatagagtctattaaccagaattcccagaagggagctttatttctttctaatcatgtagcagagatagaccctgtcattttagagcacgtgttttggttaaagtttcacgtgcgacctatagcggtagattatttattcaataatccagtggtaaaatggtttcttaattctgtaagagccatccctgtgccttccgtcgtccctggaagggatgataagcgtttgcttgaaaggatggagcgcttttatgtatgcgtgacccaagcactagatcgtaaggaaagcttgcttctttatccatcaggacgattgtctagaaatgggaaagaagagatcgttaatcagcaggcagcctacactattttgcaccgggcgaaggaatgtgatgtattcttagttaagatcacaggactttggggaagttctttttcacgctatagaacaggaagcacaccaaaattaggtaaagtgtttaaagaggcggtgaaagccttgttgcggtgcggaattttcttcatgcctaaacgcgaggtacgggtttcggtttgccctgcagattacttggtattgaagcagttcccgacaaaacaagaattcaatactttcctctcggattggtttaatcaagagggaggggagactcctttagaggtcccctacgcatag

SEQ ID NO: 100 - DNA, 1590 bp, Chlamydia trachomatis
atgcatcacaggaagtttttagcagtttccattgctttcgtaagtttagcttttgggctaacatcttgttatcataaaaaagaagaaccaaaagatgttttgcggattgcgatctgtcatgatccaatgtctttagatccgcgtcaggtttttttaagcaaagatgtttctattgtaaaagctctctatgaagggttagtccgggaaaaagaagctgcgttccagctagctttggcagaaagatatcatcaatctgatgatggttgtgtttatactttttttctaaaaaatacattctggagcaacggagatgttgtaacagcatatgattttgaagagtctattaaacaaatttatttccgagaaattgataacccttcgttacgctctcttgcattaattaaaaattctcatgctgttttaacaggagctctccctgttgaagatttaggtgttagagctttgaatgcgaaaactctagaaattgttttagaaaacccgtttccttattttctagagatattggcgcacccggttttttatccggtgcacacctctttacgagaatattacaaagataagcgtaacaaacgcgttttcccgataatttctaatggtccttttgcgattcaatgttatgagccgcaaagatatttactaatcaacaaaaaccctctgtatcatgccaagcacgatgttctgttaaattcggtatgtttgcagatagttcctgatatccatacagctatgcagttattccaaaaaaatcatatcgatttagttgggttaccctggagctcctccttttctttagaagaacaaagaaatctccctagagaaaaattatttgattatcctgtattgagttgctctgttttattctgtaacattcatcaaacacctttaaataatccctcgctgagaacagccctctctttagcaatcaatcgagaaactttattaaaactagcaggtaaaggctgtagcgctacgagctttgttcacccacaattatctcagatacctgctactactttgtctcaagatgagcggattgctttagcaaaaggctacttgaccgaagctttaaagactttatctcaagaagatttagaaaaaattacattaatttatcctatagaatctgtttgcttacgagccgttgttcaagaaattcgccaacaattatttgatgtactgggatttaaaatttctacattaggattagaatatcattgttttttagacaaacgttccagaggagaattctccttagcaactggtaattggattgcagactatcatcaagctagtgctttcctgtctgtcctaggtaatgggacaagatataaagactttcaattgattaactggcagaaccaaaagtacacaaatatagttgctcaacttctgattcaagaatcaagcgacctacagcttatggcagagcagttgttgcttaaagaaagtcctcttattcctctataccacctcgattatgtgtatgcgaaacagcctcgggtgtctgatctccaaacctcttctcgtggagaaattgatttaaaaagagtttcattagctgaaggatag

SEQ ID NO: 101 - DNA, 942 bp, Chlamydia trachomatis
atgctcgccggatccaagagaaaacacaagactcccgaagacacttcttcttcctcctctaaacgagcccgatcttcttcgagccaagtcgtacctagactcttacagcatcacgaactgatccaactctactctgctcatcagcaaagaaataacgaacctgtgaaaatgatttgtgaaacaattctacaagctaagcgcagcgttctattaaaaatatttaatatcggatcccccagaattcttgcagccttagctgaggcttctaatagagcgcctgtctccgtacactatcaaatggggcctttttcaaaacactgtactgaaggaaatgtgcagtttagacctcgaagagggtgttctcttttacatagaaaaacccttcttatagacaataatattgtcgttacgggaacagcaaactatacagaggcctctcttgaaaaagatgtgaacttaacggctaaaatatttagcgaacacctatatcgctgggctttccgacacgatcgaggagaggtgcgagtaggctcgcaacaagtatcctactattcgctaagtcaaatacgaagagacttgtgcgtcaaagctatcctcgaagctaacggcattgtcctacgagaacgtacatgcgaaggcattctgcataccaaagtctgctgtattgatagctcgacactcattataggatccgtcaactggagtagaggaggtcttacattaaatctggaagagttcttgattatcaatccgcttacagagacacaactcgaatgctataacgagctttgggcacatatagaaacaaacagtagattgatgactaaagagctgattcagttacatgagaagagaaaaaaatccataacagaccctaagcaaatctcttcttctactcaagacgaagagaatgcttccacatcagcagaacagcagttctga

SEQ ID NO: 102 - DNA, 2373 bp, Chlamydia trachomatis
atgctcgttcctttatccctattacaaaaattcttctcttctcctctctctattgaggaaattttacaagcctgtgatcgtattggaatcgaagcggaatgctcaaatgtttttccggattcccttaatactgttgtaacagggaaaattttaagtgcctctcctcatcctgatgcagaaagacttactgttgccatcgtttttgacgggaagggtgagcgccaaatcatctgtggagctcccaactgccgagcaggcatcattgttcctatagccttaccaggagctaagttacgtaatgcttcgggagaaatcactacaatcaaaaaagctaaaattcgtgggctcgaatcacaaggaatgtgttgtggagcagacgaattagggttccctcatttacaaaaagcagagcgtggtatttttgagttccctgcagatactcctcttggagaaagtgcctgcatgctacttgctggagctcccctagaatgctcgttaacccctaatttaggccattgcgcatccttactaggcctagcaagagaaatttcctttctatcccctgtatctcttaatatcccggaagaattttcttttgcctcgcttcctcaagaaacttctatttgtgatatgcacgatgctggagcttgccccgtattctattccgtcaaaatctcgggtctatcttgtcgacgatccccagaatatctgcaagcagccttaacagctcttggacaaaaacctcttaacgccatcgttgatattacgaattatgtgatgctgtcattaggtcagcccctacatgcttacgatagccaagcggtggagcaaaagtctctccatgcagccactctacagtcagctcaaccacttactcttctaaatcaagaaacctataccctccccgcaggatctttagtcgttgctgatcagcataatatcttaggattagctggggtcatgggaagtgctgcatcttcttgctcagaaaatacaacagaaatcattctggaagccgcttacttccagcctcaggctgtaagaaaatatcaacgcacgatacagctgcacacagaagctgcctatagatttacccgaggagtggatcctcaaggcgtattacccgtgttacatgctgctatccatatgattcaatctctatttccggatgctcaaatctctcctattcaaaaaattggagatgatagcttttctcctttgtctttaagcgttcgccctaaaacgattaaaagacttctggatattgagctctcgaccgcagaaatagttgccaaactctcttctttaggatttcaaacagcagtagaagagcaggccgttcgagtcgaggtcccctcttaccgtcatgacattcaggaagagaccgaccttgttgaagagatctgtcgcaccacaccattcgttcaaaaaacacaaaaaatactcccgacctatactccgatctattctttgaaacgtgaactcacagctttcttagcaaatggtgggctacaacaattctttacctactccttgctggatacagaagtatcttccttatctttgcaagaaagctctttgattcctgtacagaattcttcctggaaattacgcgactcccttcttcctgggatgctaaaaagtgcggctactaacctccataggcaagccccctatgtttatgcttttgaaattggaaatgtgtactcgaaggagcagaaccgttaccaagaggaagagcgtgttgctattttactctctcgccaagtaatggatgattcctggcaagggaaaacacctctctctttttatacaatcaaaggatgggtggaaaaactattatgccagtcaggagcttctatagaagacttttctcttcaacccagccagcatccgaactttcacccttatcagcaagctgccttgtatcagaaaaaacatctattaggcatctttgggacgttgcacccacaactatgtagaaaagctcagatcaaacacgatgtagtctttgctgagctttctttaaatgtgcttttgtctttaaaaaagaaatccggacctcattacgtaccctatccaatctatccggcctcttccagagacattacaattacaatagatagagatcttccagcagatctcgtacgcagagaacttttaagtttcgaatctaaatggcttgaaagtgttcatattgtcagtgtatatcaagggagagactctgcatctcaaagtaaaaacgtttctcttcggatggtcttccgtgatcacgaacgaaccttatccgggcaggaaatagaagaagaatacgaacgtttaactgcattacttgataagaaattagccaacataggacaaggcaactcatga

SEQ ID NO: 103 - DNA, 2565 bp, Chlamydia trachomatis
atgtttgagaagtttaccaatcgcgcaaagcaagtgattaagttggcgaaaaaagaggctcaacgactcaatcacaactatctaggaaccgagcacatcttgttaggattactaaaattaggccagggtgtggctgttaatgtattacgcactcttggtgttgacttcgatacagcaaagcacgaagtagaaaggctgattggctatggccccgagattcaagtttgcggagatccagctcttactggaagagtgaagaaatcttttgaatccgctaatgaagaagctgctcttttagaacacaattatgttggaactgaacatcttttgttaggtattctcaatcaatccgatggagtggctttgcaagtattagaaaatttgcacgtggatcctaaagaaatccgaaaagagattttaaaagaattagaaacatttaatttacaactccccccctcttcctccatcactcctagaaatactaatagctcttcttcttcgaaatcttcttctcctttaggaggtcatactttgggcggagataaacctgaaaaactttcagctttgaaggcgtacggttacgacttaacagaaatgtttaaagaatcgcgcctcgatccagtgatcgggcgttctgcagaagtcgaacgattaattttaattctttgtcgtcgaagaaaaaataacccagtcttagttggagaagctggagtagggaaaacagccattgtagaaggcctcgctcagaaaattgtctcaggagaagttcctgaagctcttcgtaaaaaacgcttgatcactttagatttggctctaatgattgcaggaaccaaatacagagggcagtttgaagaacgcatcaaagctgtgatggatgaagttcgtaaacatgggaacatcctcttatttattgatgagcttcacacgattgttggtgcaggagctgctgaaggagctatcgatgcttctcatattctaaaaccagctctagctcgaggagaaattcagtgcattggcgctacgaccctagatgaataccgtaaacatatagaaaaagatgccgctttagaacgtcggttccaaaaaatcgttgttcaacctcctagcgtcgatgaaacagtagaaattctcagaggtttaaagaaaaaatacgaagagcatcacaatgtcttcatcactgatgaagcactagttgctgcagctaaactctctgaccaatatgttcatggtcgatttcttcctgataaagcaattgacctcttggatgaagctggagctcgtgtacgagtcaatacgatgggacaaccttcggacctggtgcgtctagaagctgagatagaaaaaacaaaacaagctaaagagcaagctatcggaactcaggaatacgaaaaagctgctagccttcgtgatgaagagaagaagttaagagaaaaattaggaaatatgaaacaacagtgggagtccaataaggaagaacatcaagttcccgttgatgaagaagctgttgcacaagtagtttccgtacaaacaggaattcctgcagctcgtttaacagaagctgaaagcgaaaaacttttaaccttagaaacgactctacaaaagaaagtaatcggccaaagtcaagctgttgctagtatctgtagagctatccgtcgttctagaacaggcatcaaagatcctaatagacctatgggatccttcctatttttgggcccaactggtgtcgggaagactttattagcgcagcaaatcgcaattgagatgtttggaggagaagattctcttattcaagtggacatgtctgagtacatggaaaaatttgcagccactaagatgatgggatctcctccaggatatgtcggtcatgaggaagggggtcatctaacagagcaagtccgtcgccgcccttactgtgttgtgttgtttgatgaaattgaaaaagcacatccagatattatggaccttatgcttcagattttggagcaagggcgtttaacagactcatttggacgtaagatcgattttcgtaataccatcatcattatgacttctaacctaggagcagatttaattcggaaaagtggtgaaatagggtttgggcttcgttctcacatggactatgcagttattaaagagaaaatagatgctgcagttaagaagcatttaaaacccgagtttatcaaccgtttagatgagagtgttatctttaagcctcttgaaaaagaagctctttcagagattatccatctagaaattaataagctgggctctcgtttacaaaactatcaaatggatcttaatattcctgattcagtgatttcctttttggtaactaaagggcattcgccagaaatgggagctcgacctttaagacgtgttgtggagcagtatctggaggatcctttggcagaaatgctattgaaagagtcttgccgacaagaagctagaaaattacgcgcgcgcctcacagaagaacgcgttgttttcgaaagagaagaagaagctgtttctgctttagctatagaaggcgatggctcagagcctattacagctgatgaatcatag

SEQ ID NO: 104 - DNA, 1281 bp, Chlamydia trachomatis
atgtatgttcgctctatcttttttagtattatcgccttcctaacggtcggatgctccttttctcctccagaatcgggcttaatcatagccattcacgatgatcctcgctctctttctccagaaaaaggagaaaatgctttccatttttctttgtccaaggctttatttgctactctcttcagagaagagctctctggattaacccctgctctggtctcctcctatcaagtttcggaagacgggcggttttatcgtttttgtattcgtaaagatgctaagtggagtgacggctctcttttacttgcagaagatgtaatagctgcttgggaacacactaaacaagctgggcgatattccctactttttgaaaagctatcttttcgagcctcttcttcttcagaaatccttattgaactcaaagaacccgagcctcaactattggcgatattagcctctccgttttttgctgtgtatcgtccagaaaatccttttctttcttctggaccttttatgccaaaaacctatgtgcaagggcaaacgctcgttctacaaaaaaacccttattactatgaccatgcgcatgtggaattacattccatagactttcgcatcattcccaacatttacacagctctacacctcttaagaagaggtgacgtggattgggtggggcagccttggcaccaagggattccttttgagcttcggactacctctgctctctacacccattactctgtagatggcacattctggcttattcttaatcccaaagatcctgtactttcctctctatctaatcgtcagcgattgattgctgccgtccaaaaggaaaaactggtgaagcaagctttaggaacacaatatcgagtagctgaaagctctccatctccagagggaatcatagctcatcaagaagcttctactccttttcctgggaaaattactttgatatatcccaataatattacgcgctgtcagcgtttggccgaggtattgcaagaacaatgccgagacgcaggtatccagctgactcttgaaggactcgaataccatgtatttgttcaaaaacgagccactcaagatttctctgtctccacagcaacttctatagctttccatccccttgctaaatctaagttcgatcaaacggctctagacaatttcacttgtctgcccttgtaccacatagaatatgattatattttgagcagaccgctagatcaaattgttcactatccttcaggtagtgttgatttgacctatgcacactttcactag

SEQ ID NO: 105 - DNA, 708 bp, Chlamydia trachomatis
ttggctttgccaaaggatccctacctcgtcagaaggatgaaatttttcagtttaatatataaagaccaagaggtcgtccctaataaaaaagttctctccccagacgcctatactgcggtgttaactgcccaagaacttctggagaaaacacaagaagattgtgaagcttatacacagaatactcatgaagaatgcgcaaagctaagggaagaggctaagaatcaaggcttccaggaaggaagcaaagcttggagcaaacagctcgcctttcttattacagaaacacaagcgatgcgagaacagattaaagcctccctcgtgcctttagcgattgctagcatcaaaaagatcatcggcaaagaattagaaactaaaccagaaactgtggtttctatcatttcagagtctttaaaagacctcacgcagaataaacggattgtcatccacatcaatcctcaggatctcgccattgtcgaacaacatcgtcctgagttaaaaaaactcgtggaatatgcagatgtgcttttactctctcccaaagccagtgtatcccctggaggttgtatcattgaaacagagaccggaattgtaaatgctcagcttgatgtgcaactcgctgccctggaacaagctttctctgccatcctaaaacataaaaaacctgcggacgcctctacaatagatcagcctcaaagcaagaaagactag

SEQ ID NO: 106 - DNA, 1818 bp, Chlamydia trachomatis
atgcgaaaaaaaactgcgtattcagaatcttctatcatttctttagcttccttggatcatatccgcctacgcgcgggaatgtatattggaagattaggcgacggatctcaagctgaagacggcatttacacgttatttaaagaagtagtcgataatgctattgatgaatttgtcatgggatatggacacaccatccacataacaggagacgcacacgaactgtctattcgtgatgaaggccgcggcattcccttggggaaggtgattgattgtgtttctaaaatcaatactggcgcgaaatacactcaggatgttttccatttttctgttggattaaatggcgtgggactgaaagccgttaatgccttatcgcaacatttctctgtacgttctgtgcggaacaaaaaattcctcaaagcttctttctccaaaggcattctcttacacacagaacagggcgctacccaagatcccgacggtacagaagtcgttttctctcccgatcatgaactattcgagaatttttctttccaagtggagtttctaaaaaagaaaatccgtcaatacacctatctccatcccggactgacaattatttataacggagaacgtattgtttccactcgtggtcttcttgatctttttgaagaagaagtgcaaacccctcttctgtattcccctattacgtttcaacactccgatctcgcatttcttttctcccatacagaaacgtcttccgaacaatatttttcctttgtaaatggccaagaaacaactgatggcggaacccatctcgttgcctttaaagagggtatagtcaagggcgttaatgagttttttggaaaaaacttttcctctcaagatattcgtgagggacttgctggctgtatcgccattaaaattgcttctcctatttttgaatcccaaaccaaaaataaactagggaatacgaatatccgcgcagagttagccaaacgcgtaaaagaagctgtgctctcttctttgaaaaagaatccttccagcgcggaacggatccaagagaaaatcaaactcaacgaaaaaactcggaagaatgcgcaatttctcaagcaagagctcaaagataaacaaaaaaaactccactataaaatccctaaacttcgggattgtaaatttcatctcacagacaattctctgtatggtaaaaattcctctattttcattactgaaggagaatcggcttccgcttcgattctagcttcgcgaaatccgctcacacaagcagtgttctctttgagagggaaacctatgaacgtcttttcctccaaagaagaaaccatctataaaaatgacgaacttttttaccttactacagccctcggcctgcacaaagactctctacaaaatcttcgatacaaccaggtgatcttagcaacagatgcggatgttgatggtatgcatattcgtaatcttatgattactttcttcctaaaaacctttcttcctcttgttgcaagcaaccacctatttatcctagaaacccctctttttaaagtacgccacaaagatgccactttctattgctactcagaagaagaaaaactctcgaccatagaacacatcggtaaaaaagaatcgtctcttgaaattactcgtttcaaaggcttaggagaaatttctcctaaagaattcaaatcttttatcggcgcagacatgcgcttaactccagtttctcttcctgatacagaaaccctcgacacacttttacaattctacatgggaaaaaacacaaaagagagaaaactatttattattgagaatcttgttaccaacctctag

SEQ ID NO: 107 - DNA, 1029 bp, Chlamydia trachomatis
atgttctccctcctcaatgactgccaaaaagctgcggaaacagttgtcatgcaggctatgctgtcactgatgcgctatcgaaaagctcataaatttattcccttttggaaaaagcctgaccagacatacgttactcccgctgattacgctatccaatattatttttaccaaaaactgacatctttatttccgcatatcccgctggtcggagaagaaactctcaatcctgccacggatcaccctaggatccctcaaatcctacaatttgcgcagcagctcgatcccaaagtctcctgtcaagatttgtatcaggccctctctccggaaagctcccactcttcactattttggttaacggaccctatcgacggtacttcgggattcatcaaacaacgttgtttcgccatcgctctttctctattttacgaacatactcctgttctctccgttatcgcttgcccttcttctgaaaataactctttcaaaatctattcggcagccaaaggcgaaggacttacgatttgtaatcctacccattccttccccttctccctgcacgaagatttccaacctacctgtaaattctgtgaagcctccttatcagctagaaaccatcaacatttaactacacatattctcagtaaacatctcccttggaatcctcagcctatccgagcagatagtcaatgcaaatacgcttgggttgcagataatacggtagacttcttcattcgcatcccgtattcccctccacgagcccactacagagaccatgctcccggtgtgtttcttatagaagaagctggaggcctcgttacagatatatcggggaatcctctccctttttctaatccaaatttatatctagatagacatcctttgattcttgcttctgctaacgagcaaatgcatagcacgattttagagactctatacaaactacgtcaccaagctactcaaaacatgctcccccttgccactcatatctctgctactaagcataaacagctaagcgctcttcaactatag

SEQ ID NO: 108 - DNA, 855 bp, Chlamydia trachomatis
gtgttcaagtttttcggaaacaaactccgctctctttttaaaaaaacgctttcttctgacttattagaatatgctgaagttctcttgtatgagggagattttggccctaagctgacagaagctttttgcgaagaattacgccgatgtaaaaatcctgatgaacgggcagtgaaagagcttattcgctctttcttatcaaagattatttctaagctcccccaacgagagcctctttctgttcgtcctttttctactcttgtcctaggaacgaatggctcggggaaaacaaccacagttgcgaaactggctcactattatctttctcaaaaccaaaaggttttaattgtagctactgatactttccgttccgcaggaatggaccagatgcgttgttgggcagaaaccttaaactgtgggtttatctcagggaaacctggaggagatgctgctgcgatagcttttgatgggatttctgcagcagtcgctagggactatgatcatgtgatcatagatacttcgggaaggttacacacacacacgaatcttcttaaagaacttcagaaaatcgctaccgtttgcaacaaagcttttcctggagctccgcatgagacactgatgacgatcgatgctactctgggaagcaacacattgagtcaggtgcaactattccatgaagccgtacctatcaatggacttattttcacaaaagtggaaggttctgccaaaggaggctctctcttccgtatcgcagatgaactcaaaatccctactcggttcgtaggctatggagaaacaattcatgatttcgaaccttttgccatagaccgttttctagataaattactggatgtttga

SEQ ID NO: 109 - DNA, 1266 bp, Chlamydia trachomatis
atgttaaagttccagttatgtttgttatttctgttcggatatctcacgatcgtttttgaacatattgttcgagtaaacaagtctgccgtatctcttgctatgggaggcttgatgtggttagtttgtttttcccatataccgcacattgatcacgtcatgatgttcgaagaaattgcagacatggcacaagtcattttcttcttatttgctgcgatggcaattgtggaactcatcgatgctcatagaggattttctattgtagtgcgttgttgtaatgttgaatccagaagcgtgctgctttgggtattacttaccctttcttttttcctttccgcagccttagataacctgacatccattattattattatttctattctaaagcgtctggtgaaagctcgagaggatcggctgctactaggagctctatgcgtgattggagtaaacgcaggaggagcttggactcctttaggggatgtgactacgactatgctttggattaacgataagatttctacgtcgggtattattactacgctatttcttcctagtgttgtctgtgtagtgatcgcaggaatctgtgggcaattattactaaaaaaacgtcgttgttcaggattgtcagaggatttggatagagaaccggcgctccctaagagtaacctgattgcctgtgttggctttggttctttgctcatggttcctatgtggaaagctgtattaggagttcctccttttatgggggctttattgggactgggtttagtttggttggtgagtgattggattcactctcctcatggtgaggggcgtaatcatttgcgtatgccgcatattttaacgcgtatcgatatttcttctgttacattctttatagggattctgttggctgttaatgcgctaacctactctcatgtattacgggatttatctgtgagtatggatgcgctattttctcgtaacacgcttgctgttcttttaggtttagtctctagcgttttagataatgtgccattagtcgctgcaacaataggtatgtatgacttacctatgaacgatcctctttggaaactcattgcctatacagcaggcacagggggaagtattctcatcattggatccgctgcaggtgttgcctacatgggaatggaaaaagtgagtttcggctggtatgtcaaacacgcttcttggattgctttagccagttattttggaggtctagcagtctattttctaatggaaaattgtgtgagtttgttcgtctga

SEQ ID NO: 110 - DNA, 303 bp, Chlamydia trachomatis
atggctagaaaagatcgtttaactaatgaaagactgaataagctatttgatagcccctttagtttggttaattacgtaattaagcaagctaagaacaaaattgctagaggagatgttcgttcttctaatgtcgcgattgaggcgctgaacttcctggatctttatggcattcagtccgaatacgctgaaagagatgatcgagagagacatttgtctgctacaggagagagacgaagagaacaaggtttcggaacatccagaagaaaagatccttctctgtacaactggagcgacgtgaaatag

SEQ ID NO: 111 - DNA, 1653 bp, Chlamydia trachomatis
gtggaatcttcccgtattcttattacttctgcgttgccttacgcaaatggtcctttgcattttggacatattaccggtgcttatttgcctgcagatgtttatgcgcgttttcagagactacaaggcaaagaggtcttgtatatttgtggttctgatgaatacggaatcgcaattacccttaatgcagagttggcaggcatggggtatcaagaatatgtcgacatgtatcataagcttcataaagataccttcaagaaattgggaatttctgtagatttcttttccagaactacgaacacttatcatcctgctattgtgcaagatttctatcgaaacttgcaggaacgcggactggtagagaatcaggtgaccgaacagctgtattctgaggaagaagggaagtttctagcggaccgttatgttgtaggtacttgtcccaagtgtgggttcgatcgagctcgaggagatgagtgtcagcagtgcggtgccgattacgaagctagagatctgaaagagcctcgttctaaattaacgggggcagctttatctttacgtgatacggaacatgcttacttgcatttggagcgcatgaaagaagatttgcttgctttcgtgcaaggtatttatctacgtcctcatatgcgtaatttcgttacggattacatcgagcatttacgtcctcgagcagtgactcgagatttgtcttggggaatacccgttcctgatttggaaaataaggtattctatgtatggttcgatgctccaattggttacataagtggaactatggattgggcagcatcgattggagaccctgaagcttggaagaagttttggttggacgatactgtgacctacgcacagtttataggtaaagataatacttctttccatgcggctattttccctgctatggaaataggacaatctcttccctataagaaagtggatgctcttgtaacatcagaatttttattgttagaaggtttccagttcagtaaatcggatgggaattttatagacatggatgcgtttttagaaacgtattccttggataaactgcgttatgtgttggcagcgattgctccagagacttcggatagcgaattctctttccaagagttcaagacgcgatgcaattctgagcttgtagggaagtatggaaattttgtgaatcgagttctagcttttgctgttaagaatggatgcacagagctttcttctcctcaattagagcaaaaggatttggattttatctcaaaatctcaaaaacttgctaaggatgcagccgaacattacgcacaatacagtttgcgtaaggcgtgttccacgattatggaattagctgctttagggaatggctatttcaatgatgaagctccatggaaattggctaaagagggtaactggaatcgggtacgcgctattctattctgtgcttgttactgccagaagttgctagctctcatttcctatcctattatgcctgaaacagcattgaagattttggaaatgatagctccacattccttagatctaggttcccaagatccagatagattacaatctctttggacagattccttttttgattactcggaagagaaattttctctgaaagagcctgaattattgttcacaatggtagagtga

SEQ ID NO: 112 - DNA, 1098 bp, Chlamydia trachomatis
atgcgggttctttctttatttcttaaggatttcagaaattatacagatcttcgtttagaattaggaccggagatgaattccatcttcggtcttaatgcacaaggaaagacgaatcttcttgaggctctgtacattctatcactaggtaggtcttttcgaactagtcgactgaccgatgccatacgttttggagcatcccacttttttatagaagctgtcttctcgcataaagaggtttttcacactctttctattcaagtggataaaaaagggaagaagatcctgtttgacggagctcctattacgaaactctctgagctagtagggttatttcctgttattttgttttccatcaaagacatagcaattattgaaggctccccttcagaacgtcgtcgttttttagacctcttactagcacaagcttcagataaatacaccgagcatatttctctctaccacaaagctttagaccagcgtaacgcttctattaaagctcagaatcagaaagctatttctgcatggaattccccattgattgcttatggtagtctggtagccttcttacgcaatgaatgtactaaaaagctaaatacgatctttcaaactctttgggataatacgttaaaagagactctatctcttcgttatgagagttctctcattacagaggaatcccctaccctcaatgacatagcaagtaattactacgaacaacttcgcatagcaaatactaaggacctcgatcttggctatactatggtagggccccatcgggacgaactccttctaactatcaatgatcttcctgtcgctaaattttccagcgaaggccaaaaacattcgcttttagccgttcttcgatttgctgaatgcgtgtatcttcaagaagagttttgtatccatcctcttctgtgcatggatgacatccatgcttgtctagatcaacagcgtttagatcagcttctgcaactctcaaactctttagggcaagtggttacaacttctactatttgtccagatcaccgcagcacaacatcctgcatttttcatgtgacacaagctcaagtatcccttgtagctcctcaatctctataa

SEQ ID NO: 113 - DNA, 759 bp, Chlamydia trachomatis
ctacgatgaattaaaacagaaatatgaaaagaaagttgcggagacgacttctcaagcagaaaaagaccaatatctcaaagatctaaaggatcttttagagaaatttacaaaaatcagtgatgagattcgtggaaaatttgatcggctgtttcttaagaatgtgattgttgctcagaacggattctttaatttctgcttgctgaaaggcctcggcaatatcaatgacgaaacgcgtgcagagtatttagagaaagaactcaaacttcctactgaggagatcgaacagtataaaaagcttaaagagacgaacaaagagaagatagccgctattgtaaaacaactaaacgagaaacttggatcggatcgggtaaaaatcaaagacattaaagagctgcaatctatgaagcaagctcgaaatgtctacaattatgaacaggaaatgtttctgcgctggaactatgcagccgcaacagatcagattcgtatgtatatgttggaggaacttggaggtctttatactgatctggatatgatgccttcatactctcaggaagtattggagcttatcaaaaagcacagtgatggaaaccgaatgtttgaggatatgagctctagacgggcgatttctgatgcggttttaaagatggctgtaggtaaggcgacaacagtttccatggaagaggtagcaaaggatatcgatgtttctcgcttaacagaagaggataagacaaaattaaatgctctatttaaggatctaga

SEQ ID NO: 114 - DNA, 378 bp, Chlamydia trachomatis
aaaggtatgaaaaagagctttttccagcccatagatctgaatattgtcagaaataccatgcctatcttgagacgctatcatcactatcctgagttaggatggtttattcgaggattgaacggattgatggtctctcataagggaagcactgcggtttctgctgtcattgtagggcaacaggctgcctaccaggaactagcagcacttagacaagatgtcctttcaggggagtttttccattctttagaaaatttgacacatagaaaccataaggagcgtattggaaatcatctcgtcgctaattatttggctaaaagtctcttttttgattactgccaagattcagtgatgccggaggctgtaagtaccttaggtatt

SEQ ID NO: 115 - DNA, 86 bp, Chlamydia trachomatis
ggagcgtttagggaacgttcttctctatccaatcgttcgaggatttgggtctcttgtatctgttaaaactttaggagaaaaacgat

SEQ ID NO: 116 - DNA, 2499 bp, Chlamydia trachomatis
atgaaccgtattcatcgtacacaaggatctttaacggattataatagcactcttgaggctattgcaaagaagattgccaaaccagattctgcaacaatagtatctcaggttgctcaatatgagcagttcaaaatggaacaagaagctttaaaagcacttcttgtttccttcgatcaaaaagcagaccagcgatatcgaaatttgattcaacgactggaacagttggatgtagatagacaaacaggacgctctacagagagccaacacatccaagaaaaaccgatggcttctctgcaatctgaaaaccaggttgttgcacaagctgtagttcagtctgattctagcatgcccatttttacaggtattaaacaaagttgggcagtacgattagtacagggtatacgtgaaattttggatcaagtcttagtggacacatccttgtttacggaagaagaacggggagatttacttgctatccgtatggatgccgcttccctacaggacaagcaagagcgtttgtctacggaagatattcgttctcttctttctctttcgaatgatgttatgcgagttttgcagaaagcttctgtatctagcacgcggcagctagaattgatccaatctttgatagatatttttggtacggaagagaatttagagcagtccttcgcccaggtacgattggagaacttccaagcgattttatctgtgattaaagagcgtttgacagaagaagagtttcgggtattccaagaagtctcggaagaaatttcctctatccaacgcacgagtgaatctcatctcagtccagaacatatagaggcgattgctcgagtcggtgggcatctttctgctaagatcgtggagtctgagctgaaggcaagtcagaaggtggatctctgtcaacgaatagcagctatgtaccaagaacaagtggatgcggtacaagcgtatcatagtctcgaacaagatgctctatttgtgaattctaggcaacatagtcacttcgtgcaggtgatttctttagtatcctctcttatgcactcattgtctcctactagtgaagaggagcgtattttactgaaccctgccatgatggtaagtgttcttcccactgtgcgtgcaataggtttacgttttgactttttaacagctgagcagcagcaaatggtgaatgctgcggtatcatcgttgcaacaacagcagttagacgaatttttaggagttctctgtgcgcatttggttgttgttaattgccagaataaagaaacaggtctcttagaaggtttagaagaaagcttttcagaaacgttaagtggtttgagtaataactttgttttgacggccaaaatgcaagacattttgcaggtttgttctttacaaggctttgtcacattagcaaacggggatcgatacgagttattttcctataatgattcaggagaagcggtatgtgatgagatcgctctaggcgatggatttcataaagtgctaggaacgatgttagcagtagcgctttctcaagcagaggtatttaagcaagagtgcgatcgattcattctgcaagctgattcagagaagaatatgattcacaaacgtatggttcaaggggaacagaagagtctattcttgaccaagatgcagacagagttgaatgctgggaaaactattgcacaaactaaggaagtagaggcttctccgctcccttctgccgttgcttctgtattgattgaccactacatgccaaaggaagtggagtttttagaaaagatctcgtctcgtctgtattacggaaataaaggttcggatataggcaacaccattttagatgcgattagtttgtatgtaaactccgcaacctactttggttttgctaactatattgggcaacctcctgttgtaggtaaaacaagggagaatatttttgcagggtctgcagataatgctaaagccaagttagacgaagaaaagaaacaagtagatgtgtttctagaaattacggaagcagctaagactaccgtcactaatcaacaaagtgcggttactaatgatgacaagttatctacagagcagaaagcaaaaattaaggcagagttgacacagtataccgatatgcttaacgcaatcagcaattctttaacatcattgaaaactcaattggcccctttatcagtgagtactgttgaaggtgtggatggtgtttttgaggttaaaaatggtataccaggagaaaacggaaagaactggagattagttttacagactttagaggatactgtggtatccggagaagtcggtagcccaactaatattggaatgttccaaatgcaagctctggtacacttgaatcagcaggcctatgctgatatgggacagaattttcagttagagttgcaaatgcatctcacttctatgcaacaggagtggatggtggtagcaacatctcttcagctattaaatcaaatttatttgggattagcgcgtaatctcttaagatag

SEQ ID NO: 117 - DNA, 1587 bp, Chlamydia trachomatis
atgcatcacaggaagtttttagcagtttccattgctttcgtaagtttagcttttgggctaacatcttgttatcataaaaaagaagaaccaaaagatgttttgcggattgcgatctgtcatgatccaatgtctttagatccgcgtcaggtttttttaagcaaagatgtttctattgtaaaagctctctatgaagggttagtccgggaaaaagaagctgcgttccagctagctttggcagaaagatatcatcaatctgatgatggttgtgtttatactttttttctaaaaaatacattctggagcaacggagatgttgtaacagcatatgattttgaagagtctattaaacaaatttatttccgagaaattgataacccttcgttacgctctcttgcattaattaaaaattctcatgctgttttaacaggagctctccctgttgaagatttaggtgttagagctttgaatgcgaaaactctagaaattgttttagaaaacccgtttccttattttctagagatattggcgcacccggttttttatccggtgcacacctctttacgagaatattacaaagataagcgtaacaaacgcgttttcccgataatttctaatggtccttttgcgattcaatgttatgagccgcaaagatatttactaatcaacaaaaaccctctgtatcatgccaagcacgatgttctgttaaattcggtatgtttgcagatagttcctgatatccatacagctatgcagttattccaaaaaaatcatatcgatttagttgggttaccctggagctcctccttttctttagaagaacaaagaaatctccctagagaaaaattatttgattatcctgtattgagttgctctgttttattctgtaacattcatcaaacacctttaaataatccctcgctgagaacagccctctctttagcaatcaatcgagaaactttattaaaactagcaggtaaaggctgtagcgctacgagctttgttcacccacaattatctcagatacctgctactactttgtctcaagatgagcggattgctttagcaaaaggctacttgaccgaagctttaaagactttatctcaagaagatttagaaaaaattacattaatttatcctatagaatctgtttgcttacgagccgttgttcaagaaattcgccaacaattatttgatgtactgggatttaaaatttctacattaggattagaatatcattgttttttagacaaacgttccagaggagaattctccttagcaactggtaattggattgcagactatcatcaagctagtgctttcctgtctgtcctaggtaatgggacaagatataaagactttcaattgattaactggcagaaccaaaagtacacaaatatagttgctcaacttctgattcaagaatcaagcgacctacagcttatggcagagcagttgttgcttaaagaaagtcctcttattcctctataccacctcgattatgtgtatgcgaaacagcctcgggtgtctgatctccaaacctcttctcgtggagaaattgatttaaaaagagtttcattagctgaagga

SEQ ID NO: 118 - DNA, 128 bp, Chlamydia trachomatis
gttctcttttacatagaaaaacccttcttatagacaataatattgtcgttacgggaacagcaaactatacagaggcctctcttgaaaaagatgtgaacttaacggctaaaatatttagcgaacaccta

SEQ ID NO: 119 - DNA, 1905 bp, Chlamydia trachomatis
atgcttacatccgaattaaatgcagcacaagttactgccgtcacggcgccactacaaccagtcctggtcttagctggagctggagcagggaaaactcgagtagtcactcatcgaatcctttatttaatagaagaatcccggctcgatcctaaacaaattttagctattacgttcacgaacaaagcagctaacgaactaaaagaacgtgtccaatcgcaatgtcgtgagctaggatattcagacgtacctatggtaagcacattccatagtcttagcgtttatatcctgcgacgctctattcatttgttagatagacagtcgaattttgttatttatgatcagagtgattctgaaaaattaattaagcaatgcttacgcaagctgaacctcgataaaaagctttgcaatgcaatgcaatttactatctctcaagcaaaaaatcgcttgcagagtcctgaagatctcgattccaaagaatatccagaccctactcgcaccgtttatgctgaataccaagaacagttacgtgctgcgaacgctctagatttcgatgatctgttattcctaacagaaaaactgttgcgcacacctgaagtccagcaagaatacgctaatcattggaaagccttgttgatagatgagtatcaagacaccaaccacgcgcaatatctcattgccaaacgtcttgctgcggctcataacaatatctttgtcgttggagatcccgaccaatctatatattcttggagaggcgccaacatctctaatatcttaaactttgagcaagattattcgcaggctttagttgttcgcttagaagaaaactatcgttcttgtgggaccatcctagaagccgctaatgctctgattcaaaataattctgcacgcttagaaaaaacattgcgtagtgtaaaagggcctggagataaaattttctgttttacagggaaaaatgaccgcgatgaggcagagcaagtcttagaggaaatctctaatcttcattcctataaagatatccctctctctgacatttgtattttatatcgaacgaactttcagtctcaatcgttcgaagcggctcttcttaagcgaggttatccctatgaaattataggtggcatttccttctacaagcgtagagaaatccaagatattcttgctttccttcgcttattttctaataactacgacatggcagcctttgagcgaaccattagcctgaaaaaatgcggtattggagctaccacattagccgctcttatgaactatgccaaaacaacagacctacctatattgcaggcttgttgggacgtattagagaaaaaatctatccgtttaactaaaaaacaacagcaagggcttttctcttatttgactcatttccatcaaatggagcagctatacggaaactgcgatcttcatgaattcatcaatgaaacgatccgcatcacggattacctctccatcctcaaagaagatcctgaaacctatgaagatagaaaaaacaacttagagcaattattagcagaaacgcaaacatggggtaaaagcagtgaaaacttgcctggtttcctagaagatctagctctaaaaagttccgcagatgaaacagcaagttcccatgatcgcctcaagctcatgacgatccataacagtaaagggttagagtttcctgtagtctttctggtaggtcttgaagaaaaccttcttcctcatgccaactcgaaaggtatgcatgaaaatattgaagaggaacggagattgtgttacgtgggaattacccgagcacaagagtatctctatttatctcgagcaaaaacccgattcctttggggaacggaacgcaccatggtccccagcagatttattagcgagcttccgagggctcttttaaaatttgtttaa

SEQ ID NO: 120 - DNA, 1950 bp, Chlamydia trachomatis
atgcgcgtagatgtggataaatatctatttattggacgtgagaagtctgaatttttctctgcatgtcgagagattggggctgtcgaatttttatcaaaaagtaaactcaaagattcagaaaaagtacgaaagctttctgagggattaaaggttttaaatctgctaactaagagctgttccccagcagatttagtatcaacgaagtcaggctatttggtaacagagcagttgcttcaagagatctttgatcttaatcaggagattacaactcttacagagtctttaaaagctctaggtaaagaaatagttcgagttaagcctcttggagatttttcatccgaagaaatccgagagttgacgttaaaaacaggtttagcagttcgatttctttataagagacatatagaaggagctcctttagaagtcgaagaagagaatgttttctacttagcaacagcgtacaactatgattactatgctgtgattgggatagtttctctgtctaaggatatctttactgagatagaggcgccgcgttctgtcaatgaattaagagaggaggaggggcatcttcaagctcttttacgtaaaaagaaggctcgagtttgtgagttatatgcttatcgagaggatcttttagaggctttatgtgaacaatgcaatgaacaaagtttacagcatgcagaagttagtgcagaagatctgtttgatgataaggtttttagcgctttaggctgggtgattgtagatcgtttagatgaagtgaagaaactttgtgatagtttaggcatttatctagaacgggtgcagcctgatccagatgaggtgattcctacatatttagagaatcatggtctgggagcattaggggagtctttggttaatatttatgatactcctgcatcaacggataaagacccttcattatgggtctttttctcttttttcgtttttttctcgatgattattaatgacgcaggatatggattagtttttttagcaacatcgttatttttatcatttaaagctcggaaacagattaagcgctctatagcattaaaacgctttctgcagatgtttatgatattagggcttggatgtgtgtgttggggaggagctacaacatccttttttggcgtttctgtgagttacaccagccctttccgggagtattcactaacacattttctggctttgaagaaagcggagtattatctgaaggagcgtcctaagggttataaggaattggtgcatgattatcctattcttaaagagaagaaaactcctaaagagtttcttctagcccaaagtactagtagcggggactcagtatataaagccgttgtttacgataagtttatagataatattttaatggaaatagctttgctagtgggggtagtacatctgtcattgggaatgctacgctattgtagacagcggtattcttctataggatgggtaatctttatgtgcggagcatatatgtatctgcctatctatttgcaagccgtatctttaattcattatgctctacatattccttatgaattaggagggttggtggggtattatgtagcgtttattggcttaggggttgccattttaggtggggtaatccaaaggggattaagaggattagatgagataacagctgttatccaagtgttttcagacgttttatcctatttgcggttgtacgctctgagtttagctggagcgatggttgggaatactgtgatggttatgagtgaaagattttcccccgcagtaggaattctaattattatcttcgggcacacggtaaatattgcgctttccattatgggaggtgtgatccatggtttacgccttaatttcatagagtggtatcactatagcttcgatggtggtggaaagcttttgcatccattgaaaaaagtaatttgtcaaaagtcgcaaaatctttaa

SEQ ID NO: 121 - DNA, 876 bp, Chlamydia trachomatis
atgagaatgcaaattccaagaagtgttggcacacatgatggttcttttcacgcagatgaagtgacggcttgcgctttattgatcatgtttgatcttgttgatgagaacaaaatcgttcgtaccagagatccccaaaagttggcgcaatgtgagtacgtctgtgatgtaggaggccgctactctacagagcataagcgtttcgatcaccaccaagtttcttacacaggatcttggagtagcgcagggatggttctggattatttgcatggtctgggatttctttctcacgatgagtacgagtatctgaataacacgttagtacatggtgtagatgagcaagataatggacgttttttttctaaagaaggcttctgttctttctctgatattatcaaaatctacaatcctttggaagaaggaggaaatacagacaaagaatttttctttgctttacggtttgcaatcgatttactaactcgtttgcgagagaagttctgttatgatcgtgtgtgtcgtgatattgtgaaacaagtcatggaaaaggaaagtgtttgtttgcgatttgatcgtccgttagcatggcaggagaatttcttctctttgggaggagaaagtcatccggcagcttttgtcagtttcccttgctcggatcaatggattttgcgaggcattccacctactttggatcgccggatggaagtccggattcctttccctgaggagtgggcaggattgcttggagatcagttggttcaagctacgggaattcctggtgctattttttgtcacaaaggactatttctttctgtatgggatagtcaagagagttgtgaagaagcactgaatttagtgttaaaacagcagagattagtatga

SEQ ID NO: 122 - DNA, 671 bp, Chlamydia trachomatis
atgcctctgtacttaggcctcaatccatcctctgctaaacaatattctgctgtttttcagcctattattcgcatagtccctttttcaaaacacgttcctcaaatacgacatgctaggaagttccttgtatcaacttcccatattttgttaacaagcccctcttctaccgcttgttttttttcttccattaggaaaagaatccctttatccctgttcaggaataaatccttcgttaccataggagaagccacttcttctcgtgtaaaggcctttctccccaaagctaatattgtgcaaagcgcattccctcaagcagaagcatttcttcccatcctatcgactcttccttccagctcctccctactctatcctcactctgtactcgctcgtcccattctccgctctcttctacaaacactccctttctcctccttttcctatcctcattacacagttaaacccattacactttccgtaagtacctttcttccccataaaactattattctcactagcccttctatcgttcgtgcctatgcacaactatttccctgcctccctgataaagaacactggtgtctaggagagattagtgcagcagaattcaaacagatatttcacactcctccttccaaaattcttttttcaactaaagaaccgacaggatgttg

SEQ ID NO: 123 - DNA, 529 bp, Chlamydia trachomatis
atgaccgaaatcccttcttcttttgttcttccagatcctgaatggatatatcgtgtaggtattggtcaggacagtcaccgttttctccctgacgaagatcccaagccctgcatattaggtggcattatttttgaaaatactccaggattcgaagctaattcggatggagatgttgtcttccatgctatatgtaatgcgttttcgtctgtaacgcacaaaggtattttgggaggactggctgacgagcttttgaaaacaaaaggcattaccgatagcgtagtctatctacaagaagctgtcgcctctttaaaacctacacaacgagtctcgcatctagctattactatagaagggaagcgtcctaaactgctccctcagttaccgagtatgcgcaagagaatagccgaggttcttcacattcctctagattccattaacattacagccacttctggagaaggcttaactgccatgggacaaggatatggtgttcaatgcttttgtgttttaactattatggaatattgtc

SEQ ID NO: 124 - DNA, 2373 bp, Chlamydia trachomatis; identical to SEQ ID NO: 102

SEQ ID NO: 125 - DNA, 1866 bp, Chlamydia trachomatis
atgcgcagatctgtttgttacgttactccttcagttgctagggctggtcaaatttctacctggcgtttcgaatattcttcagctaatttccttcccgaaggcacattgctaaaatttgacctgggaatagacggacgccctatagactgggagattccttctacagatctttctcaaccatgtaatacaatttatttagaaacgccttccgaggatattgtggctgcaaaagctgtgtatgctcccggaggctatatccctactttcgaatttactctcccttgtgatgtggaagctggggacactttctctattattcttggctcctctcccaacttccctcaagaggactcttcaggtaatggtgctcaattatttactcaacgccgtaaacctttctctctttatgttgacccatcagggaaaggatcttttgaagatcccgatatcttcacaatggatatcagaggaaatgtattaaaaaatatccggatttttgctccttcttatgtgatcaaaaacaaacgctttgatattacagtgcgcttcgaagatgaatttgggaacttaaccaatttctccccagaagagacccatatcgagctttcgtacgaacatcttcgcgaaaacctcaattggcaattgttcatccctgaaacaggctttgtgatccttccaaacctgtatttcaatgaaccaggtatttatcgtattcaactacgcaatcaagcaacaaaagaggtctttacatcagcgcctatcaaatgttttgcagaaacctcctctcatcttttgtgggggcttctacatggagaatctgaacgtgtcgactctgaaggtaatatcgagtcttgcttgcgttattttcgtgatgactgcgcgttaaacttttttgcaacatcctctttcgaaattcaagatggcctgaccccagaaaccattaaaaccattaaccaaaccgttgctgattttaatgaagaagatcgtttcattgccttatccggagcacagtacctttctgaagagcctggcgagggaattcgtgaagtattgctgatgaaggaacccaaatccccagggaaacataaagaatgcaaactatttcctttatctaagctatataagcaatcaactagtcatgagttaatctcaatccccagcttcactgcttcaaagaaatttggatacaattttaataatttccatcctgaatttgaaagagttgttgaaatttataatgcctggggatgctctgaaagaactgaagctgaaggaaaccctttccctattaaaggttctatcgactcagaaaatccagagggaactgttctatctgctttaaagagaaacctgcgttttggattcgtagccggtggtcttgatgatagaaatctatacaatcacttttttgattccgatcaacagcaatactcccctggattaacagctgtgatctgcaataaatattctcgggattccttactcgaggcattataccaacgacaatgctatgctacaaccggccaaagaattatcgtgaatttccagattacatctgctcctatgggctccgaactctccacagccattaaaccagggctcgtgatcaatagacatatttcgggatatgtagcaggaactgccaagattgcgtcgatcgaaatcatccgcaatgaggatattctccataccttccacccagatggaaataactttgagtatgagtacgacgatctctctccttttgcacaagtcactctaaaagatcctcaaaatggagctccttttgctttttactacttacgagtcactcaagagaatggagctatggcttggagctctcctatttggatagatcttaactaa

SEQ ID NO: 126 - DNA, 921 bp, Chlamydia trachomatis
atgcgattgattgttcgaattttcttctttctctgcattcttagcgctccctatagcttcgccagtacttgttccgcagcacaaggcgcctcttcttgccttccgtgcgcgaccgctcctcagcaagaacttccagcatctccagaaaaaactgaaaagccttttcgttgtccatcttatcgaccaacggtagaagctcaacacctgctacctcctcaagaagatctttcctccggagccttttcagaaacgtatccagatttgactacgcaagccgttatccttctatttcttgcgttatctcctttcctggttatgctcctgacctcctatttaaaaatcattattacactagttctccttagaaatgctttgggagtacaacaaacacctcctagccaagtcctcaatgggattgctctcattctatcgatctacgttatgttccctacaggagtggctatgtataacgatgctaaaaaaggtatcgagtcgagtgctgtccctcgagatcttttttctgcagaaggagctgaaacagtatttgttgctttgaacaaatcgaaagaacctctgcgatctttccttattaaaaatacacctaaacctcagatccaaagcttttacaaaatttcacaaaagactttccctccagaacttcgccaacaattaacgccttctgacttcatgatcatcattcctgctttcatcatgggacagattaaaaatgcttttgagatcggagtcttgatttacctccctttctttgtcatcgatttggtcacggcaaacgtcctagttgctatgcaaatgatgatgctttcccctctatccatctctctccctcttaaactgctccttgtagtgatggtagacggatggaccttgctactagaagggttgatgattagctttaaataa

SEQ ID NO: 127 - DNA, 1041 bp, Chlamydia trachomatis
atgcaagcagatattttagatggaaaacagaaacgcgttaatctaaatagcaagcgtctagtgaactgcaaccaggtcgatgtcaaccaacttgttcctattaagtacaaatgggcttgggaacattatttgaatggctgcgcaaataactggctccctacagagatccccatggggaaagacatcgaattatggaagtcggatcgtctttctgaagatgagcggcgagtcattcttttgaatttaggttttttcagcaccgcagagagcttggttgggaataatattgttctagcaatttttaaacatgtaactaatccggaagcgagacaatatcttttaagacaagcttttgaagaagcggttcacacgcacacatttttgtatatttgtgagtcactcggattagacgagaaagaaattttcaatgcctataacgagcgtgctgcgattaaggccaaagatgatttccagatggaaatcactggcaaggtattagatcctaattttcgcacggactctgttgagggtctacaggagtttgttaaaaacttagtaggatactacatcattatggaagggattttcttctatagtgggtttgtgatgatcctttccttccacagacaaaataagatgattggtattggagaacaatatcaatacatcttaagagatgagacaatccacttgaactttggtattgatttgatcaacgggataaaagaagagaacccggagatttggactccagagttacagcaagaaattgtcgaattaattaagcgagctgtcgatttagaaattgagtatgcgcaagactgtctccctagagggattttgggattgagagcttcgatgttcatcgattatgtgcagcatattgcagaccgtcgtttggaaagaatcggattaaaacctatttatcatacgaaaaacccattcccttggatgagcgaaacaatagaccttaataaagagaaaaacttctttgaaacaagggttatagaatatcaacatgcagcaagcttaacttggtag

SEQ ID NO: 128 - DNA, 732 bp, Chlamydia muridarum
atgctccgcgtcttagcctatcgacaggttgctttttctaagtttccttatgccctcagatcgtttttagattttttgcactctctcaagcagcattattcgtttattctgcccggagatcctctccctaataggaaagcaatcatactgacattcgattatgcttctgttgatttttacaagcatgtctttccttttcttcagaaattccagattcctgcggttgttggagttgcttggagatacgtatcccgtttagaaagtgaaaatcttcccattgatatgcgaatttcgccatcggattttctagcttttcaagacgaaatattttcttattatcagcccttttgttcggttaaagaactttgtcatatggctcaaagttcgattattcgatttgcctcttcagggtttgctattagaaaccttaaaaactcccctccttatttacatactgaaatttcattgtctaaaattttactagaagaggctatacaggctcctgtggaagtctttttttatccttttggaaaaagtgatttggtgagtcaacattttgttcaggaggtttaccgctattcttttgtgttaggggatgctgcgagttttttttactcgatgcaatcacagcatagcattcctcgtattgatatggctttagatagtcgagggatcccatctctttatcagctctctttccgccaattaaaacgattccttattttgcattaa

SEQ ID NO: 129 - DNA, 162 bp, Chlamydia muridarum
atgccgatctccttacctacttctgaagtctgttctacggaaacagcagagcttactcctgagggaaaagagcgtaatctaacgatcatttccggacaggtagtcatttatccagaggacccttttgcaattccttcagttgatccatgctccgcgtcttag

SEQ ID NO: 130 - DNA, 723 bp, Chlamydia muridarum
atgacagaacctcttacagatcaacaaataatacttgggacatcgacaactcctgtcgcagcaaaaataacagcttctgaagggatatccttaacaataacaaacaatgctcaagctaactcttcagtaaatattggattagatgctgaaaaagcgtaccaacttattttagataagcttggcgaccaaatctttgatggaatcacaggatccatagttgagagtgctgtacaggacattatagataagattacctcggacccttctctaggattgttgaaggctttctataacttccaaatcactgggaaaattcaatgtaacggcctattcacatctagcaatgtaacaactttattaggaggaacagaaataggtagatttacagtaactcctagaagttctggaagcatgtttttagtttctgcagatatcattgcatcaagaatggaaggtggagttgtattagccttagtaaaagaaggagatacacaaccatgtgcgattagctatggctattcttctggtgtgcccaatttatgtagcttaaaaacctgtgttactaattccggatcgacacccacaacttattcattacgaataggaggattagagagcggagttgtatgggttaatgctctatccaatggtaatgatattcttggaataacaaatacttctaacgtttcttttttggaggtgatacctcaaaaaaacacttaa

SEQ ID NO: 131 - DNA, 309 bp, Chlamydia muridarum
atgcaaaacaaaagaaaactgagaaacgattttattaaaattgttaaagatgtagaaaaggatttccccgagctagacttgaaaatacgggtgaataaggaaagggttacttttttaaattcacccttagaactctaccacaagagtatttcattaattttaggcttgttacaacaaatagaaaagtctttggaattatttccagattcccccgttcttgaaaaattagaggataacagtttaaagctaaaaaaagcgttgattatgcttattctgtctagaaaagacatgttttctaaggcagaatag

SEQ ID NO: 132 - DNA, 828 bp, Chlamydia muridarum
atgatggattacgaattattagatagcggggatggtaagaaacttgagcgatttaaggatgttgttttaattcgtccttctgctacggctatttggccaaaaacttcaccagctttgtggaaacaatattctgctgagtttgttcgtgttggggagaaaggagagtggaaataccgcaattcacatcttaaggaatggtggataactattgattctgtgtcatgcttgttgaagcttactccctttggtcatgtggggattttccctgagcatgctacattctggcaagatctaagcccctctttatcgaagccttcttgtaaggtcttgaatttatttgcttatactggagcttcttctattttttgtgcccagcagggggctgctgtctatcatgtagatgcttctaaggctgctgtaaaatgggctcaaaaaaatgtggaagggaattctttttcagaaaaaagaattttttggatcatagaagatgttttttcatttctaaaaaaagaaattcgtaggggaaaaacctatgatgtgattcttttggaccccccaacttatggacgaggtcctgatggagaaacctttaaaatagatcgggattttttcccattattggaggcctgttcacagctgctttctagttctttttcgcatatgttgattacttcgcatacgccaggacatacccctgaatttttgcattgcttagctaaaagggcgcttcctatgctttcttcccaaaaatggcggttgggagagaatttttgtggtgagggagatcaacggttgccttcgggagtttttgcgcaatggagtttatag

SEQ ID NO: 133 - DNA, 597 bp, Chlamydia muridarum
atgttttcaggcattattcaagaggtagcacgtgtagaccttattcatcattacggggattccatggagataggaatttttgcacgtaatctagtagatggggttccgggaagtagtatcgctgttgatgggatctgtttgactttagtcaaaagagaattcgaattactattttttgatgtgacagaagagaccatggcttgtaccacgattaaaaattacaccgtggggagtatggtgaatttagagcgttctgttcgattaggggatgaaataggtgggcattttgtttctggtcatgtctgtggtgtgggtaccatcattgccgtagaaaaatcttatatgttttttaaggctccaacaaatttggtgccttatgtactcgaaaaaggctttatcgctattgatggtattagtttgacgatcgctcaggtaaggggagatatcttttccgtcagcgtgattcctgaaactcgagctcggacttctttgggttacaaacaggttggttctcacgtaaatatggagcccgatatgatgacaaaaatgcaggtggatacagttatgcggttccaggccgaaaagataggcaaatga

SEQ ID NO: 134 - DNA, 9678 bp, Chlamydia muridarum
ttggaacaagcattgtgtactatgactctttcgaatacagcctcttccacagtttcacctaagtctacgcaacttaaccatactttatccaatcaaactcaccctgttgctcaaagcaataggaatatagatttccaaatctatgatgaacagattcataataatcattctacagaagatgtagttgctataggacgtcgtattcagcaagaatatgcgaatcttaccacatcaaaacaggttaatttctcggcatctccatctaaccatactggaaactggaagatttctttgctttataatttatcgatgctggttgcaaatctattccctacaactattcagcctgtgcaacctcaaaaagtcatctttaagaatcaagactcttctaaggaaacccaactgaccaaatcatccaaagtagtttcttctcgctcttctttaaaagagattttttcgaagaaatctcctcctttgagaaagtctttttcttcctctaaaatttctagagcaatccctagaagaaaaaaacgatcaaacgatgatccagttcaagccacaaatacttatgatttgaccgctgaaaatattcttgaaaaactctctttaacacaagaacaacaaattaagcacgataatctgattagcaatctcaaagaagcaattaatcgatatagtgatcttaatcggaaaaattcccggaaaggacaatcccttcttgttaggcaagccaaaattctagatgaaattctttcacagactaaatctacagaggaacgagcttccaattctgtaatgacgactattaagaaggaatttacctcccaccgcgtacctgtagagaaaaatattcacggaatttggattgcaggttcaccgccagaagggaccgatgaatatattaaattgttccttcacacttatccagagttttccttccttttttgggtggataaaacagcttatggcgcagcaaaattctcttctactcttaaacgaattgcttttgatgcagcagttaattccttacgagaggctactccagagcctgtcaagcagtttgttcaacgttatgataagctaaaaaaatcatacgatacttcgcgagattttgatgaaaaacagcggttatctgagcagttagttgagctatacgataattacaataaatttagtaaggagattcaaagtaactttgatgttttgctattgcatgaaatgatcactattcaagatagcttttttaactactgccagctcaaaggagtaggtgcaataacagatgagacacgtatagagtatcttgagaaagttttgaaagtaaaagaagaggatctatctcactataaagaaactatcaaacgcaacaaagagtctattgaaaaacttgtaaaagagattaatgactctactggacgggagagagtagttatcaaagacatccgagatttgaagtctttacaagatcttactaatagctacaactatgaaacagaaatgttactccgatggaattatgcagcggctaccgaccagcttcgtatgtacatgcttaaagaatatggggggatttatacagacctagatattatgccacaatactctcaggatgttttgcaaaaaattatggatgttggtgggagtaggttttttgagcatgataagttacggcggactctttcttttgcagctctaaaattaggaagcggtaagcaaacaactgtttcctttgaagaggctaaaaaagctatgactctccccacatttactcttcaggataagagtcaaatttctgaaatttttaaatatctagaaacggaaacgcaagcaaaaaaatcacttttccaaccgatggatgttaccgtcgtccgtgatttcatgcctattttacaacgttatcataagtggcaaacaggatggaatgttcggggattaaatggtttaatgatggctcataaagatagtgctgttgtcgatgctgttattgctcgccagcgagcggcctatgatgagatgcgtgcacttcgtcagaatgttgttagtggagaattttttcgttctcttggagatcttgaacatgtaaatcgtgaaaagaatatcggcggctatttagcgaaaaattatcttgggggcagtctttttttcgattttcgtcaagattcagtgattccaggagctattagtaccttaggaatttcaggtcccgatatcatcatggataccatgagcgattattttacaaatcttggaccagttggagaagattttttatatgaagggaaattaggaaaagcagctttccttggagcttatcaagcacaaaaaactccgaaaggagaacttacctatgattggctccatcctttatccattggagctaatgacgttactcctgctgatgcaagcacctggtgtgaaactcgacagcattgtgccgctgagcttcttttatcggactctatatcctcagacgaacaccctaagggtattcgtcgagaaagagtaaacccgaatgatttctctaaattatggagtaaggaagctcagggaatcctttctagtgatttcgcggatcttttaccacgctttaacttactgatagaatcatctgcgttagatattcatactttatcggctcttgatcgagatatccaacacctttttacaaaagttcaaaaagaccctgtagcatctgtagctgtattttctttacagttacaattagctgaaatgattcgtgcgattcctttccctatacgtaatcaggtacatatcttgcctgaagcacaagcacatttcgaagcagattggaagaaagcaatccaattatatcttcacagtcatccccaaacagaagtggttatttggtacagttctactcatactcaaattgtttttggaaaagatttgcttgctgttgcagaacgagttgcagcagcaaaatccttaatgtctgatcatgatccatcacttataaccagttatctaaaatataaaactcaatctcatcttggggtgcttactgagtttgatcaagaagatttctttgaactgatggtagatattgcagaggaaccagagcttcataaacaactattgaaaatagaagaacaagttaattcaggactctactctcatgtagagcattctttaggagagtggttaaaattatccaaagaagaaagaaaatcaaagtttcttaagattttaaaagaaacgtttcaagaggaggaaagagaagactctcaacaacaacataaaacatggtttgaagaactctatgagaaaagacatcaggagcgcgtaaaggatccagcaaaaaaaatccaagaactaatcacagtttttcaagaaagtcagcgcgttcaagctcaagacatagatacttattttgctcataaaccgttttatcaagatctgatgaaagatggttacgcttttgaggacatttctgtgattacgaagtatcttttagcgagtgacggtgtttcagggattattactactgatcctatatttcctcctccatctaaacagctaattgacgctatgaaacaatctttgggagaagattttggtgagctacactatacgttacagatggtctatgattggttatctaaagagactaacagcgtaacttctgaacaggcaaaacaaaaattaccacagaaactacatgaaaaactagaaggttatacaacacatgatttgcttattccgcctattgatggaagcgtcagcgctttaggattgcgattttccacggaggaggggaaggtatccgatcgcgttttaacaagcatagcacctggagttcctaattctgctagctatgctatgactagttatttgtatggtttgttcttaattacgaaagatatccaaagtggtcgattaacccatgaaatactaaaagagagattacaaacctatggaggcgcctattttatcaatgaatcaaaaattgatgtgctacttgctttatcccggaaaaaagctcagatatccctaatagacgcccataaggctcttacaggtttttcttcttttagcgaagcatctctggctttacttacaggaaggatgcccggaacaagcagagttctatcaagagaagtagagtttggacgaccttcagctatcgttatggaaggagcaacagctattcgtgctcagagttacgatgctgttggattaaggaaagattttttcttgttacctcatacagttccttccatacagtctattgttgagcaagcgaaatatacagtattatcctggccagaattttatgaaaatcatgcggataaatggaacgatcttgctaatcgttttggagctgaggaccttagtgttcatccgcaaacatttttatatgacacagaaggtcgctgtatggggcttgctttactgtatatgttagcagatgactctgtatcctacagattattacaacaaaatctgatgactttagcttcgttatttgatgagcaaaacagaagaaatatccctttaactccagctgatcagaaatttttaaataaggggctttctttaattgagtggttacagttcaaaggaaatcaacaacttcaaacagagggatttttccataccttagactgggatatccctcaattaatgaagcactttgcttcttcaactgtaaaaagttggttaattactacgcctgcgcatagtcttgttctttctttaatggggaatttttttagagtaacagatccaaactatggccatacagactttccttcattagaagctgctattacttttttagaaaggatggttcaagtatccccagctgttttggagcgttatggatttgataaggaaaagtctgtaactagccaactcaaagtccactctttagagacttcagaattgcaaaatgcagtttttgcttcctcagatcttggctttactagtcgctattttacgacaacgttagaagagatgaccgtcagaggccctattacgatgaatcaacgtcacacggattgggccactctttataagattggggggactgttcaaggtaaacgtattgatagtagaacacgggaatcggatttaaattttttaaaaattaatggggatattctagaggaatttttaacaagaactgttctagatagtgacttggtggagttaatacaatctctgctcaaaacacatggccttgagccaggcactacccttattagtccaagttctattgtagagaccgcaatagaccacgtctcgctattacaagcggtgaaaacaaaaacctctcgaatgcacactattttacaaagcctaggagaaagaatttttaaattgtttaaaaattctggggtacaagactcagacaaaatttctatagacagagtccaactagttgatgaatctgatagcgcaacgatagacttcacagtgattaaagataaacagcgttctcagaaaaaaagtattactgttggcatagaaagtttagctggatcctttagaaaatttagcgcatcgatgcacgaagttattggaactggagtgcttgatttagacttaggaatgactgtcgtctctttagttcagtatgtccgtttagtagaagcaggacaaggaaaagatgctttagcagttgctaatttagtgatgaatcttaaaatagctctcgaggtctctatagggaatgtaattcaggctctgggtaaaaaactgctcactcaggaggggcttaatactttccgtttagaaacagaattagctcgtcaactacacaaagtgggagcacgagttggaggaactgttggtaagacgttaaccagagtagcgcatgtattagaactccccatattagaaagtgcaattggaacatggaacctctataatagtgtgaacgagttattacatgctgattcttggagtgaccaggttgctgctagagtacaagtagcttttgattcaatctctttaggaatcactatagcttcggttgtttctccagcacttatgatggcagcaggccccattgctgccatagggatgggggcagcatccattgctcgtaatgttgccagaaaagaagagcggcactctcaatggctcaagtacaagagttttcttgataatggatcaaaacatacagttgccgcattccctcataaagggcttatagatttatctgagaatttagtacttgggaacattgtgctgaatttaagggtttacccacctctgcttacaggcgatcgctcttataatgcaaatcggtggtttgggaataagcctggatggtctgattggcaagttcgagaaagattaggttatgcctatcgaagcagtccatcttatgcgttagccagaggacacgctaatagtttttggccactatctatgcctagtattgataaaggtgtatatcgcacagtaattctaggatatggcattcaatataaagcggttacagaagttgtctacttgtctaatgaaatggtttggagagaagctgtgatgcagtttgactctcgatactatgtagagccattaacagcggaaaaggagtgtgcaacagtactagcaggagatacgccactatcagtgattcctgtacgtttattagaagaggagtctctggaacgcgagaagaacgcggcttcttataagaactatcagatcattattgagggagggaaaggaggacttactgtacagataggaggagcaggttattacaagttaacagcacagccaggaaaagggaatatcctctctttccgagcaatacctggatatctttctgtaacgtttgatcttagccgattggaacaagaggtgcctctcaccaaacaaaatggaacagccttaaagatacttaaagtacgtcaaactgggtttgatactattgttggatcttcaggaggtagtgataatttaacaggcaatcacaatacaaaattttatctaagcaccggaggaggacatgtcacctcaggatccggtaagaactggtataacattccatctttaacacgtagccttggttttgattttacttctaatgctatagagcataaggcttctgtagagatgcttttagcagatttctctcctgctaataatttaagtttaatagttgaacgatttgcaaatgttagcattcatgtttttaattatagcaataaaacagccccttatactgtgaatttgaaagacggggtgtctttacaagcttctaagcaaggctttagcggtagtttattagaggtggcttcctttgatcaaacgctatggcaaaagagctatccagaagatagaggatttgtagaagatattttatcttggttattaaaactgcaatggtcgttagcttctcgagtaaggatctttttacagggaggcacagcacaatacgaaacaacgcagaaaagtcttatttatagacctgatccacactcaagtatatctattcaggctacagattcttataatacacaagtttatggtaacgttggctgttcttatattctattctcttcaccaggtgtgaccgccaaaacactcgatattttgttattcgaagattcggggttaccacaaattattgacttaagcacacttgttccaacttctatccaagggttcctctatccaggagggtttataaactttcaagtctcttctgcaagatatgcatttcctttttctgtcagttggagaaggcatcattacactttccctgctcaaacgattattcaagttctacctcgcttgcggtttgaactaggggattggtttacaattttgcaaaagagtgtaggaaaatgggtaacattgtaccaacacgacatgattatccctgaacgtattgaaggtgttttaagtctaaataacacagcgacattaatgtcacaccacaatcaaacaacccatatgcttggagtagaaaacaggggagatttgagtctaaaagttttaggaatactccaatctggtcaaattaaaggatctaaatcggaaaatagcgcgagtggtcatttttctaaattgttatctgtcccagcacatacgatcaaaaatctttcgtttaaagaggaagagggaactaagagtaaaaatattttattctacagtgtgctggaggaaacattccttaaagctacagataaacctttaactattattaaacgtgataaatggtctctgtatgatgaaatccaagtatttgcaacaactcttaatctgcaaaatttcttgcgttatcatatttcggaagagactccaatgttatctagactgctcatgtatgcgcagaaacgcgtttccatccaaaatagagatctcgcattgaaatttttctatgttcgagagcgaagtggtattggtgcaattagattggtattcaagaactttttcgaagagagcttgcaggggatattacatggaaccttagagcgagaagttaaacctatgctagctgaaaatccaaatacactaattcattcatcgtatcggaatcatttagaactcatcctaggggaggaaaccttagatctcgcaattatcgttcaagagtttagctcttcaaaaaatattgttgagttgcaaaaagatctagcaacccatcggcttatttatcctcaaggaaaagaagccttgtctcttgcaatctatacccatactttcagtaaagagagcttagtcacatctaaacaacgaagcgaagggttaaaattcatcgatccatcaatgcaggaatatcgtttacctgagacaacgattctcgaaaactcttattatttagacccttccacaggagatttgtatttaacaaagataattgcgcaaccttcacaaagtagagcatttcttatcaaatttaaaaaatacaaacgccactggctagatttccaaaaattagttctttctggatctcatttagaacttatcgcatctactggaacagctttaacatttgtaggccccgaattacggcatttagagattgacttcccaggcgcgttaaatgggactattgccaaagaacacatctcatctcgggcaagtattgtaatcccaacaaacaatcaggtgacacactatgacccaagaatagcgaagcagcattatagctctatcaattatatgctgtgggatctgcgtgatcgtgcgacactatctaagcgagcaaaaactttagatagctatttgttagaagtttgtatgaacttagactcttctaatccaacgtggaatattccaaatgatgtgttgaactacgcggtaggatattatcgaacaatccttcctagctgggtaaaaaataaaatccgtgtaggttccttaattaaagtagaagctgatactgcaaaaactttgagtttctcactaattacgacacaaaatgatctattccaacctgtagtggataaaggattctatatttactacagcgtttcgaaactagccaatcatgttaaactgcgaaatgttactggagatatgttgcttgatatcgataaggaaaccacatttattgttcgaggagtagatgaatctgattatgataagaaacaaatctatgttgttttagatctgacaacagaagaagaacgtaaattgcgaacggataagaacatcattattattcctggaggagaacgattgaaccgatga

SEQ ID NO: 135 - DNA, 795 bp, Chlamydia muridarum
atgcgctgttctgcttattgctctgcttcgtcctatcatctacacgttctttttcatcttttaaaggcacgctacccttctgtcttatcccgagaatacgtgctcatttcctctgaagagctgaatgaaagcgacaaagccgctgtctttttcccatttggagttagtgtgttttggggatgggaagaagctgaagaacttcaagtgatccaagctatcacctcagcatcagtggatcctctccctaacccagaaattgatagttatgacttccattatggagagaaactacagatccgtcgcgatagacttgttctcaccaactctaatttaaacacaaaacttgctatttcctttgggttagcccaatctatcaaacttaccgtctttgaagaaacgatttacaaaacagttgaaaactctaaatctttgcctcaagaacttgcctctaaagggaaaatttctctatcccgtaaaacgatcgcaaaaaagattggagagcttttcctggataaagcttccgtaaacctacactctgacattctcgatgagcccgactttttctgggaacatccagaaacccaacctttttatattgatgtgttgacctgcctagatgtaaacgcgcgagtaaatgtattaaatcacagacttgctatcctaggagatgttctcgagattttaaatgaccaacttaatcatcagcattcttcagctctagagtggaccgtgatctggttaattgcactagaggtattggtcaccttacttaaagacgtattcaacatcatctaa

SEQ ID NO: 136 - DNA, 2961 bp, Chlamydia muridarum
gtgccacaagcgcttataataaaggaacttatgaatcaagtcataaaaaccattgctctttgctatcaaaaatatatctctagagcatctaacaaaaccttctcaatacataatactctttccctatctcttcttcccaagtgtctacttggatccctaatcatctatacctcacatgcatttggagaaatggaattagccatttctgggcataaatatggcaaagacagagatgcttttactatgatctcctcatgcccagaagggactaattacatgattaaccgtaagctcattctgagcgaattttcttcgctagataaattctcatcaggaggagcttttaagaatcttgcaggaaaaatagcattcttaggaaaacactcctcttcctcgatccacttcaaacacctcaacattaacggattcggatccggaatcttttccgaatctgctatcgaattttctgatttacgcaaacttgttgcttttggctctgaaagcactggaggcatttttacagcaagagatgacatctcatttaaaaataatcactacatcgctttccgcaacaatatagccaaaggaaatggcggagtaattttacttcaaggagatgaaagaggaactgtatcttttacagatcaacaaggagctattatttttgccaataaccaagctttagtctctccatctataaaacatagtgggcgtggaggagctattagcggagacttcgctggatctcgaattatttttctcaataatcaacagatcacatttgaagaaaatagtgctgttcacggaggagctatctacaataaaaatggggtggtagaatttcttggcaatggaggcactctttcttttaaagagaacagcacaagagccaatggaggtgctatatacactggtaaattcaaagcaaatcaacaaacggctcctattatattttcacaaaataacgctaatcaaaaaggcggcgctatttacgctcaatacgttaacttagaacaaaatcaggatgctattcgttttgaaaataattctgctaaagaaggcggaggagcaatcagctcatctcaatgcgcaatcacagcccacaatagcatcactttttcaaacaattttgcgggagatcttggaggcggagctatcctattaggagggaaacaaccatcgctatccttggtcgctcacaatggaaatattgcatttattggcaatactatgcttcctgcaacaaaaaaggcctctttacctcgaaataattcgatcttagtcaaagaatctccctataaaattcaattcgcagcgaataaaaatcgctctattattttctttgacccagtaattgctttgtcaccttctacgtctcccgtagagattaattctcctgaacacgaaacgcctttcttctctcctaaaggaactatcgttttctctggagctaatcttatagacgatgccaaagaggatattgcaaatagaacttctattttcaatcagcctgttcttctccataatggaaccctttctattgaaagcggagctcatcttgttgttcaaagcttcaaacaaactggtggacgaattagcttatctccagggtcctcactagctctttatacaaccaatacactctttcatggcaatgtttctagcacagaccctttggaaattaatggtttaagccttggagtagatacttctccttccaacctttattctgaaatccgcgcaggcagcgctcctctcaaattatcagggtctccagatattcatgatcctgaaagactgttttatgaaaatcgggattccgcagcatctccatatcaaatggaaattcttttatcctctgacaaaatcattgatgtttctaactttacaatcgacgctcccgtaccaaataaagaagctgggtttcaaggatcttggcacttcagctggcagcccaatactgtcaataacactaaacacaaggtattgcgtgcttcctggatcccaactggcgaatatatccttgaaccaagccgtgttggaaatgctatccctaattccttatggagcacatttttacttttgcaaacggcttctcataacttaggagatcatttatgcaataactccgatctagtccctacttcatacttagggcttttaattggaggaattggagcagaaatgcgcacttattccgcagaaaaagaaagttttatctcccgttcaggcactacaggaaccaccattatacgcttaactccaaccctcactctatcgggaggtgcgacgcatatgttcggagattcctttgttacaaaattaccagaattcattgcttctgaaggaatggtacaaaatgttggtcttacccaaatcttaggaccattaactgtaaaatccacattatgtgcagccttagatcataatgctatgattcgcctatccgctcaaaaaaatcatacacgtgcaaaatgggacacttttggaatacgtggaacattaggtgcctcttattcattgctagactatgaaaatatggtacgtattttcacatttgctaatgttgaagccacaaatgttttacagaaatcatttacggaaacgggatacaatccaagaagttttgcaagaacaagactcacgaacatcgcagttccggtagggattgggtatgaattctgcttaagcaaccactcttttgctctattagggaaaggacatattggttactctcgagatatcaaacgaaaaaatccagtaacttttgctcaattagctatgaataacttctcctggactgctaatggctgccaagtcccaacctcagcacatacaatagcgaaccaactaatccttcgctataaagcctgctccctatacgtaaatgcagtagctaccaaactagaaagcacatatctctcaagcagcttatcttgcggaggctatgttggtttctaa

SEQ ID NO: 137 - DNA, 2415 bp, Chlamydia muridarum
atggacgcacaagaaaagaaatacgatgcatcagccattaccgttttagaaggattacaagccgttcgtgagcgtcctggaatgtatattggggacacaggcgttactggattgcaccatctggtttacgaagttgttgataacagcatcgacgaggctatggcggggttttgtaccgaggttgttgttcgtatattggaagatggaggcatctctgtctcggataatggtagagggatccctgttcaaattcatgaaaaagaatccgccaagcaaggtcgagagatttctgctttagaagtcgtccttactgtattgcatgccggagggaagtttgacaaagacagctacaaagtttccggagggttgcacggggttggggtgtcatgcgttaatgctttatctgaaaaatttattgctaacgtatttaaggatgggcaagcgtatagcatggagttttcaaaaggggctccactaacagctctacaagtattgggatctacggataaacgagggactgagattcttttttatcccgacccaactgtattctctacatgcgtttttgatcgcgcgattctaatgaagcgtttgcgagagttggcatttttaaatcgtggaataacgattatttttgaagatgatcgtgatgcaggttttgataaagtcaccttcttttacgaggggggaattcaatctttcgtcagctatttaaatcagaacaaagaaagtcttttcccgacccccatttatattcagggatcaaaacgtggagatgatggggatatagagtttgaagctgctttgcagtggaattcaggctattctgaactcatttattcttatgcgaacaatattcctactcggcaaggggggactcatttaacaggattttctacagctttgacaagagccgttaactcctatattaagtctcacaacttagctaaaagcgataaattgtctctgactggagaagatattcgagaggggttggtagcgattgtttcagtcaaagttccaaatcctcagttcgaaggacaaacaaagcagaagttaggaaatagcgacgtaggctctgtagctcagcaaattagtggcgaagtattaacaacatttttcgaggaaaatcctcaaatagccaaaactattgtagacaaagtctttgttgctgcccaagcaagagaagctgctaaaagagctagggagttgactatccgaaaaagtgctttggatagcgcccgtttaccaggaaagttagtggattgtttagaaaaagatcctgaaaaatgtgaaatgtacatcgtagagggagactctgcaggggggtcagctaagcaaggaagagaccgacgattccaggctattctgcctattcggggtaaaattttaaacgtagaaaaagctcgtctacagaaaatttttcagaaccaagaaatcggaagtattatagctgccctaggctgcggtattggaacggataattttaacctgagtaaattgcgttataaacgtgtcattatcatgacagatgctgacgttgatggctctcatatccgaaccttattattgacgttcttctatagacatatgtcagcattgatagagaacggatgtgtttatatcgctcagcctccattatatcgggtaagtaagaagaaagatttccgctatatcctctctgaaaaagagatggatggctatcttttaaatttaggaacacgagaaagccgagtggttttcgatgatacggcaagagaactagagggaacagctttagaatcgtttgttaaccttattttggaagtggaaagctttattattgctctagagaagagagcgattccattttctgagtttttagatatgttccgtgatggggcgtaccccttatattattaccctccagaaagtggaaaacagggaggaaattatctttattcgaatgaagaaaaagaggcagcagttgccgctaatgaagaagctgcggcaagaatcatagagttatataaggcttcagcctttgaggagattcagcataatttgaatgagtacggctatgatattcgccactatcttcatccaaaagggacgggaatcacggtttctacagaagattccaaaactcctaattacacttgttatactttgaaagaagttatagattacttgaagggccttggacgaaagggaattgagattcagcgatataagggacttggagagatgaatgctgaccagctgtgggataccacaatgaatcctgaacaaagaacgcttgttcgcgtctctttgaaagatgctgtggaggcggatcatatctttacaatgttaatgggagaagaagtccctccgcgacgtgaatttattgaaagccatgcgctatccattcggatgagtaatttagatatttag

SEQ ID NO: 138 - DNA, 2379 bp, Chlamydia muridarum
atgcttggaatacgaaaaaaaacgattctgcaactcgctattttactgttgctccccttttcacaaaacgcctttggaacatctcctgaaggacgtatggtggtacaatccatcactattgagactcgaggagaaaatgctcaaaataagcgggctattcccaaaataaagacaaagcaagggacgctgttctctcaaactgattttgacgaagatctaagaactctctcgaaagatttcgaccgcattgatcctattgtagagtttcgcaatgggcaagctgttatttcccttattctgacagcaaaacccgttatccgacacattaacatctcagggaatgaagcaattcctactcataaaattttaaaaaccctagagctttataaaaacgatctttttgatcgagaggtttttttcaaaaattttgatgctttaagaaccctttacttaaaacgcggatactacgattctcaattgtcctattctcacgatcataatgagcaagaaggttatattgatatctcaatccaaattaaagaagggccctacggacggataaaaaaattaacaatttcaggtattactcgtacagaagcctctgatttgggagacatagttttaaccaaacagtactctacaacaacaagttggttcactggagcaggggtataccatccagacatggtggaccaggatctctttgccataacaaactattttcaaaataaaggttatgcagacgcaaaagtcacaaaagaagtctccacagatgctaaaggaaacctctctcttgttatcaacgtagataaagggcccctgtatactttaggtcacgtacatatagaaggtttcacagctttatccaagcggttacttgataaacaactgttagtgggccccaattccttatattgcccagaaaaagtttgggctggagctcaaaaaatccgtaatgcctacgctagatacggatatgtaaataccaatgtagacgtatcttttgctgttcatccaacgctcccagtttatgatgttacttacagggttagcgaaggagctccttacaaaatcggactaatcaaaatcaaagggaacacacacaccaaacatgacgtaattttacatgagacaagtctgttccctggagatacttttgatagattaaaactagaagatacggaaactcgattacgtaacacaggctactttaaaagtgtgagtgtctacacggtacgctcacaattagatcctttggactcaaataacctttatagagacgttttcattgaagtcaaagaaaccgaaacaggtaacctaggactgttcttaggattcagttctatagaccatctgttcggaggagcagaaatttcagaaagcaattttgatttatttggcgcccgacacttattcaaaaaagggtttaaatccttaagaggtggtggggaatatcttttcttaaaagccaatttaggagacaaagttactgattatactgttaaatggacaaaacctcatttcttaaacaccccatggatcctaggagtagaattagataaatctattaataaggctttatcaaaagattattccgttgatacctatggaggaaacatcagcacgacatacattcttaacgacaaactgaaatatgggatgtattaccgcgggagccaaacgagcctaagtttaagaaaaaaaacagaaaatcctaacaaacctggaccagatctagatagtaacaagggatttgtttccgcggcagggcttaacgttctttacgattctattgacaatcctagaaaacctactatgggtattcggagctctttgaactttgagttgtctggattaggaggaacctatcagtttacaaaattaacagccagcggctctatctatcgcctgctaactaaaaaaggtgttttgaaaattcgtggagaagctaagtttattaaacctttcggtacaaccacagcccaaggaattcctgttagtgagcgcttcttcttaggaggagaatctaccgttcgtggatataaaccatttattattgggcctaaattttctccaacagagcctcaagggggcttatcttccttattgctaacagaagagtttcaatatcctctaatttctcaacccagcatcaatgcattcgtttttctagattcgggctttattggcacggaagagtacactattcgactcaaaaatctctgtagcagtgccggatttgggttgcgttttgatatgatgaataacgtgcccattatgttaggttggggttggcctttccgcccaacagaaattcttaataatgaaaaaattgatgtatctcaaagattcttcttcgctttgggaggagtgttctag

SEQ ID NO: 139 - DNA, 1305 bp, Chlamydia muridarum
atgaatgataaaacgataattcataaagaatctgttgcttttgcggcaatccctcctacccagcctgcagctcctgttaccaaaatgttgttggaatgtactcagacagggggaaatactggtgttgatgggatggtcaccacagcttatgttaatttagatgccaagaatcagcaagtacgtcaggttggtgctgggactgaagaccaggatgttgtcgtgttgtcacaactggatggctacttacagaaaggggctaaatcaatagccggattatttctttcgacttccgggggaatgatggttggagatattaatatgggaaccaaccaacagaataccattcgaggtcttccttcttcctggggagaagcgtcatttctaggaaatgattatgcagcatctgttgggatagtaaatgatgtcttaggaaactatgaggaccgattaaatgatctgattgctaaaatcaatatgtatgctgggaccggagagggcagcatggccaaactcatcacagatctaggaagtcctaaagagaccacgtcaggatcgacgactaccataacatttgataagcctgaagaggctaagtggctgttattaagcgctaagaatgctatgacagggactttgcgtttcgagcaacagcagggagctttgccaacagctgccgcgcctacaataaaaaatctaaaaactggtgacataggagtcgtgggaacggcaacgcctaaagaaaagttacagaaaattgtcactgtctcagatttaacgactatactgaatgatttacaaaataattcctctggaggttcaggatcaggaacggggtcaacgacaacacctactttcccaaattggacagggcaagaagttccttattttgtggagcagaatcccaatgggacttcaggtagtggtactcaaagtcctattaaatctactaatgctgataagtatattaaaaaggagaattctacctattctctattgagacgaggcgtttatctcgtaacatttgcttatgaatttacttcctctcaaaatcaggctgtcacatgtaccggaacacttacagaaaaaacaacgggaggaacaggatctaatcctgaaaccctattttctgtagtaggttcctcagatggtttgactgtagtgggtagtttctatattctggttcctggagagaaagcattttcagaacatacgttagagttgaaattaactaccggaactgctggaacagctgctacagcttctaagagttatgtagctatttcttatatgggagctggatattaa

SEQ ID NO: 140 - DNA, 1476 bp, Chlamydia muridarum
gtgtggaagcactggattcgcattattgggataggtagtcttctttctccaacagtcttacttgcattgggagagaagacgccagccttttttttagaaaggaatcgtaatttttggaatatagatccctattgtttagagtctatttgtgcttgttttatttctaatcaagatcctttaagtgctgaacggttagaggttttgtttccacaactatctcaagaggatctattggtttttgctaagtgcattttgtcatcaaaaaatgcagagtaccttttctcggatgcagaagagaaaatttttgctaagctagctcttcctcgcgtttccttggggtgtaatcgagctgatgatttggctaaggtgttagtattagccgaagccggttccactgcaaaagagcagaaagctcgatactattctttatatttggatgttctagctttgcgagcctatgttgagagagcgcgtttagtaaatgcaacttgtgaagttgctagtcaaatagatttagcaagtatagaggcgattaatactattctttttgaaaaagaaggagttcgttacccctccaaaaaagagatgttcgaaagccgattttctgagttggcagctgttacagatagcaagtttggagtttgcttggggagtgttgtgctttatcaagctgtagctcaacgactcaatctctcccttgaggctgtaactcctccaggccatatttatttgcgttataaggatgtggtaaatattgaaacgacttctgggggaaggcatattcctacggaaaagtattgtgagtgtataccggaatctcagttgcgagtgcgatcgcaggaagagctgataggtttaacctttatgaaccggggggctttttttttacaaaaaggagaatttcttcaagcttctatagcctatgagcaagccaaaaagtatttggcggatgagcagatctctgacttgttaggtataacatacattcttttgggcaaaaaaaaagagggagaggatattttaaaaaaatcttcagaaaggactcgcaaaagctcggctatacacgattattttgaacgcaatatttctcctgaaatattggcagtattatttgctgattcgggtgtaacctatcaagaaactttggattatcacaaaaaacttcttgtgcttgttgagaagttccctaggagtggtgctttgagattacgacttgctgcgacagctttggaactaggattagtgaagaagggggagcagcttttagaaaagagcgtggaagatgctccggatgatctcttcttgcgacttcagctttgtaaaattctttgtaataggcacgactatacgcgagcaaagcaacactttgatcaggctaagaagatcttagctgaggaagggatgctttcggaaaaatcttcctatactcttttaaaaatactcgagaaaaaactagctcttgtttctccccactga

SEQ ID NO: 141 - DNA, 927 bp, Chlamydia muridarum
atgagtaattccccagaatcctcaactcccaagcagtccatcccagcacgattccccaaatggttgcgacaaaaacttcctctagggaaagtattttctcgaactgatggcactataaaaaataaggggctccccactgtttgtgaagaagcctcttgtcccaatcgcacgcattgttggtccagacataccgctacataccttgcattgggtgatgcatgtacacgtcgctgtggcttttgtgatattgatttcactaaaaaacctcttcccccagatcctcaagaaggagagaaaatcgctgcttccgcaaaagcattgggcctaaaacatatcgtgatcaccatggtttctcgcgatgatttggaggatggcggagcagatgctctagctcgtatcattacaaccttacatatagaacttccagaagctactattgaagttctagcctctgattttgaagggaacattgatgctttacaccatctacttgatgcacgtattgccatctataaccataatgtagaaactgtagaacgactatctccccttgttcgtcataaagcaacctaccgacggtctctgatgatgttagagcaggctgcacaatacctcccagaccttatgattaagtctggtataatggtaggattaggagaacaggaaagcgaaatcaagcaaacgttaaaagatcttgcagatcatggcgttaagatagttactataggacaataccttcgtccttcgcgaagacatattcctgtgaaaagctatgtttctccagaaacatttgattattatcgatccgtaggggaggctttgggtcttttcatttatgcaggacctttcgttcgttctagctttaatgcagatgctgtttttgaagctatgagtcaacgagagcgcctgtccgcttctatacaatag

SEQ ID NO: 142 - DNA, 762 bp, Chlamydia muridarum
atgaagatgggggggtggcgtaagctgtacgaggtggtttactcttctcttatagggtgcgctttaaagcttcgctatcgtgtgtcggttgaggggttagaggctatcagtccgaattcccaaaaaggtgctttgttcctttcaaaccatgtagcagagatagaccctgtcattttagagcatgtgttttggttgaagtttcacgtgcgtcctatagcagtagattatttgtttaatagtccagttgttaagtggtttcttgactctgtaagagccatacctgttccttctgttgtccctggaagggatgacaagcgtttgcttgaaaggatggagcgcttctatgcatgcgcaacacaagcactggataacaaggaaagtctgcttctttatccatcgggaagattatctagagatggaacagaagagattgtgaatcaacattctgcctatactattttgcacagagcaaaggaatgtgatgtattcttagttaagattacggggctttgggggagttctttttcccgctataaaacgggaagcactccaaagttaggcaaggtgttcaaagaggctgcaaaggctttgttgtgtcgaggaatttttttcatgcctaagcgtgaggtgaaagtttcagtttgcccagcggattattcggtattgaagcagttcccgacaaaacaggaattcaatactttcctctcggattggtttaatcagaagggtggggagactcctttagaagtcccctacacatag

SEQ ID NO: 143 - DNA, 1584 bp, Chlamydia muridarum
atgcctaaaaaaaagtttttatatatcttgatccccttcttgagccttacttttggcttaacgtcttgtcaccagaaagaagaaaacctaagaaatattttgcgtgttgctatttgccacgatccaatgtctttagatcctcgccaagtttttttgattaaagatgtttgtatagcaaaagcactttatgaaggattggttcgagaaaatgatggcagtcttcatttggctctagcagaaagatacagcttgtcccaggatcggtgtgtctacactttttttctaaaaaaaactttttggcataacggagatcttgtaacagcatatgattttgaagagtctataaagcaattttatctccatgaggttgataatgttgcgttacgattgcttgcgttaattaaaaattctcatgctgttctaaaaggagacattcctgttgaaaatttaggtgttcgcgctttagatgaacatactcttgaaatcactctagaacacccctcttctcattttttagaaactctcacacatcccgttttttatccagtgcatgcttctttgagagaatattatagaaatcgaagtaaacgctctcttccaataatttctaacgggccttttatcattcggtgttatgagccacaaaattttttgttgcttgataaaaatcctttttatcatgaccaaaaaaatgtttctttagatgctgtacgcttacaaatagttcccgatattcatacagcggtgcaattgtttcaaaaaaaatatgtggatttagtggggttgccttggagctcctccttccctctagaagaacaaaaaaatctttctcaagattttttgtatgattatccggtgctaaattgcacagttctattctgcaatgttaaccacaaaccattagataatccttcgctaagagcggcactctctttagccattgatcgagaaacattattaaaattagctggcaaaggaagcattgctaccagttttgtacatcctagtctatctaagatgcctttagatgttctttcgcaaaaagagagaatctctttagctaaaaattacctagcggaagctttaaaaactgttcctcaagaagagttaaaaaaaatcacactcatctaccccatagaatctattgtcttgcgagcagttgttcaagagattcgtcaacagttatttgatgttttaggatttaagatttctactttaggattagaataccacagttttttggataaacgttctaaaggagagttttctttatcaacaggaaattgggttgctgattatcaacaagccaaagcttttctatccatccttggtaatgggactaaatataaagattatcaagtgattgattggcaaaatcaagaatatacggatatcgtatctcgacttttggttgaggattcaacagatttacaaattctagcagaacaactattgctcaaagaatcccctcttattcccctttatcacttggattatgcttacgctaaacatcctaaagtctctaatctccaaacctcctcactcggagaaattgatttaaaaagagtctcgttagtggagtaa

SEQ ID NO: 144 - DNA, 1077 bp, Chlamydia muridarum
atgacttcacctttatcaacagctgcttcatcctatctccgcacattacaaagagcgtttcctctgggggggggggggtatcctactaatcctaactccgctcaaactgcactaagagtacagacagcagcaacatctttagctatagttccttacccccgtcctctgtcctcagagccggtaagctttttctctaaacactgtaaaaatgatgctcagagcattattgaaaatgctattctttccgcatcttcttctgtttttctgaaaatctttagtctatcttctgaacccattgtccaggatcttatctgcaaatcggaagaaagtataccagtaactatacattaccagcatataccagaacctattgtgaataaattggaggaagccggggcagaactaatccgttgtcacagaaatagacgctctctactccatagaaaaactatgatccttgatgaaaaacaagtgattactggaagcgcaaacttcactagaaattctataaataatgacgtaaatcttttggctcgagtaaataacgtgcacatagctaatttgatgcagaaaaatcaaaaaggcaaagcgattatagaaaattctactaaaggaaaacaaactgtctgttacttgcctataaatcataaaaaatgcgggaatgaaaggcaaattgtgaaagcaattaataatgccacgagttctattcagattggaatgtgtattcttacacaccgaggcattctccaagctcttaatgaagcggccacacaacgatctgttcttgttacaatcattatcgattctctagaaagtcaacaaaccatagacatactaaaagctcttggatctaaattaagagtacgagtcggaaccggcgatcgcattcactgtaaagcatgtattctcgatcataacactgccattattggctccgctaactgggctgccagcggactaaaagctaataaagaagatattataattgtaaatcctcttacagagcgtcaaagagaagatttaagcatttggtggcggtacctttgtgataatagtgctatcttaccagaagactaa

SEQ ID NO: 145 - DNA, 2373 bp, Chlamydia muridarum
atgctcactcctatccccttattacaaagattcttttcttcccctctttccattaaggaaattttacaagcctgtgatcgcattggaattgaagcggaatgctcgaatgtctttccagactctctaaatacagtcgtaacaggaaaagtattgagcacctccccccatcctgatgccgaacgactttcagttgctattgtttttgatggtaaagaagaacgccaaattatttgtggggcccccaattgtcgcgcaggaatcatcgttcctgttgcgttacccggagctaaaatccgcaatgctttaggagaagtaaccacaatcaaaaaatctaaaattcgtggtctagaatctctaggaatgtgttgtggagcagacgaattagggtttccacatttacaaacaggggagcgcggcatttttgaattcccggaaaatactccccttggagaaagcgcctgcttgctgcttgctggagcttctttggaatgctctttgacccccaatttaggccattgcgcatccctgcttggcctagcaagagaaatctccttcttatctgacacttctcttaccattccagaggaattctcttttagtcctctaccgcaagaatcccattcttgtgatacacataacctcgaagcgagccctgtattctactccgttaaaatctctggattgtcttggaaacaatcccctgaagatttgcaggcatccttgatcgcccttggacagaaacctttgaatgcaatcgtagatatcacaaactatgtgatgctctctttaggacagcccctgcatgcttacgatagccaagccgttgatcaaaaatcgttacacgcaaccactgtacgctctccccaacaacttactcttttgaataaggaaacgtatactcttccagaaggcgctcttgtcgtcgcagatcaacataacattttaggactagctggagtaatgggaagtgccgcatccgcatactctgaaagcacaacagaaattattttagaagcggcttattttcttcctcaagcagttagaaaatatcagcgtactattcagctacatacagaagcggcttatagatttactcgaggtattgatcctcaaggagttttgccagcattgtatgcggccatccacatgattagagccttgttcccagaggcacaaatctccccgattcaaaagattggagagtataaatcggatcctttatcgctaaacatccgcccaaaaacacttaaacgacttctggacctctctctttctccttctgagatagcagagaaactctctcctttaggatttcaaacaatagcagaggaatcttctgttcgtgtagaggtccctctttatcgtcatgatattcaagaagagaccgacctagtagaagagatctgtcgcacaacaccttttgttcaaaagacacaaaaaatacttccgacctatactccaatctatgctttaaaacgctcgttatccacattcttagcagatagtggtttgcaacaattcttcacttgttctctcctggattcagaaatagcagctctctccttacaagaaagctctctcatccctgtgcaaaattcctctctgaaattacgagactctcttctttctgggatgctaaaaagtgcagcaactaatctcaacagacaagctccttatgtttatgcctttgaaattgggaatgtatactcaaaagagcacaatcaataccgagaagaagagcatattgcaattctgctaactcgccaagtaatggatgattcttggcatgtaaaaactcctctttccttttacacaatcaaaagctgggttgagaagttattatgtcatgcaggagtctccatcgaagattttgttcttcaagctagccaacatccgaactttcatccttatcggcaggctgctttataccataaaaaacagttgttaggcgtctttgggacattgcatccgcaattgtgtaagaaaagccagatcaaacacgatgtagtttttgctgagctttctttaaatgttctcctgtctttgaaaaagaaaacggaaccacattatgtcccctatcctatctatccatcttcttctagagacattacaattacaatagataaggatcttccagcggatcttgtacgtagagaacttttaagtttcgaatctaaatggcttgaaagtgttcatattgtcagtgtatatcaagggaaggacgtcacatctcaaagtaaaaatatctcccttcgactggtcttccgtgatcacgaacggaccttgtctgggcaggaaatagaaaaagaatacgaacgtctcactacattactggataagaacttagccaacataggaaaaggcaactcatga

SEQ ID NO: 146 - DNA, 2613 bp, Chlamydia muridarum
atgtcgtatatgctgaagagtattccacctcgccatattcaggtttttatgtttgagaagtttaccaatcgcgcaaagcaagtgattaagttggcgaaaaaagaagctcaacgactcaatcacaactatctaggaaccgagcacatcttattgggattactaaaactaggccaaggagtggctgttaatgtattacgcactcttggcgttgacttcgacacagcaaaaaacgaagtagaaagactcattgggtatggaccggagatccaagtgtatggagatccagctctaacaggtagagtgaaaaaatcttttgaatcagctaatgaagaagcctctattttagaacacaactatgtcggaacagaacatcttttactcggaattctcaatcaagctgatggtgtggctctgcaagttttagaaaacttgcatatagatcctaaagaaatccgaaaggaaattttaaaagagctagaaacgtttaatttgcagctccctccttcttcctctattaccccaagaaacacgaatagttcttcgtcttcctcttcaaaatcttcttctttaggaggacattctttaggtggggataaacctgaaaagctttccgctctcaaagcgtatggatatgacctaacagaaatgttcaaagaggctcgtctagatcccgttattggtcgatctgcagaagtcgaacgattaattttgatcctttgtcgtcgtagaaaaaacaacccagttttaattggagaagctggtgtaggtaaaacagctatcgttgaaggattagcccaaaaaattgtctctggagaagttcctgaagctcttcgtaaaaaacgattgatcaccttagatttggctctgatgatagctggaaccaaatatcgaggtcaatttgaagaacgaatcaaagcagttatggatgaagtccgtaagcatggcaatattcttcttttcattgatgagcttcacaccattgttggcgcaggggctgctgaaggagcgatagatgcttcccatattttgaaaccagccttagctcgtggagaaattcagtgcataggggctacaactctagatgaatatcgaaaacacattgaaaaagatgctgctttggaacgtcgattccaaaaaattgttgttcaaccacccagtgttgatgagactgtagaaattcttagaggcctgaagaaaaaatatgaagagcatcataatgtcttcatcaccgatgaagctctcgttgctgctgctaaactatccgaccaatacgttcacggtcgctttcttcccgacaaagctattgaccttttagatgaagctggagctcgtgtacgtgtcaatacaatggggcaaccttcggagcttttacgtttagaagctgaaatagaaagcaccaaacaggctaaagaacaggctataggaactcaagaatatgagaaagctgccagccttcgtgatgaagagaaaaagcttagagaaaaactaagcaacatgaaacaacagtgggaatcaaataaagaagaacatcaagttcctgttgatgaggaagctgttgctcaagtggtttcagtacaaactggtattcctgcggctcgcttaacagaagcagaaagcgaaaaattattaatgctagagaatactctacaaaagaaagtaattggccaagatcaagctgttgctagtatttgtcgagctattcgtcgttcaagaactggcattaaagatcctaatagacctatgggatccttcctatttctaggtcctactggagttgggaaaactttgctagctcaacaaattgctgttgaaatgtttggaggagaagattctcttatccaagtagatatgtccgaatatatggagaagtttgctgcgaccaaaatgatgggatctcctccaggatatgtaggtcacgaagaaggcggacatttgacagaacaggttcgccgtcgcccttattgtgttgttctctttgatgaaattgagaaagctcatccagatattatggatcttatgttgcagattcttgaacagggccgtctaacggattcctttggacgcaagattgatttccgtaataccattattattatgacttccaatttgggagctgatttaattcggaagactggagagataggttttggtcttcgttcccatatggattatggagtgatcaaagagaaaatagatgctgcagtcaaaaagcatctaaaaccagaatttatcaaccgtctagatgaaagtgttatctttaaacctcttgagaaggaagccctttctgaaattattcatctagaaattaataaactgggatctcgtctacaaaaccaccaaatggctcttaatattcctgattcggtggtctcattcttggtagataaaggacattctccagaaatgggcgcccgaccattaagacgtgtagtggagcagtacttggaagatcctttagcagagatgctattgaaagagtcgtgccgacaagaagcaagaaaattatgcgctcgcctaatagaagagcgtgttattttcgagcgggaagacgaaatttctgctttagctgtagagggggatggccccgcctctgttacagctgatgaatcatag

SEQ ID NO: 147 - DNA, 1287 bp, Chlamydia muridarum
atgagtttccgctgtatctttcttgttattgtcatatttttgataacaggatgctcttattctctcccaaaacacacactaactatagccattcatgacgatcctcactccctttctcctgaacaaggggagaatgcgttcaatttttctttatccagaatcttatttgctacgctctttcgcgaggagccttctggattaatccctgctcttgcctcttcgtatcaaccttctgaaaacggactatgttatcgattttttattcgcaaagacgccaagtggagcgatggttctcttttactggcagaagatgttgttgctgcatggacacacacaaaaaaaacaggtcgctaccccgagttatttgacaatctaactttttacgctccttcttcttcagaagttgttgtggaattaaaggacccgagctctcaactactggctatattggcctcacctttcttttctgtatatcgccctgagagccccctcctatcttccggaccttttatggtccacacttatttacaaggagaaactctcgttctgcacaaaaatcctcattactacgatgacgcgcttgtagagctacaatctatcgattttcgaatcattccgaatatttatacagccttacatctattgcggagaggagatgtggattgggtaggacaaccttggcaccaaggaatccctttcgaactgcgttcggcttctcctttctatgctcactatcctgtagacgggaccttttggttaattctgaattctaaagaccccatattatcgtcattagccaaccgaaaacgattagttgctgccattcgaaaggaagaactcgtgagatatgctttgggagaccaatataaagtggttgaaagtccctccccaaatgaggacgaaagattttgttgctatgaagactcttccgcacaatttccagggaaaataactctaatataccccaacaacattactcgctgccaacgattagctgaagtgttgcaggaacagtgtcgcaatgcaggcattaacttagtactacaaggactagaatatcatgtatttgttcaaaaacgcgccatgcaagatttttctatttccacagcgacttctataactttccaccccttaacctgcgcccaattcgatcacacagcactaaacgacttcacttgcctacctttataccatatggaatatgattacatcttaggcaagccactggatcatgtgattcaccacccttcaggaagcgttgatttgatatacgcacgttttcattaa

SEQ ID NO: 148 - DNA, 708 bp, Chlamydia muridarum
ttggctttgccaaaggatccccacctcgtcaaaaggatgaaatttttcagtttaatatttaaagaccaagaggtcgtcccgaataaaaaagttctctctccagacgcctatactaccgtgttaaatgcccaagaacttctagaaaaaacacaagaagattgcgatgcttacacacagcatacgcatgaagaatgtgcaaatctccgagcagaagcaaaaaatcagggatttcaagaagggagtgaggcttggagcaaacagctcgcctttctcattacagaaacacaggctatgcgagaccaaatcaaatcctctctcgtgcctttagctattgcaagtgtgaagaaaattatcggtaaggaattagaaacaaaacctgagaccgtagtatctatcatttcagagtctttaaaagagctcacacaaaacaagcggatcgtgattcatataaatccccaggatcttgccattgtcgagcaacatcgcccagagttaaaaaaactcgtagaatatgctgatgtgcttttactctctcctaaagctagtgtgtcccccggaggatgcattattgagactgagactggcatcgtgaatgctcagcttgatgtgcaactagccgccttagaacaagctttctccgctgccttgaaacaaaaacaacctacagaaaccttttcaaccgatcagactcaaagcaaagaaggctag

SEQ ID NO: 149 - DNA, 1818 bp, Chlamydia muridarum
  modified_base (6)..(13): a, c, g, t, unknown or other
atgcgnnnnnnnnctgcgtactcagaagcctccatcatttctttagcttctctagatcatattcgcctccgtgcaggaatgtacattggaagacttggagatggttctcaatccgaagatggtgtgtacactttattcaaggaagtggtcgacaatgctattgatgaatttgtcatgggacatgggcatactatttttataactggagactcacaaaaactttccattcgagatgaaggccgagggatccctttaggtaaggttattgactgcgtctccaaaattaacactggggcaaagtataaccaagatgttttccatttttctgttggactaaatggtgtagggcttaaagcggttaacgcattatcacaacacttttctgtccgttctgtgcggaacaaaaaatttctcaaagcatctttttctaaaggcatccttttgcataccgaacaaggagacactcaagatgctgatggcacagaaatttccttttctcctgataaagaattattcgaaaatttctcattccatgatgaatttttaaaaaagaaaatacgtcaatatacctatctccatccaggtctcacaattctttataatggagaaacctttgtctctaagcttgggttagcagatcttttcgaagaggaaattcaaacaccccttctttatcctcctattgctttccaacatcctgatctctcctttcttttttcccacacagaaacctcttcagagcaatacttttcctttgtaaatggccaagaaaccactgatggaggaacccatctcattgcttttaaggaaggagttgttaagggcattaatgaatttttcaagaaaaatttctctgctcaagatatccgtgacggcattgctgggtgcattgccattaaaattgcttcccccatttttgaatcacagaccaaaaataaacttgggaacactaacattcgtgcagatctagctaaacgtgtgaaagaagcggtgatccttacattaaaaaagcacccgtcaagcgcggatcgcattcaagaaaaaatcaaactcaatgagaaaactcggaaaaatgctcagtttctaaaacaagagctaaaagacaaacagaagaagctccattataaaatcccgaaactcagggattgcaaattccatctcactgacaactctttatacggcaacaattcttctattttcatcactgaaggagaatctgcttctgcctctattctagcttcccgtaatccgctcacacaagcagtcttctctttacgagggaaacccatgaatgtcttttcttcaaaagaagagactatttataaaaatgatgaacttttttaccttgccacagctctggggctccataagaattctctacagaatctccgctataatcacattatcttagctacagatgccgatgttgatggcatgcacattcgtaatttgatgattacttttttcctaaaaactttcctgcctgtagtcgaacaaaatcatctattcatcttagaaacaccgctttttaaagtccgctacaaggatgaaacgttttattgctattcagaagaagaaaaactctctaccatagaacggctggggaaaaaagaatcttcgcttgaaatcactcgatttaaaggtcttggtgaaatttctcctaaagaatttaaatctttcatcggacccgacatgcgcttaactccagtctctccccccaatgcagatactctagatgcacttctacagttctacatggggaaaaatacgaaagagcggaagcactttattattgagaatctagttactaacctatag

SEQ ID NO: 150 - DNA, 1029 bp, Chlamydia muridarum
atgttccccctcctcattgactgccagaaagctgcagaaacagttgttatgcaggctatgctagcgcttatgcgttatcgccaatctcgcacattcattccttcatgggaaaagcctgatcagacacatgttactcctgctgactacgctatccaatattattttcaccaaacacttacatctttgtttccgcatatccctttagttggtgaagagactctcaatccggatacggatcaccacaaaattcctcaaattctaaactttgcaaaacaacttgacccacaagtctcgtgtcaaaacctttacaaagctctttcttcccaaaactcccattcttctctattttggttaacagatcctatcgacggcacctcaggattcatcaaacaacgctattttgctgttgctctttccttgttctatgaacactcccctattctttctgtcatagccaaccctacatcaaacaatgcgtccttcaaaatgtattcagcagctaaaggtgaagggctcacaatttatacacccacccgttctatccttttctctcttcaagaagacttccagcttacccatcaattctgcgaagcttctctggcagctagaaaccaccaacatttgaccacacacattcttagtcaacacctcccatggcatccccgacctgttcgcgctgatagccaaagcaaatatgcatgggttgcagacaataccgtagacttcttcattcgtattccttattcccctccacgggctcattacagagaccatgctcctggagtatttcttatagaagaagctggaggccttgttacagatatatccggatccccacttactttttctaatcctaacttatatttggaaaaacaccctttgattcttgcttccgctaacgaacgcatccacaacactatcctggaaactctatatagactccgtcaccggtctactttaaacagactatctccagaagagcagtcttccttcactaagaataagcagctaagcgctcttcaactatag

SEQ ID NO: 151 - DNA, 855 bp, Chlamydia muridarum
gtgttcaagtttttcgggaacaaacttcgctctctttttaaaaagactcttccttcggacttattggaatatgctgaagctcttttatatgagggggattttggtcctaaactaactgaagccttttgtaacgaattacgtcggcataaacatcctgatgagcgcttgattaaagagcttatccgctcttttctatctgatattatagctaagttacctcagaaaaagagctcttctgctcacccccatgtgactcttattttaggaacaaatggctcagggaaaacgactacagtagctaaactcgcccactattgtctttctcagaaccaaacggttttaatcgtagcaacagacaccttccgctctgcaggaatggaacaaatgcgttgttgggcagactctttaggttgtggatttatttctggtaagccaggaggggatgctgccgctatagcttttgacggcatctctgctgcagttgctagaaactatgaccaagtaatcatagatacctctggacgactacatactcacacgaaccttctcaaagaacttcaaaaaattgccactgtttgcaataaggcttttcctggagcgcctcatgaaactctcatgaccgttgatgcaaccttgggaagcaatacgttaagtcaggtaaaattattccatgaggcggtgcctatcgatggattaatttttacaaaaatagaaggctccgctaaggggggctccctctttcgaattgctgacgagctaaaaattcctacacgatttgtaggatacggagaaacagtttacgatttcgaaccattttctatagatcgctttctcgataaactgctggatgtttaa

SEQ ID NO: 152 - DNA, 1266 bp, Chlamydia muridarum
atgttgaagtttcagttgtgtgcgttgttccttttcgggtatctcgcgatagtttttgaacacatagttcgggtaaataaatctgcggtctctcttgctatggggggcttgatgtggttagtctgtttctcccacatgccccatatcgatcacgttataatggccgaagaaattgctgatatggcacaggtcatcttctttttatttgcagccatggccattgtagagctcattgatgctcataaaggattctctattgttgtgcggtgctgtaatgttgaatccagaagctttttgctttgggtattgctcactctttcctttttcttatctgcagctctagacaacctcacgtccattattatcattgtttctattttgaagcgtcttgttaaagctcgagaagatcggctattgctgggagcactttgcgtgataggggtaaatgcaggaggggcatggacacctttaggagatgtcacaacgacaatgctttggattaatgataaaatttcgacggcagggattattacctctctatttcttcctagtgtggtttgtgtagttgttgcgggagtttgtggtcagttactgttaaaaaagcgtcgttgttcaggattattagaggatcttgatagtgagcccgctctccctaaaagtaacctaattgcttttgttggttttggctctttacttatggttcctatgtggaaagctgtattgggagtgcctccctttattggggctttgctaggacttggtttagtttggcttgttagtgattggattcattctcctcatggtgagggacgaaatcatctgcgcatgccgcacattttaactagaatagacatttcctctgttacatttttcattggaattttgttagctgttaatgcactagcgtactctcacgtattgcaagatttatccgtgagtatggatgccgtattttctcgcaatacccttgctattttactaggcttagtttcatctgtcttagataatgttccgctcgttgctgctacaatagggatgtacgaccttccaatgaacgatcctctttggaaactcatagcctatgctgcaggcacaggaggaagtattcttattattggatctgctgcaggagttgcctatatgggaatggaaaaggttagcttcggttggtatgtgaaacatgcttcttggattgctttaattagctattttgggggattagcagtgtattttctgatggaaaatttcatgggattattcgcctga

SEQ ID NO: 153 - DNA, 303 bp, Chlamydia muridarum
atggctagaaaagagcgtttaactaatgaaaaactaaataagctattcgatagcccttttagtttggttaattatgtgattaaacaaactaaaaacagaattgctaggggtgatgtacgttcttccaacgtagcgatagaggctctgaatttcctagatctttatggtattcaatctgaatgcttagaaagggatgatcgggagcaatatgcgtccggagcaggagaaaagcgaaaagaacaaagttctggaaactccagaagaaaggatccgtctctgtacaattggagcgacgtgaaatag

SEQ ID NO: 154 - DNA, 1653 bp, Chlamydia muridarum
gtggcatcttctcgcattcttataacttctgctttgccttatgcaaatggacctcttcatttcggacacattacgggtgcttatttacctgcagatgtttatgcgcgttttcaaagattgctaggaaaagaagtattatacatttgtggttctgatgagtacggaatagcgattaccctaaatgcagaattagcaggtatggggtatcaagaatatgttgatatgtatcataagcttcataaagatacccttaagaagctcgggatttctgtagatttcttttccagaaccacaaaccctcatcatcccgctattgttcaggatttctatagaaatttgcaggacaaagggttgatggaaaaccttgtaacggatcagctttattctgaggaagaagaaaagtttttagctgaccgttatgttgtggggacctgtcctaagtgtgggtttgatcgggctaggggagacgaatgtcagcagtgtggagccgattacgaagccagagatttaaaagatcctcgttctaaattaactgggaaagctttgtctttgcgcgaaacagaacatgcttattttcatttagaacgcatgaaggaagaactgcttacctttgtagagggcatttatttacgtccccacatgcgtaactttgtaatagattacattaaaaatttgcgtcctcgagcagttactagagatttgtcttggggggtacccgttccaggtttagagaacaaagtattctatgtatggtttgatgctccaatcggatacattagtggaactatggattgggcagcgtctatcggagatccagacgcttggaagaaattttggttagatgacaaagtaacgtatactcagttcgttggtaaagataatacatctttccattcggttatttttcctgctatggaaatgggacagtcgcttccctataagaaagtcgatgctttagtgacatccgaatttttacttttggaaggattccaatttagtaaatcggaagggaatatcatagatatggatgcgtttttagaaacgtattccttggataaattacgctatgtgttagcggctattgctccagaaacttcggatagcgagtttgcattccaggagtttaaaactcgatgcaattctgatcttgtaggtaaattcggaaattttgtgaatcgagttgttgcttttgctgctaagaatggatgcaccgaactttctcatcctcaattagaacaacaagatttagagttcatagctgaagctcaaaaacttgctaaagaagctgctgatcattatcagcaatatagtttgcgtaaagcttgctcaacaattatggaattagctgccttggggaatggttatttcaacgatcaagctccatggaaactagctaaagaagatctttggaatcgagtgcgatccattctattttgtgcttgctattgccaaaaattgttagctcttatctcctatccaattatgcctgaaacagcgtggacaatttggggaatgattgctccaggttctttagatttgcgttctcaagatccggaragattgcagtcaatttggacagaagcctttttcgattactcagaagagagcttctcgttgaaagaacctgaattattgttcactgtagttgagtga

SEQ ID NO: 155 - DNA, 1098 bp, Chlamydia muridarum
atgcgagttcattctttatttcttaaagatttcagaaattactcggaactgcgtctagaattaggaccagagatgaattctatctttggtcttaatgctcaggggaaaacaaatattcttgaggcattgtatatcttatctttaggaagatccttccgaacaagtcgtttgactgaggctatacgctttggatcgtctcatttttttatcgaagctgttttctcccaaaaccaagtttttcataccctctctattcaagtagataaacgagggaagaaaattctttttgatggagcccctattactaagttgtccgcactagtaggactatttcctgttattcttttttctgtaaaagacacaacaattattgaaggctccccagcagaacggcgccggtttctagatctcttattagcccaagcttcagaaaaatatacaggtcagattgccttgtatcacaaagctttggaccaacgtaatgcagctattaaaacacaagactataaaaccatcgctgcctggaactcccctcttattgcttatggaagcctggtagccttgttgcgctatgaatgcgctaaaaaactgcataaaatttttcaaaatctttgggataacacattaaaagaaactctttctcttcgttatgagagttctctcattacaacagagtctcctacactcaatgacattgcaagtaattactatgaacaacttagattagctaataccaaagactttgaacttggctacacaacagtaggccctcatcgagatgaacttatcatcacgcttaatgatttacctgtttctaaattctctagtgaaggccaaaaacattctcttttagcagttcttcgatttgctgaatgtgtatatcttcaggaagaatttcttatccatccacttctatgtatggatgatattcacgcttgtttagatcaaaatcgattagatcagctcttccaactctcaacttctttaggacaaactgttactacttccaccatttgtccgaatcatctagattctaactcttctatttttcacgttacacaagctcaagtatcgcttgtcacttccaattttttataa

SEQ ID NO: 156 - DNA, 749 bp, Chlamydia muridarum
caacgttatgataagctaaaaaaatcatacgatacttcgcgagattttgatgaaaaacagcggttatctgagcagttagttgagctatacgataattacaataaatttagtaaggagattcaaagtaactttgatgttttgctattgcatgaaatgatcactattcaagatagcttttttaactactgccagctcaaaggagtaggtgcaataacagatgagacacgtatagagtatcttgagaaagttttgaaagtaaaagaagaggatctatctcactataaagaaactatcaaacgcaacaaagagtctattgaaaaacttgtaaaagagattaatgactctactggacgggagagagtagttatcaaagacatccgagatttgaagtctttacaagatcttactaatagctacaactatgaaacagaaatgttactccgatggaattatgcagcggctaccgaccagcttcgtatgtacatgcttaaagaatatggggggatttatacagacctagatattatgccacaatactctcaggatgttttgcaaaaaattatggatgttggtgggagtaggttttttgagcatgataagttacggcggactctttcttttgcagctctaaaattaggaagcggtaagcaaacaactgtttcctttgaagaggctaaaaaagctatgactctccccacatttactcttcaggataagagtcaaatttctgaaatttttaaatatctaga

SEQ ID NO: 157 - DNA, 84 bp, Chlamydia muridarum
gaagcttttggaaacaagcttctctatcctgtgacccaaggatttggaacccgtatttcagtaaagcctgttttagtaatcgat

SEQ ID NO: 158 - DNA, 377 bp, Chlamydia muridarum
aaacgcaagcaaaaaaatcacttttccaaccgatggatgttaccgtcgtccgtgatttcatgcctattttacaacgttatcataagtggcaaacaggatggaatgttcggggattaaatggtttaatgatggctcataaagatagtgctgttgtcgatgctgttattgctcgccagcgagcggcctatgatgagatgcgtgcacttcgtcagaatgttgttagtggagaattttttcgttctcttggagatcttgaacatgtaaatcgtgaaaagaatatcggcggctatttagcgaaaaattatcttgggggcagtctttttttcgattttcgtcaagattcagtgattccaggagctattagtaccttaggaatt

SEQ ID NO: 159 - DNA, 1583 bp, Chlamydia muridarum
  modified_base (7)..(14): a, c, g, t, unknown or other
atgcctnnnnnnnngtttttatatatcttgatccccttcttgagccttacttttggcttaacgtcttgtcaccagaaagaagaaaacctaagaaatattttgcgtgttgctatttgccacgatccaatgtctttagatcctcgccaagtttttttgattaaagatgtttgtatagcaaaagcactttatgaaggattggttcgagaaaatgatggcagtcttcatttggctctagcagaaagatacagcttgtcccaggatcggtgtgtctacactttttttctnnnnnnnnctttttggcataacggagatcttgtaacagcatatgattttgaagagtctataaagcaattttatctccatgaggttgataatgttgcgttacgattgcttgcgttaattaaaaattctcatgctgttctaaaaggagacattcctgttgaaaatttaggtgttcgcgctttagatgaacatactcttgaaatcactctagaacacccctcttctcattttttagaaactctcacacatcccgttttttatccagtgcatgcttctttgagagaatattatagaaatcgaagtaaacgctctcttccaataatttctaacgggccttttatcattcggtgttatgagccacaaaattttttgttgcttgataaaaatcctttttatcatgaccaaaaaaatgtttctttagatgctgtacgcttacaaatagttcccgatattcatacagcggtgcaattgtttcnnnnnnnntatgtggatttagtggggttgccttggagctcctccttccctctagaagaacaaaaaaatctttctcaagattttttgtatgattatccggtgctaaattgcacagttctattctgcaatgttaaccacaaaccattagataatccttcgctaagagcggcactctctttagccattgatcgagaaacattattaaaattagctggcaaaggaagcattgctaccagttttgtacatcctagtctatctaagatgcctttagatgttctttcgcaaaaagagagaatctctttagctaaaaattacctagcggaagctttaaaaactgttcctcaagaagagttnnnnnnnntcacactcatctaccccatagaatctattgtcttgcgagcagttgttcaagagattcgtcaacagttatttgatgttttaggatttaagatttctactttaggattagaataccacagttttttggataaacgttctaaaggagagttttctttatcaacaggaaattgggttgctgattatcaacaagccaaagcttttctatccatccttggtaatgggactaaatataaagattatcaagtgattgattggcaaaatcaagaatatacggatatcgtatctcgacttttggttgaggattcaacagatttacaaattctagcagaacaactattgctcaaagaatcccctcttattcccctttatcacttggattatgcttacgctaaacatcctaaagtctctaatctccaaacctcctcactcggagaaattgatttaaaaagagtctcgttagtggagta

SEQ ID NO: 160 - DNA, 128 bp, Chlamydia muridarum
gctctctactccatagaaaaactatgatccttgatgaaaaacaagtgattactggaagcgcaaacttcactagaaattctataaataatgacgtaaatcttttggctcgagtaaataacgtgcacata

SEQ ID NO: 161 - DNA, 2673 bp, Chlamydia muridarum
ctaataacgtcgagaagactgtttccattcaggacgacaaaaacgctttttccttataaaggaattatgtgatttgacgaattgtggtcgtttcgacaatcctactttttgcagcttgcgtaatcttgctgttatagcagttgggctacgtcccgtttgcgtagcgatgtccttaacactcatccccttacaaagttgtaacaaaagagactcctcgctgcgtgtccaaatcttataggcatttacgggcgttctaggaacttgagctcttttctcgtccacttttataaactctttaacaaactctgaagggaattttttatcataaaggactgtagttttgatcttagctcgtgttattgcaacatagaataatctccgctcctcgctaaacggaaaagaatcgctttctgataaaacatagttaaacactttgttatctgtaacaagactaggaaatccataacggtctgcgttgcattgaagcaaaattacgtaatcagcttcaagtccttttgacttgtgtacagtaaggaattctatttttctatttccgattatataaaaaaaattgctccctgttttaacaagctgatactttttggatagataatagtcatcaaatgaatacctccctaataaaaagatggatttatcttctggaatcttagaaatctccagctctaaacaagagcaataatccttttcagacctgtgataataagagcgaaatactaactcagtcctaatatctggattaaaggaacggcaatcttttcgtatttgaaaattattacgttgtacaaagctagaagacaaagagaccaaaggctctcccaacctgtaggtagtctctagtctatggatctcttgtggaccaaaataatcggaaaactgattgaacacagtgatattacttcccgcaaatcgatagatagactgccaatcatccccaacgcaatacatcttagcgggaggatttcctttacgcaattcttgcagaaagcggcaacaatcaatagaaatatcttgaaactcatcgacgataatataatcgtacttcccagaactagagtgacgacaaatttctgttgcttgcaaaatagcgtcagtaaaatcaatctgcccccttttagctaatgtatccacataacgttcgtatacgggtttaaaaaccttttcaataagccaagaacttgggatatctttagcgattttggcgcaatatagcactttcttaatagatttatgatttgatttcattaaagaaataaagcgtactataagtttaataagtgctttctcttgaacgctttttttaggaagaagagcgttatataaatcctcttctttcttttcttgaataggaatgcctgctctttctaagagttttcttaatgtatcccaaatattctcccgaatattcgaattgtagaaatcctcactagttaaagtaagcagttctgttttgaacttcttatgagtttgctttttccaagctatcccatctaaatatatttgattcacttctttataagtcttatttgtcccttcaacaaaccatggaggaactcgtccttgtttattaatagcaaaaagctccaggtagatacgtcttaaacttccattctgcgcagtaacgtaaagagaaaaatccggtctgtattgggaatgcgtttcatctgctacttgaaattcataaggttcttcatatcggaacgaaatgcctaaagacgataaaatgaagcaggtggtttgctcttgtttgcttcggacataaatcgttttcccatccatatcagggaatacagctttcaagaatggtttcttttgttcagacagctgatctcgaattttcctttcagattcgcaatcatcataaaaaacaaaatattttaatacgttctttagaaaatcctttgttttcaataatttttgacagatctcctcaataagctcctcaggattgctacagatagagggtttaactcctgttatgcttccaattaggtcaacagccaacttatgaaaggtatatccccttaatccatccgtagccaatctttttgtaagttctgatgaagctttattggtataactaatcagagctatacgacgaggatctattcccttaatctctataagatatctcaccttccctaagatagaagacgttttcccacttcctgcagagctaatgactaaacaattctcttcttctgatataatagagcgtctttgttgttcatccaatgggtaatgtagaacgcgatcaaaaaaatccttataaagatccagtaaataacttataatctcactattatgtttcttaataagagttgttatcgactcaaaatcttgtatgaattttaaaagaatgggatttgaagaaatgccaaaataacgaaatcttcggcaaaatgtgcgaaccctttgataataggcaacataataatctgtgacccttttctcttgttttgcgtagataaccttgccacagaaaccatgatgtaaaatagtgggaatgtcgatgcgttttagctgctgagataatttcaaacagcttcttttaacaagaaaaaaagaataaatgtaaattatcgcgcatacaaaatagaaaactagactaactaaaacaagctggctaataactccactccttgatacccacaa

SEQ ID NO: 162 - DNA, 1950 bp, Chlamydia muridarum
atgcgcataaacgtggataaatacttatttattggacgtaaaaagtctgaatttttctctgcgtgccgagagcttggagctgtcgaatttttggcaaaaaacaaactcaaggattcagaaaatgttcgtaggatttctgaagggttaaaaacactcaatttgttaacgaataaatattcgccatcagatttagtgttagtcaaatctggatatctgacaacagaacagcttcttcaggaaatttttgatcttaatcatgaaattacaacgattacagattctttaaaggccttgagtaaagaaatttttcgcgttaaacctctagggaatttctcctctgaggaaattcgtgaattaacgttgaaaacaggtttatctgttcgatttttctataaaaaacatatagaaggagctcctttagaggttgaggaagagaatgttttctatctagcaacagcatacaactatgattactatgttgttattggggttgtctctctatccaaagatatttttacagaaatagaagctcctcgttctgttggagaattgcgagaagaagaagaacatcttcaaacccttttgcgtaaaaagaaagctcgtgtttgtgagttatatgcttatcgagaagaattattagaagctttatgtgagcaatgtaatgagcaaactttacagcatgcagaagctagtacagaagatttattcgatgacaaagtgtttagtgctttgggttgggttattgtcgatcgtttgactgaagtagaaaagctttgtaatagtttaggagtttatttagaacgggtgcaacccgatccagatgaagtaatccctacatatttagagaaccatggactgggagctttaggggaatctttggtcaatatttatgatacccccgcatccacagataaggatccttccttatgggtctttttatcctttttcgtattcttctcaatgattattaatgatgcagggtacggtttaatatttttagcaacttcattattcttgtctttcaaagctcggaagcaggtaaagcattctctggcattaaaacgttttctgaaaatgtttatgatattagggggtggatgcgtttgctggggtggggctacaacatccttctttggcgtgtcggtcagctatacaagtccttttcgggaatattcgttgacgcattttcttgctatgaagaaagctgagtactacctgaaggagcgccccaaaggctataaagaattggtgcatgattatcctgtgctcaaggataaaaaaactcctaaagagtttcttttggctcaagggactagcagtggagattctgtatacaaagctgttgtttatgataaatttacagataatattctaatggaaatagctttgttggtaggggtagttcatttgtctttagggatgttgcgctactgtagacaaaggtattcttctataggctgggtagttttcatgtgcggagcgtatatgtatctgcctatctatttacaagccgtttctctgattcattatgccttacacgttccttatgaattaggggggcaagtaggatattatgtaacatttataggcttaggcatagctgttttaggcggaattatccagagaggattacgaggcctggatgaggtaacagctgttattcaagtgttttcagatgttttgtcctatttacgactgtatgctcttagtttagcaggagctatggtggggaacactgtcatggtaatgagcgaaagattttcccccgcagtaggggtcttgataattatctttgggcatacagtaaatattgcactttccattatgggaggtgtgatacatggtctccgtcttaattttatagaatggtatcactatagctttgatggcggtggaagacttttgcacccattgaaaagggtaatttgtcaaaagtctcaaaatatttga

SEQ ID NO: 163 - DNA, 876 bp, Chlamydia muridarum
atgagaatgcaaattccaagaagtgttggcacacatgacggttcttttcacgcagatgaagtgacggcttgcgctttgttgattatgtttgaccttgttgacaaggacaaaattattcgcaccagagattctgaaaaattagcgcaatgcgagtatgtttgtgatgtaggggggcggtactctatagcggataagcgttttgatcatcatcaggtgtcttatactggttcatggagtagtgcagggatggttcttgattatttgcatcatctaggatcccttccgcatgaagagtacgaatatctcaatagcacattagtgcatggtgtagatgaacaggataacggccgttttttctctaaggaaggattttgctctttctctgatattattaaaatttataatcccctggaagagggagggaacactgataaggaatttttctttgctttgcattttgcgatagatctgcttactcgtttacgggaaaagtttcgttatgatcgtatttgtcgtgacgttgttaagcaagtaatggaaaaagaagatgtctgtttgcgctttgatcgtccattagcttggcaagaaaattttttctccttaggaggagaaaatcatccggcggcttttgttagtttcccctgttcagatcaatggattttgagaggaattcctcctactttggatcgtcgaatggaggtgcggattccttttccagaggattgggcagggttgcttggggatcaattggttaaggctacagggattcctggagcgattttttgccataaagggttattcctctctgtttgggatagtcgggaaagttgcgaagaagcactgaatttggtgttaaaacagcagggggtagtatga

SEQ ID NO: 164 - DNA, 671 bp, Chlamydia muridarum
  modified_base (174)..(181): a, c, g, t, unknown or other
atggcactgtatttagggctcaatccatcctttgctaagcaatattctgctatttttcagccaattattcgcataattcctttttcaaagcatgcccctcagatgcgtcaagcgaggcaattccttgtatctgcttcccatgttttattaacaagcccttcatccacccactgnnnnnnnncttccattaagaaaacagtttctccgtctgcgttaatgagcaaatgcttcgttactattggagaagctacctcttctcgcgtacgcgcccaccttcctaaagctaatattgtgcaagttccattccctcaatcagaagcttttcaacctattctatcctccctcccatccaatacctctatattatatcctcattcttcccttgctcgacccattatccgatctctgcttaacaaactacagtatcccttcttttcctattcccactacactgtcaaacctatcaaactttctctaaacatattttccccacacaaaattattattcttaccagtccttcgattgttcgcgcatatgcgaaattattcccatcccttcctagtaaagaacactggtgcctaggtgaaatcagcgcagaggaatttaaaaaaatattccactgccctccatctaaaattctcttcccaataaaggaatcccaaggctgttg

SEQ ID NO: 165 - DNA, 529 bp, Chlamydia muridarum
atgtctgaaccatcctcttgttttgttcttccggatcctgaatggatttaccgtgtcggcattgggcaggacagtcaccgttttcttcctagtgacaatcctaaaccttgtgtcttaggagggattatttttgaaaacaccccaggttttgaggcaaattcggacggagatgtcgtttttcatgctatttgcaatgctttttcatctgtaacacatcaagtcattttaggagcattggcggatgaactattaaaagcaaaggggatttctgatagtgcagtttatctgaaggaagcggttgcatctttaaagcctacacaaaagatctctcatctggctattactatagagggacaacgccctaaactacttcctaaaatatctgctatgcgagaaaaaatagcagagatacttcgaatctctttagattctgttaacatcacagcaacttctggagaagggctaacgagcatgggccagggacatggcgtacaatgtttctgcgttttaaccgttatggaattctgcc

SEQ ID NO: 166 - DNA, 2373 bp, Chlamydia muridarum; identical to SEQ ID NO: 145

SEQ ID NO: 167 - protein, 122 aa, Chlamydia muridarum
KKSLFQPMDVTVVRDFMPILQRYHKWQTGWNVRGLNGLMMAHKDSAVVDAVIARQRAAYDEMRALRQNVVSGEFFRSLGDLEHVNREKNIGGYLAKNYLGGSLFFDFRQDSVIPGAISTLGI

SEQ ID NO: 168 - protein, 134 aa, Chlamydia muridarum
LKEIFSKKSPPLRKSFSSSKISRAIPRRKKRSNDDPVQATNTYDLTAENILEKLSLTQEQQIKHDNLISNLKEAINRYSDLNRKNSRKGQSLLVRQAKILDEILSQTKSTEERASNSVMTTIKKEFTSHRVPVE

SEQ ID NO: 169 - protein, 283 aa, Chlamydia muridarum
KRIDSRTRESDLNFLKINGDILEEFLTRTVLDSDLVELIQSLLKTHGLEPGTTLISPSSIVETAIDHVSLLQAVKTKTSRMHTILQSLGERIFKLFKNSGVQDSDKISIDRVQLVDESDSATIDFTVIKDKQRSQKKSITVGIESLAGSFRKFSASMHEVIGTGVLDLDLGMTVVSLVQYVRLVEAGQGKDALAVANLVMNLKIALEVSIGNVIQALGKKLLTQEGLNTFRLETELARQLHKVGARVGGTVGKTLTRVAHVLELPILESAIGTWNLYNSVNEL

SEQ ID NO: 170 - protein, 6 aa, Artificial Sequence
  Description of Artificial Sequence Synthetic 6x His tag
HHHHHH

SEQ ID NO: 171 - DNA, 20 bp, Artificial Sequence
  Description of Artificial Sequence Synthetic oligonucleotide
tccatgacgttcctgacgtt

SEQ ID NO: 172 - protein, 123 aa, Chlamydia trachomatis; identical to SEQ ID NO: 31

SEQ ID NO: 173 - protein, 135 aa, Chlamydia trachomatis
VKTISASAVPPTAAVLSRKKRAIEEKKEEASSGKIENLDASKYDLTPKNIEEKLGITPEQKSTVKDLLNKLKKVISAYNSMPDKNSEAGQNSLIQQGKYVDAIQKKLPASSQAQPKQAKAKEQKAEEKPKTTPIE

SEQ ID NO: 174 - protein, 294 aa, Chlamydia trachomatis; identical to SEQ ID NO: 39

SEQ ID NO: 175 - protein, 64 aa, Chlamydia muridarum
VDKGFYIYYSVSKLANHVKLRNVTGDMLLDIDKETTFIVRGVDESDYDKKQIYVVLDLTTEEER

SEQ ID NO: 176 - protein, 62 aa, Chlamydia trachomatis; identical to SEQ ID NO: 40

SEQ ID NO: 177 - DNA, 752 bp, Chlamydia trachomatis
  misc_feature (279)..(286): a, c, g, or t
caagattttttgtatgattatccggtgctaaattgcacagttctattctgcaatgttaaccacaaaccattagataatccttcgctaagagcggcactctctttagccattgatcgagaaacattattaaaattagctggcaaaggaagcattgctaccagttttgtacatcctagtctatctaagatgcctttagatgttctttcgcaaaaagagagaatctctttagctaaaaattacctagcggaagctttaaaaactgttcctcaagaagagttnnnnnnnntcacactcatctaccccatagaatctattgtcttgcgagcagttgttcaagagattcgtcaacagttatttgatgttttaggatttaagatttctactttaggattagaataccacagttttttggataaacgttctaaaggagagttttctttatcaacaggaaattgggttgctgattatcaacaagccaaagcttttctatccatccttggtaatgggactaaatataaagattatcaagtgattgattggcaaaatcaagaatatacggatatcgtatctcgacttttggttgaggattcaacagatttacaaattctagcagaacaactattgctcaaagaatcccctcttattcccctttatcacttggattatgcttacgctaaacatcctaaagtctctaatctccaaacctcctcactcggagaaattgatttaaaaagagtctcgttagtggagta

(19) United States
(12) Patent Application Publication
(10) Pub. No.: US 2009/0028891 A1
(43) Pub. Date: Jan. 29, 2009

(54) Chlamydia Antigens and Uses Thereof

(75) Inventors: Peter Timms, Queensland (AU); Ken Beagley, Queensland (AU); Louise Hafner, Queensland (AU)

Correspondence Address: PROSKAUER ROSE LLP, 1001 PENNSYLVANIA AVE, N.W.,, SUITE 400 SOUTH, WASHINGTON, DC, 20004, US

(73) Assignees: QUEENSLAND UNIVERSITY OF TECHNOLOGY, Brisbane (AU); UNIVERSITY OF NEWCASTLE, Callaghan (AU)

(21) Appl. No.: 11/718,743

(22) PCT Filed: Nov. 11, 2005
(86) PCT No.: PCT/AU05/01724

§ 371 Date: Jun. 22, 2007

(30) Foreign Application Priority Data
Nov. 11, 2004 (AU) 2004906459

(51) Int. Cl. A61K 39/00; G01N 33/569; C12Q 1/68; A61P 31/00

(52) U.S. Cl. 424/190.1; 435/7.36; 435/6

(57) ABSTRACT

The invention provides protein and nucleic acid sequences of Chlamydia species which are particularly suitable for diagnosis, treatment and/or prevention of Chlamydia or Chlamydophila infection in mammals, particularly humans. The invention particularly provides a pharmaceutical composition and method for preventing or treating Chlamydia or Chlamydophila infection.

### FIELD OF THE INVENTION

THIS INVENTION relates to Chlamydia antigens, nucleic acids encoding said antigens and uses thereof, in particular in relation to diagnostic reagents and pharmaceutical compositions. In one form, the invention relates to a vaccine against Chlamydia.

### BACKGROUND OF THE INVENTION

Chlamydiae are obligate intracellular gram-negative bacteria. Chlamydia trachomatis infections are the most prevalent bacterial sexually transmitted infections in Australia and it has been estimated that 89 million new cases of genital chlamydial infection occurred worldwide in 1995 (Peeling and Brunham). Lymphogranuloma venereum (LGV) is a sexually transmitted disease caused by C. trachomatis serovars L1, L2, and L3 and resulting from dissemination of the infection in the genital tract of women. LGV is a disease of lymphatic tissues that is a serious public health concern particularly in South East Asia and parts of Africa.

In developed nations the most common sexually transmitted C. trachomatis infections are caused by serovars D-K. The majority of these infections remain localized in the genital tract and often cause only mild inflammation or are asymptomatic. Without symptoms many affected individuals remain untreated and, in a large number of cases, the infection can progress to serious upper genital tract complications. Also, without symptoms and treatment, the spread of infection may occur unchecked.

C. trachomatis is a major cause of urethritis and cervicitis in women and the sequelae of these diseases include pelvic inflammatory disease, ectopic pregnancy, tubal factor infertility, epididymitis, proctitis and reactive arthritis. In humans, a majority of infections caused by C. trachomatis occur in the genital tract, however, serovars D-K can also infect the rectum and pharynx by sexual transmission or autoinoculation as well as an eye of neonates via the birth canal causing inclusion conjunctivitis. C. trachomatis infections have led to major medical, social and economic problems (Marra et al. 1998).

In women, C. trachomatis infections most commonly manifest in mucopurulent cervicitis (Brunham et al. 1984) that can lead to a number of complications. Intraluminal spread of the infection from the cervix can result in pelvic inflammatory disease (PID), a condition that affects about one million women annually (Cates and Wasserheit 1991). A further complication of mucopurelent cervicitis results from ascending infection during pregnancy that can cause rupture of the membranes, premature delivery (Martin et al. 1982; Gravett et al. 1986) and neonatal conjunctival infections (Gencay et al. 1995). Further sequelae resulting from C. trachomatis cervicitis can lead to development of cervical neoplasia. Although most cervical carcinomas are caused by human papilloma virus (HPV) it recently has been suggested that C. trachomatis may play an important cofactor role (Koskela et al. 2000). The impact that these infection sequelae impose on female reproduction is severe considering that PID caused by C. trachomatis is the most important preventable cause of infertility in women (Paavonen and Eggert-Kruse 1999).

C. trachomatis is the most important agent for non-gonococcal urethritis, the most common clinical genital syndrome in males (Zelin et al. 1995). The effect of urethritis and epididymitis caused by C. trachomatis infection on male fertility is largely unknown. There is some epidemiological evidence to suggest an association between asymptomatic infections with C. trachomatis and unexplained infertility in men (Greendale et al. 1993). C. trachomatis infection in males can lead to stenosis in the infected organs, may induce autoantibodies and consequently inflammation and may also impair sperm quality. These factors indicate that C. trachomatis infections in males may impact on fertility (Wolff et al. 1991; Purvis and Christiansen 1993). However, it is more commonly believed that the significance of male C. trachomatis infections lies in the transmission of the pathogen to women and the infection sequelae of C. trachomatis infection in women.

Antibiotic therapy effectively eliminates chlamydial infection, however, it does not always affect established pathology. The presence of asymptomatic infections in both males and females makes control of C. trachomatis infection by treatment of symptomatic individuals alone unlikely to succeed. If left untreated C. trachomatis infections are most likely to lead to chronic inflammatory conditions and the severity of the disease is related to the persistence of infection or frequency of reinfection. Prevention of C. trachomatis infections by vaccination could have a serious impact on health worldwide.

Previous vaccines against Chlamydia comprise live or attenuated pathogens. However, a disadvantage in using live vaccines includes the risk of vaccine induced infection. Studies have shown that live or attenuated pathogens vaccines provide only short lived protection and subjects immunised with insufficient antigen suffered a hypersensitivity reaction upon re-exposure to C. trachomatis.

An alternative to live or attenuated pathogens vaccines is randomly assessing specific antigens of Chlamydia for immunisation. However, this approach is limited as there is no assurance that antibodies produced in response to such an antigen will provide protection against subsequent infection by the pathogen. Consequently, often a large number of antigens must be assessed before an antigen that is protective against disease is found. Some disadvantages of conventional vaccines were overcome with the development of “genetic immunisation” (Tang et al. 1992), wherein host cells are inoculated with plasmid DNA encoding a pathogen protein. Although promising, the problem of identifying a particular gene or genes of a pathogen that will express an immunogen capable of priming the immune system for a protective immune response to challenge infection by the pathogen still remains a challenge.

### SUMMARY OF THE INVENTION

The present invention arises, at least partly, from the inventors realization that there is a need for protein and nucleic acid sequences to facilitate methods for diagnosing and preventing infection by Chlamydia, in particular C. trachomatis.

The present inventors have identified Chlamydia nucleic acids encoding protein antigens, including full length proteins and fragments thereof, that may be useful in diagnosis, treatment and/or prevention of Chlamydia or Chlamydophila infection.

In a first aspect, the invention provides a pharmaceutical composition for preventing or ameliorating Chlamydia or Chlamydophila infection comprising an isolated protein comprising an amino acid sequence selected from the group consisting of the sequences set forth in SEQ ID NOS: 1 to 85 and a pharmaceutically-acceptable carrier, diluent or excipient.

Preferably, the pharmaceutical composition comprises an isolated protein comprising an amino acid sequence selected from the group consisting of the sequences set forth in SEQ ID NOS 1 to 43.

More preferably, the pharmaceutical composition comprises an isolated protein comprising an amino acid sequence selected from the group consisting of the sequences set forth in SEQ ID NOS 20, 34 and 35.

In a second aspect, the invention provides a pharmaceutical composition comprising a protein homolog, variant, derivative and/or fragment of a protein of the first aspect.

Preferably, the pharmaceutical composition comprises a plurality of protein of the invention, including a protein homolog, variant, derivatives and/or fragment thereof.

Preferably, the homolog is an ortholog.

More preferably, the ortholog is obtainable from the group consisting of Chlamydia suis, Chlamydia trachomatis, Chlamydophila abortus, Chlamydophila psittaci, Chlamydophila caviae, Chlamydophila felis Chlamydophila orum and C. pneumoniae.

In one embodiment, the protein fragment comprises an amino acid sequence as indicated in FIG. 1.

In a third aspect, the invention provides a pharmaceutical composition comprising an isolated nucleic acid encoding a protein of the first aspect, including fragments thereof, and a pharmaceutically-acceptable carrier, diluent or excipient.

Preferably, the nucleic acid comprises a nucleotide sequence selected from the group consisting of the sequences set forth in SEQ ID NOS: 86 to 166 and a pharmaceutically-acceptable carrier.

Preferably, the nucleic acid comprises a nucleotide sequence selected from the group consisting of the sequences set forth in SEQ ID NOS: 86 to 127.

More preferably, the nucleic acid comprises a nucleotide sequence selected from the group consisting of the sequences set forth in SEQ ID NOS: 105, 120 and 121.

In a fourth aspect, the invention provides a pharmaceutical composition comprising an isolated nucleic acid encoding a protein of the second aspect, including fragments thereof.

In one form, the pharmaceutical composition of the first, second, third or fourth aspects is an immunotherapeutic composition capable of eliciting an immune response in an animal administered with the pharmaceutical composition.

Preferably, the pharmaceutical composition when administered to an animal is capable of preventing infection, reduce severity of infection, reduce symptoms caused by infection or ameliorate infection by one or more species, biovar and/or serovar of Chlamydia or Chlamydophila.

More preferably, infection comprises infection of the genital tract, rectum or pharynx.

Preferably, the pharmaceutical composition is capable of treating or preventing a disorder.

Preferably, the disorder is selected from the group consisting of atherosclerosis, sexually transmitted disease, urethritis, epididymitis, cervicitis, pelvic inflammatory disease, ectopic pregnancy, infertility, tubal factor infertility, epididymitis, proctitis and reactive arthritis, conjunctivitis, including neonatal conjunctivitis, mucopurulent cervicitis, rupture of the membranes, premature delivery, cervical carcinomas, stenosis in an infected organ and inflammation.

Preferably, the sexually transmitted disease comprises Lymphogranuloma venereum (LGV).

Preferably, the immunotherapeutic composition is a vaccine.

More preferably, the vaccine when administered to an animal is capable of providing protective immunity in said animal against one or more species, biovar and/or serovar of Chlamydia or Chlamydophila.

Preferably, the animal is a mammal.

More preferably, the mammal is human.

In a fifth aspect, the invention provides a protein fragment selected from the group consisting of a protein fragment comprising an amino acid sequence as indicated in FIG. 1 with an underline and described herein.

In a sixth aspect, the invention provides a nucleic acid comprising a nucleotide sequence encoding the protein of the fifth aspect.

Preferably, the nucleic acid comprises a nucleotide sequence as indicated in FIGS. 2 and 3 with an underline and described herein.

In a seventh aspect, the invention provides a method for inducing an immune response in an animal, including the step of administering the pharmaceutical composition of the first, second, third and/or fourth aspect to an animal.

The animal is preferably mammal or avian.

More preferably, the mammal is a human, mouse, rat, hamster, swine, cattle, sheep, goat, feline, canine, guinea pig, koala or horse. Even more preferably, the mammal is a human.

In an eighth aspect, the invention provides use of the pharmaceutical composition of the first, second, third or fourth aspect to prevent infection, reduce severity of infection, reduce symptoms caused by or improve recovery from infection by Chlamydia or Chlamydophila spp in an animal.

More preferably, the Chlamydia or Chlamydophila spp is selected from the group consisting of Chlamydia trachomatis, Chlamydophila pneumoniae, Chlamydia muridarum, Chlamydia suis, Chlamydophila abortus, Chlamydophila psittaci, Chlamydophila caviae, Chlamydophila felis Chlamydophila pecorum and C. pneumoniae.

More preferably, the Chlamydia or Chlamydophila spp is selected from Chlamydia trachomatis and Chlamydophila pneumoniae.

Preferably, the Chlamydia spp comprises Chlamydia trachomatis.

More preferably Chlamydia trachomatis comprises a biovar and/or serovar thereof.

Preferably, the serovar is selected form the group consisting of serovars A, B, Ba, C, D, Da, E, F, G, H, I, Ia, J, K, L1, L2 and L3.

In a ninth aspect, the invention provides a method for detecting one or more species, biovar and/or serovar of Chlamydia or Chlamydophila in a biological sample including the steps of:—
(I) combining with the biological sample an antibody or antibody fragment capable of binding to one or more proteins of the first or second aspect, or a fragment thereof; and
(II) detecting specifically bound antibody or antibody fragment which indicates the presence of said Chlamydia or Chlamydophila.

In a tenth aspect, the invention provides a method of detecting one or more species, biovar and/or serovar of Chlamydia or Chlamydophila in a biological sample including the step of detecting a nucleic acid comprising a nucleotide sequence of the third aspect, which indicates the presence of said bacteria in the biological sample.

In an eleventh aspect, the invention provides a method of diagnosing infection of an animal by one or more species, biovar and/or serovar of Chlamydia or Chlamydophila, or absence of infection, including the steps of:—
(a′) contacting a biological sample from said animal with a protein of the first or second aspect or a fragment thereof; and
(b′) determining the presence or absence of a complex between said protein and Chlamydia or Chlamydophila-specific antibodies in said sample, wherein the presence of said complex is indicative of said infection.

Preferably, the biological sample comprises a cell, tissue, biological fluid, mucus sample, blood sample, serum sample, respiratory wash sample, lavage sample, coronary sample carotid plaque sample, blood vessel or plasma sample.

In a twelfth aspect, the invention provides a kit for detecting Chlamydia or Chlamydophila in a biological sample or diagnosing Chlamydia or Chlamydophila infection in an animal, wherein said kit comprises one or more isolated proteins according to the first or second aspect and/or antibodies capable of binding said isolated proteins.

In a thirteenth aspect, the invention provides a kit for detecting Chlamydia or Chlamydophila in a biological sample or diagnosing Chlamydia or Chlamydophila infection in an animal, wherein said kit comprises one or more isolated nucleic acids, including fragments thereof, according to the sixth aspect.

Preferably the kit of the thirteenth aspect further comprises a thermostable polymerase.

In a fourteenth aspect, the invention provides an antibody that binds a protein of the first aspect, or a fragment thereof.

In a fifteenth aspect, the invention provides a protein having an amino acid sequence selected from the group consisting of the sequences set forth in SEQ ID NOS: 1 to 85, or an immunogenic fragment thereof.

Preferably, the protein has an amino acid sequence selected from the group consisting of the sequences set forth in SEQ ID NOS: 1 to 43.

More preferably, the protein has an amino acid sequence selected from the group consisting of the sequences set forth in SEQ ID NOS: 20, 34 and 35.

In a sixteenth aspect, the invention provides a nucleic acid sequence selected from the group consisting of the sequences set forth in SEQ ID NOS: 86 to 166, or an immunogenic fragment thereof.

Preferably, the nucleic acid has a sequence selected from the group consisting of the sequences set forth in SEQ ID NOS: 86 to 127.

More preferably, the nucleic acid has a sequence selected from the group consisting of the sequences set forth in SEQ ID NOS: 105, 120 and 121.

### BRIEF DESCRIPTION OF THE FIGURES AND TABLES

In order that the invention may be readily understood and put into practical effect, preferred embodiments will now be described by way of example with reference to the accompanying figures and tables wherein like reference numerals refer to like parts and wherein:

FIG. 1 shows amino acid sequence alignments for C. trachomatis serovar D proteins (“CT” for human infecting form of Chlamydia) and corresponding C. muridarum proteins (“TC” for mouse infecting form of Chlamydia) for CT481 (SEQ ID NO: 1), NT01CT0505 (SEQ ID NO: 2), CT344 (SEQ ID NO: 3), CT404 (SEQ ID NO: 4), CT405 (SEQ ID NO: 5), CT166 (SEQ ID NO: 6), CT472 (SEQ ID NO: 7), CT412 (SEQ ID NO: 8), CT190 (SEQ ID NO: 9), CT241 (SEQ ID NO: 10), CT049 (SEQ ID NO: 11), CT114 (SEQ ID NO: 12), CT558 (SEQ ID NO: 13), CT775 (SEQ ID NO: 14), CT175 (SEQ ID NO: 15), CT155 (SEQ ID NO: 16), CT475 (SEQ ID NO: 17), CT286 (SEQ ID NO: 18), CT139 (SEQ ID NO: 19), CT561 (SEQ ID NO: 20), CT 661 (SEQ ID NO: 21), CT774 (SEQ ID NO: 22), CT820 (SEQ ID NO: 23), CT857 (SEQ ID NO: 24), CT031 (SEQ ID NO: 25), CT032 (SEQ ID NO: 26), CT047 (SEQ ID NO: 27), CT166 (SEQ ID NO: 28), CT174 (SEQ ID NO: 29), CT621 (SEQ ID NO: 30), CT155 (SEQ ID NO: 32), CT608 (SEQ ID NO: 33), CT305 (SEQ ID NO: 34), CT386 (SEQ ID NO: 35), CT433 (SEQ ID NO: 36), CT434 (SEQ ID NO: 37), CT475 (SEQ ID NO: 38), CT425 (SEQ ID NO: 41), CT562 (SEQ ID NO: 42), CT828 (SEQ ID NO: 43), TC0767 (SEQ ID NO: 44), TC0768 (SEQ ID NO: 45), TCA04 (SEQ ID NO: 46), TCA05 (SEQ ID NO: 47), TC0684 (SEQ ID NO: 48), TC0685 (SEQ ID NO: 49), TC0439 (SEQ ID NO: 50), TC0757 (SEQ ID NO: 51), TC0693 (SEQ ID NO: 52), TC0462 (SEQ ID NO: 53), TC0512 (SEQ ID NO: 54), TC0319 (SEQ ID NO: 55), TC0390 (SEQ ID NO: 56), TC0847 (SEQ ID NO: 57), TC0156 (SEQ ID NO: 58), TC0446 (SEQ ID NO: 59), TC0447 (SEQ ID NO: 60), TC0760 (SEQ ID NO: 61), TC0559 (SEQ ID NO: 62), TC0416 (SEQ ID NO: 63), TC0850 (SEQ ID NO: 64), TC0032 (SEQ ID NO: 65), TC0155 (SEQ ID NO: 66), TC0207 (SEQ ID NO: 67), TC0247 (SEQ ID NO: 68), TC0300 (SEQ ID NO: 69), TC0301 (SEQ ID NO: 70), TC0346 (SEQ ID NO: 71), TC0439 (SEQ ID NO: 72), TC0444 (SEQ ID NO: 73), TC0445 (SEQ ID NO: 74), TC0446 (SEQ ID NO: 75), TC0447 (SEQ ID NO: 76), TC0490 (SEQ ID NO: 77), TC0579 (SEQ ID NO: 78), TC0665 (SEQ ID NO: 79), TC0717 (SEQ ID NO: 80), TC0718 (SEQ ID NO: 81), TC0760 (SEQ ID NO: 82), TC0708 (SEQ ID NO: 83), TC0851 (SEQ ID NO: 84), TC0215 (SEQ ID NO: 85).

For the murine TC sequences, the actual fragments obtained are indicated as underlined regions of the full length protein sequences.

FIG. 2 shows nucleotide sequence alignments encoding the amino acids sequences (SEQ ID NOS: 21 to 37 and 65 to 82) set forth in FIG. 1. FIG. 2 shows C. muridarum (“TC” for mouse infecting form of Chlamydia) shown on a top line and C. trachomatis serovar D (“CT” for human infecting form of Chlamydia) shown on a bottom line for CT 661 (SEQ ID NO: 106), CT774 (SEQ ID NO: 107), CT820 (SEQ ID NO: 108), CT857 (SEQ ID NO: 109), CT031 (SEQ ID NO: 110), CT032 (SEQ ID NO: 111), CT047 (SEQ ID NO: 112), CT166 (SEQ ID NO: 113), CT174 (SEQ ID NO: 114), CT162 (SEQ ID NO: 115), CT621 (SEQ ID NO: 116), CT175 (SEQ ID NO: 117), CT155 (SEQ ID NO: 118), CT608 (SEQ ID NO: 119), CT305 (SEQ ID NO: 120), CT386 (SEQ ID NO: 121), CT433 (SEQ ID NO: 122), CT434 (SEQ ID NO: 123), CT475 (SEQ ID NO: 124), TC0032 (SEQ ID NO: 149), TC0155 (SEQ ID NO: 150), TC0207 (SEQ ID NO: 151), TC0247 (SEQ ID NO: 152), TC0300 (SEQ ID NO: 153), TC0301 (SEQ ID NO: 154), TC0346 (SEQ ID NO: 155), TC0439 (SEQ ID NO: 156), TC0444 (SEQ ID NO: 157), TC0446 (SEQ ID NO: 159), TC0447 (SEQ ID NO: 160), TC0490 (SEQ ID NO: 161), TC0579 (SEQ ID NO: 162), TC0665 (SEQ ID NO: 163), TC0717 (SEQ ID NO: 164), TC0718 (SEQ ID NO: 165), and TC0760 (SEQ ID NO: 166).

For full length nucleic acids comprising at least part of an isolated nucleotide sequence of the invention, underlined nucleotide sequences indicate isolated nucleotide sequences and a corresponding region for human infecting form of Chlamydia for encoding fragments of proteins of the invention.

FIG. 3 shows nucleotide sequences of C. trachomatis serovar D nucleotides (“CT” for human infecting form of Chlamydia) and corresponding C. muridarum nucleotides (“TC” for mouse infecting form of Chlamydia) for CT481 (SEQ ID NO: 86), NT01CT0505 (SEQ ID NO: 87), CT344 (SEQ ID NO: 88), CT404 (SEQ ID NO: 89), CT405 (SEQ ID NO: 90), CT166 (SEQ ID NO: 91), CT472 (SEQ ID NO: 92), CT412 (SEQ ID NO: 93), CT190 (SEQ ID NO: 94), CT241 (SEQ ID NO: 95), CT049 (SEQ ID NO: 96), CT114 (SEQ ID NO: 97), CT558 (SEQ ID NO: 98), CT775 (SEQ ID NO: 99), CT175 (SEQ ID NO: 100), CT155 (SEQ ID NO: 101), CT475 (SEQ ID NO: 102), CT286 (SEQ ID NO: 103), CT139 (SEQ ID NO: 104), CT561 (SEQ ID NO: 105), CT425 (SEQ ID NO: 125), CT562 (SEQ ID NO: 126), CT0828 (SEQ ID NO: 127), TC0767 (SEQ ID. NO: 128), TC0768 (SEQ ID NO: 129), TCA04 (SEQ ID NO: 130), TCA05 (SEQ ID NO: 131), TC0684 (SEQ ID NO: 132), TC0685 (SEQ ID NO: 133), TC0439 (SEQ ID NO: 134), TC0757 (SEQ ID NO: 135), TC0693 (SEQ ID NO: 136), TC0462 (SEQ ID NO: 137), TC0512 (SEQ ID NO: 138), TC0319 (SEQ ID NO: 139), TC0390 (SEQ ID NO: 140), TC0847 (SEQ ID NO: 141), TC0156 (SEQ ID NO: 142), TC0446 (SEQ ID NO: 143), TC0447 (SEQ ID NO: 144), TC0760 (SEQ ID NO: 145), TC0559 (SEQ ID NO: 146), TC0416 (SEQ ID NO: 147) and TC0850 (SEQ ID NO: 148).

TABLE 1 lists C. muridarum and C. trachomatis Chlamydia proteins, the percentage homology of the proteins, C. muridarum protein function, C. trachomatis protein function, and the Genbank identification numbers.

TABLE 2 lists nucleic acids encoding the amino acid sequences for the identified C. muridarum and C. trachomatis Chlamydia proteins, the percentage homology of the nucleic acids, C. muridarum protein function, C. trachomatis protein function, the percentage homology of the proteins and the Genbank identification numbers.

TABLE 3 shows novel antigens containing promiscuous T cell epitopes using in silico analysis.

### DETAILED DESCRIPTION OF THE INVENTION

### Definitions

Unless defined otherwise, all technical and scientific terms used herein have a meaning as commonly understood by those of ordinary skill in the art to which the invention belongs. Although any method and material similar or equivalent to those described herein can be used in the practice or testing of the present invention, preferred methods and materials are described. For the purpose of the present invention, the following terms are defined below.

For the purposes of this invention, by “isolated” is meant material that has been removed from its natural state or otherwise been subjected to human manipulation. Isolated material may be substantially or essentially free from components that normally accompany it in its natural state, or may be manipulated so as to be in an artificial state together with components that normally accompany it in its natural state. Isolated material includes material in native and recombinant form. For example, isolated Chlamydia spp and nucleic acids and proteins isolated therefrom.

It will be appreciated that the taxonomic family Chlamydiaceae is divided into two genera, Chlamydia and Chlamydophila. Recently the family was reclassified into nine species, Chlamydia trachomatis and Chlamydophila pneumoniae that comprise the major human pathogens, and Chlamydia muridarum, Chlamydia suis, Chlamydophila abortus, Chlamydophila psittaci, Chlamydophila caviae, Chlamydophila felis and Chlamydophila pecorum, that, along with C. pneumoniae, are primarily animal pathogens. This new classification of the Chlamydiaceae family is based on 16SrRNA and 23SrRNA gene sequence analysis (Everett et al. 1999).

The family Chlamydiaceae formerly consisted only of one genus, Chlamydia and of four species, C. trachomatis, C. psittaci, C. pneumoniae and C. pecorum. The different species are further divided into biovars and serovars based on disease pathology and serology respectively. C. trachomatis is divided into two human biovars, the trachoma biovar which incorporates serovars A, B, Ba, C, D, Da, E, F, G, H, I, Ia, J and K (Wang and Grayston 1970) and the lymphogranuloma venereum biovar that consists of serovars L1, L2 and L3 (Wang et al. 1973).

Use of the term “Chlamydia” herein generally relates to all of the abovementioned family, genera, species, biovars and serovars unless specifically referred to as otherwise.

In one form, the invention relates to diagnostic methods and regents for detecting one or more family, genera, species, biovar and/or serovar of Chlamydia. In a preferred form, the invention relates to methods and reagents for detecting one or more human Chlamydia species, biovars and/or serovars. In another form of the invention, pharmaceutical compositions, including immunotherapeutic compositions and vaccines are used to prevent or ameliorate infection by one or more species, biovar and/or serovar of Chlamydia. In a preferred form, the Chlamydia species, biovar and/or serovar is capable of infecting a human.

The terms “C. trachomatis Mouse Pneumonitis (MoPn)” and “C. trachomatis SFPD” as used herein respectively refer to mouse and hamster isolates, namely, a biovar of C. trachomatis, which has been reclassified as C. muridarum.

By “endogenous” nucleic acid or protein is meant a nucleic acid or protein that may be found in a native organelle, cell, tissue, bacteria, organism or animal in isolation or otherwise.

### Proteins

By “protein” is also meant “polypeptide” or “peptide”, either term referring to an amino acid polymer, comprising natural and/or non-natural amino acids, including L- and D-isomeric forms, as are well understood in the art. A polypeptide comprises more than 60 contiguous amino acids. A peptide comprises up to 60 contiguous amino acids.

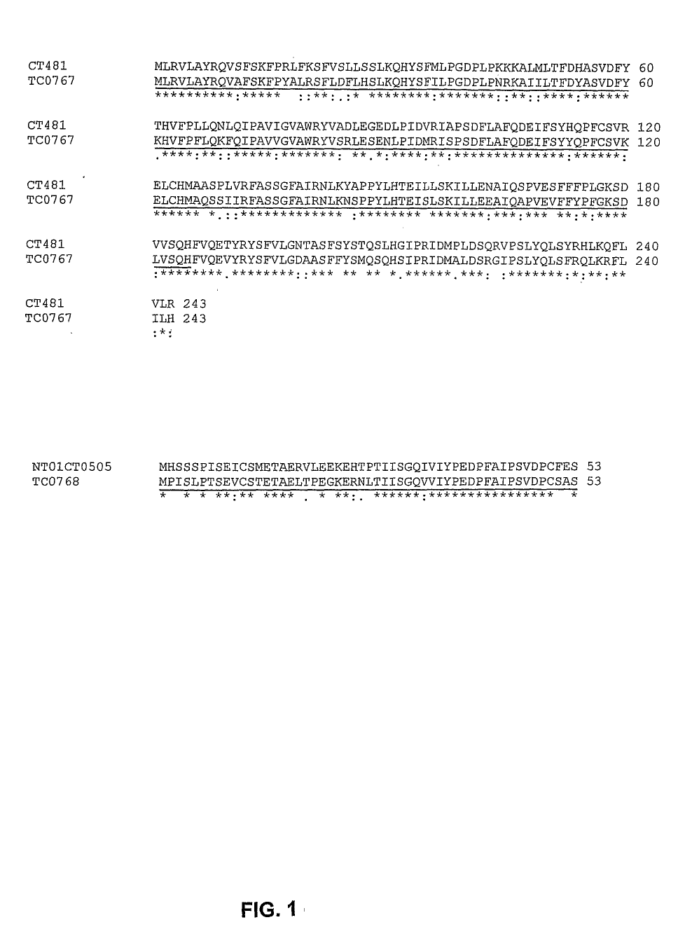

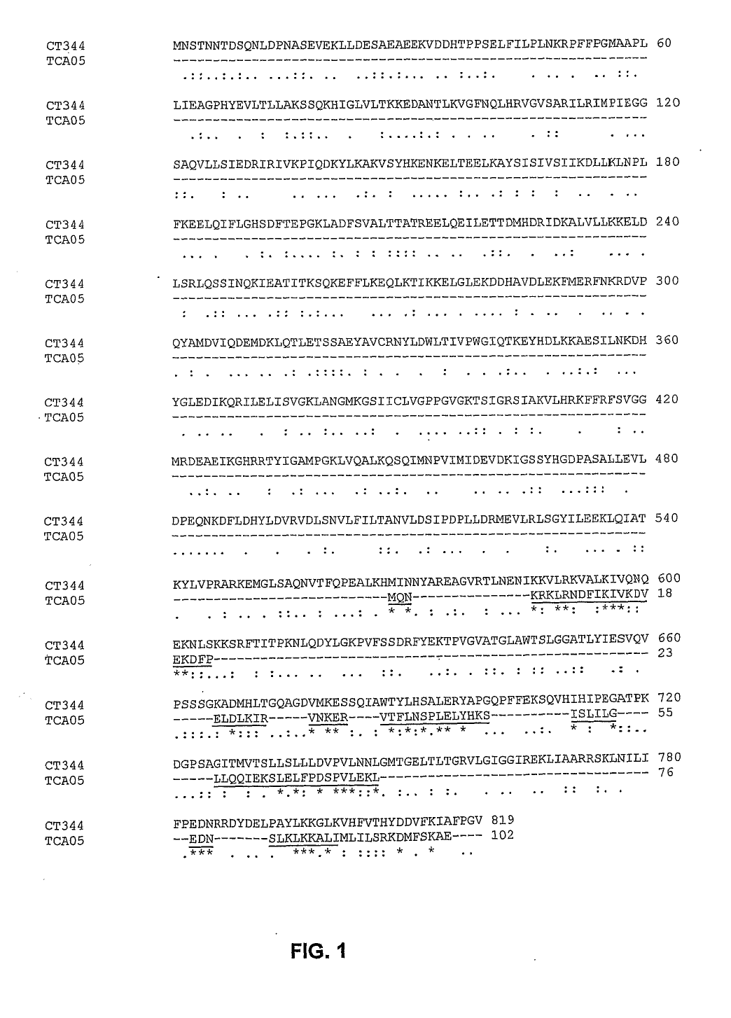

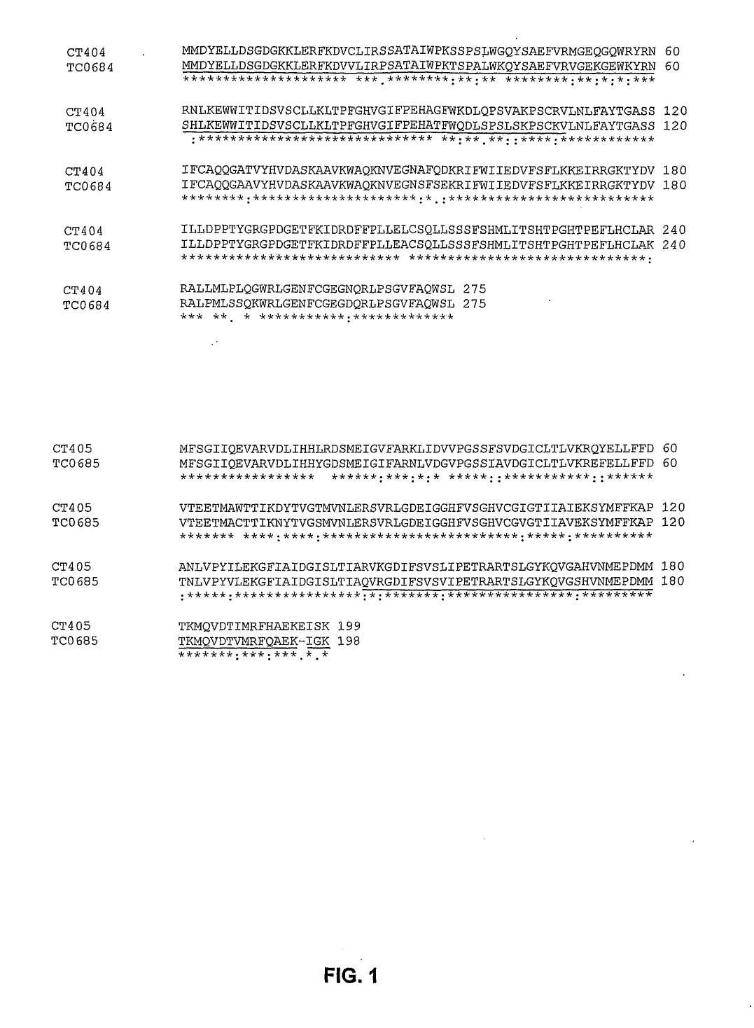

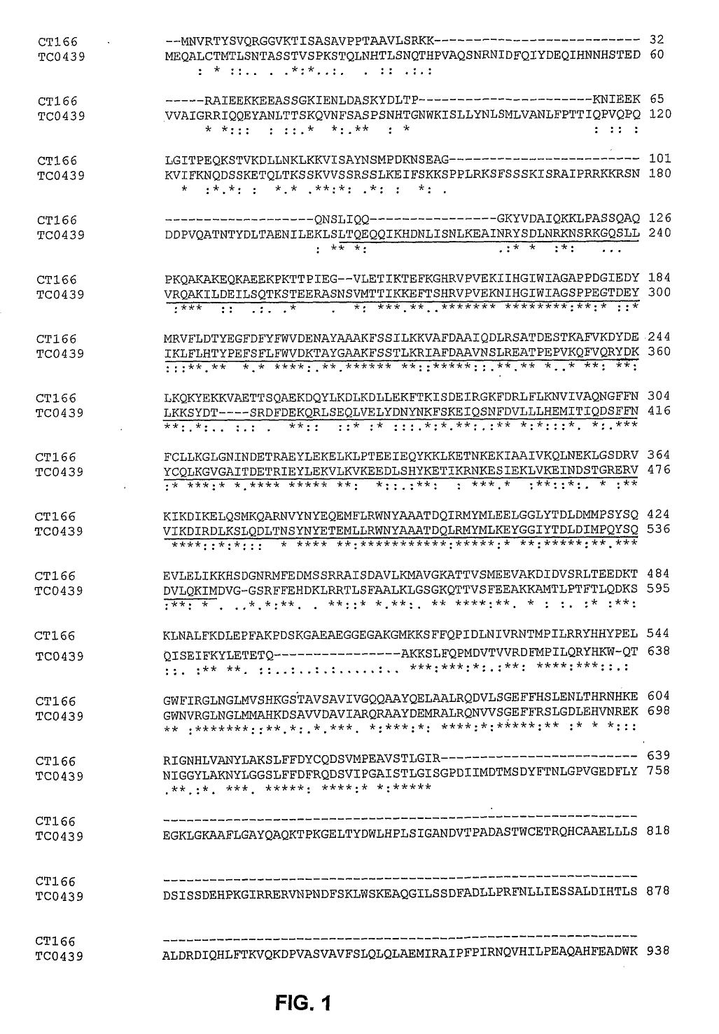

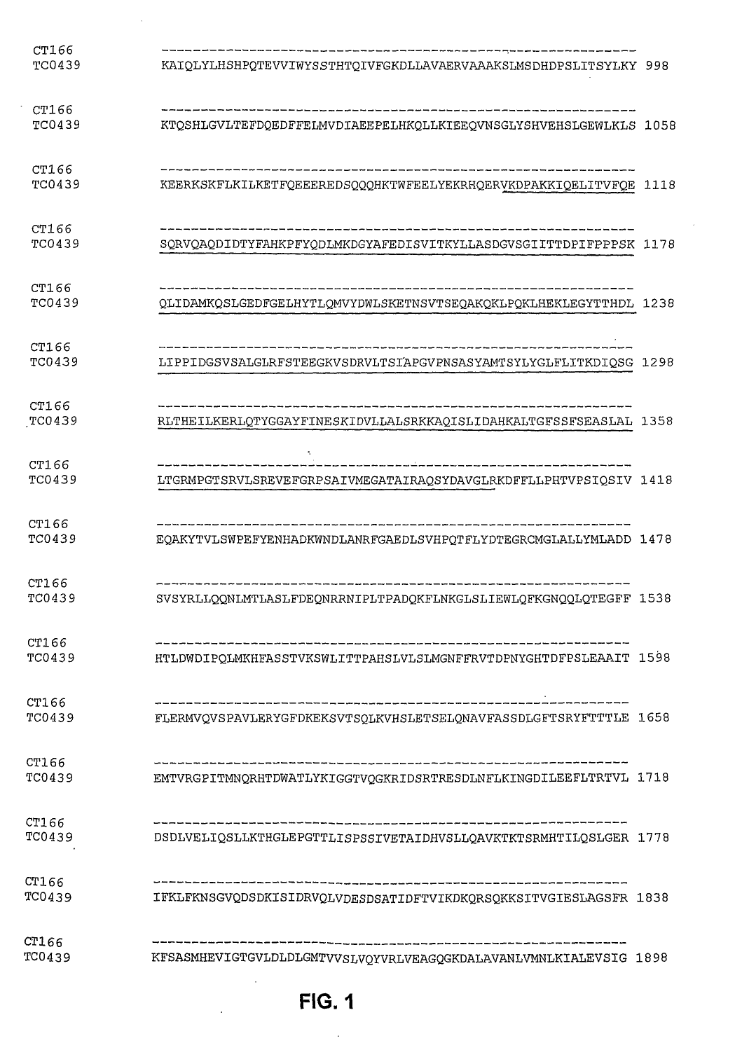

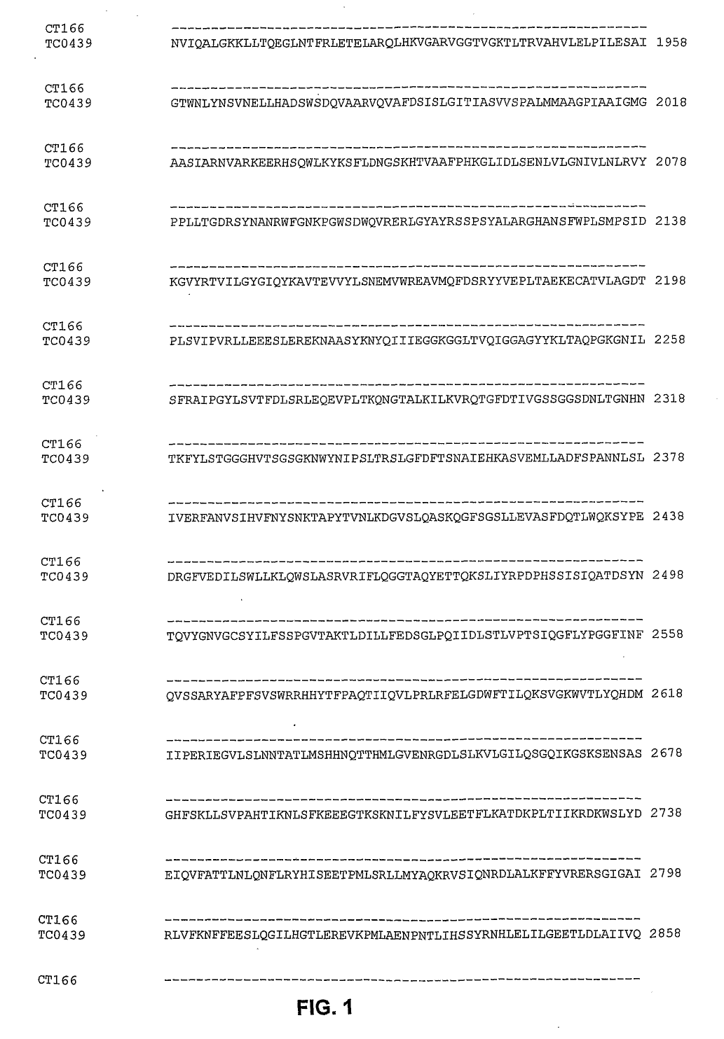

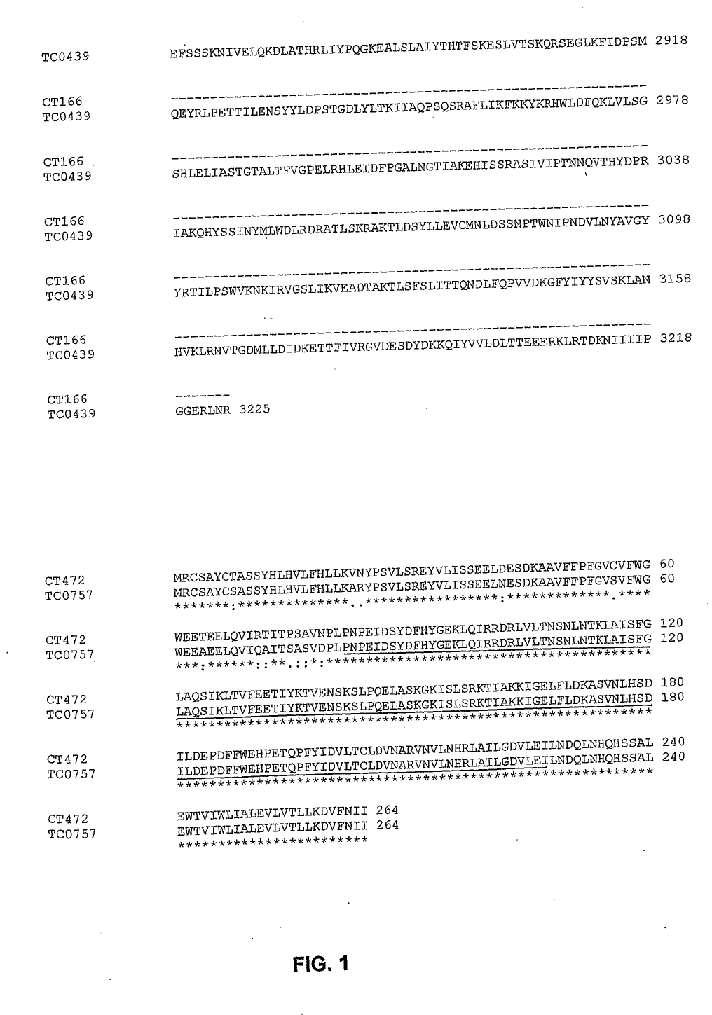

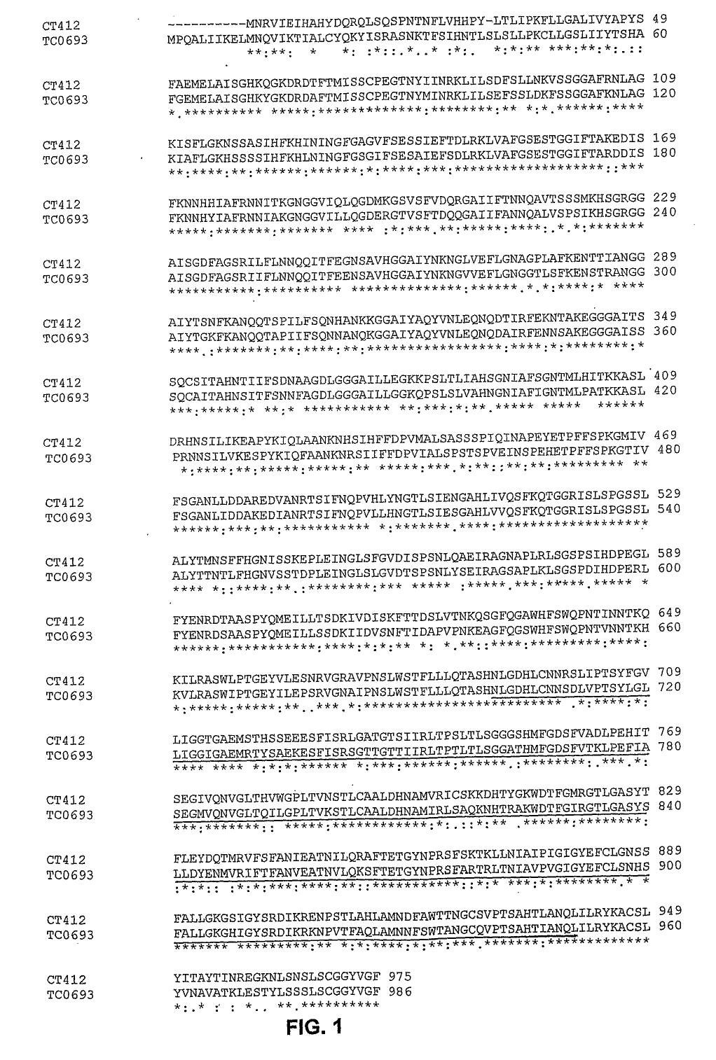

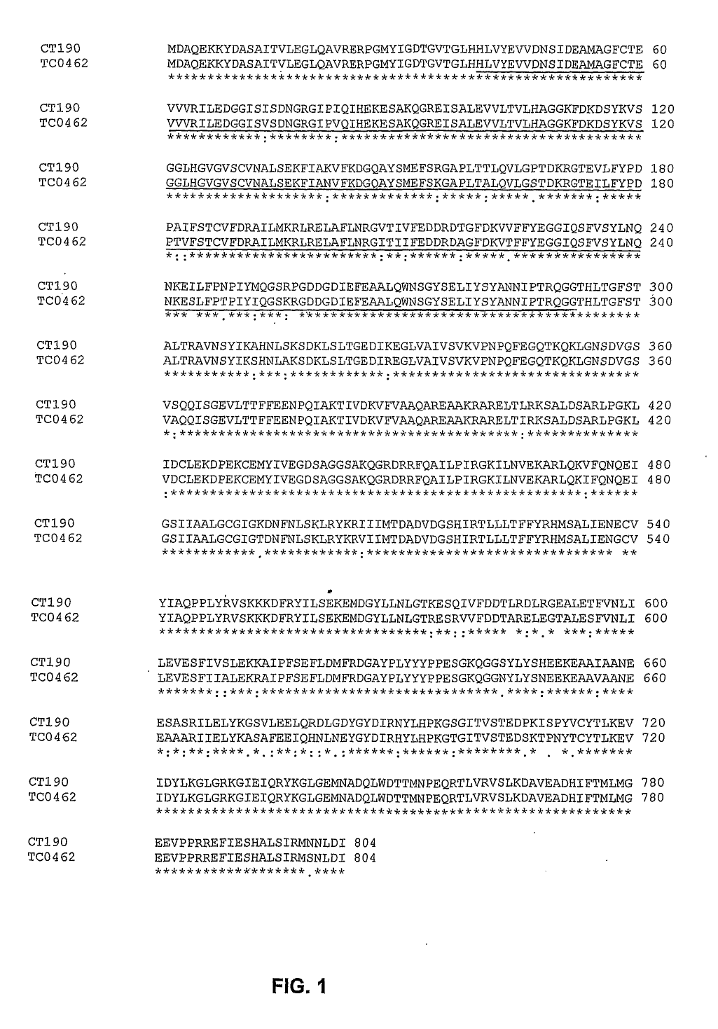

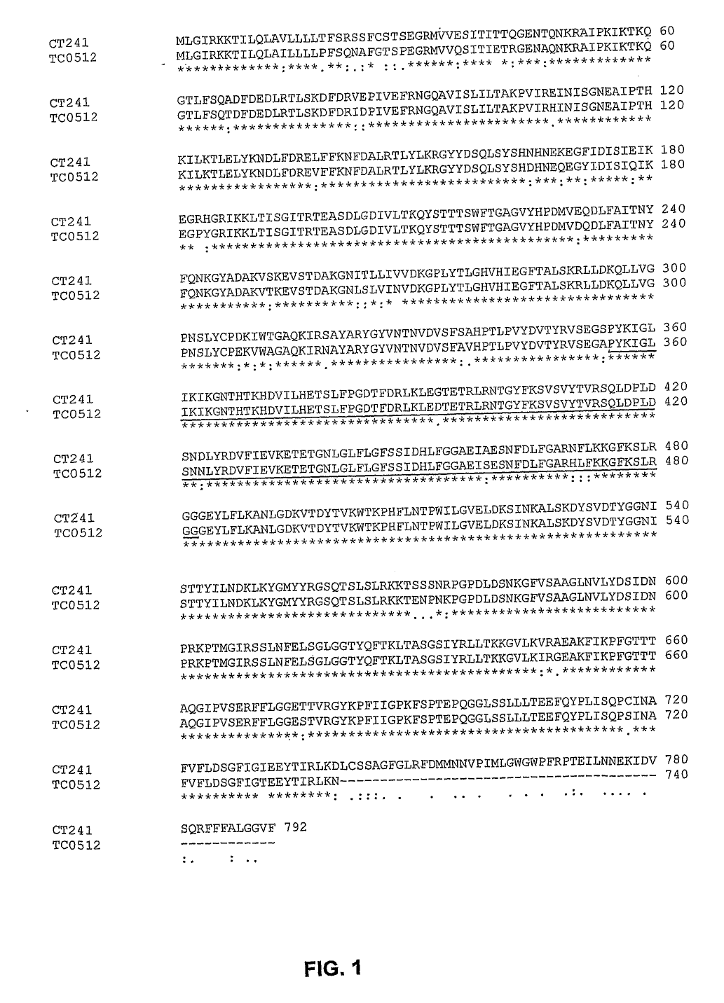

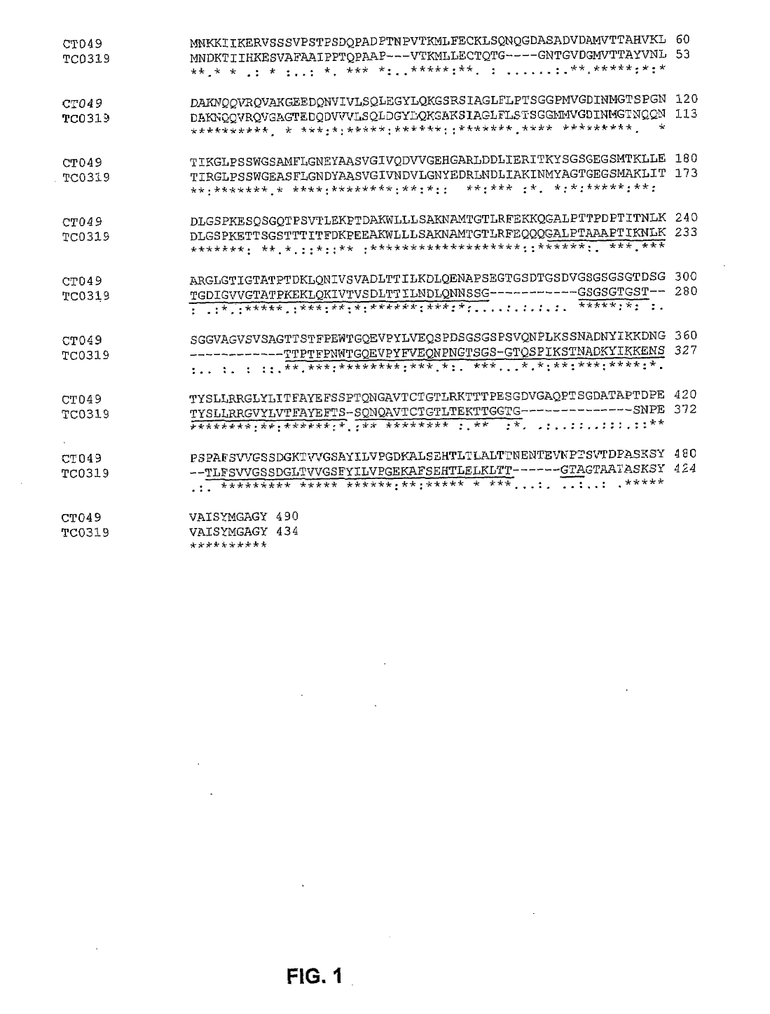

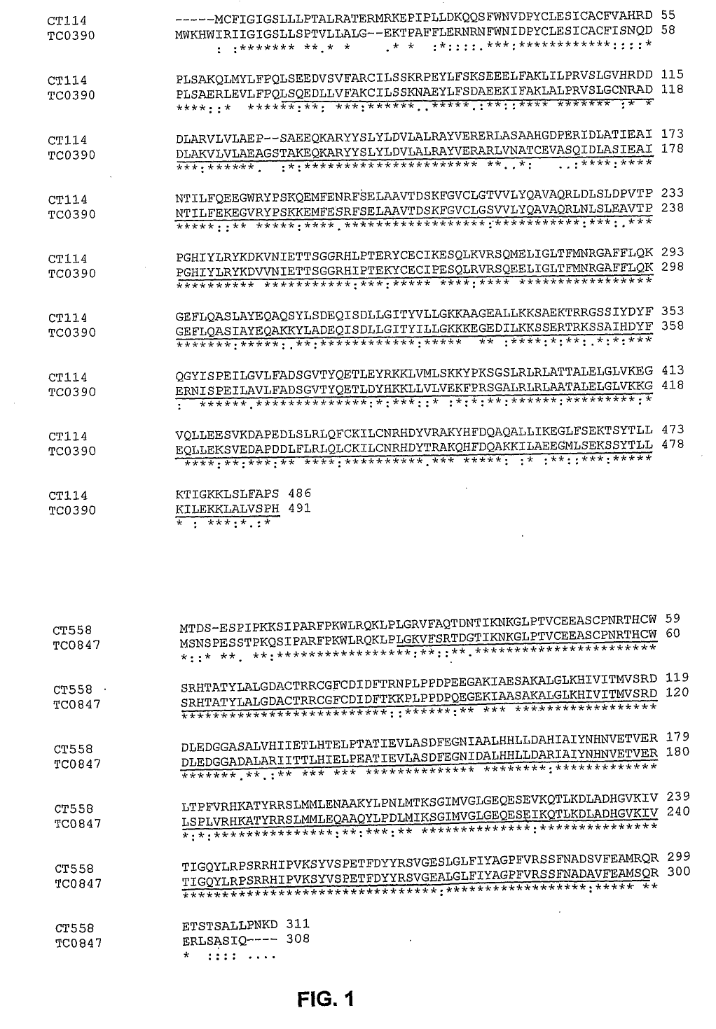

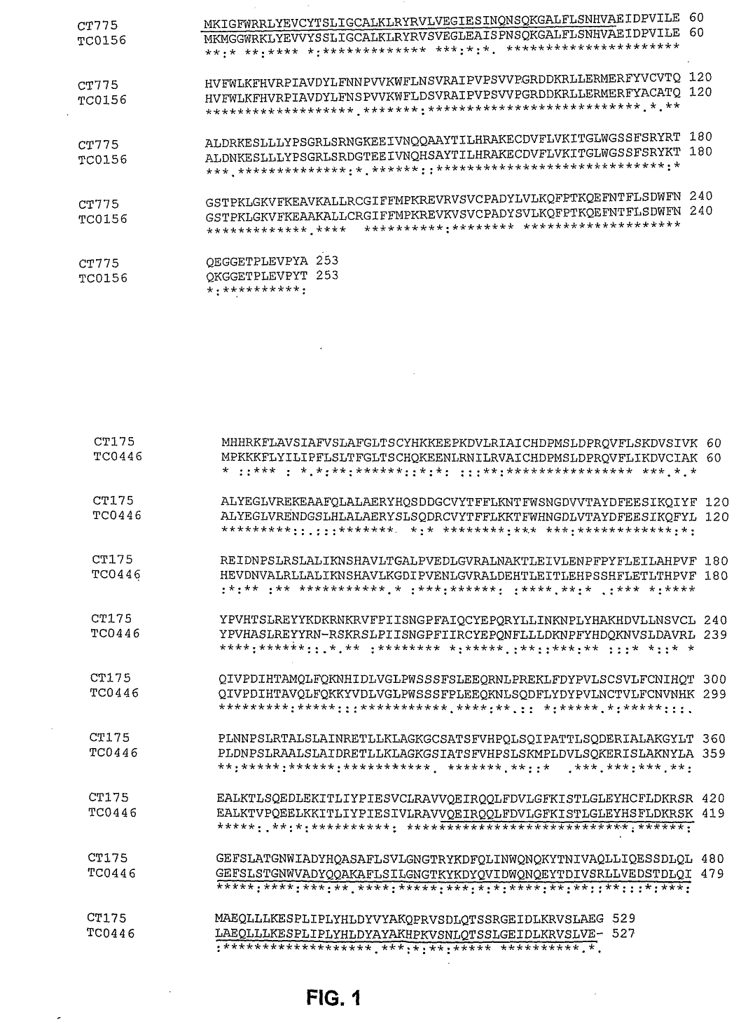

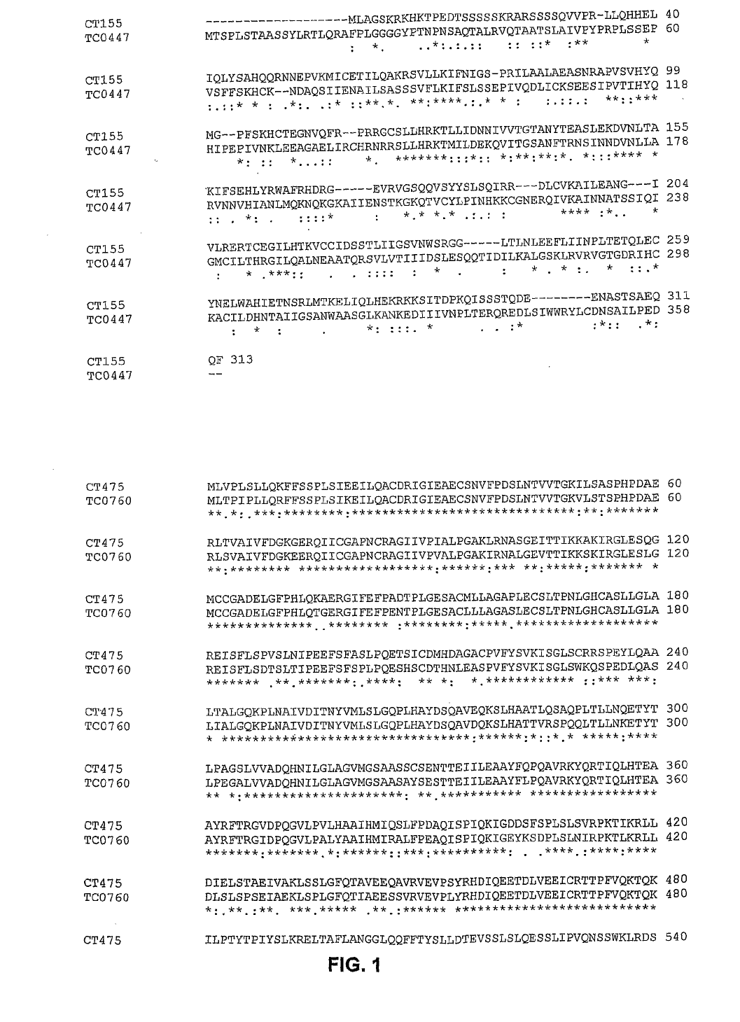

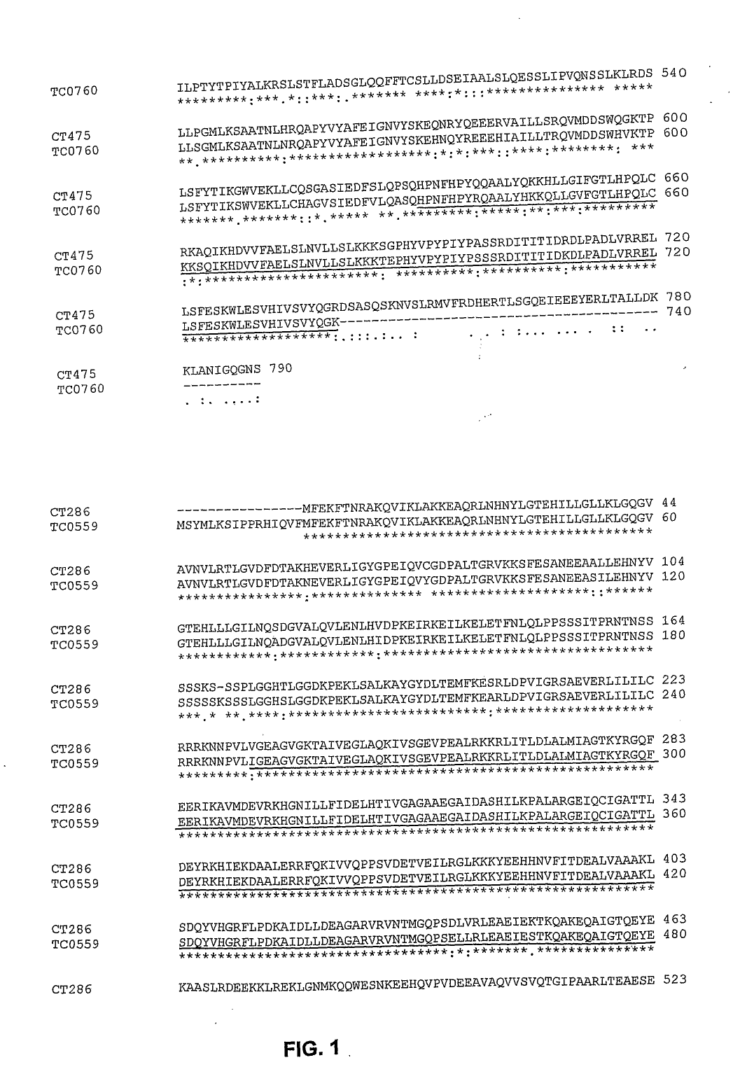

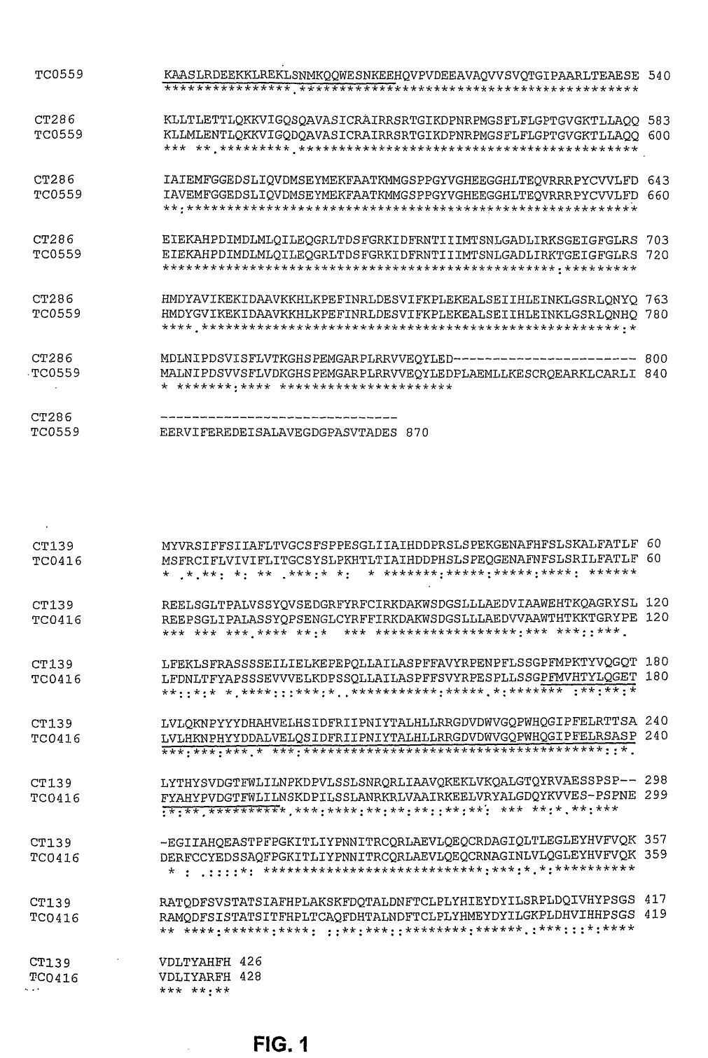

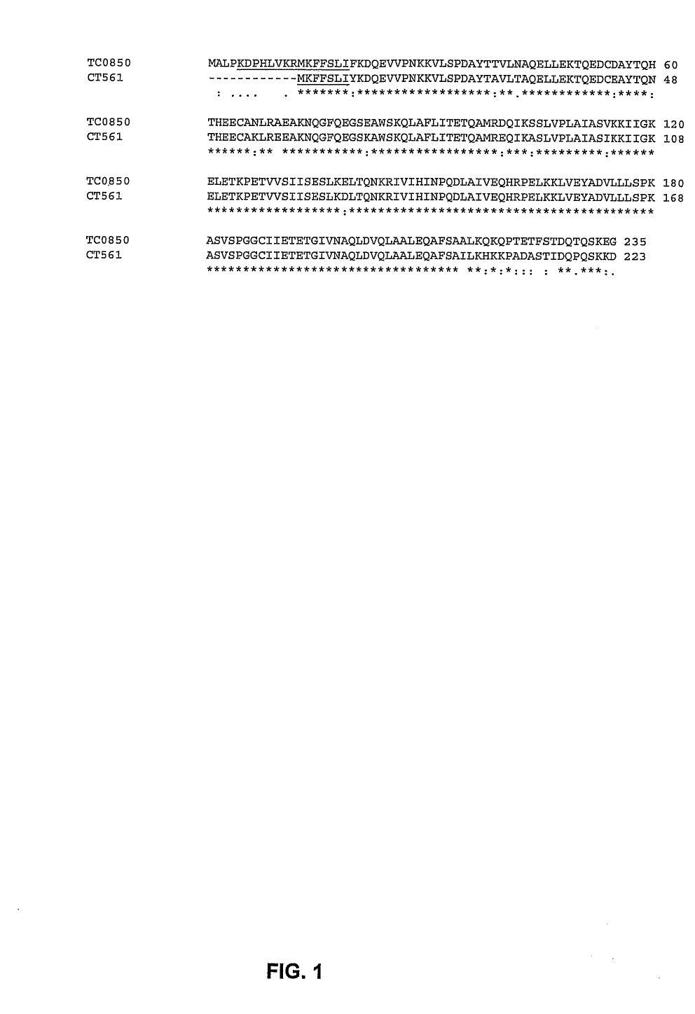

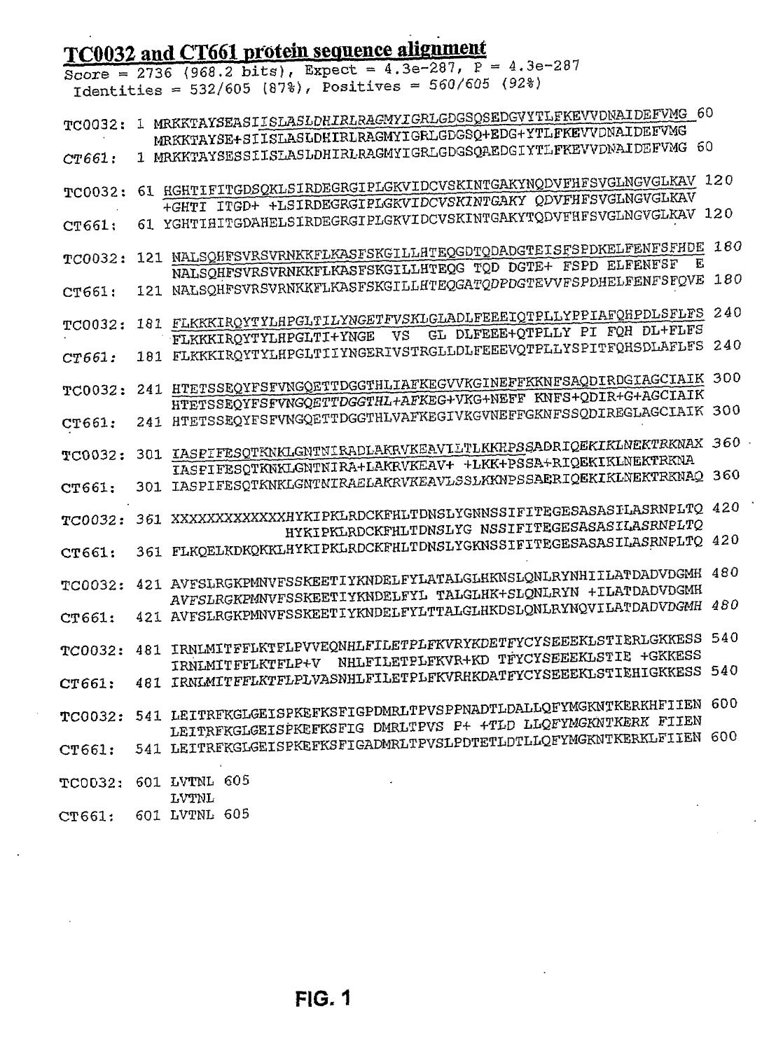

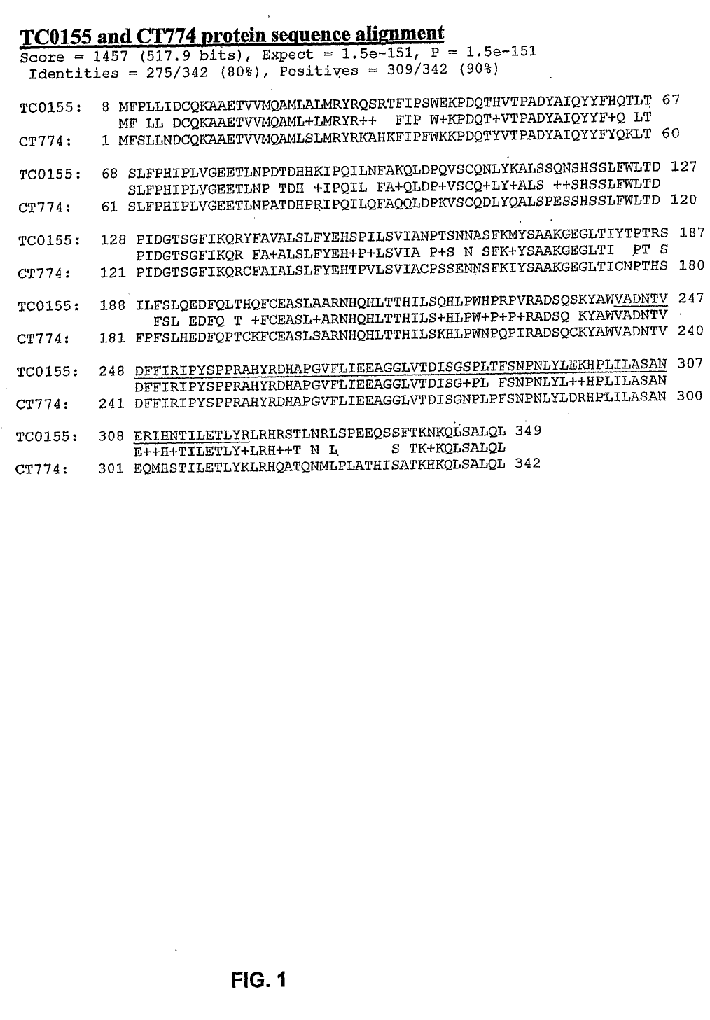

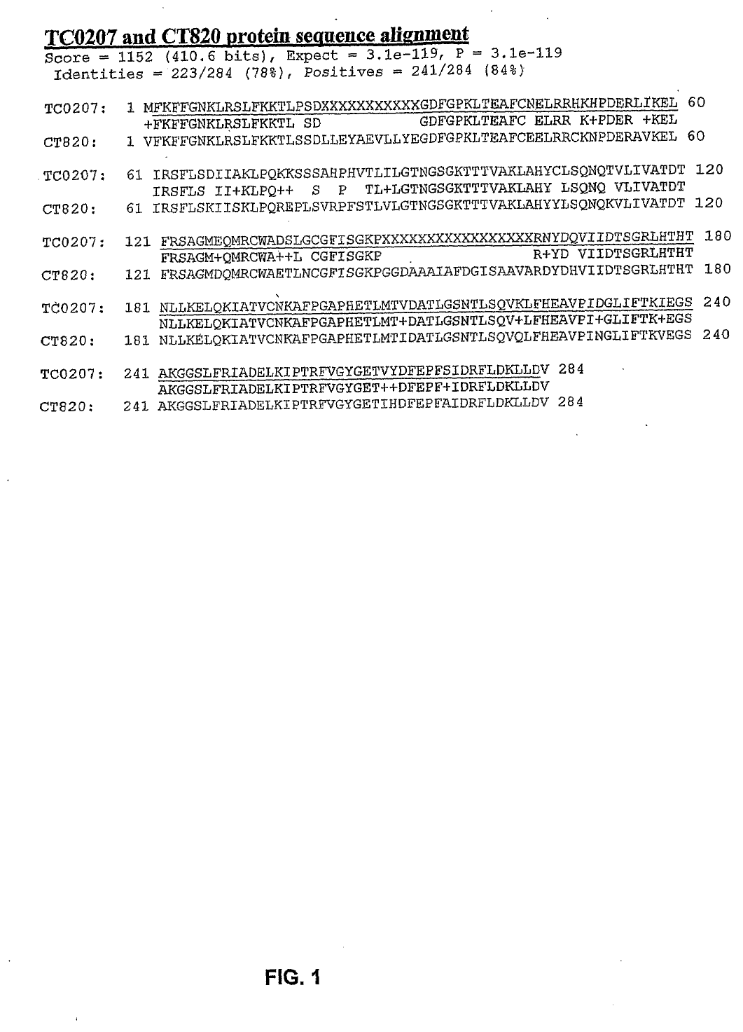

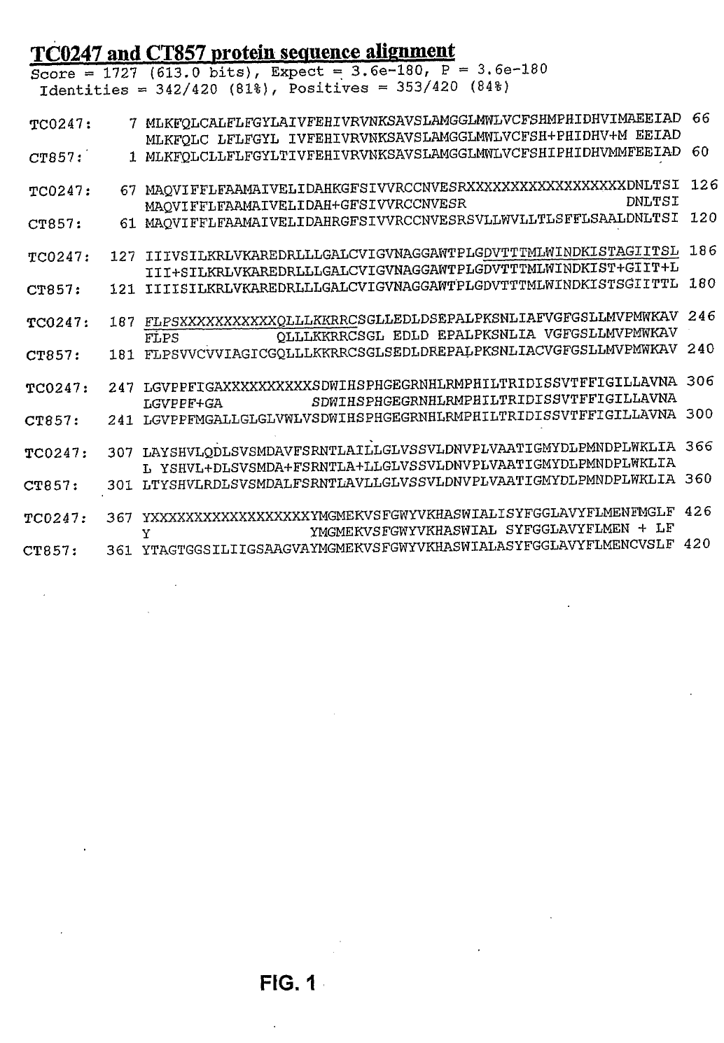

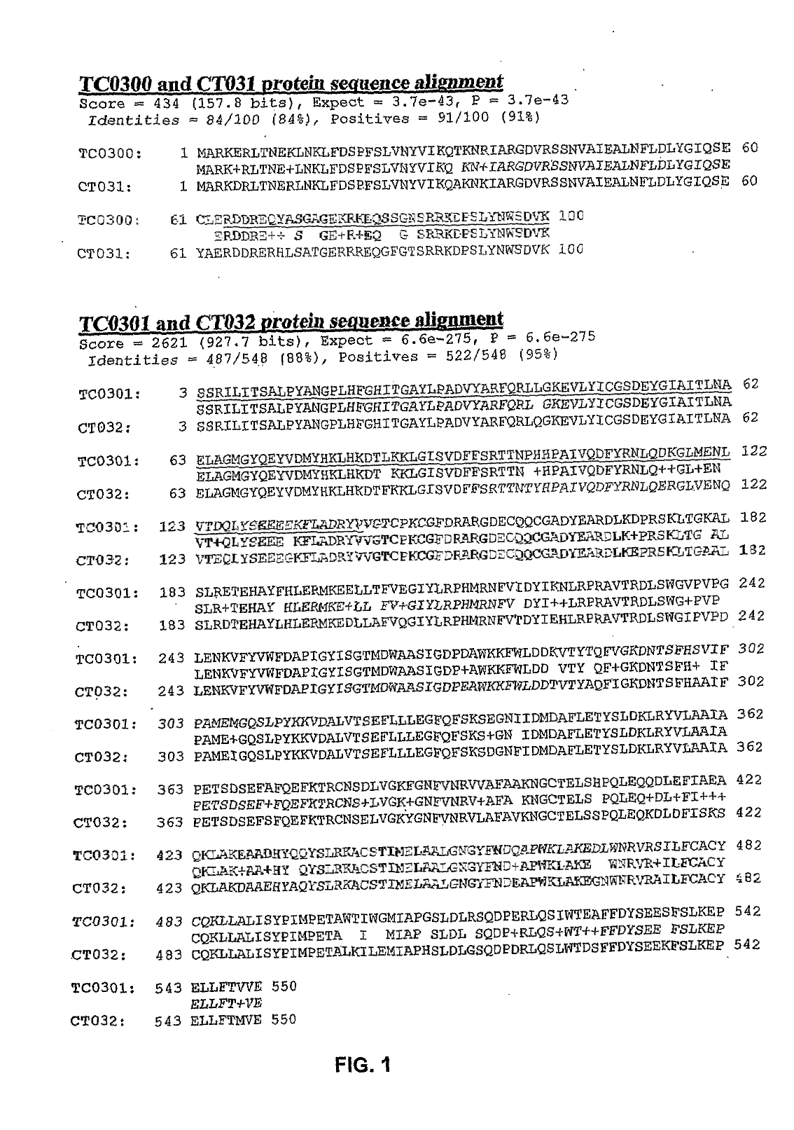

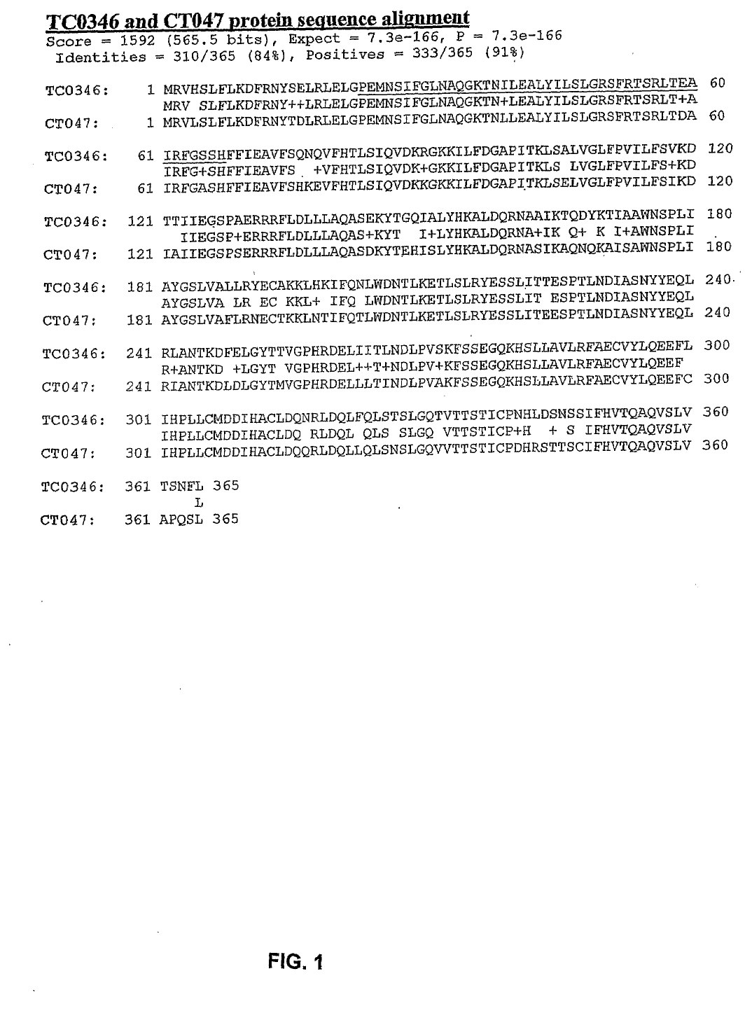

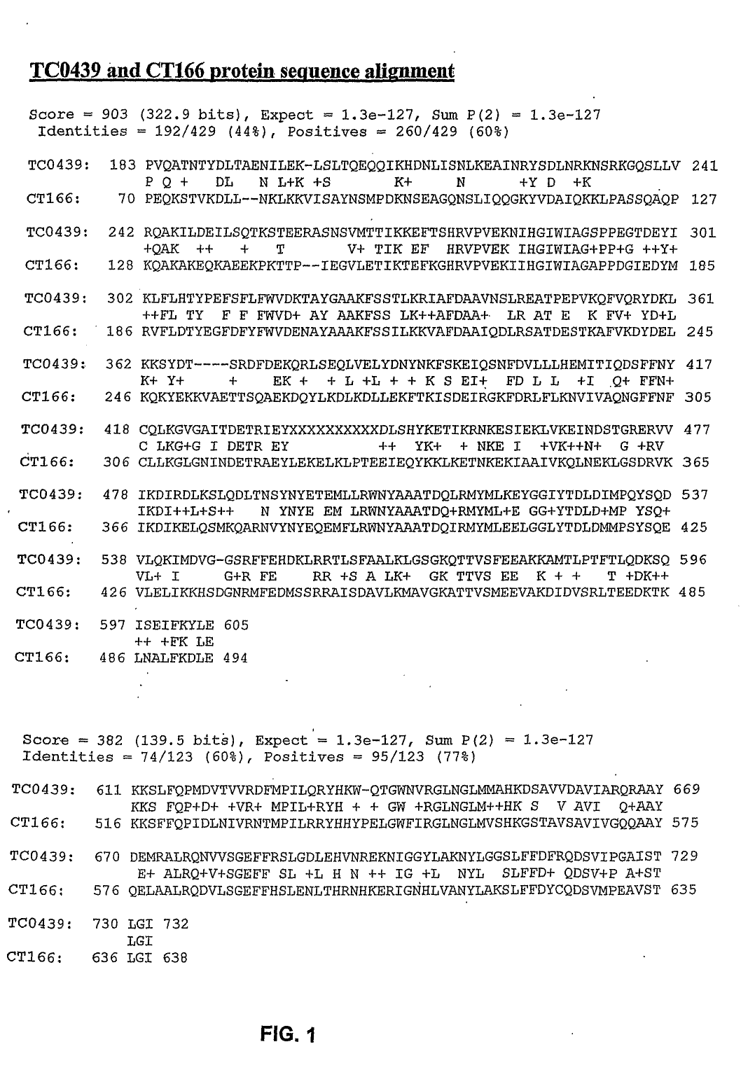

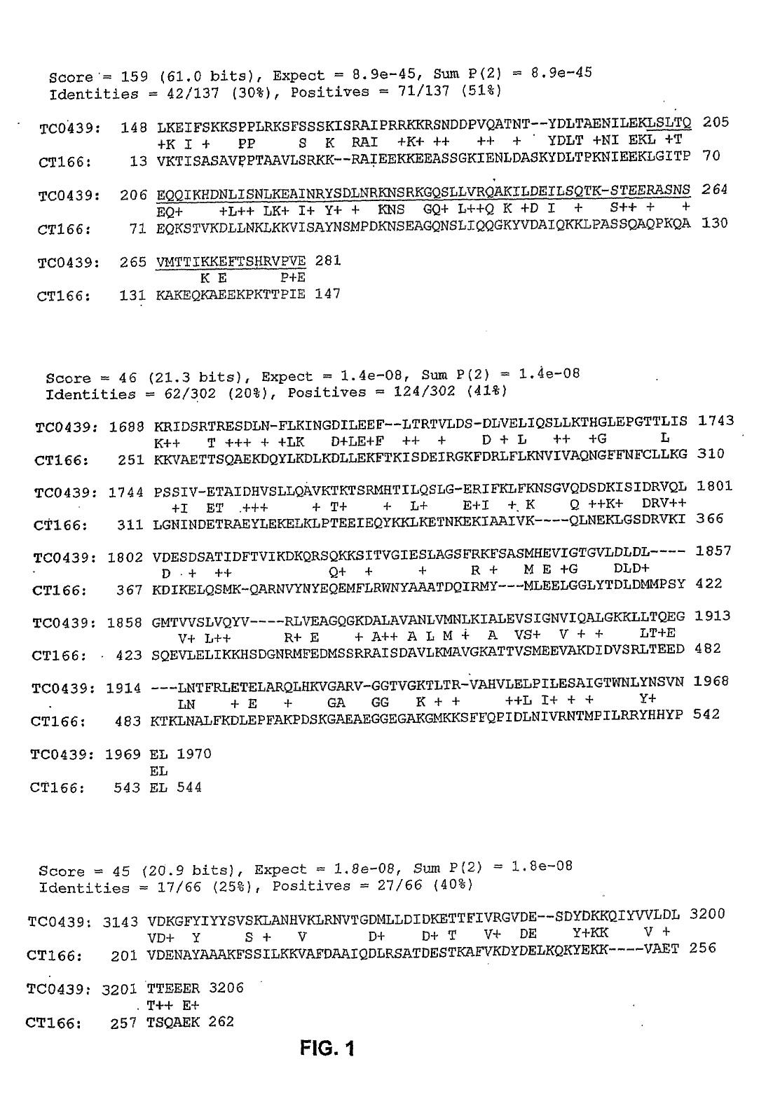

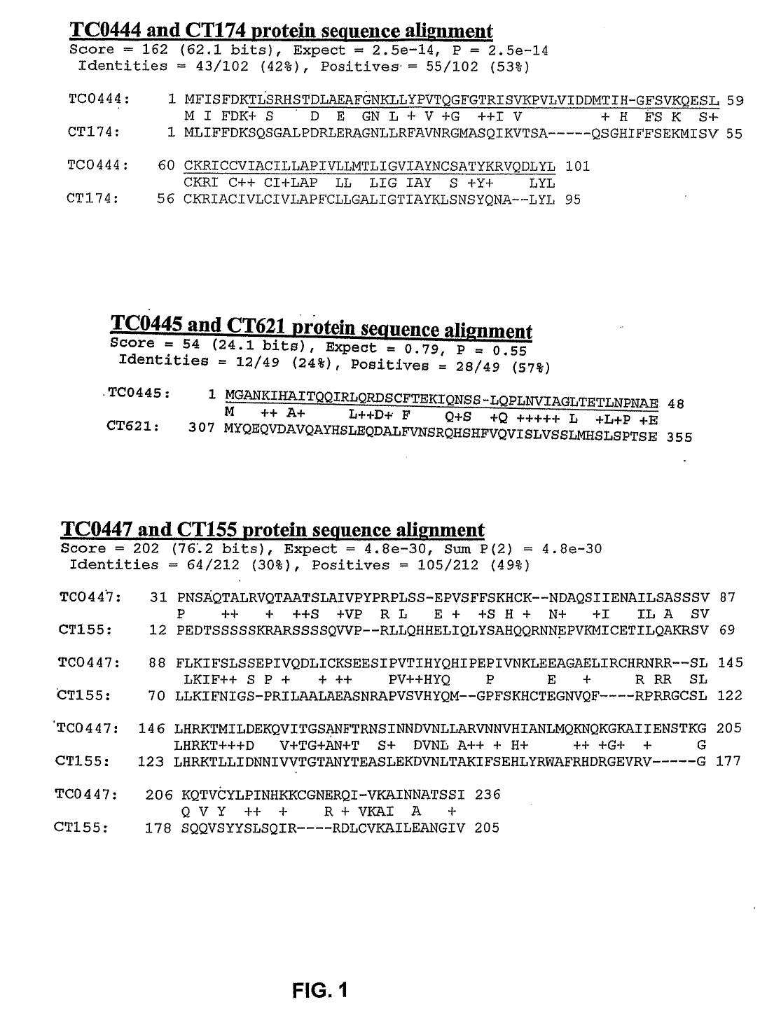

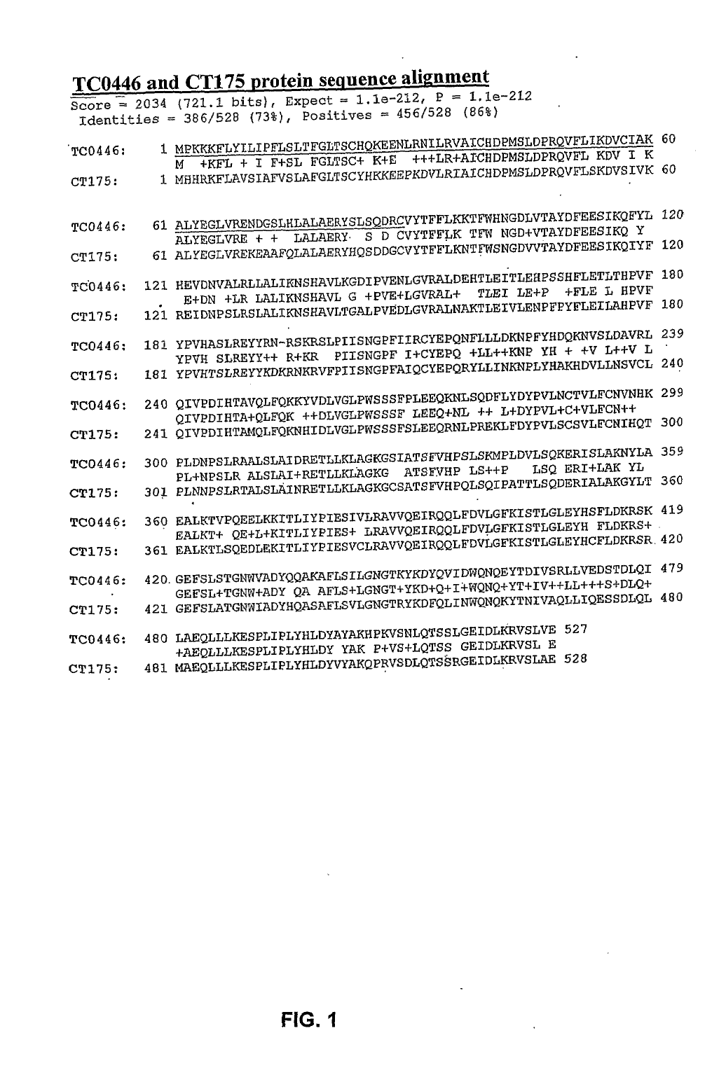

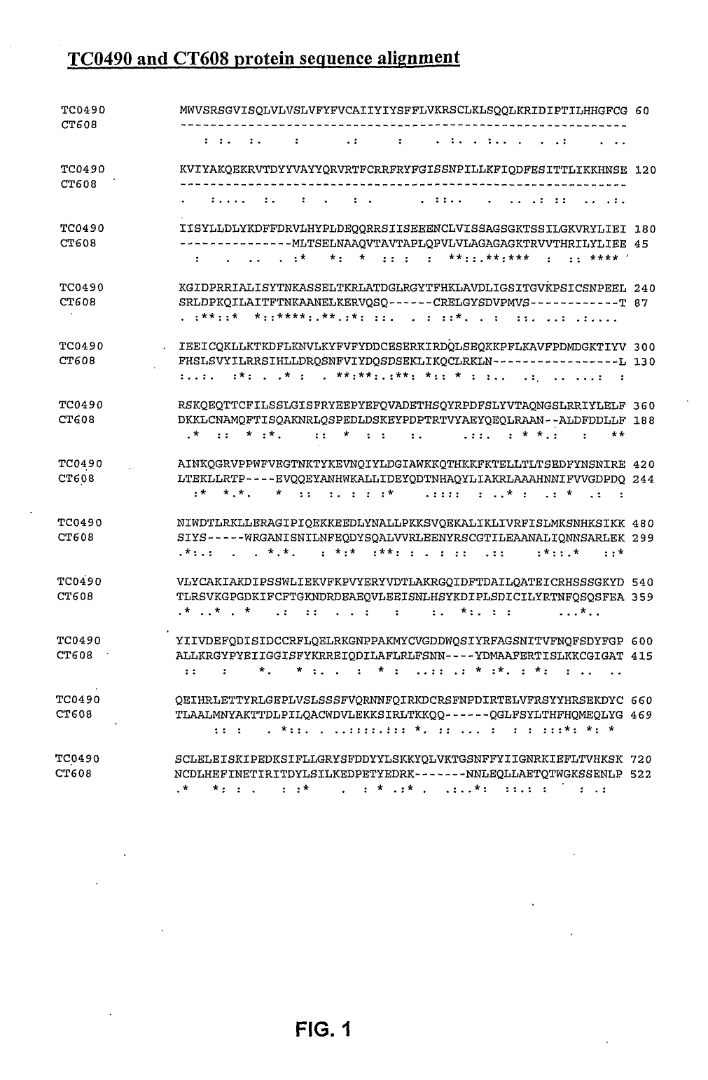

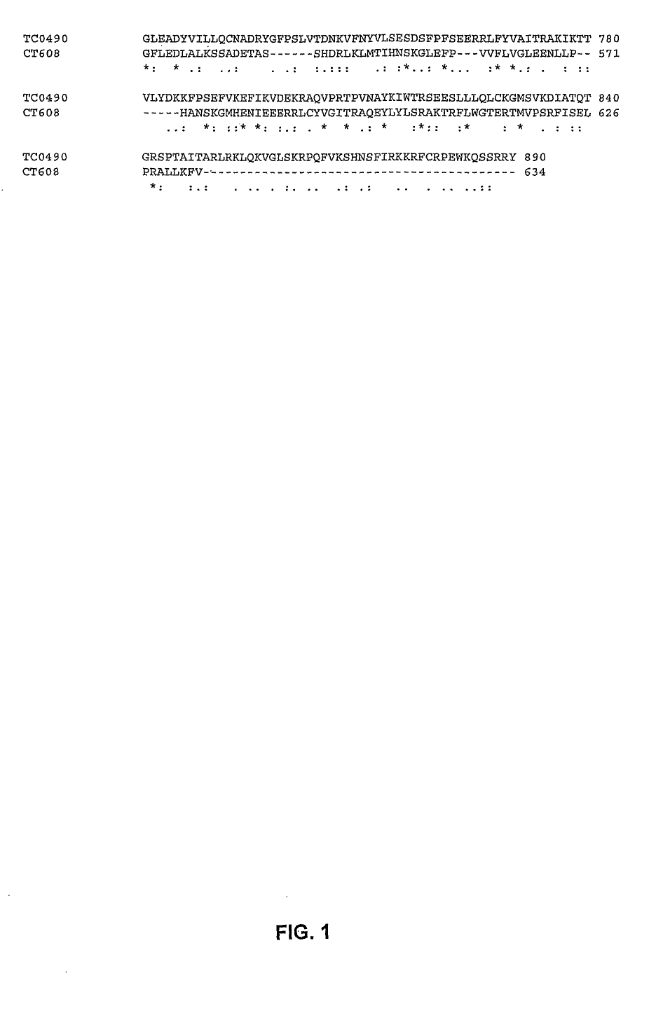

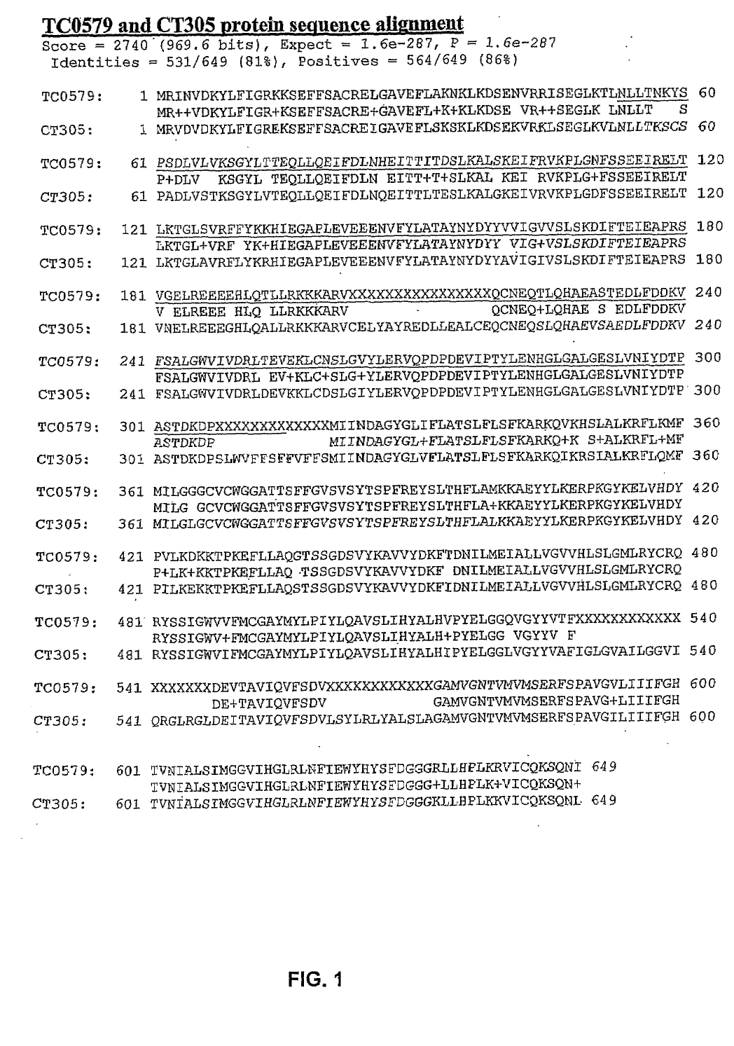

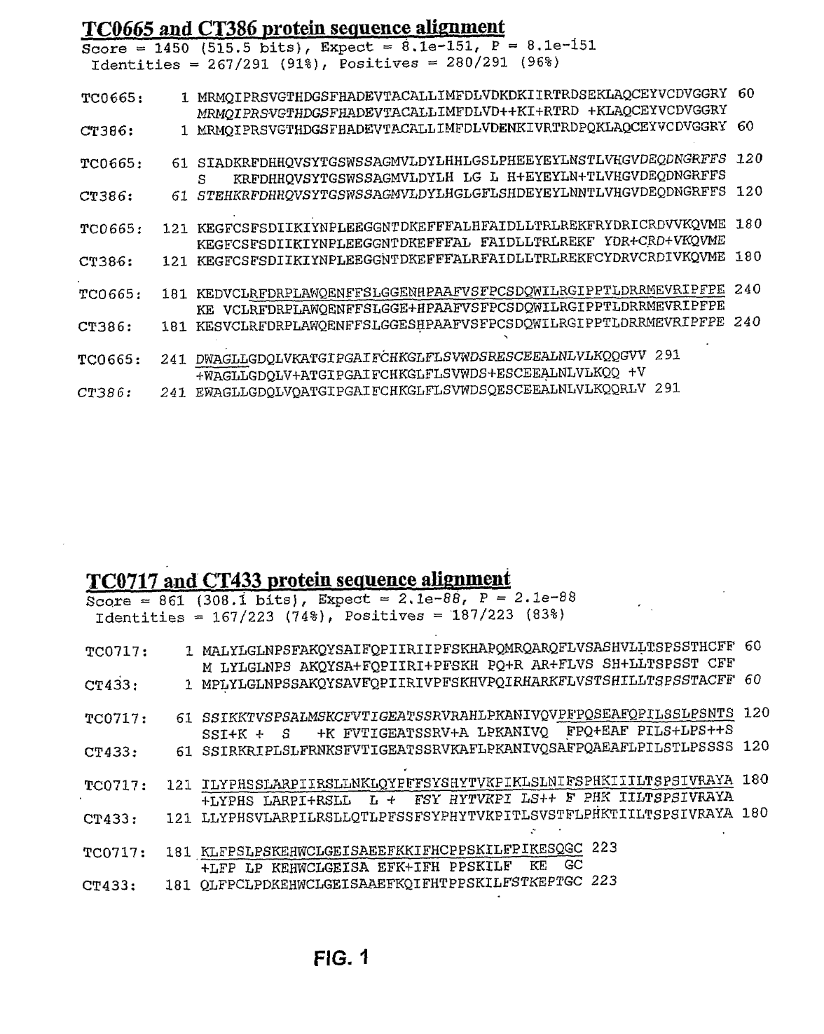

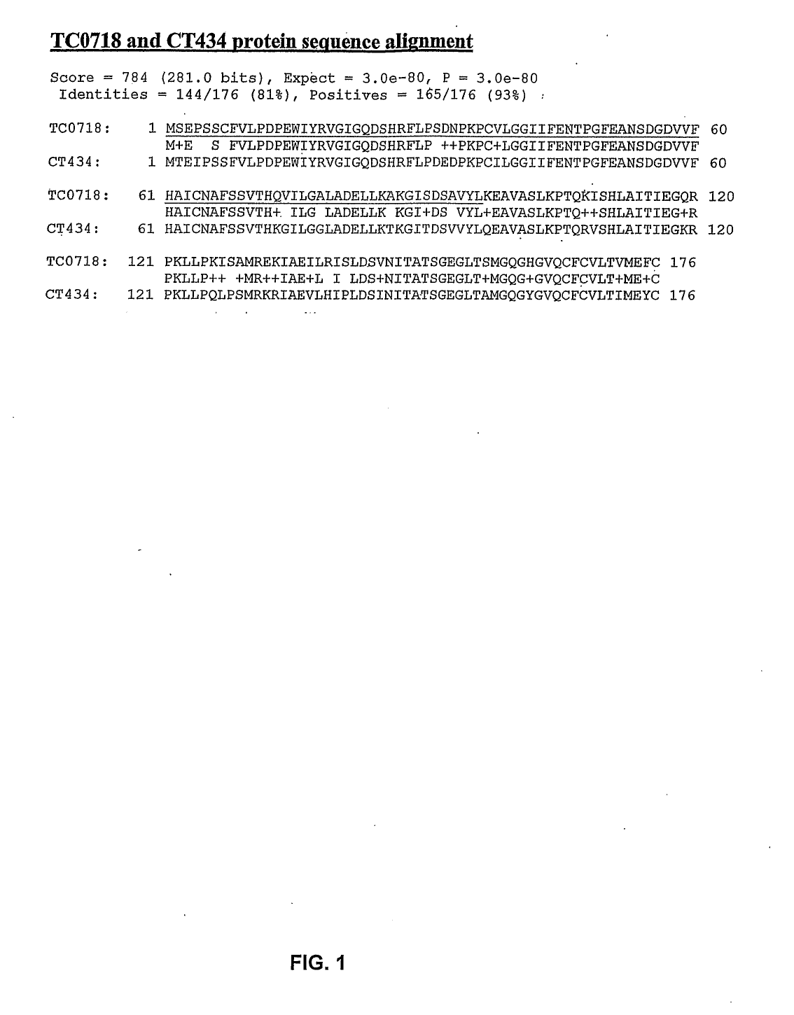

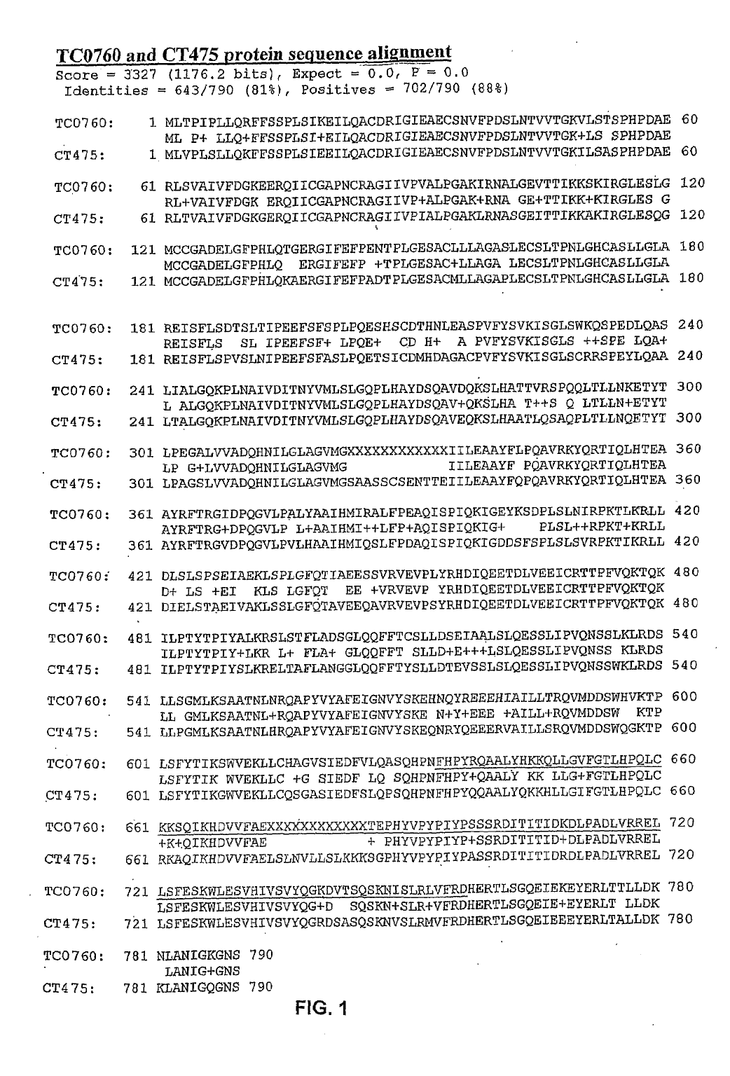

In one embodiment, a “fragment” includes a protein comprising an amino acid sequence that constitutes less than 100% of an amino acid sequence of an entire protein. A fragment preferably comprises less than 99%, 98%, 97%, 96%, 95%, 94%, 93%, 92%, 91%, 90%, 85%, 80%, 75%, 70%, 60%, 50%, 40%, 30%, 20% or as little as even 10%, 5% or 3% of the entire protein. Many of the antigens shown in FIG. 1 herein are fragments of an entire “full length” protein as shown by underlined sections of full-length proteins shown n FIG. 1. It will be appreciated that in some sequences of FIG. 1 only the mouse sequence is underlined (for a mouse infecting form of Chlamydia). We note the corresponding human sequence fragments (for a human infecting form of Chlamydia) are also considered a fragment of the invention, which fragments may be readily deduced from the corresponding mouse sequence. A fragment also comprises sub-fragments, for example fragments of the identified fragments of the full-length proteins as shown in the figures.

The fragment may also include a “biologically active” fragment, which retains biological activity of a given protein. For example, a biologically active fragment may comprise one or more functional domains of a protein described herein, for example as recited in FIG. 1, including DNA gyrase subunit B, sulfite synthesis/biphosphate phosphatase, cell division protein FtsY, methionyl-tRNA synthetase, DAN helicase (uvrD), ATP synthase subunit I (atpI) or a metal dependent hydrolase.

A biologically active fragment may also refer to a fragment that is capable of inducing an immune response in an animal, whether protective or not. It will be appreciated that a biologically active fragment capable of inducing an immune response, in particular a B-cell response, may be useful in generating antibodies for uses including diagnostics, affinity purification, as actives in a pharmaceutical composition and as a research reagent. An immune response also refers to a T-cell mediated response and a response involving antigen presenting cells.

It will be appreciated that based on, the method for isolating the antigens, the identified protein fragments are likely to be capable of inducing an immune response in an animal. Such fragments include proteins comprising an amino acid sequence indicated in the figures, in particular underlined amino acid sequences, including the corresponding human infecting form of Chlamydia as shown.

A biologically active fragment in one form is capable of being bound by an antibody that binds to a same region of the full-length protein. The biologically active fragment in another form is capable of binding an appropriate target ligand.

It is understood that the fragment may be derived from either a native or a recombinant protein. The biologically active fragment constitutes at least greater than 1% of the biological activity of the entire protein, preferably at least greater than 5%, 10%, 15% or 20% biological activity, more preferably at least greater than 25%, 35%, 45% biological activity and even more preferably at least greater than 50%, 60%, 70%, 80%, 90% and even 95%, 99% or 100% biological activity of the entire protein. It is also contemplated that a biologically active fragment may have a biological activity greater than a full-length protein, for example if a domain that functions as a repressor or down regulates a function is deleted from a full length protein.

In another embodiment, a “fragment” is a small protein, for example of at least 6, preferably at least 10 and more preferably at least 20 amino acids in length, which comprises one or more antigenic determinants or epitopes. Such a fragment may also be a biologically active fragment. Larger fragments comprising more than one protein are also contemplated, and may be obtained through the application of standard recombinant nucleic acid techniques or synthesized using conventional liquid or solid phase synthesis techniques. For example, reference may be made to solution synthesis or solid phase synthesis as described, for example, in Chapter 9 entitled “Peptide Synthesis” by Atherton and Shephard that is included in a publication entitled “Synthetic Vaccines” edited by Nicholson and published by Blackwell Scientific Publications. Alternatively, proteins can be produced by digestion of a protein of the invention with a suitable proteinase. The digested fragments can be purified by, for example, high performance liquid chromatographic (HPLC) techniques.

As used herein, “variant” proteins are proteins of the invention in which one or more amino acids have been replaced by different amino acids. It is well understood in the art that some amino acids may be changed to others with broadly similar properties without changing the nature of the activity of the protein (conservative substitutions).

Substantial changes in function are made by selecting substitutions that are less conservative or non-conservative as is known in the art. Generally, the substitutions which are likely to produce the greatest changes in a protein's properties are those in which: (a) a hydrophilic residue (e.g., Ser or Thr) is substituted for, or by, a hydrophobic residue (e.g. Leu, Ile, Phe or Val); (b) a cysteine or proline is substituted for, or by, any other residue; (c) a residue having an electropositive side chain (e.g., Arg, His or Lys) is substituted for, or by, an electronegative residue (e.g., Glu or Asp) or (d) a residue having a bulky side chain (e.g., Phe or Trp) is substituted for, or by, one having a smaller side chain (e.g., Ala, Ser) or no side chain (e.g., Gly). Variant may also comprise one or more amino acid deletions and/or additions.

### Protein and Nucleic Acid Sequence Comparison

Terms used herein to describe sequence relationships between respective nucleic acids and proteins include “comparison window”, “sequence identity”, “percentage of sequence identity” and “substantial identity”. Because respective nucleic acids/proteins may each comprise: (1) only one or more portions of a complete nucleic acid/protein sequence that are shared by the nucleic acids/proteins, and (2) one or more portions which are divergent between the nucleic acids/polyproteins, sequence comparisons are typically performed by comparing sequences over a “comparison window” to identify and compare local regions of sequence similarity. A “comparison window” refers to a conceptual segment of typically at least 6 contiguous residues that is compared to a reference sequence. The comparison window may comprise additions or deletions (i.e., gaps) of about 20% or less as compared to the reference sequence (which does not comprise additions or deletions) for optimal alignment of the respective sequences. Optimal alignment of sequences for aligning a comparison window may be conducted by computerised implementations of algorithms (for example ECLUSTALW and BESTFIT provided by WebAngis GCG, 2D Angis, GCG and GeneDoc programs incorporated herein by reference) or by visual inspection by an operator and the best alignment (i.e., resulting in the highest percentage homology over the comparison window) generated by any of the various methods selected.

The ECLUSTALW program is used to align multiple sequences. This program calculates a multiple alignment of nucleotide or amino acid sequences according to a method by Thompson, J. D., Higgins, D. G. and Gibson, T. J. (1994). This is part of the original ClustalW distribution, modified for inclusion in EGCG. The BESTFIT program aligns forward and reverse sequences and sequence repeats. This program makes an optimal alignment of a best segment of similarity between two sequences. Optimal alignments are determined by inserting gaps to maximize the number of matches using the local homology algorithm of Smith and Waterman. ECLUSTALW and BESTFIT alignment packages are offered in WebANGIS GCG (The Australian Genomic Information Centre, Building JO3, The University of Sydney, N.S.W 2006, Australia).

Reference also may be made to the BLAST family of programs as for example disclosed by Altschul et al., 1997, Nucl. Acids Res. 25 3389, which is incorporated herein by reference.

A detailed discussion of sequence analysis can be found in Chapter 19.3 of Ausubel et al, supra.

The term “sequence identity” is used herein in its broadest sense to include the number of exact nucleotide or amino acid matches having regard to an appropriate alignment using a standard algorithm, having regard to the extent that sequences are identical over a window of comparison. Thus, a “percentage of sequence identity” is calculated by comparing two optimally aligned sequences over the window of comparison, determining the number of positions at which the identical nucleic acid base (e.g., A, T, C, G, U) occurs in both sequences to yield the number of matched positions, dividing the number of matched positions by the total number of positions in the window of comparison (i.e., the window size), and multiplying the result by 100 to yield the percentage of sequence identity. For example, “sequence identity” may be understood to mean the “match percentage” calculated by the DNASIS computer program (Version 2.5 for windows; available from Hitachi Software engineering Co., Ltd., South San Francisco, Calif., USA).

As generally used herein, a “homolog” shares a definable nucleotide or amino acid sequence relationship with a nucleic acid or protein of the invention as the case may be.

“Protein homologs” share at least 20%, preferably at least 75%, 80%, 85% or 90% and more preferably at least 95%, 96%, 97%, 98% or 99% sequence identity with the amino acid sequences of proteins of the invention as hereinbefore described. It will be appreciated that a homolog comprises all integer values less than 100%, for example the percent value as set forth above and others. Protein homologs include, for example homologs of DNA gyrase subunit B, sulfite synthesis/biphosphate phosphatase, cell division protein FtsY, methionyl-tRNA synthetase, DAN helicase (uvrD), ATP synthase subunit I (atpI) or a metal dependent hydrolase. Protein homologues are also shown in FIG. 1 wherein homologs of mouse and human infecting forms of Chlamydia are shown.

Included within the scope of homologs are “orthologs”, which are functionally-related proteins and their encoding nucleic acids, isolated from other organisms. For example, DNA gyrase subunit B, sulfite synthesis/biphosphate phosphatase, cell division protein FtsY, methionyl-tRNA synthetase, DAN helicase (uvrD), ATP synthase subunit I (atpI) or a metal dependent hydrolase are human orthologs of mouse proteins as shown in FIG. 1. Orthologs in relation to Chlamydia antigens include homologous proteins of animals known to be infectable by Chlamydia and Chlaymydophila, including for example: mice, hamsters, swine, cattle, sheep, goats, birds, guinea pigs, cats, koalas and horses, see for example Beagley and Timms, 2000, J. Reproductive 1 mm 48, 47. An ortholog may comprise an amino acid sequence 100% identical with a protein from another species.

With regard to protein variants, these can be created by mutagenising a protein or by mutagenising an encoding nucleic acid, such as by random mutagenesis or site-directed mutagenesis. Examples of nucleic acid mutagenesis methods are provided in Chapter 9 of CURRENT PROTOCOLS IN MOLECULAR BIOLOGY, Ausubel et al., supra which is incorporated herein by reference.

It will be appreciated by the skilled person that site-directed mutagenesis is best performed where knowledge of the amino acid residues that contribute to biological activity is available. In many cases, this information is not available, or can only be inferred by molecular modeling approximations, for example.

In such cases, random mutagenesis is contemplated. Random mutagenesis methods include chemical modification of proteins by hydroxylamine (Ruan et al., 1997, Gene 188 35), incorporation of dNTP analogs into nucleic acids (Zaccolo et al., 1996, J. Mol. Biol. 255 589) and PCR-based random mutagenesis such as described in Stemmer, 1994, Proc. Natl. Acad. Sci. USA 91 10747 or Shafikhani et al., 1997, Biotechniques 23 304, each of which references is incorporated herein. It is also noted that PCR-based random mutagenesis kits are commercially available, such as the Diversify™ kit (Clontech).

As used herein, “derivative” proteins are proteins of the invention that have been altered, for example by conjugation or complexing with other chemical moieties or by post-translational modification techniques as would be understood in the art. Such derivatives include amino acid deletions and/or additions to proteins of the invention, or variants thereof.

“Additions” of amino acids may include fusion of a protein or variants thereof with other proteins. Particular examples of such proteins include amino (N) and carboxyl (C) terminal amino acids added for use as “tags”. Use of an N-terminal poly-His tag for isolating an expressed fusion protein is described herein.

N-terminal and C-terminal tags include known amino acid sequences which bind a specific substrate, or bind known antibodies, preferably monoclonal antibodies. PRSET B vector (ProBond™; Invitrogen Corp.) is an example of a vector comprising an N-terminal 6×-His-tag which binds ProBond™ resin.

Other derivatives contemplated by the invention include, modification to side chains, incorporation of unnatural amino acids and/or their derivatives during protein synthesis and the use of cross linkers and other methods which impose conformational constraints on the proteins, fragments and variants of the invention. Examples of side chain modifications contemplated by the present invention include modifications of amino groups such as by acylation with acetic anhydride; acylation of amino groups with succinic anhydride and tetrahydrophthalic anhydride; amidination with methylacetimidate; carbamoylation of amino groups with cyanate; pyridoxylation of lysine with pyridoxal-5-phosphate followed by reduction with NaBH₄; reductive alkylation by reaction with an aldehyde followed by reduction with NaBH₄; and trinitrobenzylation of amino groups with 2,4,6-trinitrobenzene sulphonic acid (TNBS).

The carboxyl group may be modified by carbodiimide activation via O-acylisourea formation followed by subsequent derivitization, by way of example, to a corresponding amide.

The guanidine group of arginine residues may be modified by formation of heterocyclic condensation products with reagents such as 2,3-butanedione, phenylglyoxal and glyoxal.

Sulphydryl groups may be modified by methods such as performic acid oxidation to cysteic acid; formation of mercurial derivatives using 4-chloromercuriphenylsulphonic acid, 4-chloromercuribenzoate; 2-chloromercuri-4-nitrophenol, phenylmercury chloride, and other mercurials; formation of a mixed disulphides with other thiol compounds; reaction with maleimide, maleic anhydride or other substituted maleimide; carboxymethylation with iodoacetic acid or iodoacetamide; and carbamoylation with cyanate at alkaline pH.

Tryptophan residues may be modified, for example, by alkylation of the indole ring with 2-hydroxy-5-nitrobenzyl bromide or sulphonyl halides or by oxidation with N-bromosuccinimide.

Tyrosine residues may be modified by nitration with tetranitromethane to form a 3-nitrotyrosine derivative.

The imidazole ring of a histidine residue may be modified by N-carbethoxylation with diethylpyrocarbonate or by alkylation with iodoacetic acid derivatives.

Examples of incorporating unnatural amino acids and derivatives during protein synthesis include, use of 4-amino butyric acid, 6-aminohexanoic acid, 4-amino-3-hydroxy-5-phenylpentanoic acid, 4-amino-3-hydroxy-6-methylheptanoic acid, t-butylglycine, norleucine, norvaline, phenylglycine, ornithine, sarcosine, 2-thienyl alanine and/or D-isomers of amino acids.

Proteins in relation to the invention such as those exemplified in the figures (inclusive of fragments, variants, derivatives and homologs in general) may be prepared by any suitable procedure known to those of skill in the art.

For example, the protein may be prepared by a procedure including the steps of:
(i) preparing an expression construct which comprises a recombinant nucleic acid of the invention, operably linked to one or more regulatory nucleotide sequences, for example a T7 promoter;
(ii) transfecting or transforming the expression construct into a suitable host cell, for example E. coli; and
(iii) expressing the protein in said host cell.

Preferably, the recombinant nucleic acid of the invention encodes a protein as shown in FIG. 1 herein, including fragment, variant or homolog thereof. Preferred expression methods include those described in the examples.

Recombinant proteins may be conveniently expressed and purified by a person skilled in the art using commercially available kits, for example “ProBond™ Purification System” available from Invitrogen Corporation, Carlsbad, Calif., USA, herein incorporated by reference. Alternatively, standard molecular biology protocols may be used, as for example described in Sambrook, et al., MOLECULAR CLONING. A Laboratory Manual (Cold Spring Harbor Press, 1989), incorporated herein by reference, in particular Sections 16 and 17; CURRENT PROTOCOLS IN MOLECULAR BIOLOGY Eds. Ausubel et al., (John Wiley & Sons, Inc. 1995-1999), incorporated herein by reference, in particular Chapters 10 and 16; and CURRENT PROTOCOLS IN PROTEIN SCIENCE Eds. Coligan et al., (John Wiley & Sons, Inc. 1995-1999) which is incorporated by reference herein, in particular Chapters 1, 5, 6 and 7.

### Nucleic Acids

The term “nucleic acid” as used herein designates single or double stranded mRNA, RNA, cRNA and DNA, said DNA inclusive of cDNA and genomic DNA. A nucleic acid may be native or recombinant and may comprise one or more artificial nucleotides, e.g. nucleotides not normally found in nature. Nucleic acid encompasses modified purines (for example, inosine, methylinosine and methyladenosine) and modified pyrimidines (thiouridine and methylcytosine).

The term “isolated nucleic acid” as used herein refers to a nucleic acid subjected to in vitro manipulation into a form not normally found in nature. Isolated nucleic acid includes both native and recombinant (non-native) nucleic acids. For example, a nucleic acid isolated from human or mouse.

A “polynucleotide” is a nucleic acid having eighty (80) or more contiguous nucleotides, while an “oligonucleotide” has less than eighty (80) contiguous nucleotides.

In one embodiment, a nucleic acid “fragment” comprises a nucleotide sequence that constitutes less than 100% of a nucleic acid of the invention, for example, less than or equal to: 99%, 98%, 95%, 90%, 85%, 80%, 75%, 70%, 65%, 60%, 55%, 50%, 45%, 40%, 35%, 30%, 25%, 20%, 15%, 10%, 8%, 6%, 4%, 2% or even 1%. It will be appreciated that a fragment comprises all integer values less than 100%, for example the percent value as set forth above and others. A fragment includes a polynucleotide, oligonucleotide, probe, primer and an amplification product, eg. a PCR product. Examples of fragments of a full length nucleic acids are shown underlined in FIGS. 38, 40, 42, 44, 46, 48, 50, 52, 54, 57, 59, 62, 64, 66, 68 and 70, including the human infecting form of Chlamydia as shown. A fragment also includes a sub-fragment, i.e. a fragment of a fragment such as a primer derived from a larger fragment of the invention such as a fragment indicated as underlined in the figures.

A “probe” may be a single or double-stranded oligonucleotide or polynucleotide, suitably labeled for the purpose of detecting complementary sequences in Northern or Southern blotting, for example.

A “primer” is usually a single-stranded oligonucleotide, preferably having 20-50 contiguous nucleotides, which is capable of annealing to a complementary nucleic acid “template” and being extended in a template-dependent fashion by the action of a DNA polymerase such as Taq polymerase, RNA-dependent DNA polymerase or Sequenase™. For example, primers may be used to amplify nucleic acids specific for a selected strain or species to thereby determine a presence of said strain or species of an organism, preferably Chlamydia, as is discussed in more detail herein after.

Primers may be used to amplify nucleic acids common to one or more strains or species. Such primers are example of primers that are preferably used to detect one or more strain or species of an organism, preferably Chlamydia, as is discussed in more detail herein after. A primer preferably comprises about 5 to 200 contiguous nucleotides, including all integer values inclusive and therebetween, for example, 5, 10, 20, 30, 40, 50, 75, 100, 125, 150, 150, 175 and 200. The nucleotide sequence is obtainable from any one of the nucleic acids of the invention, including fragments.

### Variant Nucleic Acid

As used herein, the term nucleic acid “variant” means a nucleic acid of the invention, the nucleotide sequence of which has been mutagenized or otherwise altered so as to encode substantially the same, or a modified protein. Such changes may be trivial, for example in cases where more convenient restriction endonuclease cleavage and/or recognition sites are introduced without substantially affecting biological activity of an encoded protein when compared to a non-variant form. Other nucleotide sequence alterations may be introduced so as to modify biological activity of an encoded protein. These alterations may include deletion or addition of one or more nucleotide bases, or involve non-conservative substitution of one base for another. Such alterations can have profound effects upon biological activity of an encoded protein, possibly increasing or decreasing biological activity. In this regard, mutagenesis may be performed in a random fashion or by site-directed mutagenesis in a more “rational” manner. Standard mutagenesis techniques are well known in the art, and examples are provided in Chapter 9 of CURRENT PROTOCOLS IN MOLECULAR BIOLOGY Eds Ausubel et al. (John Wiley & Sons NY, 1995), which is incorporated herein by reference.

For the purposes of host cell expression, the recombinant nucleic acid is operably linked to one or more regulatory sequences in an expression vector, for example a T7 promoter.

An “expression vector” may be either a self-replicating extra-chromosomal vector such as a plasmid, or a vector that integrates into a host genome. Examples of expression vectors include: eukaryotic expression vectors pC130, pC31 and pC32; and pRSET B (Invitrogen Corp.) and derivations thereof. An expression vector may include a bacterial expression vector as is commonly used to express recombinant proteins in bacteria, preferably for subsequent isolation of the recombinant protein. Such vectors include: pBEc-Q (Stratagene), pT7-FLAG-1 (Sigma-Aldrich), TNT system (Promega) and the like.

By “operably linked” is meant that said regulatory nucleotide sequence(s) is/are positioned relative to the recombinant nucleic acid of the invention to initiate, regulate or otherwise control transcription.

Regulatory nucleotide sequences will generally be appropriate for the host cell used for expression. Numerous types of appropriate expression vectors and suitable regulatory sequences are known in the art for a variety of host cells.

Typically, said one or more regulatory nucleotide sequences may include, promoter sequences, leader or signal sequences, ribosomal binding sites, transcriptional start and termination sequences, translational start and termination sequences, and enhancer or activator sequences.

Constitutive or inducible promoters as known in the art are contemplated by the invention. The promoters may be either naturally occurring promoters, or hybrid promoters that combine elements of more than one promoter. For example, the lac promoter is inducible by IPTG.

The expression vector may further comprise a selectable marker gene to allow the selection of transformed host cells. Selectable marker genes are well known in the art and will vary with the host cell used. For example, an ampicillin resistance gene for selection of positively transformed host cells when grown in a medium comprising ampicillin.

The expression vector may also include a fusion partner (typically provided by the expression vector) so that the recombinant protein of the invention is expressed as a fusion protein with the fusion partner. An advantage of fusion partners is that they assist identification and/or purification of the fusion protein. Identification and/or purification may include using a monoclonal antibody or substrate specific for the fusion partner, for example a poly-His (for example 6×-His) tag or GST. A fusion partner may also comprise a leader sequence for directing secretion of a recombinant protein, for example an alpha-factor leader sequence.

Well known examples of fusion partners include poly-His or hexahistidine (6×-HIS)-tag, N-Flag, Fc portion of human IgG, glutathione-S-transferase (GST) and maltose binding protein (MBP), which are particularly useful for isolation of the fusion protein by affinity chromatography. For the purposes of fusion protein purification by affinity chromatography, relevant matrices for affinity chromatography may include nickel-conjugated or cobalt-conjugated resins, fusion protein specific antibodies, glutathione-conjugated resins, and amylose-conjugated resins respectively. Some matrices are available in “kit” form, such as the ProBond™ Purification System (Invitrogene Corp.) which incorporates a 6×-His fusion vector and purification using ProBond™ resin.

In order to express the fusion protein, it is necessary to ligate a nucleic acid according to the invention into the expression vector so that the translational reading frames of the fusion partner and the nucleotide sequence of the invention coincide.

The fusion partners may also have protease cleavage sites, for example enterokinase (available from Invitrogen Corp. as EnterokinaseMax™), Factor Xₐ or Thrombin, which allow the relevant protease to digest the fusion protein of the invention and thereby liberate the recombinant protein of the invention therefrom. The liberated protein can then be isolated from the fusion partner by subsequent chromatographic separation.

Fusion partners may also include within their scope “epitope tags”, which are usually short protein sequences for which a specific antibody is available.

As hereinbefore mentioned, proteins of the invention may be produced by culturing a host cell transformed with an expression construct comprising a nucleic acid encoding a protein, or protein homolog, of the invention. The conditions appropriate for protein expression will vary with the choice of expression vector and the host cell. For example, a nucleotide sequence of the invention may be modified for successful or improved protein expression in a given host cell. Modifications include altering nucleotides depending on preferred codon usage of the host cell. Alternatively, or in addition, a nucleotide sequence of the invention may be modified to accommodate host specific splice sites or lack thereof. These modifications may be ascertained by one skilled in the art.

Host cells for expression may be prokaryotic or eukaryotic.

Useful prokaryotic host cells are bacteria.

A typical bacteria host cell is a strain of E. coli.

Useful eukaryotic cells are yeast, SF9 cells that may be used with a baculovirus expression system as described herein, and other mammalian cells.

The recombinant protein may be conveniently prepared by a person skilled in the art using standard protocols as for example described in Sambrook, et al., MOLECULAR CLONING. A Laboratory Manual (Cold Spring Harbor Press, 1989), incorporated herein by reference, in particular Sections 16 and 17; CURRENT PROTOCOLS IN MOLECULAR BIOLOGY Eds. Ausubel et al., (John Wiley & Sons, Inc. 1995-1999), incorporated herein by reference, in particular Chapters 10 and 16; and CURRENT PROTOCOLS IN PROTEIN SCIENCE Eds. Coligan et al., (John Wiley & Sons, Inc. 1995-1999) which is incorporated by reference herein, in particular Chapters 1, 5 and 6.

In one embodiment, nucleic acid homologs encode protein homologs of the invention, inclusive of variants, fragments and derivatives thereof.

In yet another embodiment, nucleic acid homologs are nucleic acids having one or more codon sequences altered by taking advantage of codon sequence redundancy.

A particular example of this embodiment is optimization of a nucleic acid sequence according to codon usage by a selected organism as is well known in the art. This can effectively “tailor” a nucleic acid for optimal expression in a particular organism, or cells thereof, where preferential codon usage has been established.

In another embodiment, nucleic acid homologs share at least 50% or 60%, preferably at least 62% or 70%, more preferably at least 80%, and even more preferably at least 90%, 95%, 98%, 99% or 100% sequence identity with the nucleic acids of the invention.

In yet another embodiment, nucleic acid homologs hybridise to nucleic acids of the invention, including fragments, under at least low stringency conditions, preferably under at least medium stringency conditions and more preferably under high stringency conditions.

“Hybridise and Hybridisation” is used herein to denote the pairing of at least partly complementary nucleotide sequences to produce a DNA-DNA, RNA-RNA or DNA-RNA hybrid. Hybrid sequences comprising complementary nucleotide sequences occur through base-pairing.

In DNA, complementary bases are:
(i) A and T; and
(ii) C and G.

In RNA, complementary bases are:
(i) A and U; and
(ii) C and G.

In RNA-DNA hybrids, complementary bases are:
(i) A and U;
(ii) A and T; and
(iii) G and C.

Modified purines (for example, inosine, methylinosine and methyladenosine) and modified pyrimidines (thiouridine and methylcytosine) may also engage in base pairing.

“Stringency” as used herein, refers to temperature and ionic strength conditions, and presence or absence of certain organic solvents and/or detergents during hybridisation. The higher the stringency, the higher will be the required level of complementarity between hybridizing nucleotide sequences.

“Stringent conditions” designates those conditions under which only nucleic acid having a high frequency of complementary bases will hybridize.

Reference herein to low stringency conditions includes and encompasses:—
(i) from at least about 1% v/v to at least about 15% v/v formamide and from at least about 1 M to at least about 2 M salt for hybridisation at 42° C., and at least about 1 M to at least about 2 M salt for washing at 42° C.; and
(ii) 1% Bovine Serum Albumin (BSA), 1 mM EDTA, 0.5 M NaHPO₄ (pH 7.2), 7% SDS for hybridization at 65° C., and (i) 2×SSC, 0.1% SDS; or (ii) 0.5% BSA, 1 mM EDTA, 40 mM NaHPO₄ (pH 7.2), 5% SDS for washing at room temperature.

Medium stringency conditions include and encompass:—
(i) from at least about 16% v/v to at least about 30% v/v formamide and from at least about 0.5 M to at least about 0.9 M salt for hybridisation at 42° C., and at least about 0.5 M to at least about 0.9 M salt for washing at 42° C.; and
(ii) 1% Bovine Serum Albumin (BSA), 1 mM EDTA, 0.5 M NaHPO₄ (pH 7.2), 7% SDS for hybridization at 65° C. and (a) 2×SSC, 0.1% SDS; or (b) 0.5% BSA, 1 mM EDTA, 40 mM NaHPO₄ (pH 7.2), 5% SDS for washing at 42° C.

High stringency conditions include and encompass:—
(i) from at least about 31% v/v to at least about 50% v/v formamide and from at least about 0.01 M to at least about 0.15 M salt for hybridisation at 42° C., and at least about 0.01 M to at least about 0.15 M salt for washing at 42° C.;
(ii) 1% BSA, 1 mM EDTA, 0.5 M NaHPO₄ (pH 7.2), 7% SDS for hybridization at 65° C., and (a) 0.1×SSC, 0.1% SDS; or (b) 0.5% BSA, 1 mM EDTA, 40 mM NaHPO₄ (pH 7.2), 1% SDS for washing at a temperature in excess of 65° C. for about one hour; and
(iii) 0.2×SSC, 0.1% SDS for washing at or above 68° C. for about 20 minutes.

In general, the Tₘ of a duplex DNA decreases by about 1° C. with every increase of 1% in the number of mismatched bases.

Notwithstanding the above, stringent conditions are well known in the art, such as described in Chapters 2.9 and 2.10 of Ausubel et al., supra, which are herein incorporated by reference. A skilled addressee will also recognize that various factors can be manipulated to optimize the specificity of the hybridization. Optimization of the stringency of the final washes can serve to ensure a high degree of hybridization.

Typically, complementary nucleotide sequences are identified by blotting techniques that include a step whereby nucleotides are immobilized on a matrix (preferably a synthetic membrane such as nitrocellulose), a hybridization step, and a detection step. Southern blotting is used to identify a complementary DNA sequence; Northern blotting is used to identify a complementary RNA sequence. Dot blotting and slot blotting can be used to identify complementary DNA/DNA, DNA/RNA or RNA/RNA polynucleotide sequences. Such techniques are well known by those skilled in the art, and have been described in Ausubel et al., supra, at pages 2.9.1 through 2.9.20, herein incorporated by reference.

According to such methods, Southern blotting involves separating DNA molecules according to size by gel electrophoresis, transferring the size-separated DNA to a synthetic membrane, and hybridizing the membrane bound DNA to a complementary nucleotide sequence.

In dot blotting and slot blotting, DNA samples are directly applied to a synthetic membrane prior to hybridization as above.

An alternative blotting step is used when identifying complementary nucleic acids in a cDNA or genomic DNA library, such as through the process of plaque or colony hybridisation. Other typical examples of this procedure are described in Chapters 8-12 of Sambrook et al., supra which are herein incorporated by reference.

Typically, the following general procedure can be used to determine hybridisation conditions. Nucleic acids are blotted/transferred to a synthetic membrane, as described above. A nucleotide sequence of the invention is labeled as described above, and the ability of this labeled nucleic acid to hybridise with an immobilized nucleotide sequence analysed.

A skilled addressee will recognise that a number of factors influence hybridisation. The specific activity of radioactively labeled polynucleotide sequence should typically be greater than or equal to about 10⁸ dpm/μg to provide a detectable signal. A radiolabeled nucleotide sequence of specific activity 10⁸ to 10⁹ dpm/μg can detect approximately 0.5 μg of DNA. It is well known in the art that sufficient DNA must be immobilised on the membrane to permit detection. It is desirable to have excess immobilised DNA, usually 10 μg. Adding an inert polymer such as 10% (w/v) dextran sulfate (MW 500,000) or polyethylene glycol 6000 during hybridisation can also increase the sensitivity of hybridisation (see Ausubel et al., supra at 2.10.10).

To achieve meaningful results from hybridisation between a nucleic acid immobilised on a membrane and a labeled nucleic acid, a sufficient amount of the labeled nucleic acid must be hybridised to the immobilised nucleic acid following washing. Washing ensures that the labeled nucleic acid is hybridised only to the immobilised nucleic acid with a desired degree of complementarity to the labeled nucleic acid.

Methods for detecting labeled nucleic acids hybridised to an immobilised nucleic acid are well known to practitioners in the art. Such methods include autoradiography, chemiluminescent, fluorescent and colourimetric detection.

Nucleic acid homologs of the invention may be prepared according to the following procedure:
(i) obtaining a nucleic acid extract from a suitable host, for example a bacterial species;
(ii) creating primers which are optionally degenerate wherein each comprises a portion of a nucleotide sequence of the invention; and
(iii) using said primers to amplify, via nucleic acid amplification techniques, one or more amplification products from said nucleic acid extract.

As used herein, an “amplification product” refers to a nucleic acid product generated by nucleic acid amplification techniques.

Suitable nucleic acid amplification techniques are well known to the skilled addressee, and include PCR as for example described in Chapter 15 of Ausubel et al. supra, which is incorporated herein by reference; strand displacement amplification (SDA) as for example described in U.S. Pat. No. 5,422,252 which is incorporated herein by reference; rolling circle replication (RCR) as for example described in Liu et al., 1996, J. Am. Chem. Soc. 118 1587 and International application WO 92/01813; and Lizardi and Caplan, International Application WO 97/19193, which are incorporated herein by reference; nucleic acid sequence-based amplification (NASBA) as for example described by Sooknanan et al., 1994, Biotechniques 17 1077, which is incorporated herein by reference; ligase chain reaction (LCR) as for example described in International Application WO89/09385 which is incorporated herein by reference; and Q-β replicase amplification as for example described by Tyagi et al., 1996, Proc. Natl. Acad. Sci. USA 93 5395 which is incorporated herein by reference. Preferably, amplification is by PCR using primers disclosed herein.

### Antibodies

The invention also contemplates antibodies against the isolated proteins of the invention, including fragments, variants and derivatives thereof. Antibodies of the invention may be polyclonal or monoclonal. Well-known protocols applicable to antibody production, purification and use may be found, for example, in Chapter 2 of Coligan et al., CURRENT PROTOCOLS IN IMMUNOLOGY (John Wiley & Sons NY, 1991-1994) and Harlow, E. & Lane, D. Antibodies: A Laboratory Manual, Cold Spring Harbor, Cold Spring Harbor Laboratory, 1988, which are both herein incorporated by reference.

The inventors have detected antibodies in the serum of humans with acute chlamydial infection in relation to CT0828 (SEQ ID NO: 127).

Generally, antibodies of the invention bind to or conjugate with a protein, fragment, variant or derivative of the invention. For example, the antibodies may comprise polyclonal antibodies. Such antibodies may be prepared for example by injecting a protein, fragment, variant or derivative of the invention into a production species, which may include mice or rabbits, to obtain polyclonal antisera. Methods of producing polyclonal antibodies are well known to those skilled in the art. Exemplary protocols which may be used are described for example in Coligan et al., CURRENT PROTOCOLS IN IMMUNOLOGY, supra, and in Harlow & Lane, 1988, supra.

In lieu of the polyclonal antisera obtained in the production species, monoclonal antibodies may be produced using the standard method as for example, described in an article by Köhler & Milstein, 1975, Nature 256, 495, which is herein incorporated by reference, or by more recent modifications thereof as for example, described in Coligan et al., CURRENT PROTOCOLS IN IMMUNOLOGY, supra by immortalizing spleen or other antibody producing cells derived from a production species which has been inoculated with one or more of the proteins, fragments, variants or derivatives of the invention.

The invention also includes within its scope antibodies which comprise Fc or Fab fragments of the polyclonal or monoclonal antibodies referred to above. Alternatively, the antibodies may comprise single chain Fv antibodies (scFvs) against the proteins of the invention. Such scFvs may be prepared, for example, in accordance with the methods described respectively in U.S. Pat. No. 5,091,513, European Patent No 239,400 or an article by Winter & Milstein, 1991, Nature 349 293, which are incorporated herein by reference.

The antibodies of the invention may be used for affinity chromatography in isolating natural or recombinant proteins of the invention. For example, reference may be made to immunoaffinity chromatographic procedures described in Chapter 9.5 of Coligan et al., CURRENT PROTOCOLS IN IMMUNOLOGY, supra.

Antibodies may be purified from a suitable biological fluid of the animal by ammonium sulfate fractionation, affinity purification or by other methods well known in the art. Exemplary protocols for antibody purification are given in Sections 10.11 and 11.13 of Ausubel et al., supra, which are herein incorporated by reference.

Immunoreactivity of the antibody against the native or parent protein may be determined by any suitable procedure such as, for example, Western blot.

### Pharmaceutical Compositions

A further feature of the invention is use of the protein, fragment, variant, homolog or derivative thereof, and/or nucleic acid, described herein as actives in a pharmaceutical composition. Preferably, the protein comprises an amino acid sequence as shown in FIG. 1, more preferably, the protein is a human protein (SEQ ID NOS: 1 to 43). More preferably, the protein comprises an amino acid sequence of a protein obtainable from a form of Chlamydia capable of infecting humans, including fragments thereof. Even more preferably, the protein comprises an amino acid sequence set forth in SEQ ID NOS: 20, 34 and 35.

One aspect of the invention relates to a pharmaceutical composition comprising one or more proteins and/or nucleic acids of the invention as described herein obtainable from Chlamydia. In a preferred form, the pharmaceutical composition comprises a plurality of proteins and/or nucleic acids of the invention. It will be appreciated that a pharmaceutical composition comprising a plurality of antigens may provide an improved immune response against the pathogen, namely one or more forms of Chlamydia. Accordingly, a pharmaceutical composition preferably comprises two, three, four, five, six, seven, eight, nine, ten, eleven, twelve, thirteen, fourteen, fifteen, sixteen, seventeen, eighteen, nineteen, twenty, twenty-five, thirty or more different proteins and/or nucleic acids of the invention.

The actives, in such composition, may be referred to as “immunogenic agents” which are capable of eliciting an immune response in an animal. An immunogenic agent may be in the form of an immunotherapeutic composition, vaccine or antigen presenting cell loaded or pulsed with an antigen. The antigen presenting cell may be loaded or pulsed with antigen by contacting the cell with an antigen, for example a protein, fragment, variant or derivative of the invention. The antigen presenting cell may be, for example a dendritic cell.

A preferred form of a pharmaceutical composition is an immunotherapeutic composition. An immunotherapeutic composition preferably is a vaccine.

An immunotherapeutic composition may provide partial protection or partial improvement from Chlamydia infection, and need not provide complete protection or treatment of Chlamydia infection to be useful. For example, partial protection or partial improvement from Chlamydia infection may allow for a host immune system to overcome the infection, may improve health of the patient and/or may be combined with other treatments. In a preferred form, the immunotherapeutic composition or vaccine is capable of preventing onset of symptoms of disease caused by Chlamydia infection. In another preferred form, the immunotherapeutic composition or vaccine is capable of treating symptoms of disease caused by Chlamydia infection. Such diseases include: sexually transmitted disease, trachoma, LGV, arthritis, neonatal inclusion conjunctivitis, pneumonia and urethritis and cervicitis in women and the sequelae of these diseases include pelvic inflammatory disease, ectopic pregnancy, tubal factor infertility, epididymitis, proctitis and reactive arthritis.

Preferably, the immunotherapeutic composition and vaccine are capable of interacting with Chlamydia during at least one stage of the organism's life cycle, for example during an intracellular stage and/or during an extracellular stage. For example, during an intracellular stage, a systemic cell-mediate immunity is preferred and during an extracellular stage a local mucosal IgA, and possibly IgG, response is preferred.

Suitably, the pharmaceutical composition comprises a pharmaceutically-acceptable carrier. By “pharmaceutically-acceptable carrier, diluent or excipient” is meant a solid or liquid filler, diluent or encapsulating substance that may be safely used in systemic administration. Depending upon the particular route of administration, a variety of carriers, well known in the art may be used. These carriers may be selected from a group including sugars, starches, cellulose and its derivatives, malt, gelatine, talc, calcium sulfate, vegetable oils, synthetic oils, polyols, alginic acid, phosphate buffered solutions, emulsifiers, isotonic saline, and pyrogen-free water.

Any suitable route of administration may be employed for providing a patient with the pharmaceutical composition of the invention. For example, intranasal, transdermal, oral, rectal, parenteral, sublingual, buccal, intravenous, intra-articular, intramuscular, intra-dermal, subcutaneous, inhalational, intraocular, intraperitoneal, intracerebroventricular and the like may be employed. Intranasal, transdermal, intramuscular and subcutaneous application may be appropriate for administration of immunogenic agents of the present invention. Intranasal and transcutaneous administration in one preferred form includes use of cholera toxin and CpG-oligonucleotides as adjuvants. CpG-oligonucleotides are thought to induce primarily a Th1 immune response and cholera toxin is thought to induce mucosal IgA when administered orally or intranasally, but induces an IgG response when administered transcutaneously, see for example Berry et al, 2004, Infect Immun 72 1019, incorporated herein by reference. Skin penetration enhancers, such as chemical penetration enhancers including DMSO and electrically assisted methods including iontohoresis, may also be used as described for example in Barry, 2004, Nature Biology 22 165.

Dosage forms include tablets, dispersions, suspensions, injections, solutions, syrups, troches, capsules, suppositories, aerosols, transdermal patches and the like. These dosage forms may also include injecting or implanting controlled releasing devices designed specifically for this purpose or other forms of implants modified to act additionally in this fashion. Controlled release of the therapeutic agent may be effected by coating the same, for example, with hydrophobic polymers including acrylic resins, waxes, higher aliphatic alcohols, polylactic and polyglycolic acids and certain cellulose derivatives such as hydroxypropylmethyl cellulose. In addition, the controlled release may be affected by using other polymer matrices, liposomes and/or microspheres.

Pharmaceutical compositions of the present invention suitable for administration may be presented as discrete units such as vials, capsules, sachets or tablets each containing a pre-determined amount of one or more immunogenic agent of the invention, as a powder, or granules, or as a solution or a suspension in an aqueous liquid, a non-aqueous liquid, an oil-in-water emulsion or a water-in-oil liquid emulsion. Such compositions may be prepared by any of the methods of pharmacy but all methods include the step of bringing into association one or more immunogenic agents as described above with the carrier, which constitutes one or more necessary ingredients. In general, the compositions are prepared by uniformly and intimately admixing the agents of the invention with liquid carriers or finely divided solid carriers or both, and then, if necessary, shaping the product into the desired presentation.

### Vaccines

The above compositions may be a therapeutic or prophylactic vaccines comprising one or more protein and/or nucleic acid of the invention, or respective fragments thereof. Preferably, the vaccine comprises a plurality of different proteins and/or nucleic acids as described herein. Preferably, the vaccine prevents or treats Chlamydia infection. More preferably, the vaccine prevents or treats a human infecting form of Chlamydia when administered to a human patient. A pharmaceutical composition and vaccine need not completely prevent or treat Chlamydia infection to be useful and reduced infection or reduced severity of infection is also considered a useful benefit and advantage.

Accordingly, the invention extends to the production of vaccines comprising one or more actives (protein and/or nucleic acid) of the invention. Any suitable procedure is contemplated for producing such vaccines. Exemplary procedures include, for example, those described in NEW GENERATION VACCINES (1997, Levine et al., Marcel Dekker, Inc. New York, Basel Hong Kong) which is incorporated herein by reference.

An active according to the invention can be mixed, conjugated or fused with other antigens, including B or T cell epitopes of other antigens. In addition, it can be conjugated to a carrier as described below.

When a haptenic protein of the invention is used (i.e., a protein which reacts with cognate antibodies, but cannot itself elicit an immune response), it can be conjugated with an immunogenic carrier. Useful carriers are well known in the art and include for example: thyroglobulin; albumins such as human serum albumin; toxins, toxoids or any mutant cross reactive material (CRM) of the toxin from tetanus, diptheria, pertussis, Pseudomonas, E. coli, Staphylococcus, and Streptococcus; polyamino acids such as poly(lysine:glutamic acid); influenza; Rotavirus VP6, Parvovirus VP1 and VP2; hepatitis B virus core protein; hepatitis B virus recombinant vaccine and the like. Alternatively, a fragment or epitope of a carrier protein or other immunogenic protein may be used. For example, a haptenic protein of the invention can be coupled to a T cell epitope of a bacterial toxin, toxoid or CRM. In this regard, reference may be made to U.S. Pat. No. 5,785,973 which is incorporated herein by reference.

The vaccines can also comprise a physiologically-acceptable carrier, diluent or excipient such as water, phosphate buffered saline and saline.

The vaccines and immunogenic compositions may include an adjuvant as is well known in the art. Suitable adjuvants include, but are not limited to adjuvants for use in human for example SBAS2, SBAS4, QS21 or ISCOMs.

The immunogenic agents of the invention may be expressed by attenuated viral hosts.

By “attenuated viral hosts” is meant viral vectors that are either naturally, or have been rendered, substantially avirulent. A virus may be rendered substantially avirulent by any suitable physical (e.g., heat treatment) or chemical means (e.g., formaldehyde treatment). By “substantially avirulent” is meant a virus whose infectivity has been destroyed. Ideally, the infectivity of the virus is destroyed without affecting the proteins that carry the immunogenicity of the virus. From the foregoing, it will be appreciated that attenuated viral hosts may comprise live viruses or inactivated viruses.

Attenuated viral hosts which may be useful in a vaccine according to the invention may comprise viral vectors inclusive of adenovirus, cytomegalovirus and preferably pox viruses such as vaccinia (see for example Paoletti and Panicali, U.S. Pat. No. 4,603,112 which is incorporated herein by reference) and attenuated Salmonella strains (see for example Stocker, U.S. Pat. No. 4,550,081 which is herein incorporated by reference). Live vaccines are particularly advantageous because they lead to a prolonged stimulus that can confer substantially long-lasting immunity.

Multivalent vaccines can be prepared from one or more different epitopes of one or more proteins as described herein. An example of a preferred mulitivalent vaccine includes a nucleic acid encoding one or more epitopes of one or more Chlamydia proteins, including fragments thereof. A preferred form of a nucleic acid suitable for use as a multivalent vaccine comprises an nucleic acid sequence comprising two or more of the protein fragments expressed as a fusion protein with no (i.e. contiguous) or minimal amino acids between adjacent protein fragments. For example, the protein fragment for CT661, shown as a corresponding underlined sequence in FIG. 1 may be expressed as a fusion protein with the protein fragment for CT774, shown as a corresponding underlined sequence in FIG. 1. The multivalent vaccine may encode 2, 3, 4, 5, 6, 7, 8, 9, 10, 11, 12, 13, 14, 15, 16, 17, 18, 19, 20, 25, 30, 35, 40, 45 or more protein antigens, including fragments thereof. The multivalent vaccine may comprise one or more same protein antigen, for example, 1, 2, 3, 4, 5, 6 or more of the same protein antigen including various fragments and homologs thereof. The multivalent protein may also comprise B-cell and T-cell epitopes.

A recombinant vaccinia virus may be prepared to express a nucleic acid according to the invention. Upon introduction into a host, the recombinant vaccinia virus expresses the immunogenic agent, and thereby elicits a host CTL response. For example, reference may be made to U.S. Pat. No. 4,722,848, incorporated herein by reference, which describes vaccinia vectors and methods useful in immunization protocols.

A wide variety of other vectors useful for therapeutic administration or immunization with the immunogenic agents of the invention will be apparent to those skilled in the art from the present disclosure.

The nucleic acid of the invention may be used as a vaccine in the form of a “naked DNA” vaccine as is known in the art. For example, an expression vector of the invention may be introduced into a mammal, where it causes production of a protein in vivo, against which the host mounts an immune response as for example described in Barry, M. et al., (1995, Nature, 377, 632-635) which is hereby incorporated herein by reference.

In a preferred embodiment as described herein, the invention relates to a nucleic acid vaccine. It will be appreciated that the method of isolating the nucleic acids of the invention involved steps of expressing a nucleic acid in an animal and assessing an immune response. Accordingly, the nucleic acids of the invention may be particularly useful for a nucleic acid vaccine.

### Preparation of Immunoreactive Fragments

The invention also extends to a method of identifying an immunoreactive fragment of a protein, variant or derivatives according to the invention. This method essentially comprises generating a fragment of the protein, variant or derivative, administering the fragment to a mammal; and detecting an immune response in the mammal. The method for identifying the isolated nucleic acids and fragments includes expression in a host and detection of an immune response. Accordingly, the proteins and nucleic acids encoding the proteins may be useful in preparing immunological reagents such as antibodies.

Prior to testing a particular fragment for immunoreactivity in the above method, a variety of predictive methods may be used to deduce whether a particular fragment can be used to obtain an antibody that cross-reacts with the native antigen. These predictive methods may be based on amino-terminal or carboxy-terminal sequence as for example described in Chapter 11.14 of Ausubel et al., supra. Alternatively, or in addition, these predictive methods may be based on predictions of hydrophilicity as for example described by Kyte & Doolittle 1982, J. Mol. Biol. 157 105 and Hopp & Woods, 1983, Mol. Immunol. 20 483) which are incorporated by reference herein, or predictions of secondary structure as for example described by Choo & Fasman, 1978, Ann. Rev. Biochem. 47 251), which is incorporated herein by reference.

Generally, a protein fragment consisting of 10 to 15 residues provides optimal results. Proteins as small as 6 or as large as 20 residues have worked successfully. Such protein fragments may then be chemically coupled to a carrier molecule such as keyhole limpet hemocyanin (KLH) or bovine serum albumin (BSA) as for example described in Sections 11.14 and 11.15 of Ausubel et al., supra.

The proteins may be used to immunize an animal as for example discussed above. Antibody titers against the native or parent protein from which the protein was selected may then be determined by, for example, radioimmunoassay or ELISA as for instance described in Sections 11.16 and 11.14 of Ausubel et al., supra.

### Detection Method and Kits

The present invention also provides a method and kit for detecting Chlamydia and/or one or more Chlamydia antigens in a biological sample. A “biological sample” refers to a sample derived or obtained from a biological source, for example a cell, tissue, organ, organism, including Chlamydia and components thereof, lysate, homogenate, biological fluid including mucus, blood, serum, plasma, urine or cerebrospinal fluid. A kit will comprise one or more agents described above depending upon the nature of the test method employed.

In this regard, the kits may include one or more of a protein, fragment, variant, derivative, antibody, antibody fragment or nucleic acid according to the invention. The kits may also optionally include appropriate reagents for detection of labels, positive and negative controls, washing solutions, dilution buffers and the like.

A protein or antibody based detection kit may include (i) a protein, or fragment or variant thereof according to the invention (which may be used as a positive control), (ii) an antibody according to the invention (preferably a monoclonal antibody) which binds to a Chlamydia antigen or fragment thereof in (i), and (iii) a suitable means for detecting a complex formed between a target (eg. a Chlamydia antigen in a sample) and the antibody in (ii), the detection means may include, for example colloidal gold, protein, enzyme, colour reagent and the like.

A protein or antibody in one form is capable of binding to a target protein common to one or more biovar, serovar, strain, variant or species of Chlamydia. Such a protein or antibody may be useful for identifying a range of different biovars, serovars, strains, variants or species of Chlamydia in a biological sample. Alternatively, or in addition, a protein or antibody may be specific for a particular biovar, for example a trachoma biovar or lymphogranuloma venereum biovar; or specific for a particular serovar, for example serovar A, B, Ba, C, D, Da, E, F, G, H, I, Ia, J, K, L1, L2 or L3. The binding of the protein or antibody with a target protein may be detected using methods common to the art, including biotin-avadin, biotin-streptavadin, enzymatic detection methods common for ELISA based detection methods, colour regents, fluorescent dyes, radioactive labels such ³²P or ³⁵S, and metals, for example gold, Western blotting, dot blotting, affinity columns and the like.

It will be appreciated that protein-protein interactions may be used to detect a target protein, for example a protein that may form a complex with a target protein, which may be detected. Also, substrates for a target protein or fragment thereof may be used to detect the presence of the target protein or fragment thereof. For example, a substrate for DNA gyrase subunit B, sulfite synthesis/biphosphate phosphatase, methionyl-tRNA synthetase, DNA helicase (urvD) and/or ATP synthase subunit 1. It is also contemplated that a protein or antibody based kit in one form detects antibodies a biological sample from a subject that bind to one or more Chlamydia proteins. Such antibodies may be detected following infection by Chlamydia.

A detection kit may be nucleic acid based. Such a kit will contain one or more particular agents, for example nucleic acids and proteins as described above, depending upon the nature of the test method employed. In this regard, the kits may include one or more nucleic acid encoding Chlamydia antigen(s), or fragment thereof, as described herein, in particular with reference to the figures, inclusive of primers and probes according to the invention. Accordingly, the kits may include reagents for detection of labels, positive and negative controls, washing solutions, dilution buffers and the like. For example, a nucleic acid amplification based kit may comprise primers capable of hybridising with target nucleic acids encoding one or more Chlamydia antigens isolated from a biological sample. Such a biological sample in one embodiment may be isolated from an individual, preferably a human patient. The primers may be capable of hybridising to a nucleic acid comprising a nucleotide sequence shared by one or more biovar, serovar, strain, variant or species of Chlamydia. Such primers may be useful for identifying a range of different biovars, serovars, strains, variants or species of Chlamydia in a biological sample. Alternatively, or in addition, a primer may be specific for a particular biovar, for example a trachoma biovar or lymphogranuloma venereum biovar; or specific for a particular serovar, for example serovar A, B, Ba, C, D, Da, E, F, G, H, I, Ia, J, K, L1, L2 or L3. The nucleic acid primer may comprise any suitable number of contiguous nucleic acids, preferably between 5 and 200 nucleotides, for example, 5, 8, 10, 15, 20, 30, 40, 50, 60, 70, 80, 90, 100, 125, 150, 175 or 200. The nucleic acid may be amplified according to methods known in the art, for example PCR. The amplified nucleic acid, or non-amplified nucleic acids, can be compared with reference nucleic acids using methods including for example, separation by molecular weight by gel electrophorsis, hybridisation methods including Northern and Southern blotting, microarrays and other methods common in the art. Non-amplification methods of detection may include isolation of mRNA from a sample and performing Northern blot analysis using a nucleic acid probe derived from one or more of the Chlamydia nucleic acids, including fragments, described herein. The design of the nucleic acid probe may be based on sequences that are common between Chlamydia biovars, serovars, strains, variants or species, or sequences that share some sequence identity with selected Chlamydia biovars, serovars, strains, variants or species, or sequences that share sequence identity with all known Chlamydia biovars, serovars, strains, variants or species. A nucleic acid may be detected using labels common in the art including, for example, fluorescent dyes, radioactive labels such ³²P or ³⁵S, enzymes and metals, including gold.

### Microarrays

A microarray also uses hybridization-based technology that, for example, may allow detection and/or isolation of a nucleic acid by way of hybridization of complementary nucleic acids. A microarray provides a method of high throughput screening for a nucleic acid in a sample that may be tested against several nucleic acids attached to a surface of a matrix or chip. In this regard, a skilled person is referred to Chapter 22 of CURRENT PROTOCOLS IN MOLECULAR BIOLOGY (Eds. Ausubel et al. John Wiley & Sons NY, 2000).

Each nucleic acid occupies a known location on an array. A nucleic acid target sample probe is hybridised with the array of nucleic acids and an amount or relative abundance of target nucleic acid hybridised to each probe in the array is determined. High-density arrays are useful for monitoring gene expression and presence of allelic markers which may be associated with disease. Fabrication and use of high density arrays in monitoring gene expression have been previously described, for example in WO 97/10365, WO 92/10588 and U.S. Pat. No. 5,677,195, all incorporated herein by reference. In some embodiments, high-density oligonucleotide arrays are synthesised using methods such as the Very Large Scale Immobilised Polymer Synthesis (VLSIPS) described in U.S. Pat. No. 5,445,934, incorporated herein by reference.

Murine Model of C. trachomatis Genital Tract Infection

Numerous animal models have been used for the study of chlamydial genital tract infections (Ward 1995; Bavoil et al. 1996; Rank and Bavoil 1996). The murine biovar, C. trachomatis mouse pneumonitis readily infects the female mouse genital tract (Bavoil et al. 1996) and vaginal inoculation of mice with C. trachomatis MoPn results in a cervicitis that ascends to the uterine and oviduct epithelia. Mice naturally resolve the infection without antibiotic therapy and develop long-lived immunity that protects against reinfection (Barron et al. 1981; Morrison et al. 1995). The initial inflammatory response that is induced by vaginal infection with C. trachomatis MoPn is characterized by mucosal and submucosal infiltration of polymorphonuclear cells. As the infection resolves, lymphocytes and macrophages diffuse into the submucosa (Morrison et al. 1995; Su et al. 1997). These infiltrating lymphocytes include not only B-cells, but also both CD4+ T-cells and CD8+ T-cells (Morrison and Morrison 2000).

A similarity in pathogenesis and adaptive immune responses generated following the resolution of C. trachomatis infection in the murine genital tract and that of humans establish the usefulness of the murine model for studies into vaccine development and associated protective immunity. Another advantage of the murine model of chlamydial genital tract infection is an availability of immunological reagents and immunological defects that are available.

Protective Immunity to Chlamydial Genital Infections

Animal models of chlamydial infection have established that T-lymphocytes play a critical role both in the clearance of the initial infection and in protection from re-infection of susceptible hosts (Su and Caldwell 1995). Of particular importance in the protective immune response are CD4+ T-cells that produce gamma-interferon (IFN-γ) and other T-helper 1 type cytokines (Cotter et al. 1997). The production of IFN-γ CD 4+ T-cells has been shown to prevent dissemination of chlamydial genital tract infection in mice however clearance of the infection and resistance to re-infection was shown to be independent of IFN-γ. Knockout mice lacking IFN-γ or its receptor displayed delayed clearance of C. trachomatis following primary infection of the genital mucosa, however the animals eventually controlled the infection indicating the presence and importance of alternative mechanisms for resistance to chlamydial infection which remain unidentified (Perry et al. 1997). Although several studies have suggested that CD 4+ cells play a more dominant role in protective immunity against chlamydial infection (Magee et al., 1995; Su and Caldwell 1995) CD 8+ cytotoxic lymphocytes have also been associated with protection from chlamydial infection of the genital tract (Beatty and Stephens 1994; Starnbach et al. 1994). Cytotoxic T-cells can directly lyse chlamydial infected cells and produce both IFN-γ and tumour necrosis factor α, which have been shown to exhibit anti-chlamydial activity (Murray et al. 1989; Igietseme 1996). Despite this, however, the role of cytotoxic T-cells in the resolution of chlamydial genital tract infection remains unclear. Resolution of genital tract infection in β₂-microglobulin gene knockout mice (CD 8+ deficient) is equivalent to that of immunocompetent wild type mice (Morrison et al., 1995). CD 8+ deficient mice have also been shown to have protection against secondary challenge (Morrison et al. 1995) indicating that CD 8+ T-cells play a role in the elimination of C. trachomatis primary infection or in resistance to re-infection. Early studies demonstrated a positive correlation between increased secretion or serum levels of anti-chlamydial specific antibodies with both the resolution of primary infection (Rank et al., 1979; Brunham et al. 1983) and protection against re-infection in both humans and animal models of C. trachomatis infection (Murray et al. 1973; Rank and Barron 1983). Studies using monoclonal and monospecific polyclonal antibodies have shown that the major outer membrane protein (MOMP) of chlamydial EB's is the main target for neutralizing antibodies (Peeling et al. 1984; Lucero and Kuo 1985; Zhang et al. 1987) although neutralizing antibodies to other chlamydial determinants have also been described (Peterson et al. 1998). The protective function of neutralizing antibodies is supported by results of in vitro studies showing that chlamydial specific antibodies block attachment and subsequent infectivity of chlamydiae to murine epithelial cells (Peeling et al. 1984).

It has been suggested that secretory IgA (s-IgA) is the first line of defense against chlamydial infection of mucosal surfaces (Cui et al. 1991). S-IgA is a polymeric antibody and is the predominant antibody in most mucosal secretions (Tomasi and Ziegelbaum 1963). Secretory IgA is produced locally by plasma cells at mucosal surfaces and is translocated to external secretions by a transepithelial transport mechanism involving a molecule known as the secretory component (Mestecky and McGhee 1993). Oral immunisation of mice with C. trachomatis EB's resulted in the production of specific anti-chlamydial IgA antibodies in mucosal secretions at multiple sites leading to the hypothesis that these immune responses resulted from the migration of B-lymphocytes from the oral cavity to other mucosal tissues including the genital tract (Cui et al. 1991). Further to this, Brunham et al (1983) revealed a positive correlation between elevated levels of s-IgA in cervical secretions and reduced levels of chlamydial shedding. However athymic nude mice (i.e. B-cell deficient) have been shown to be incapable of resolving chlamydial genital tract infection (Rank et al. 1985) suggesting the importance of T-cell dependent help for antibody production. These studies suggest that both humoral and T-cell mediated immunity are required for host resistance to chlamydial infection of the genital tract although the host cell mechanisms by which protection against chlamydial infection is induced remains undefined.

### The Mucosal Immune System

One of the primary functions of the mucosal immune system is to prevent the entry of potentially pathogenic organisms, such as Chlamydia, into and through the mucosal epithelium. Antigen uptake and initiation of mucosal immune responses occur predominantly in specialized inductive sites, namely the mucosa-associated lymphoid tissue that lines the respiratory, alimentary and genitourinary tracts. Microfold cells (M-cells) of the Peyer's patches or lymph nodes in the closely surrounding mucosal tissue pinocytose and phagocytose antigen and deliver it intact to underlying macrophages and lymphocytes. The germinal centers of the Peyer's patches are rich in B-cells that are committed to producing IgA (Jones and Cebra 1974) through acting as a pool of cells that can migrate to distant mucosal sites known as the effector sites. This cellular migration explains the observation that administration of antigen at one mucosal site may generate s-IgA at distant mucosal sites (McGhee et al. 1992). Classical antigen presenting cells such as macrophages and Langerhans cells are also present at many mucosal sites and are thought to play a role in secondary induction of the immune response. M-cells are not present in the female vagina or cervix, however, and thus only limited immune responses are induced in vaccinated populations by intravaginal immunisation with protein populations. Langerhans cells and macrophages are however present in the vaginal mucosa and these can act as the antigen presenting cells in the female genital tract. It is therefore possible that immunisation at a distant mucosal site, such as the nasal associated lymphoreticular tissue may induce specific immune responses at the genital surface. Chlamydiae are largely pathogens of the mucosal surfaces of humans, specifically the genital tract, respiratory tract and conjunctivae. The development of a vaccine that induces protective humoral and cell mediated immune responses at the site of entry of the pathogen is highly desirable for protection against disease in susceptible hosts.

### Expression Library Immunisation

Expression library immunisation (ELI) is an approach to vaccine production that has the potential to identify vaccine antigens. ELI has been described in U.S. Pat. No. 5,703,057 (Johnston et al) and AU Patent 764256 (CSIRO) for non-Chlamydia pathogens, however, similar methods taught by these patents may be used herewith. ELI is an attractive method to investigate vaccine antigens because unlike some more traditional vaccine strategies, it requires no prior knowledge of antigenic targets and has the potential to screen every gene in the host pathogen genome. The process of screening the genome of a pathogen through a disease model also has the ability to induce both cellular and humoral immune responses so that no assumptions regarding the best immune response need to be made.

ELI involves the construction of a genomic library by fragmenting the pathogen DNA (or cDNA) and incorporating these library fragments into eukaryotic expression vectors. Pools, or sublibraries, of these clones then are screened through a disease model to demonstrate whether the sublibrary can confer a degree of protection against a challenge infection by the pathogen. The sublibrary or libraries that offer protection can then be subfractionated to reduce the complexity of the genomic library and eventually identify protective clones (Johnston and Barry 1997), incorporated herein by reference.

ELI was first reported by Barry et al (Barry et al. 1995) who demonstrated that DNA vaccination with genomic libraries of Mycoplasma pulmonis constructed in a eukaryotic expression vector could protect mice against infection. Since then there have been several reports of the use of this method in parasites (Johnston and Barry 1997; Brayton et al. 1998; Melby et al. 2000; Smooker et al. 2000). This work with ELI has used a variety of vectors for library construction however a major problem with all the libraries has been the large percentage of unproductive plasmids. Fragments of the pathogen genome that are in the wrong orientation, wrong reading frame or in intergenic regions will not express fusion proteins and hence are not able to induce an immune response. Recently, however, Moore et al. 2001 have developed improved vectors that allow screening for clones that are expressing a recombinant protein to overcome these problems. This was achieved by using a polyHis fusion partner and screening the transformed library in E. coli using polyHis detection methods. Unproductive clones, or those not producing a fusion protein were negative in the screen because small, unfused polyHis proteins have a very short half-life within the cells and any residual protein binds poorly to the membranes used in the screening process. In this manner the clones capable of expressing protein and hence those capable of inducing an immune response were isolated. The authors used these improved vectors for ELI against Mycoplasma hyopneumoniae in pigs to reduce a library of 20,000 clones to a protective group of just 96 clones. ELI is readily applicable to pathogens with small genomes such as Chlamydia because the genetic complement of such organisms can be represented in small libraries.

Throughout this specification, unless the context requires otherwise, the words “comprise”, “comprises” and “comprising” will be understood to imply the inclusion of a stated integer or group of integers but not the exclusion of any other integer or group of integers.

In order that the invention may be readily understood and put into practical effect, particular preferred embodiments will now be described by way of the following non-limiting examples.

### EXAMPLE 1

### Methods

Isolation of Genomic DNA from C. trachomatis MoPn Strain

The Mouse Pneumonitis strain of C. trachomatis is grown in vitro in McCoy host cell monolayers, at 37° C. in 5% CO₂ Cell monolayers are infected with C. trachomatis MoPn, maintained in maintenance media comprising cycloheximide for 72 hours and then stored in SPG at −80° C. EB's and RB's are isolated and from the cell culture by gradient centrifugation on a renograffin gradient. DNA is isolated from chlamydial EB's by standard techniques known in the art, preferably using commercially available kits.

### Library Construction

The genomic DNA is fragmented by physical shearing and used for library construction. The eukaryotic expression vectors pC130, pC131 and pC132, are used to create the genomic library as these vectors have structural features important for eukaryotic and prokaryotic expression including CMV promoter, antibiotic resistance gene and polyHis sequences. The genomic fragments and prepared vectors are mixed in order to ligate the DNA to the vector again using well-established scientific techniques.

### Library Screening

Colony lifts are taken from the transformed plates onto nitrocellulose filters and treated with a series of solutions to identify polyHis positive clones. After washing and blocking the filters are incubated with anti-polyHis antibody. The polyHis positive clones are determined by colourimetric reaction with horse-radish peroxide staining solution and the positively stained cloned picked off the filters into sterile 96 well microtitre plates. Insert size are determined by DNA miniprep methods and gel electrophoresis analysis.

### Plasmid DNA Preparation

Plasmid DNA for immunisations is prepared using Plasmid Mega Kits according to the manufacturers instructions. A polyHis positive expression library and several sublibraries are prepared. To create sublibries, DNA from individual plates is used or DNA from several plates is combined so as to create sublibraries of varying complexities.

### Vaccination

Adult female virgin BALB/c mice are divided into six groups (polyHis positive library and 5 sublibraries) comprising 5 animals per group for the initial screening process. A BioRad Helios gene gun is used to administer 50-100 μl of DNA (1 mg/mL) to the animals. The DNA preparations are delivered at time 0 and again 3 weeks after the primary immunisation. Four weeks after the initial immunisation the mice are challenged intravaginally with live C. trachomatis MoPn EB's and monitored for disease progression.

### Collection of Samples

Plasma and vaginal lavage samples are collected pre-immunisation, prior to challenge infection and then at four day intervals after challenge with live chlamydial EB's for 16 days. Animals are sacrificed with a lethal injection of sodium pentabarbitone. Blood is collected from the mice by retro-orbital bleed or saphenous leg vein bleed while the animals are under halothane anesthesia (QUT UAEC Ref No 1474/2A). Serum is collected by centrifugation and stored at −80° C. Vaginal lavage samples are collected by gently lavaging the vagina of the mice with 40 μL of Phosphate buffered saline with a Gilson pipette. Phenylmethylsulfonyl fluoride (PMSF), a protease inhibitor is added to each sample and after preparing a smear of the fluid on a slide, the remainder of the specimen is stored at −80° C. Uterine and vaginal tissue samples are also be collected at sacrifice for analysis of disease progression.

### Selection of Protective Sublibraries

Mice are screened for chlamydial infection using commercially available enzyme immunoassay and immunofluorescent kits according to the manufacturers instructions as are known in the art. Vaginal lavage slides are stained for chlamydial inclusions and protection against challenge infection defined as a reduction in the shedding of Chlamydia. Serum and vaginal lavage samples from animals immunised with “protective” sublibrarys are assessed for serum IgG, and vaginal IgG and IgA antibodies by Enzyme Linked Immunosorbent Assay (ELISA). Treatment groups that show a degree of protection against chlamydial challenge compared to control animals are subdivided into smaller numbers of clones per group and new groups of mice inoculated and challenged.

### Identification of Protective Genes

DNA is extracted from any “protective” clones identified in the animal immunisation trial. The DNA sequence is determined and BLAST against the whole C. trachomatis MoPn genome to identify the gene involved. If the gene identified as “protective” is less than the full length of the gene, the section of the gene is analysed and the full gene amplified by PCR for subsequent use. The full length gene DNA will be compared with the shorter fragments to determine if better protection can be afforded by the complete gene.

### Investigation of Routes of Immunisation

The genes that demonstrate the best degree of protection via the naked DNA immunisation approach will be evaluated for use as vaccine candidates in which the relevant protein is administered to mice through various delivery methods. Vaccination schedules will be developed for various routes of immunisation, including transcutaneous, systemic and mucosal immunisation routes and combinations thereof. Research has demonstrated that the initial site of antigen administration can influence the T-cell cytokine profile elicited in the animal (Kelly et al. 1996). Serum and secretion samples are collected from animals immunised with vectors incorporating the “protective” plasmids, animals immunised with empty vectors and unvaccinated animals. The samples collected are analysed for specific anti-chlamydial antibodies and level of protection elicited following a challenge infection.

### Measurement of Immunological Responses

In addition to the overall measurement of protection against live chlamydial challenge, an overall immune response to candidate antigens is evaluated. Serum and mucosal secretions from both vaccinated and control mice is collected and analysed by ELISA for antibody and cytokine profiles. In vitro neutralization tests is also conducted to determine the level of neutralizing antibody in each animal group. Spleen cells will be harvested from mice at sacrifice and T-lymphocyte responses determined by tritiated thymidine measurements.

Improving the Protective Effectiveness of Vaccines

Investigations include possible use of various adjuvants and interleukins to determine if a better protective immune response can be elicited. Research has shown that an immune response to antigen may be enhanced if co-administered with genetic adjuvants such as interleukin-12 (Svanholm et al. 2000). The present invention contemplates inclusion of such genetic adjuvants in a pharmaceutical composition comprising one or more antigens of the invention. The genetic adjuvant may be administered before, concurrently and/or after administration of the one or more Chlamydia antigens. In one preferred form of the invention, the genetic adjuvant is co-administered with the one or more Chlamydia antigen(s).

A vaccine may also comprise a plurality of proteins as described herein. Accordingly, a vaccine may comprise 1, 2, 3, 4, 5, 6, 7, 8, 9, 10, 11, 12, 13, 14, 15, 16, 17, 18, 20, 25, 30 or more proteins comprising different amino acid sequences, including fragments and homologs thereof. The vaccine in one form may comprise a plurality of fragments of a same full length protein.

### EXAMPLE 2

Construction of DNA library of C. trachomatis MoPn

Sheared C. trachomatis DNA and E. coli vectors have been ligated and 16 transformation reactions completed to select for recombinant colonies. Over 20,000 recombinant colonies were identified and these were screened by poly-His antibody binding to eliminate clones that did not comprise C. Trachomatis MoPn inserts in a correct open reading frame (ORF) or correct orientation. In total, 3072 poly-His positive colonies were selected and divided into 8 sub-libraries of 384 clones in each per sub-library.

### ELI for Investigating/Isolating Protective Genes

### ELI Screen 1:

Plasmid DNA was extracted from each sub-library and used to construct DNA coated gold particles for immunization by particle bombardment (gene gun). Nine groups of 5 animals each were used in total for a first round screening of the DNA library. Seven groups were immunized with experimental DNA, one group with control DNA (empty vector) and one control group remained non-immunised. The animals were immunized three times at three week intervals and the chlamydial vaginal challenge infection delivered 10 days following the final immunization. Vaginal swab samples were collected every 3 days post-challenge and analysed by real-time PCR for three weeks following the challenge infection. McCoy cell culture was established to further test the swab samples by immunofluorescent methods.

Single sublibrary (SL4) containing 384 clones identified as “protective” and selected for further analysis.

### ELI Screen 2:

150 of the 384 clones were selected at random for further analysis and divided into 15 sub-libraries. A 2-dimensional matrix was established so that each sub-library contains 20 clones with at least 1 clone overlapping with a single other sub-library. Plasmid DNA was extracted from each sub-library and used to DNA coat gold particles for immunization. The immunisation comprised 17 groups of 7 animals each as follows: (1) 15 groups of animals administered experimental DNA; (2) 1 group received negative control DNA (empty vector) and (3) 1 group immunized with live chlamydial MoPn EB's. Animals were immunized and swabs collected as per ELI screen 1. Swab samples were inoculated onto McCoy cell monolayers and number of inclusion for each group of animals at several time points was determined by immunofluorescent staining. Two protective sub-libraries (SL209, SL213) were identified comprising 40 clones in total; one common clone was identified in the two sub-libraries.

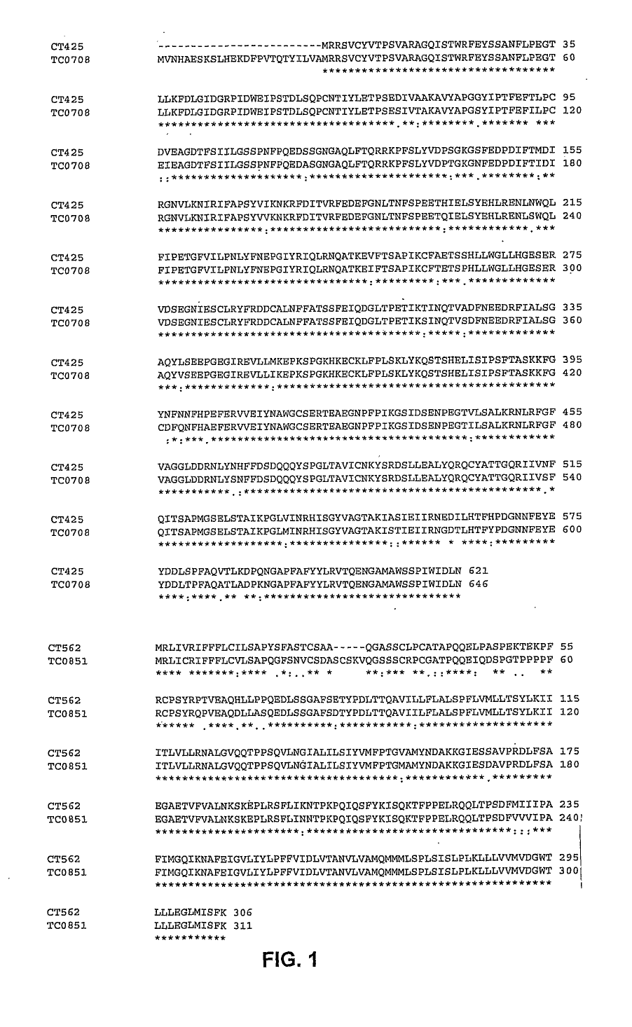

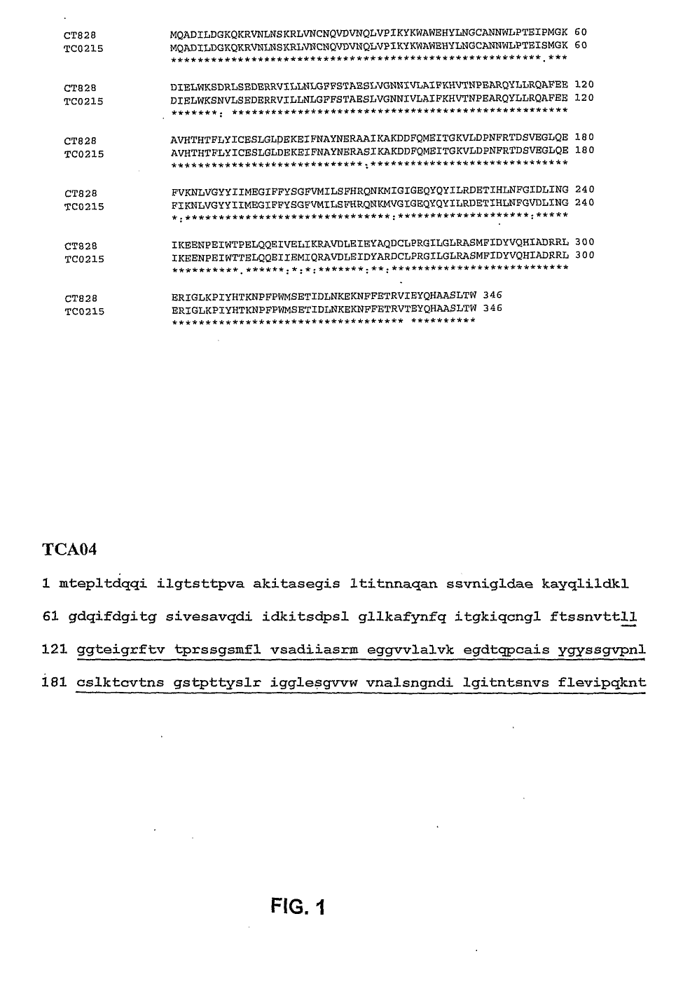

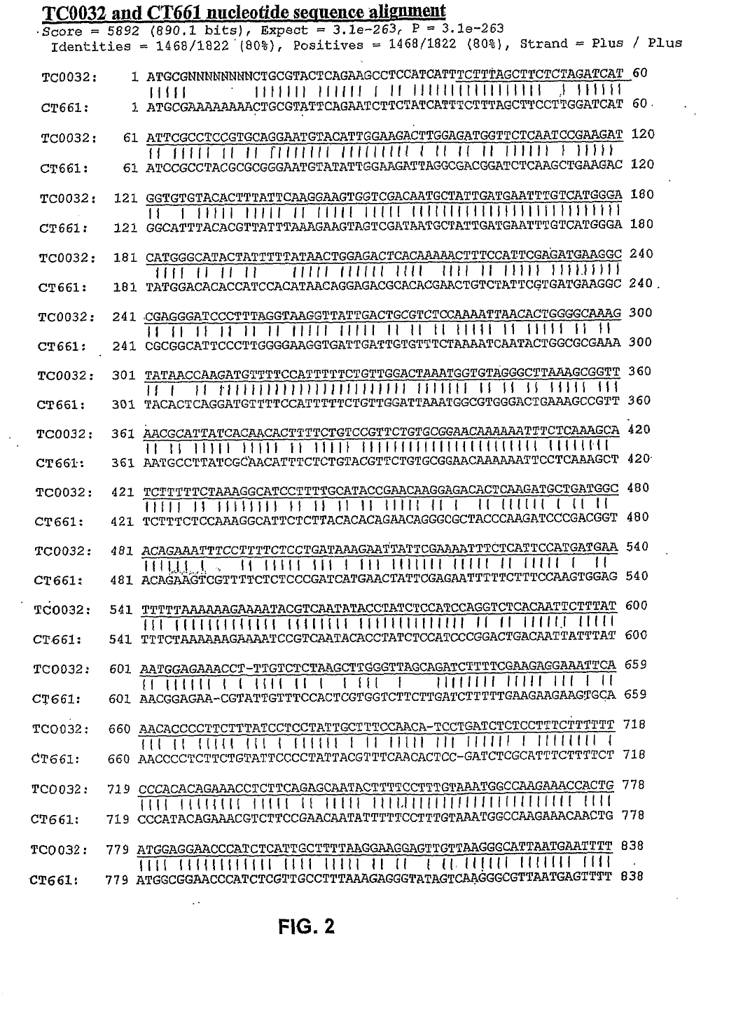

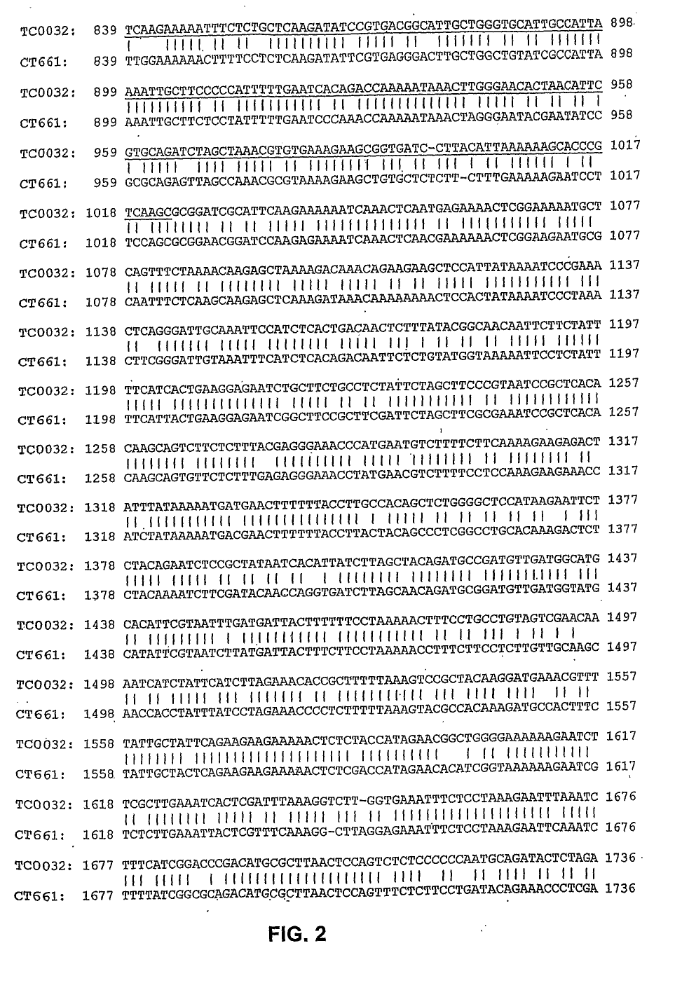

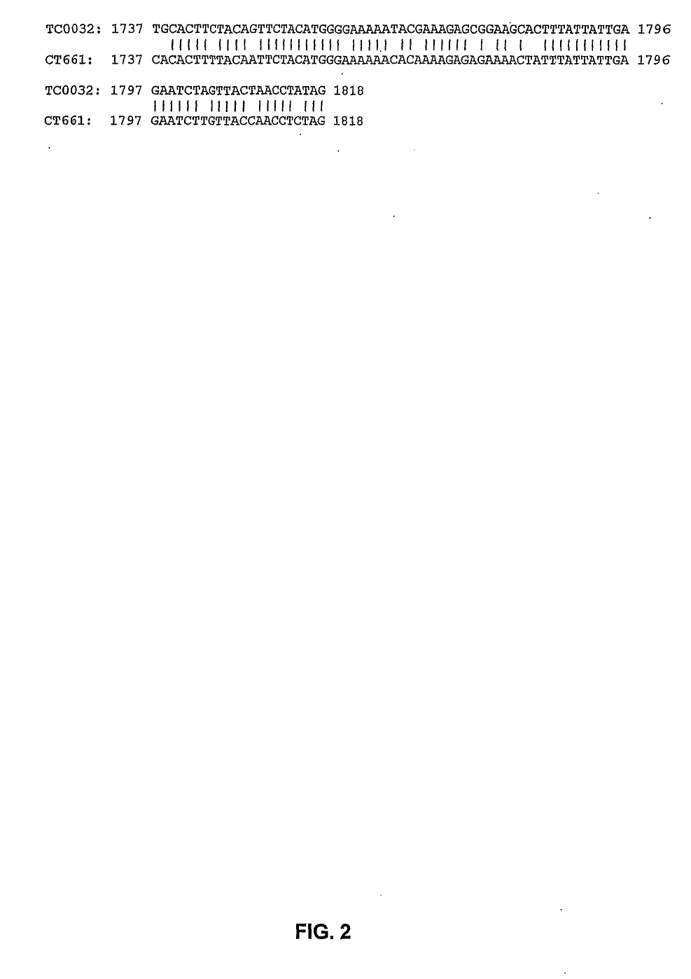

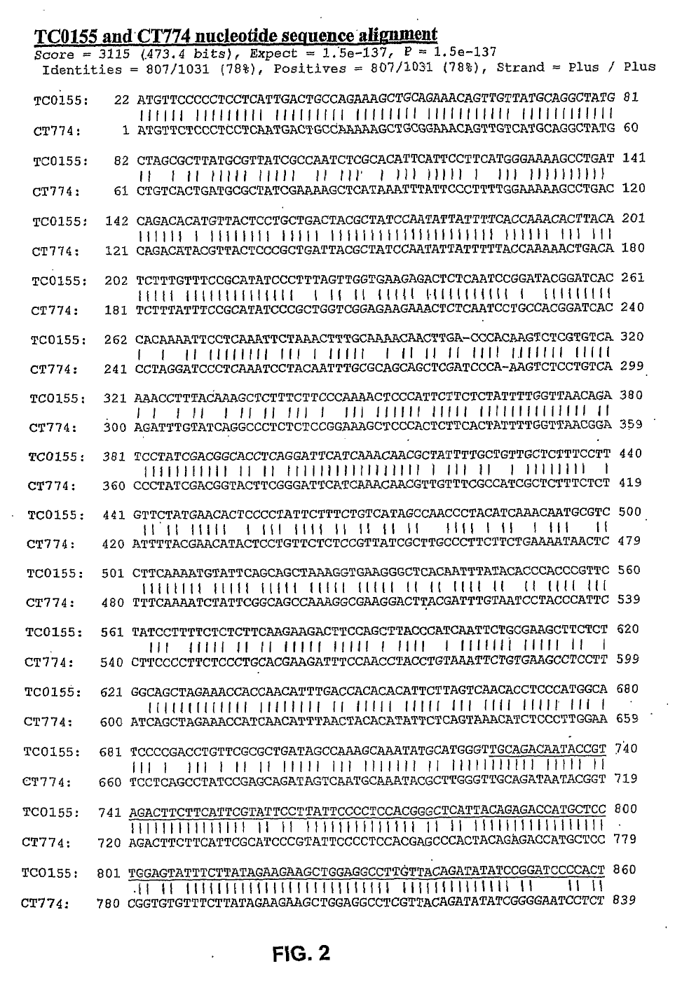

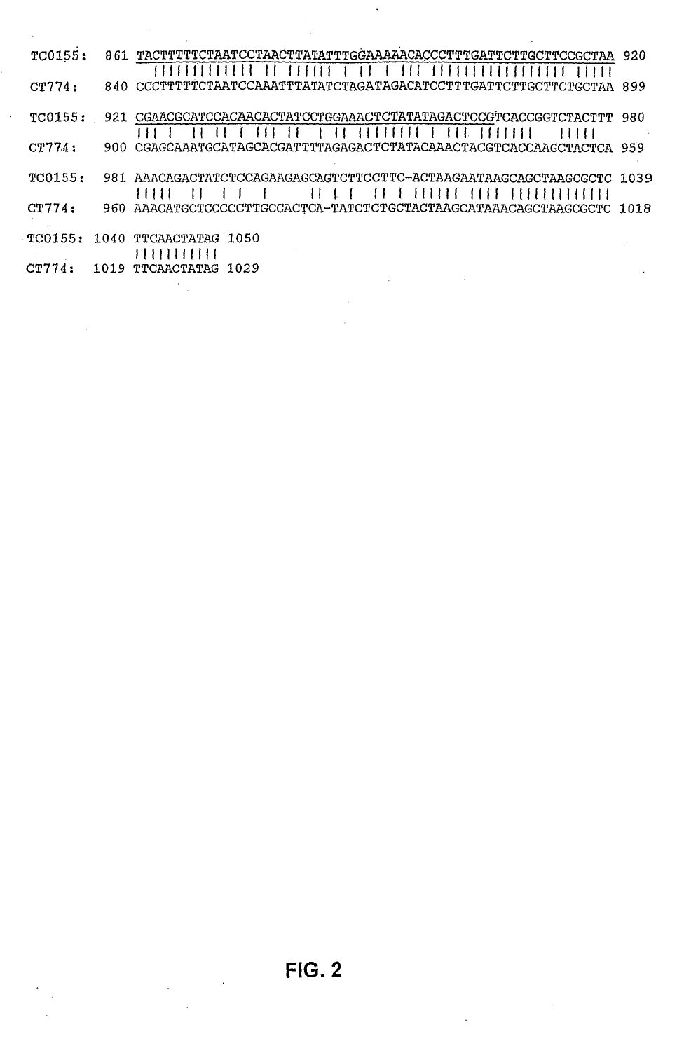

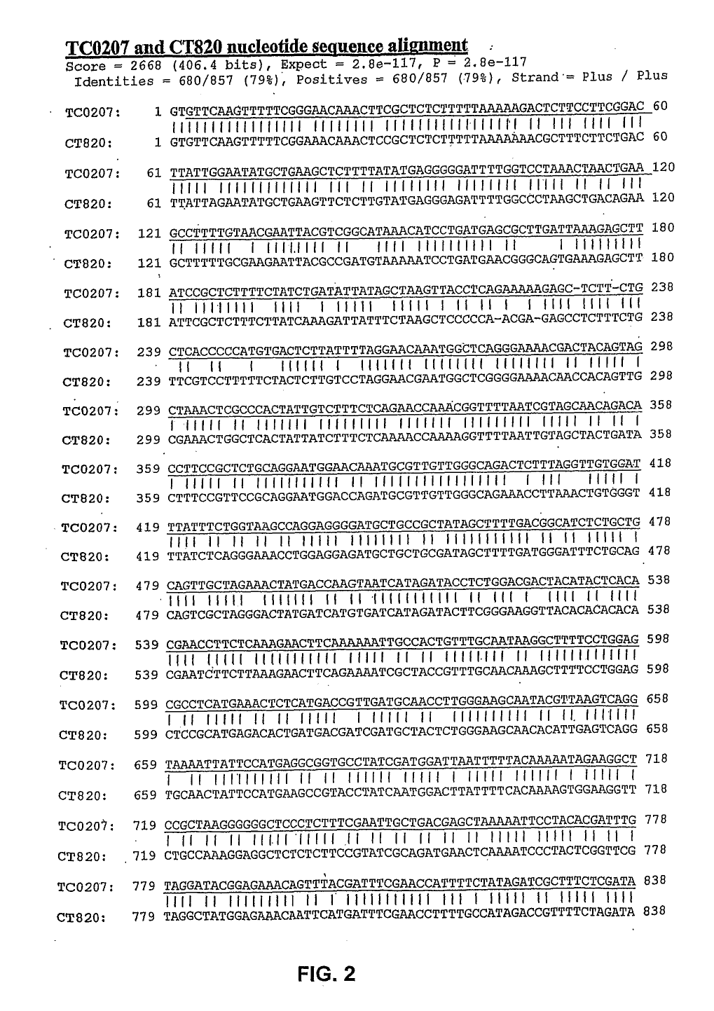

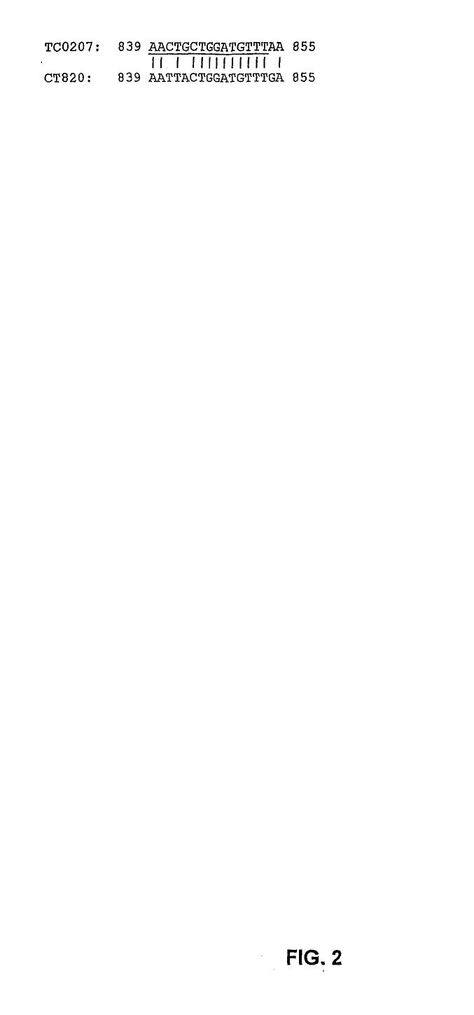

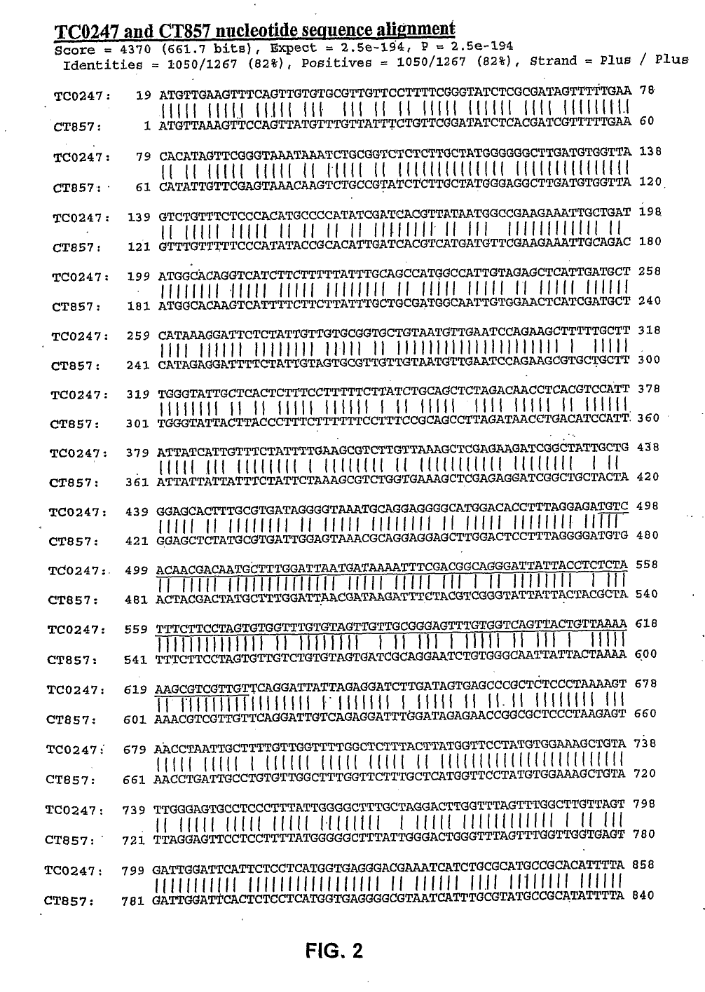

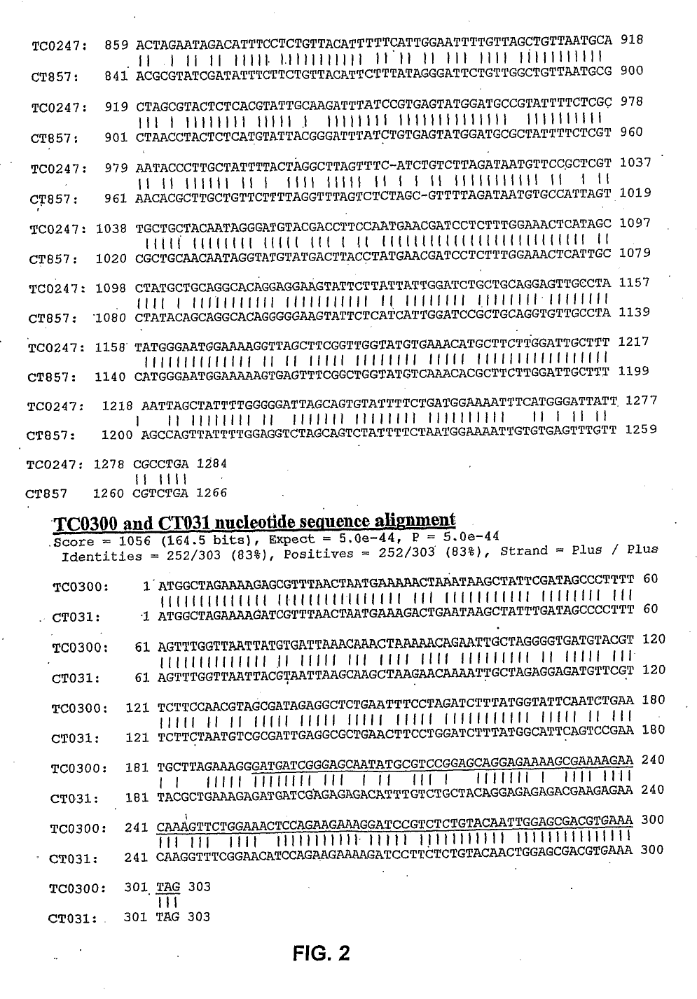

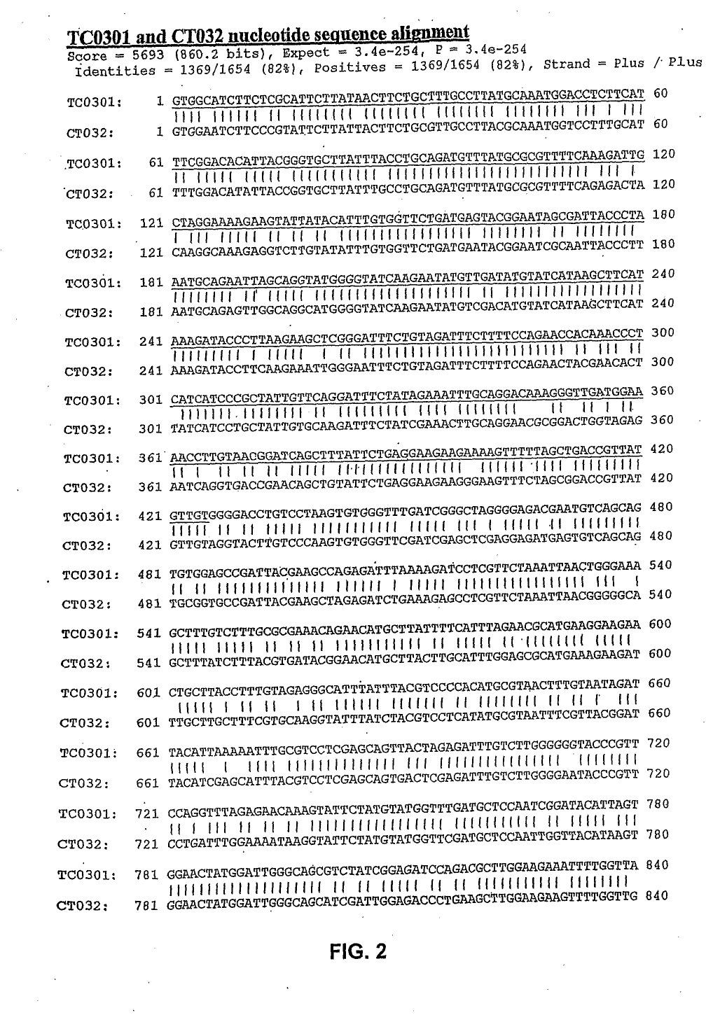

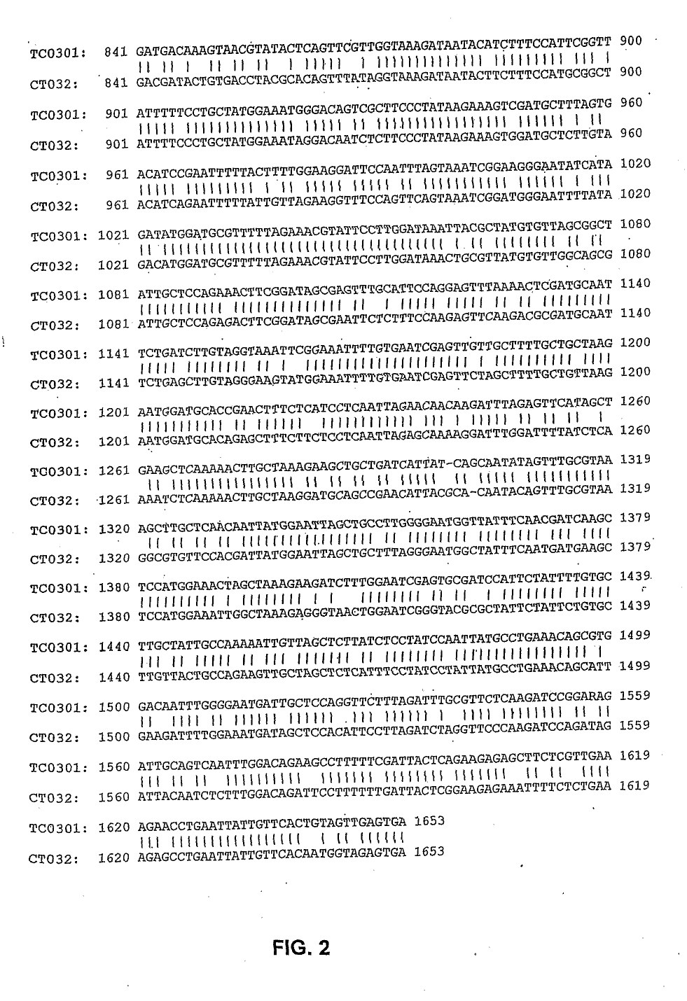

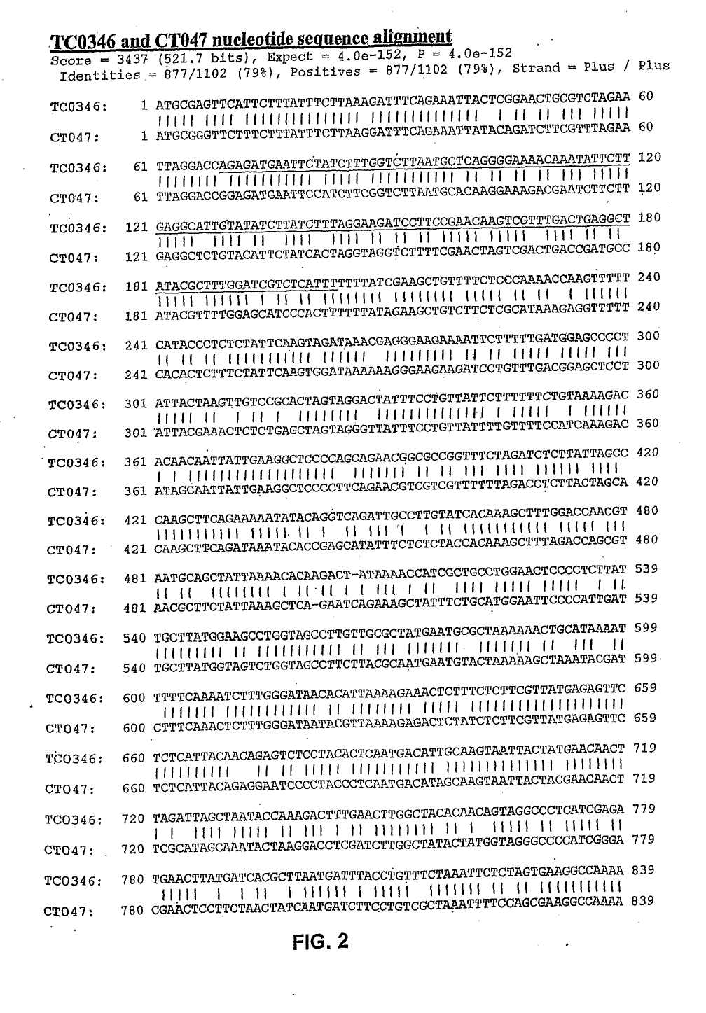

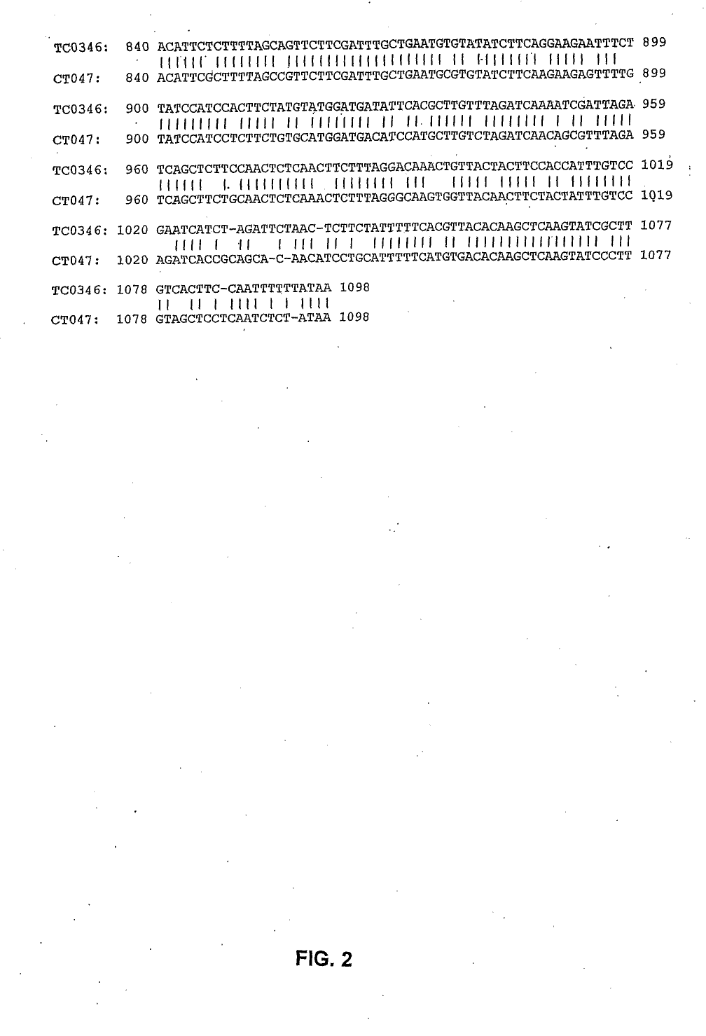

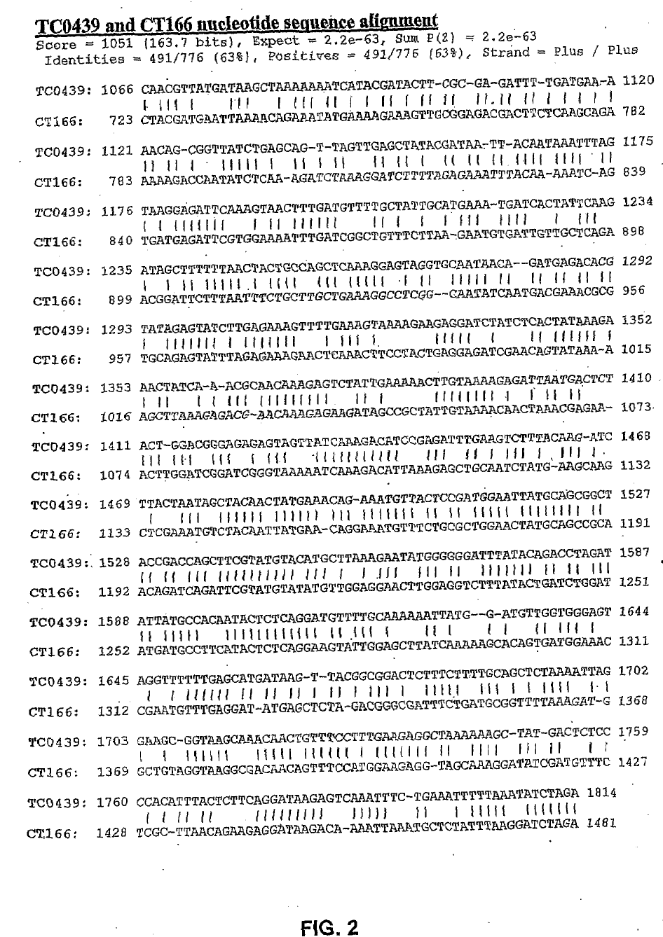

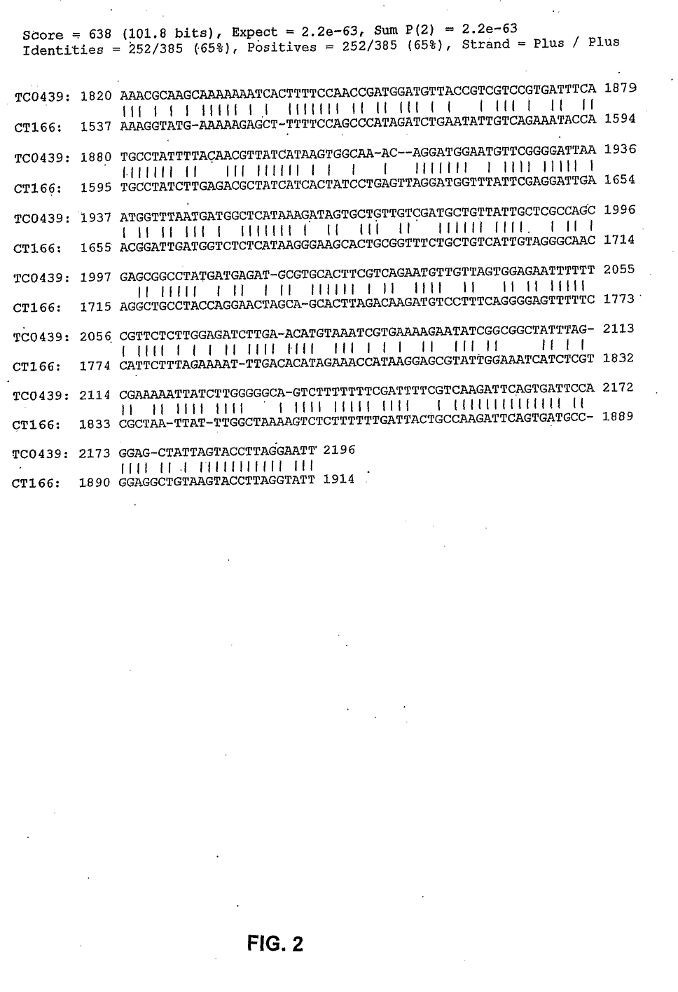

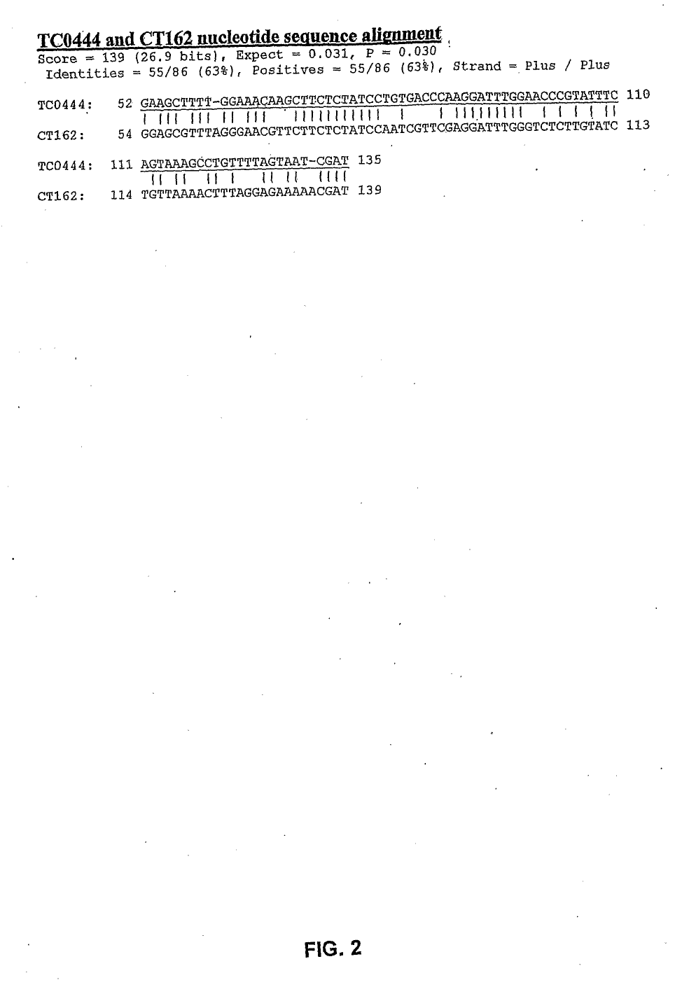

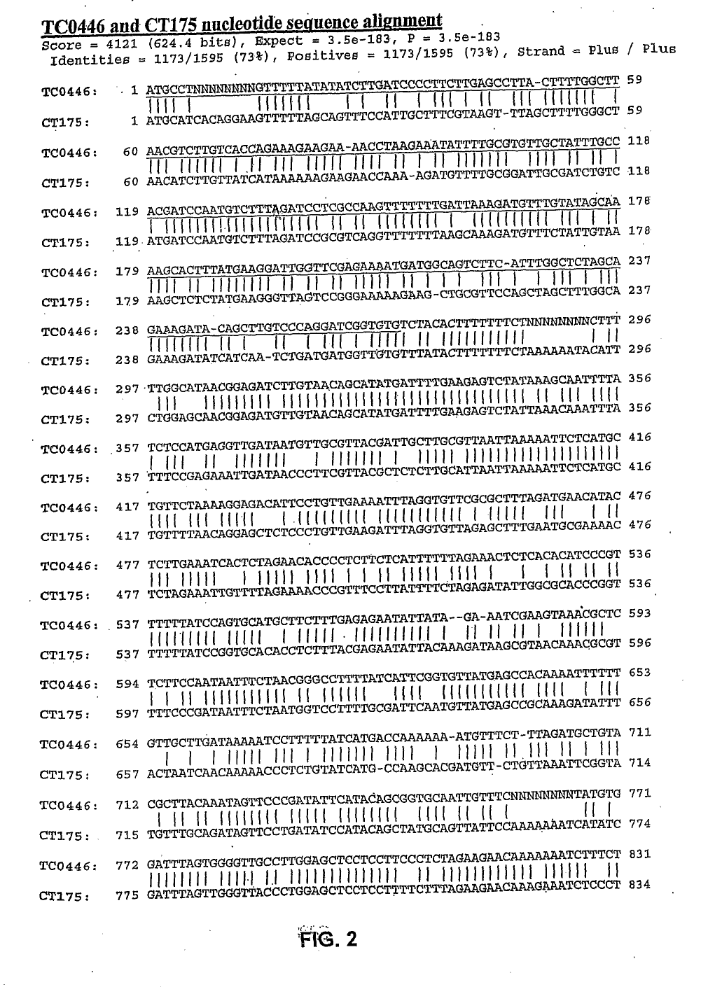

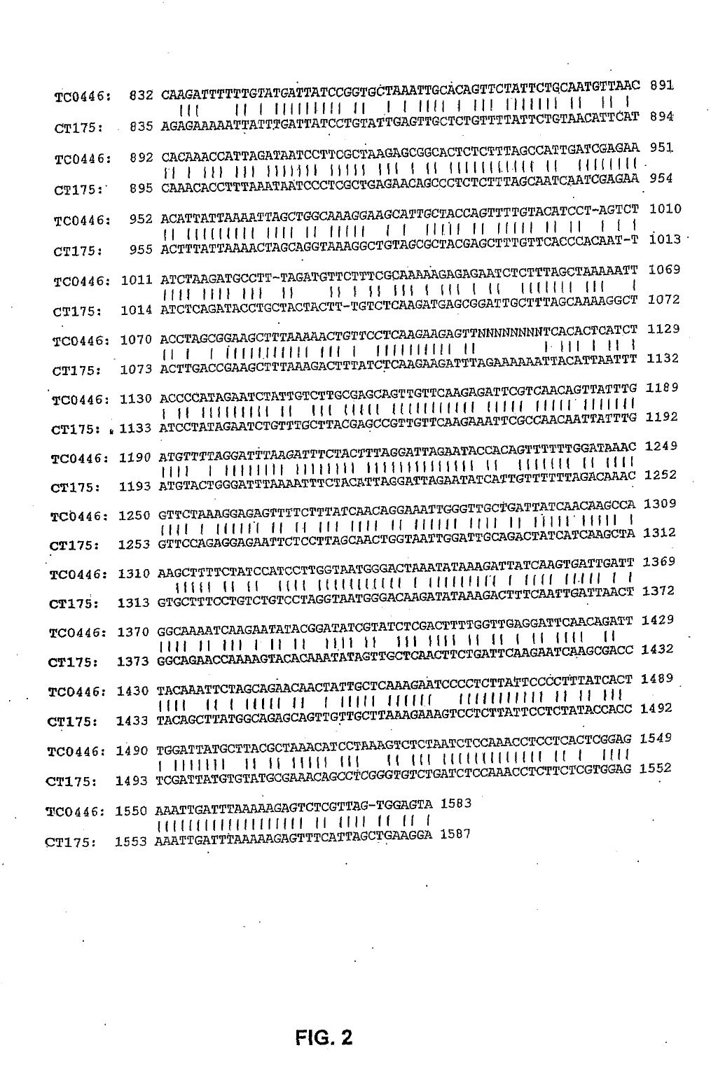

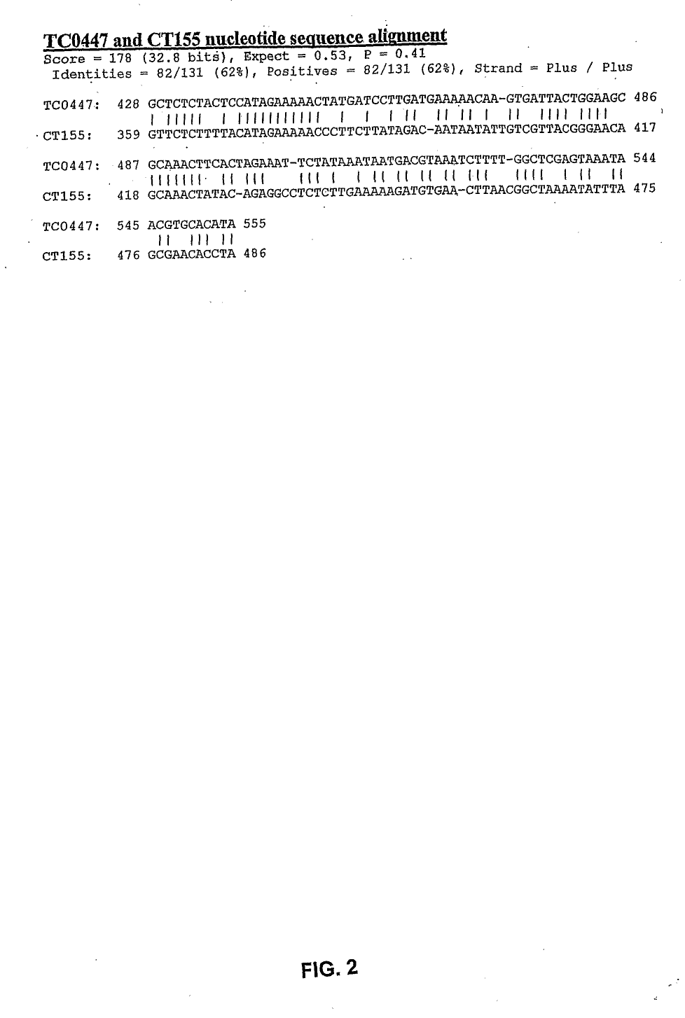

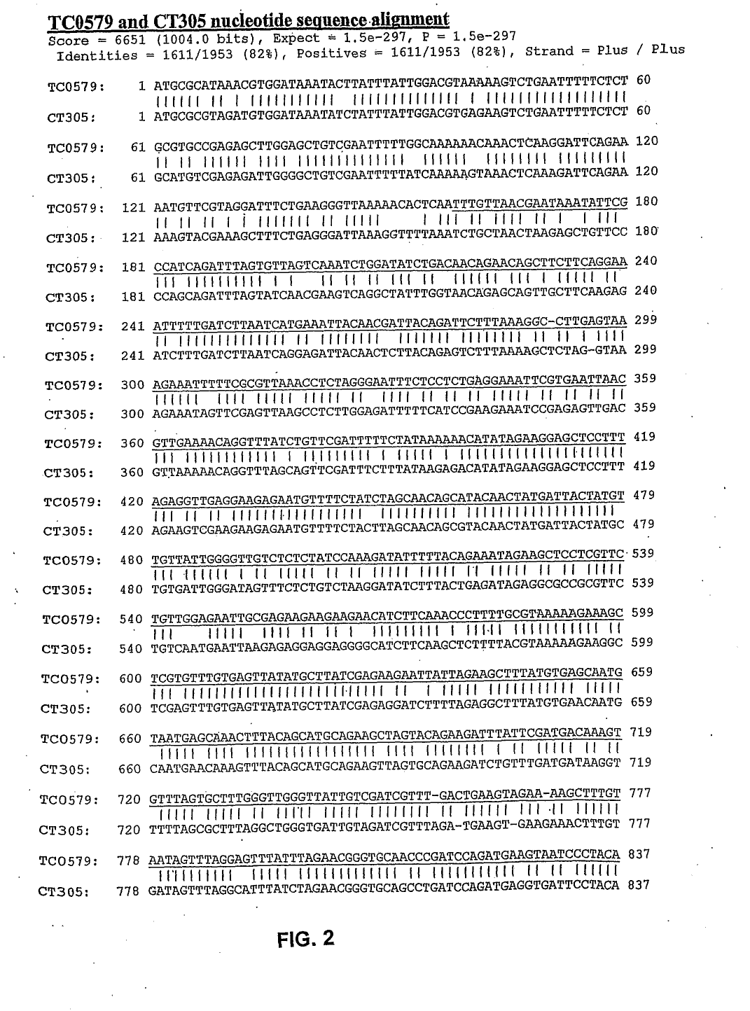

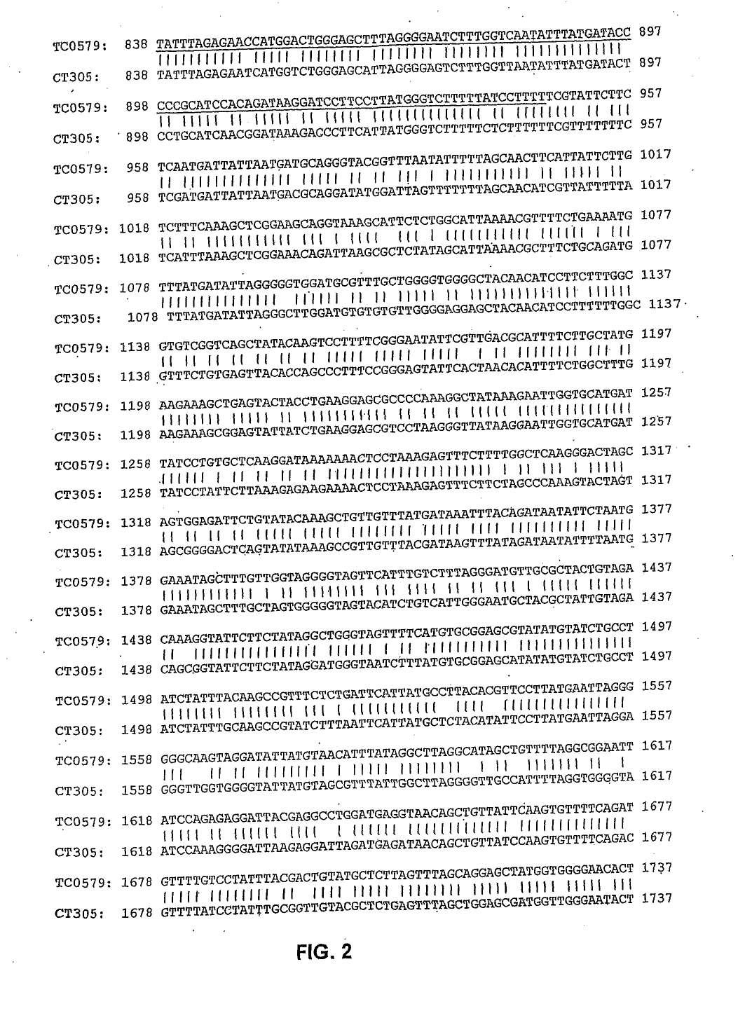

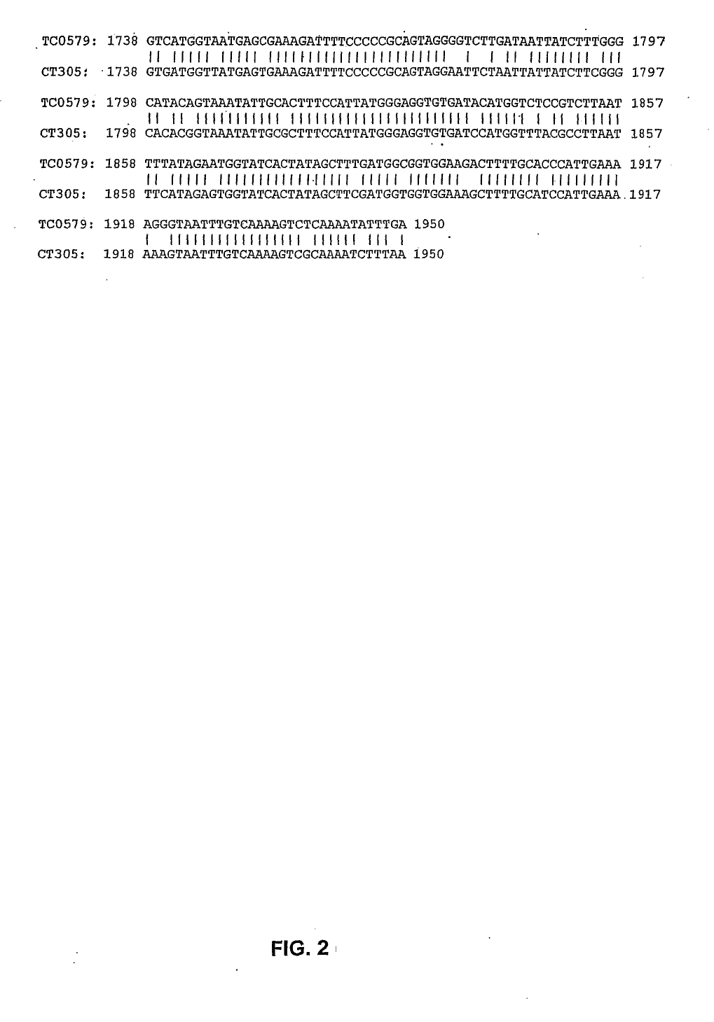

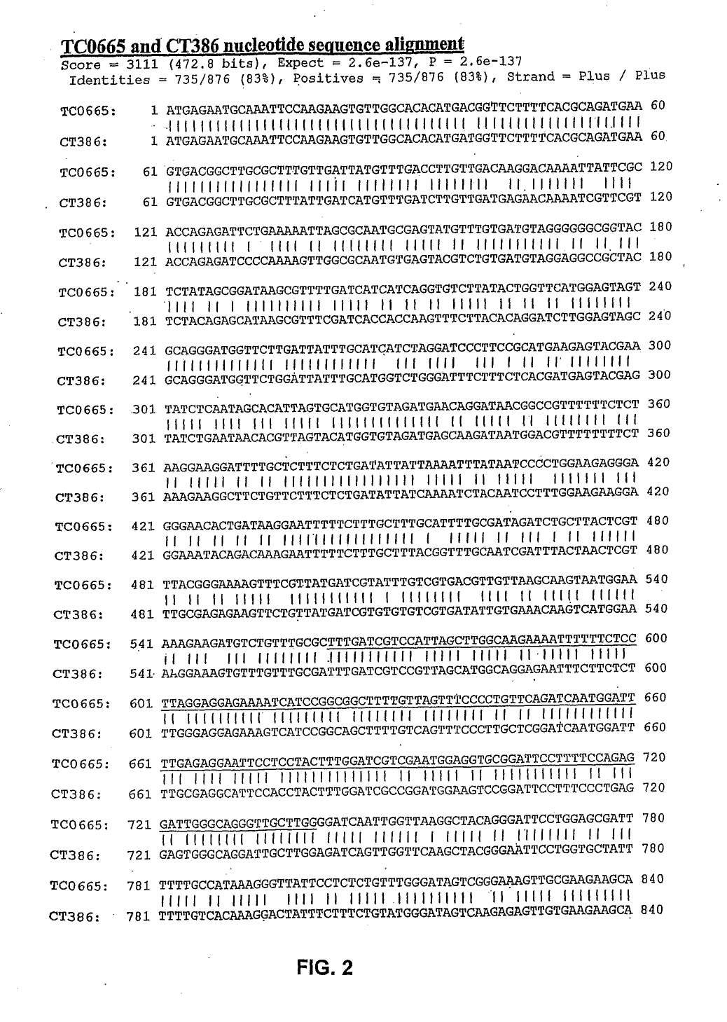

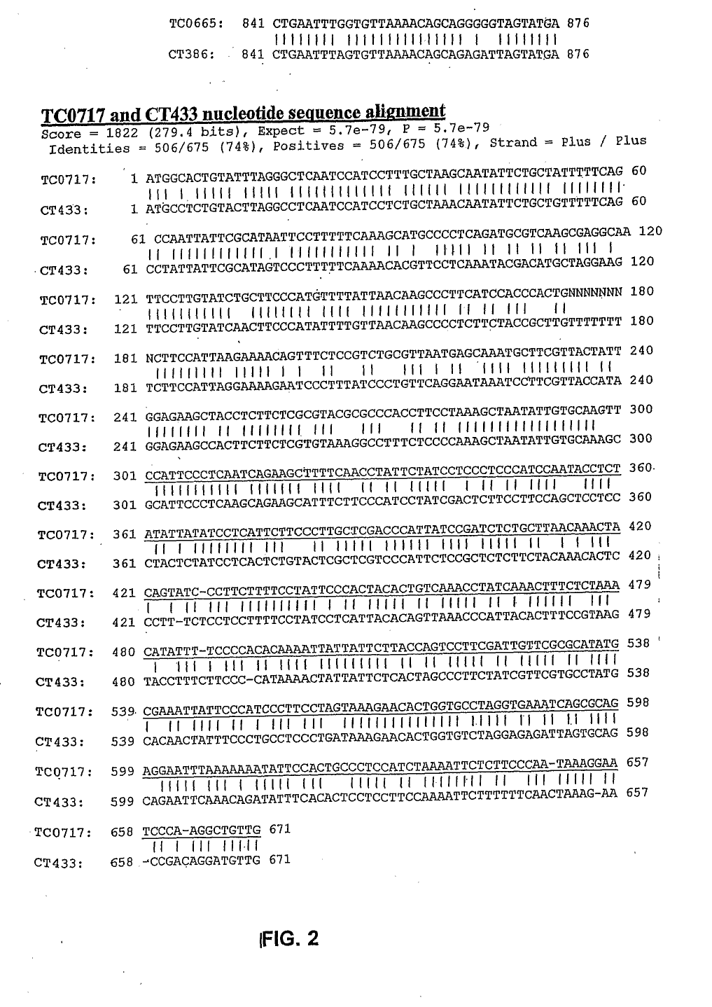

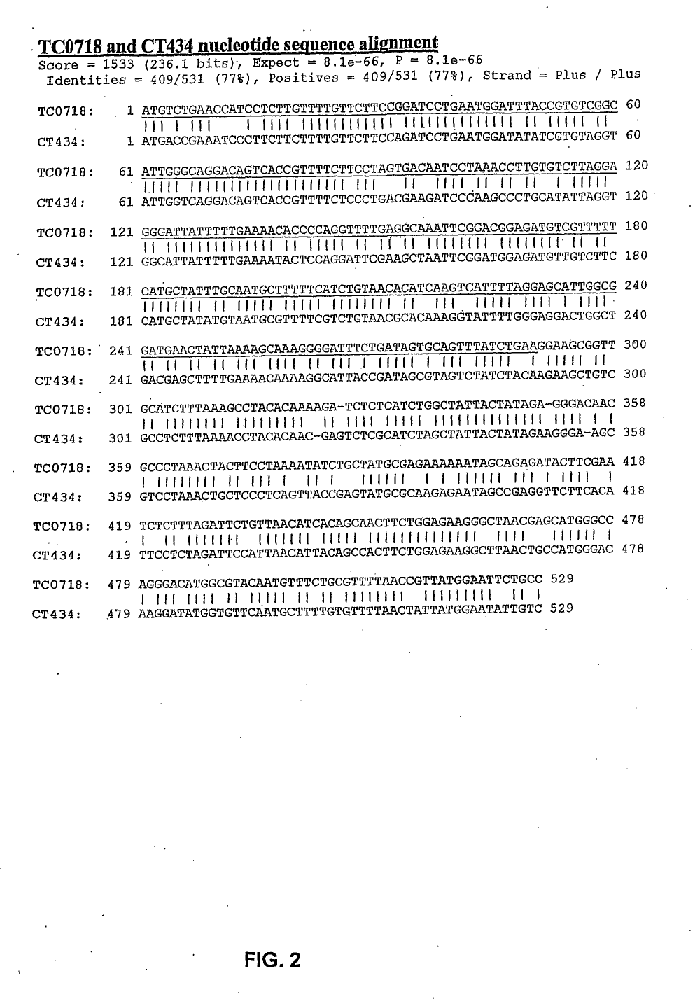

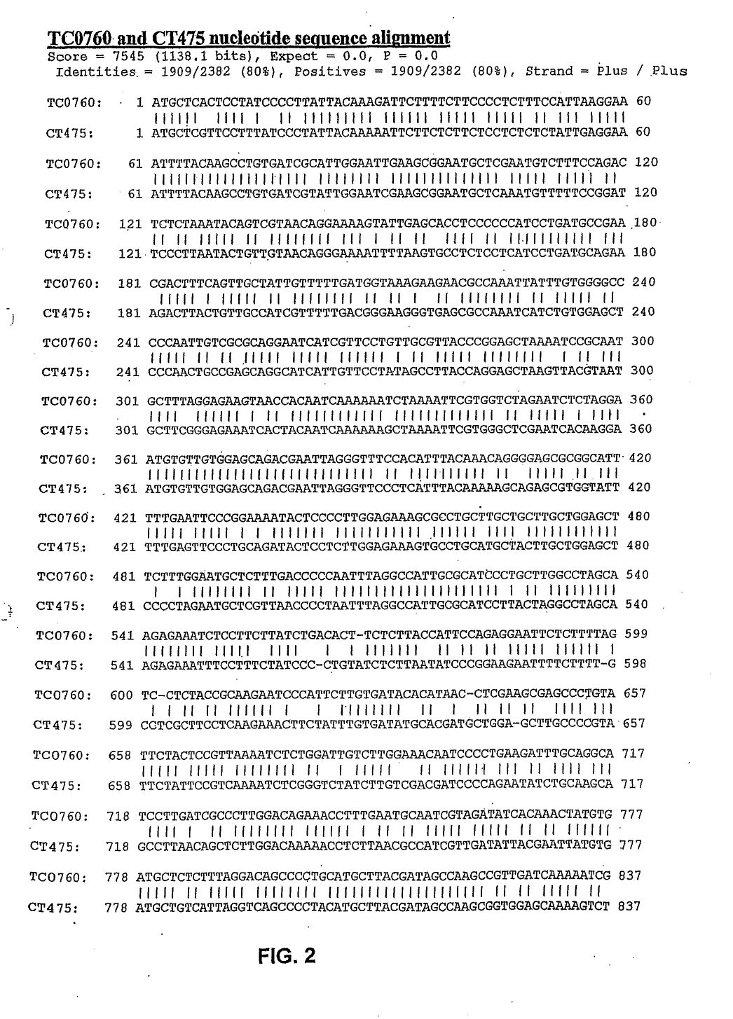

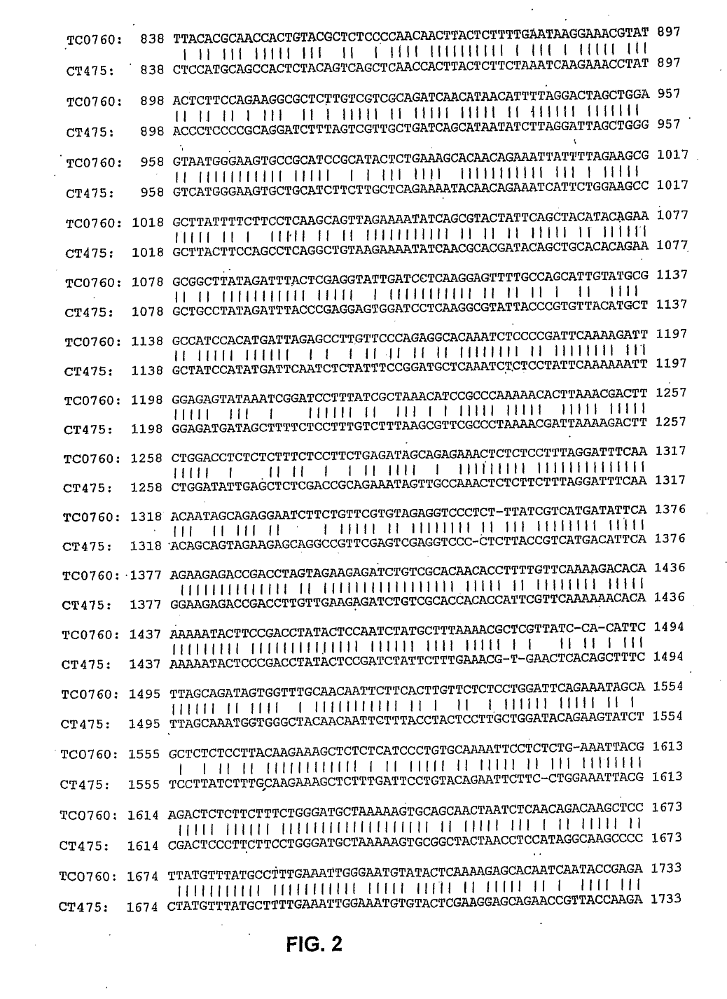

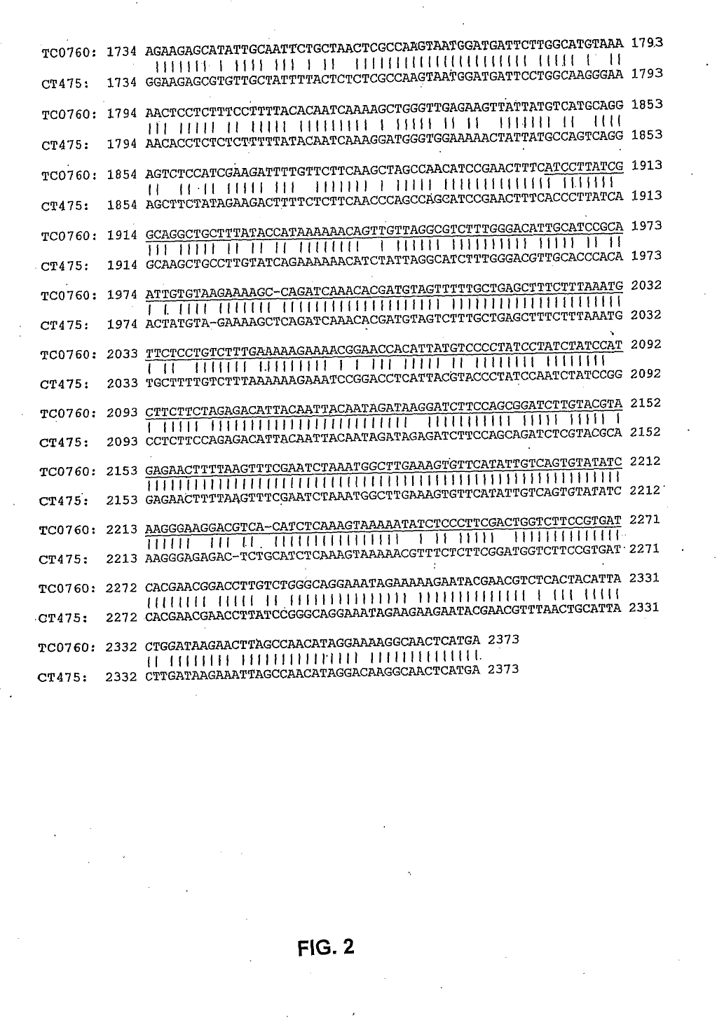

Sequence analysis and BLAST searches of the identified clones revealed 18 chlamydial genes, SEQ ID NOS: 149 to 166 in the mouse and SEQ ID NOS: 106 to 124 in human (FIG. 2), within the “protective” sub-libraries, and 19 clones comprising “junk” (host cell/vector) DNA (several genes were contained in both sublibraries hence the total number of clones identified does not equal the number sequenced (40)). Eight gene fragments represented in 7 clones were selected for further analysis.

### ELI Screen 3:

Seven clones were selected for further analysis based on gene size, homology to human genome, homology to C. trachomatis genome and T-cell epitope prediction. DNA was isolated from each of these seven clones and controls and the DNA was used to coat gold particles for gene gun immunization. The immunization comprised 11 groups of 7 animals each as follows: (a) 7 groups of animals were administered DNA isolated from each positive clone; (b) 1 group was administered with pooled DNA isolated from all clones in SL209; (c) 1 group was administered pooled DNA isolated from SL209 without the “junk” DNA clones (designated SL309); (d) 1 group was administered empty vector DNA and (e) 1 group was immunized with live chlamydial MoPn DNA as in ELI screen 2. The immunization schedule was the same as for both previous ELI screens.

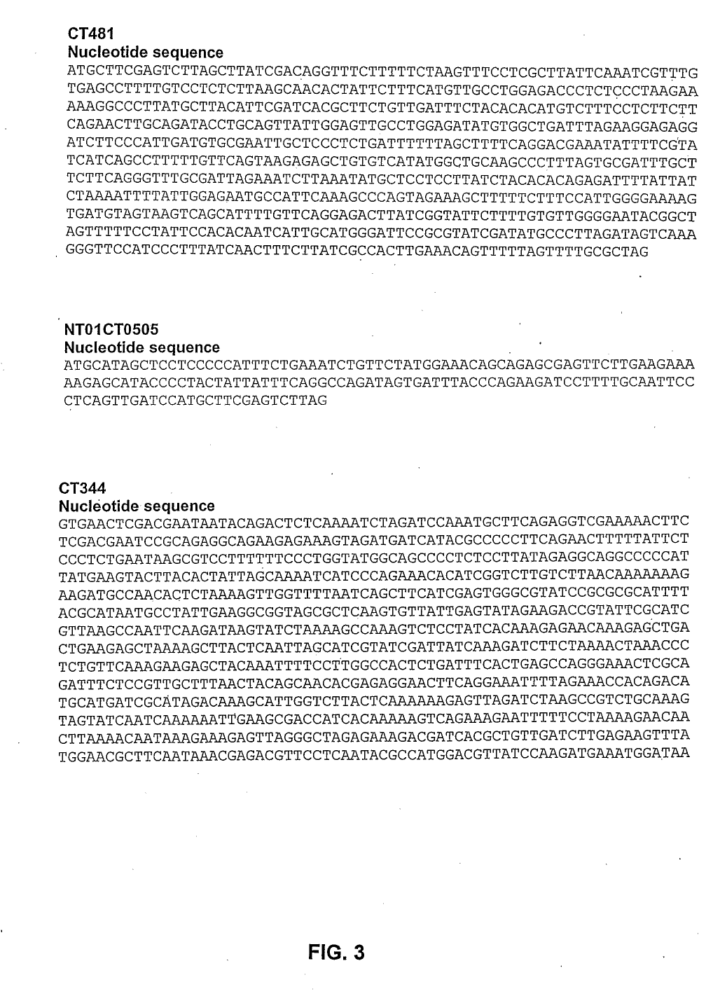

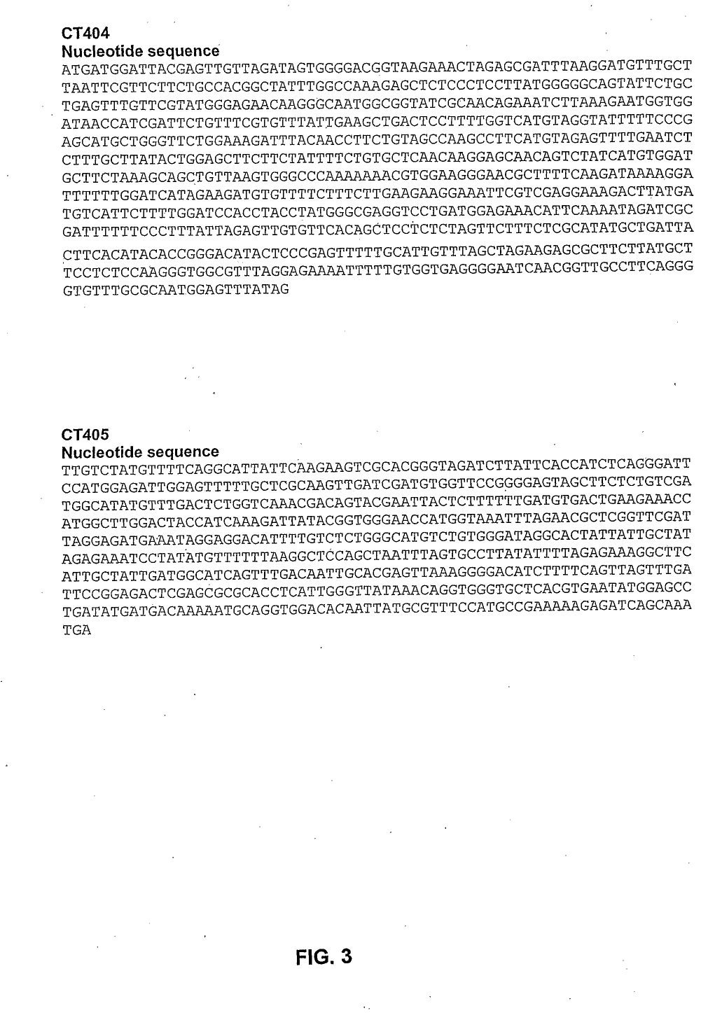

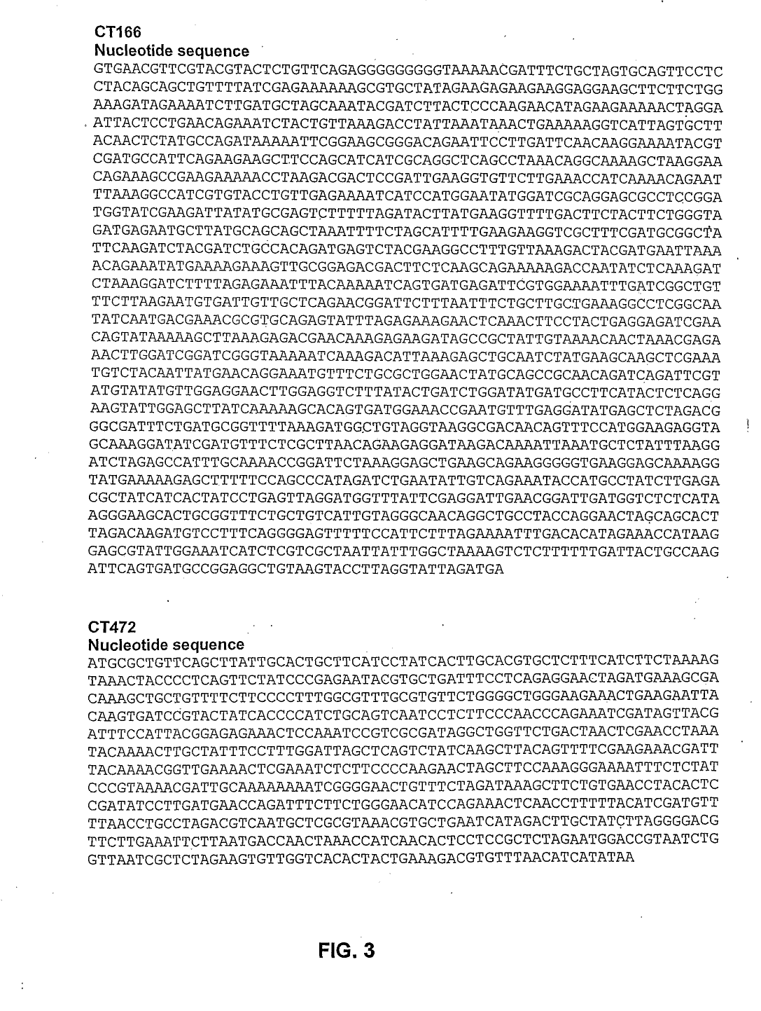

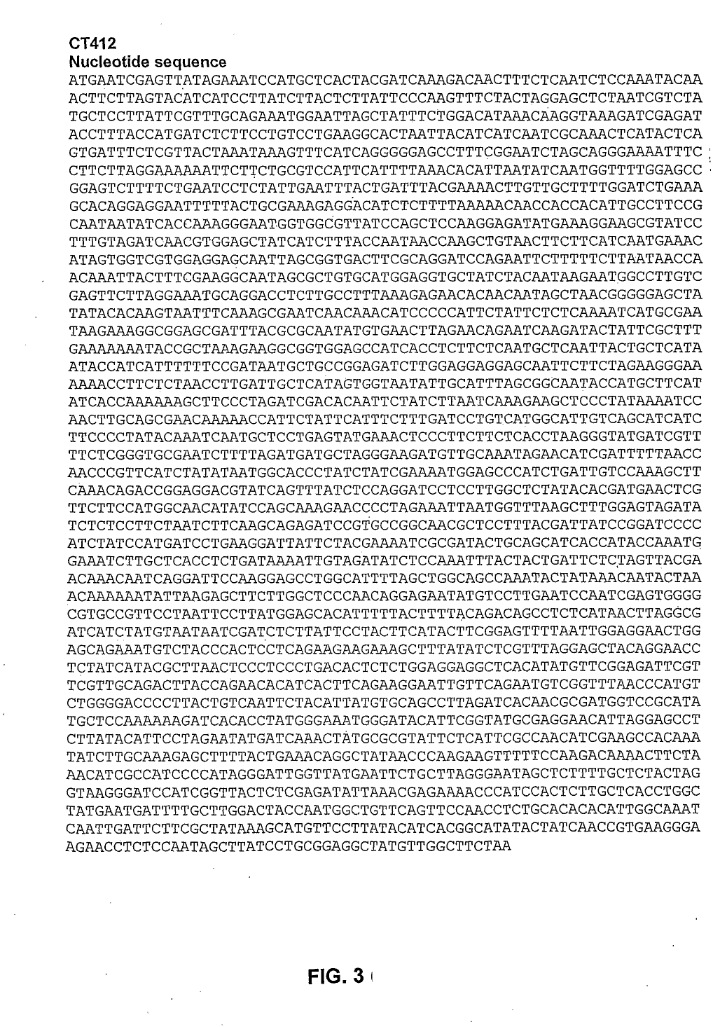

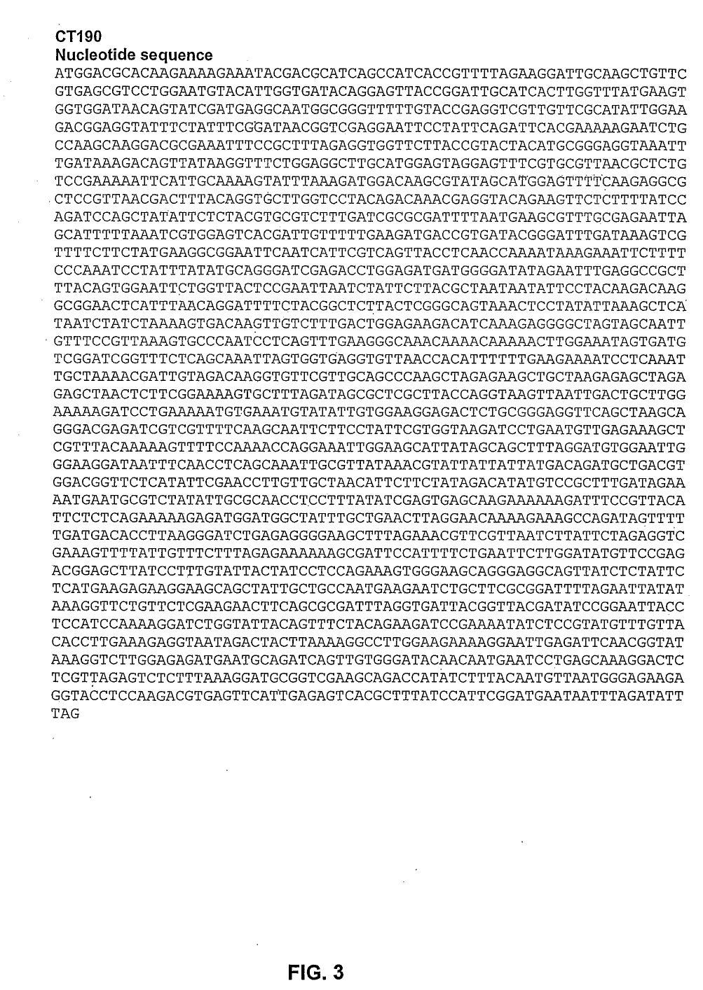

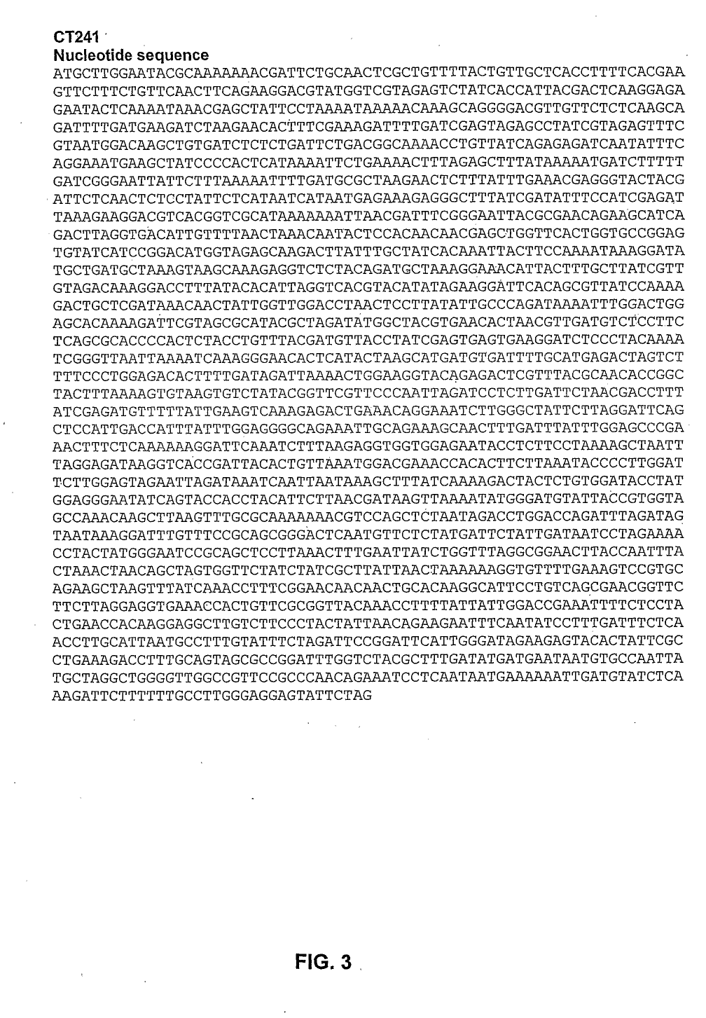

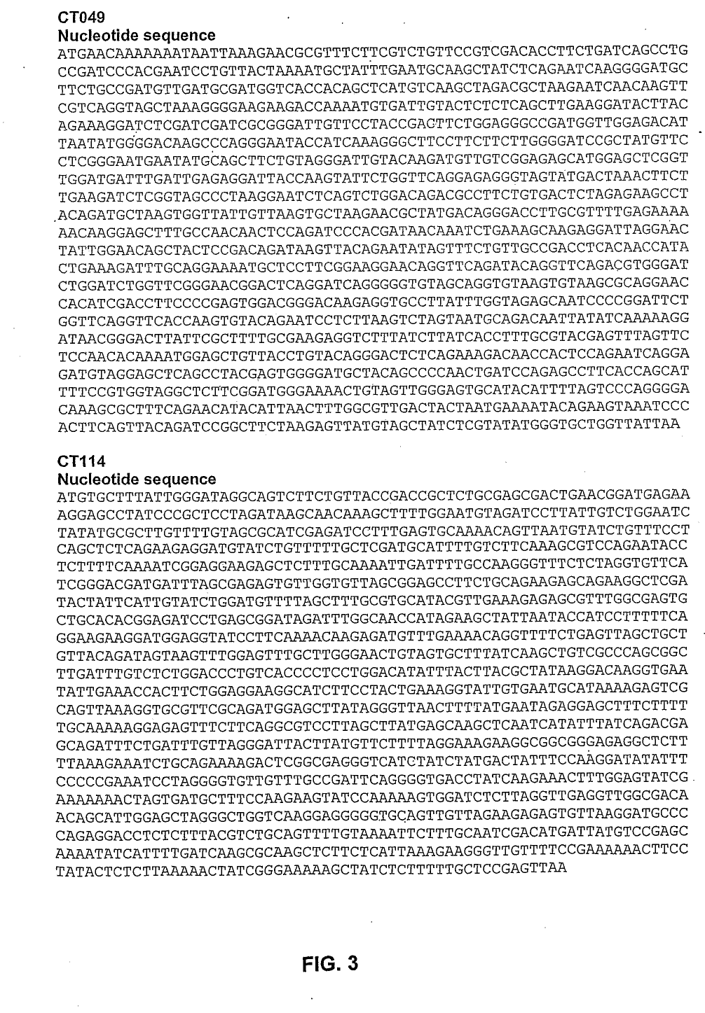

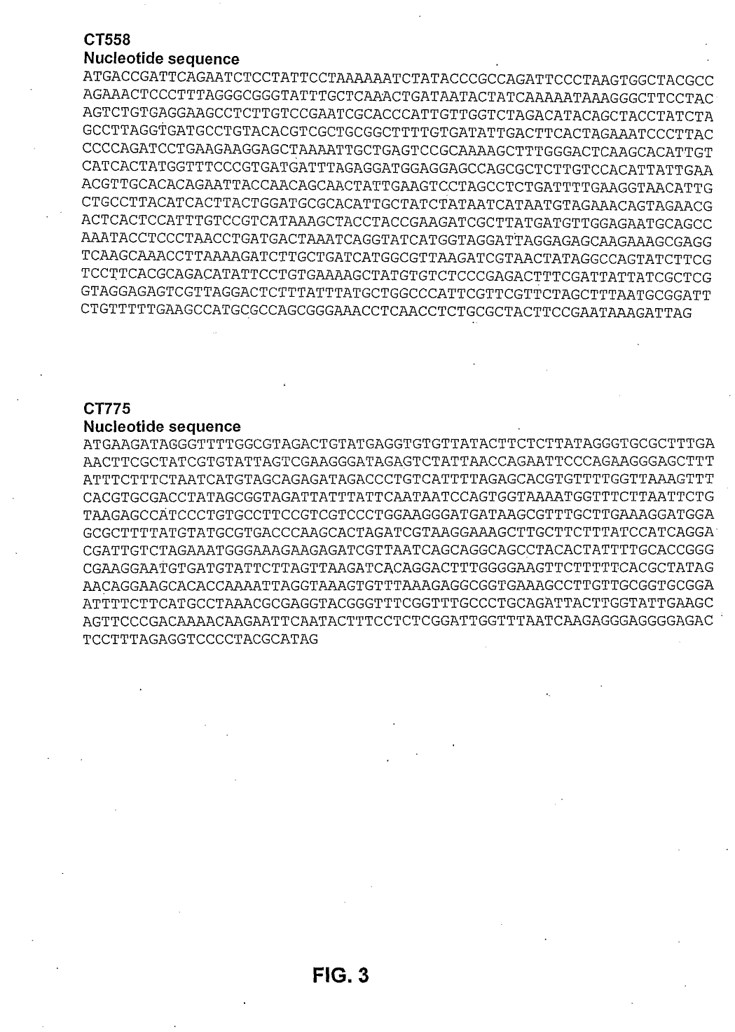

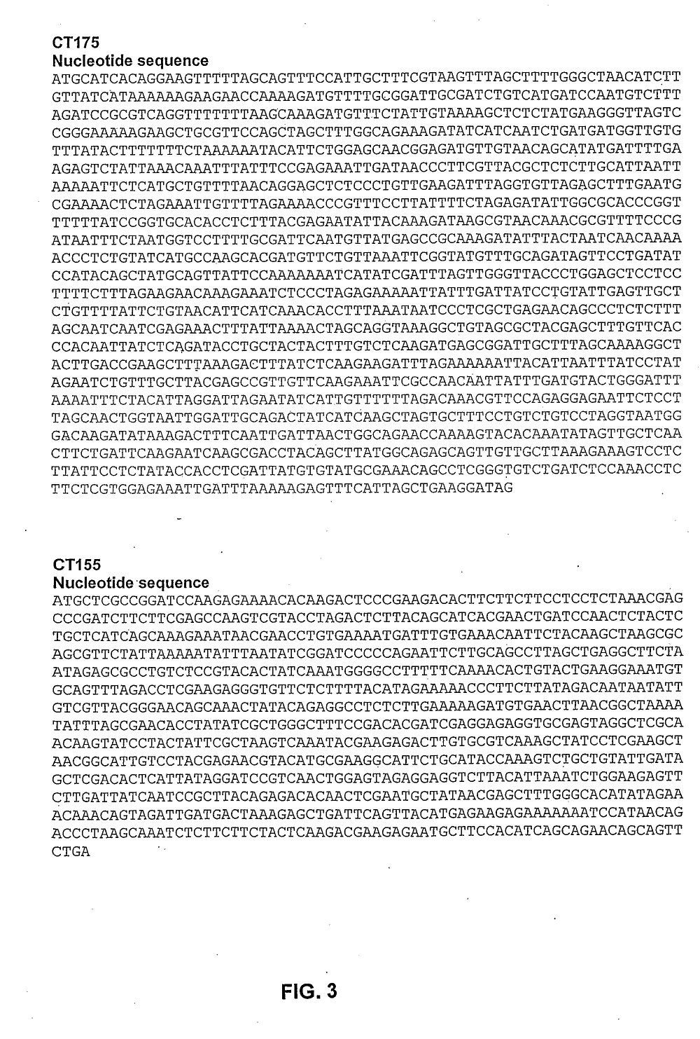

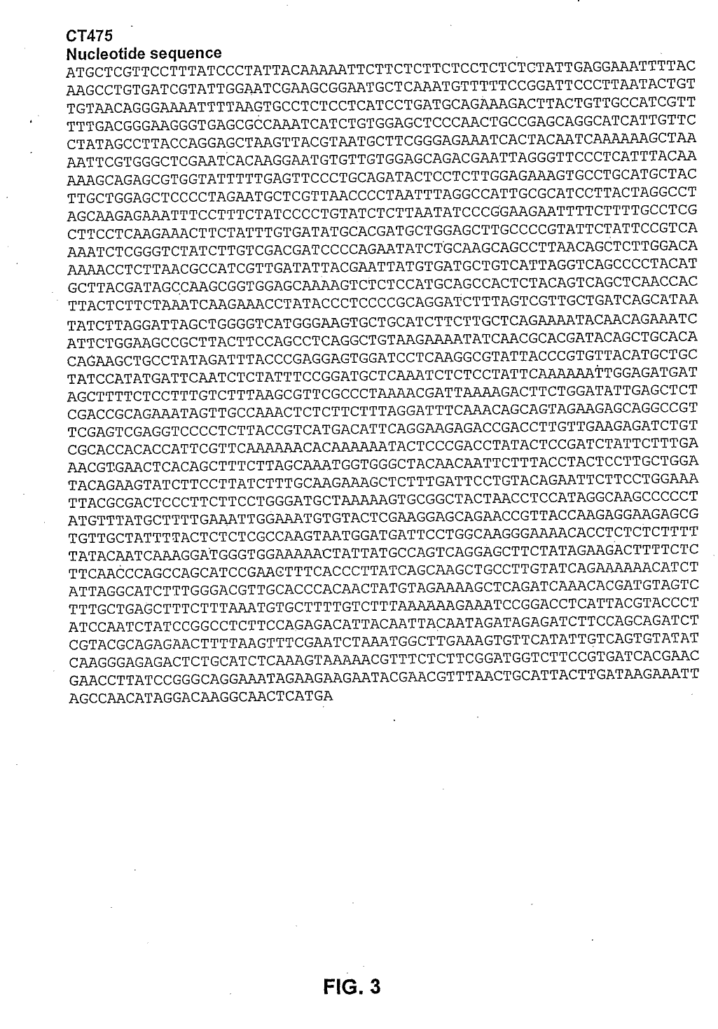

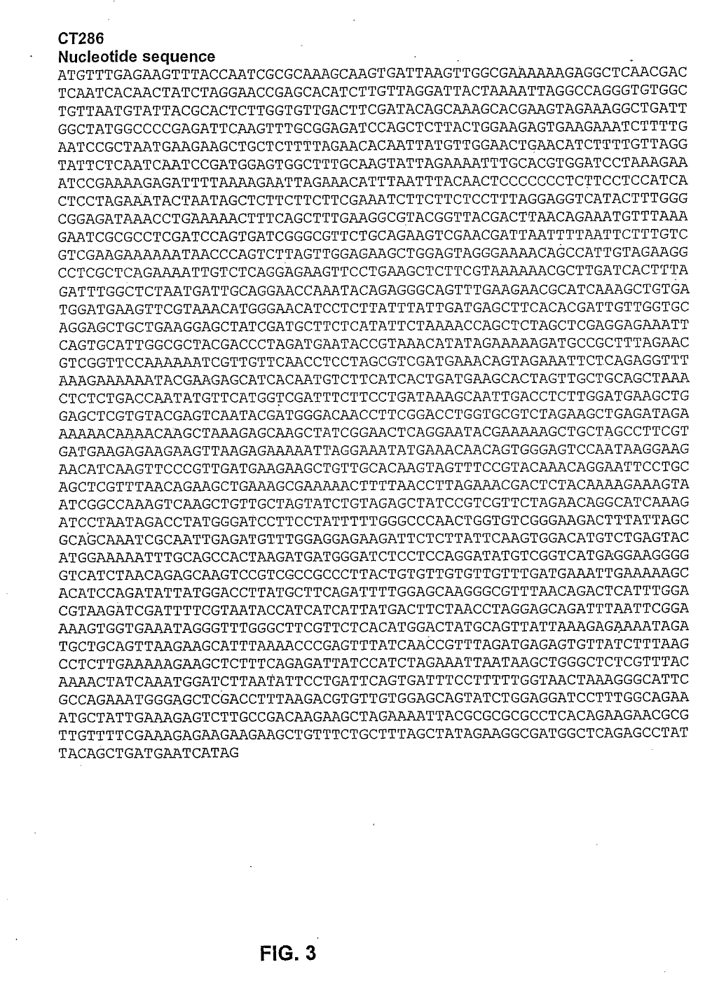

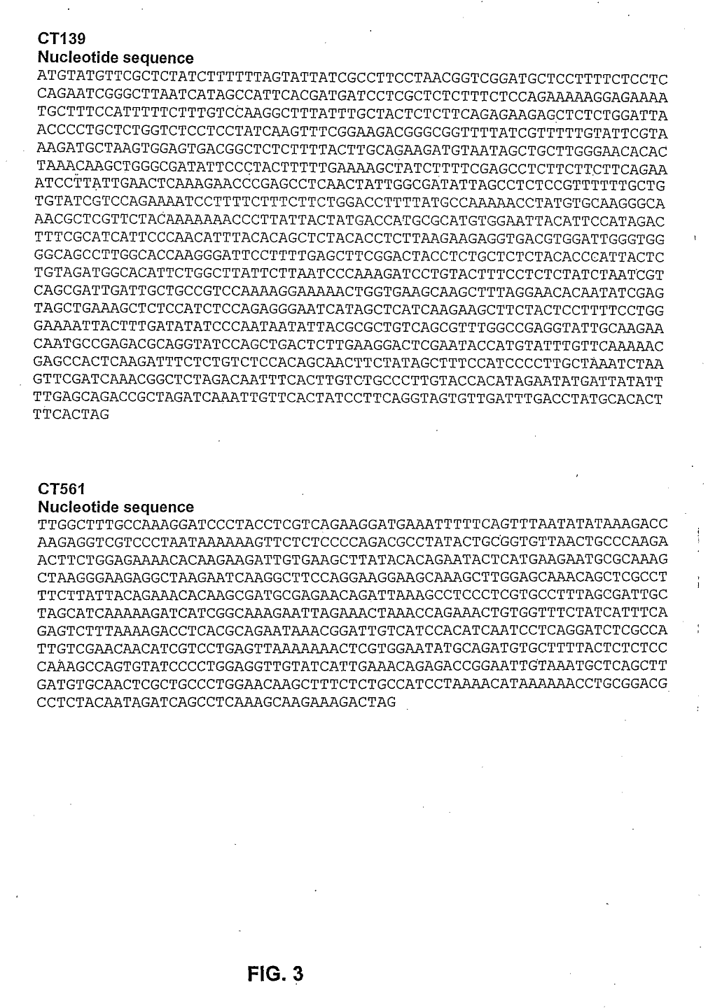

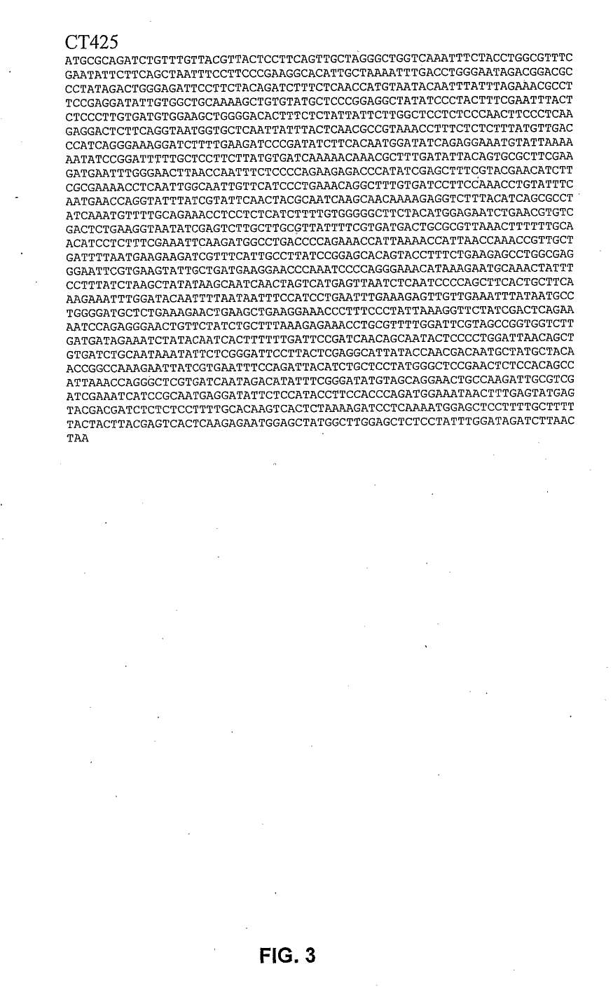

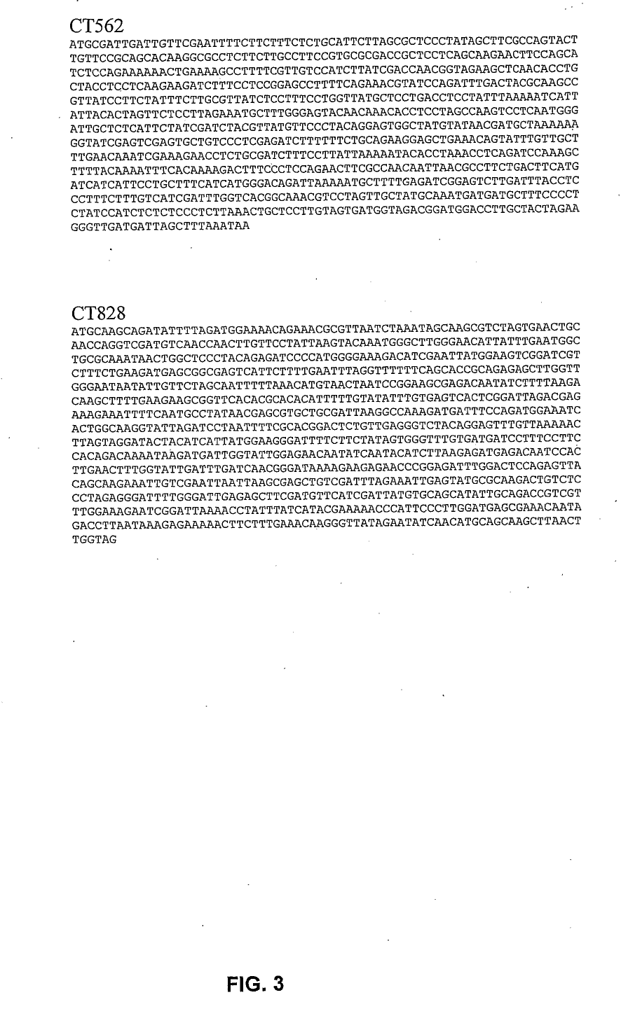

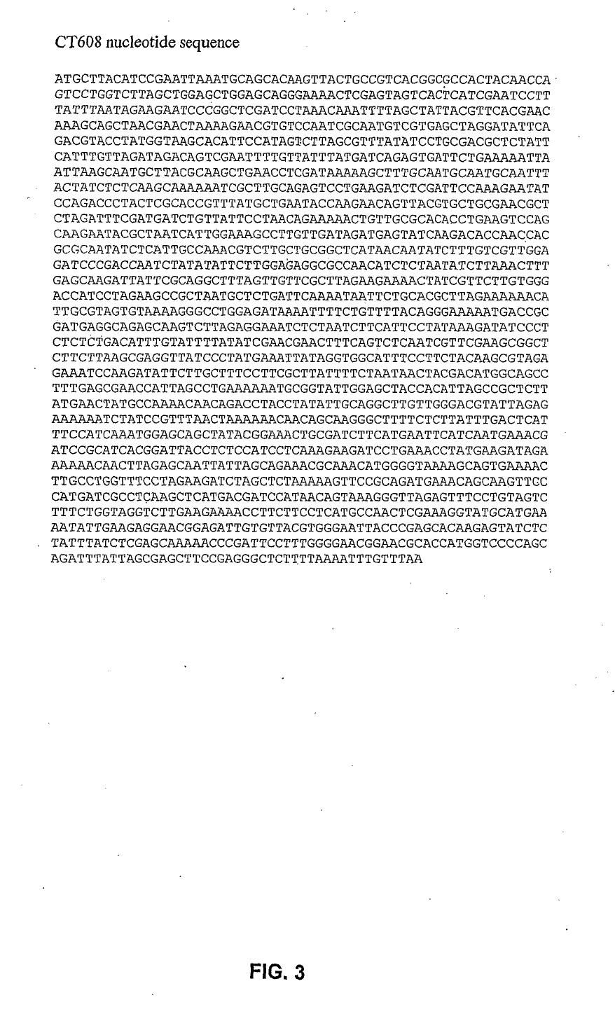

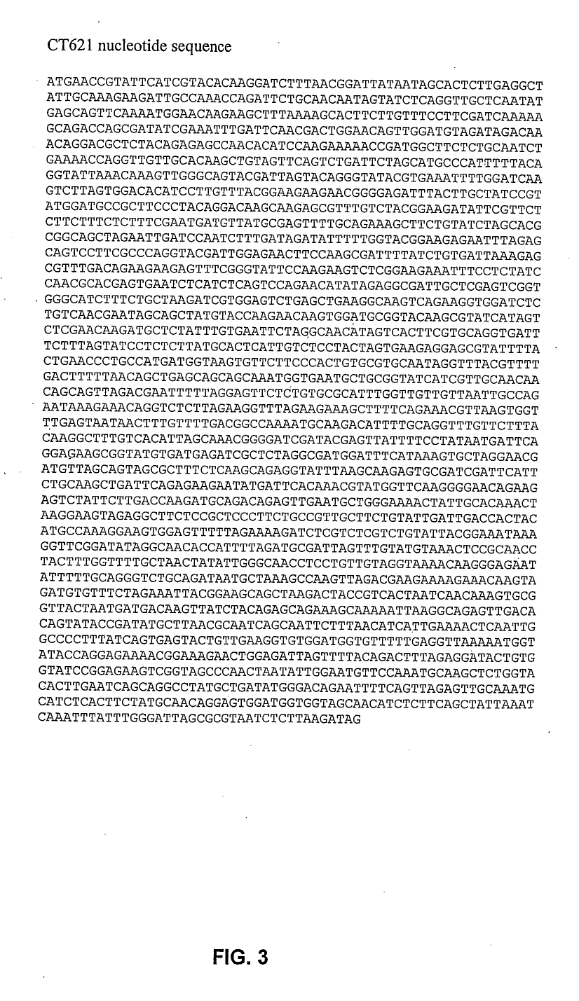

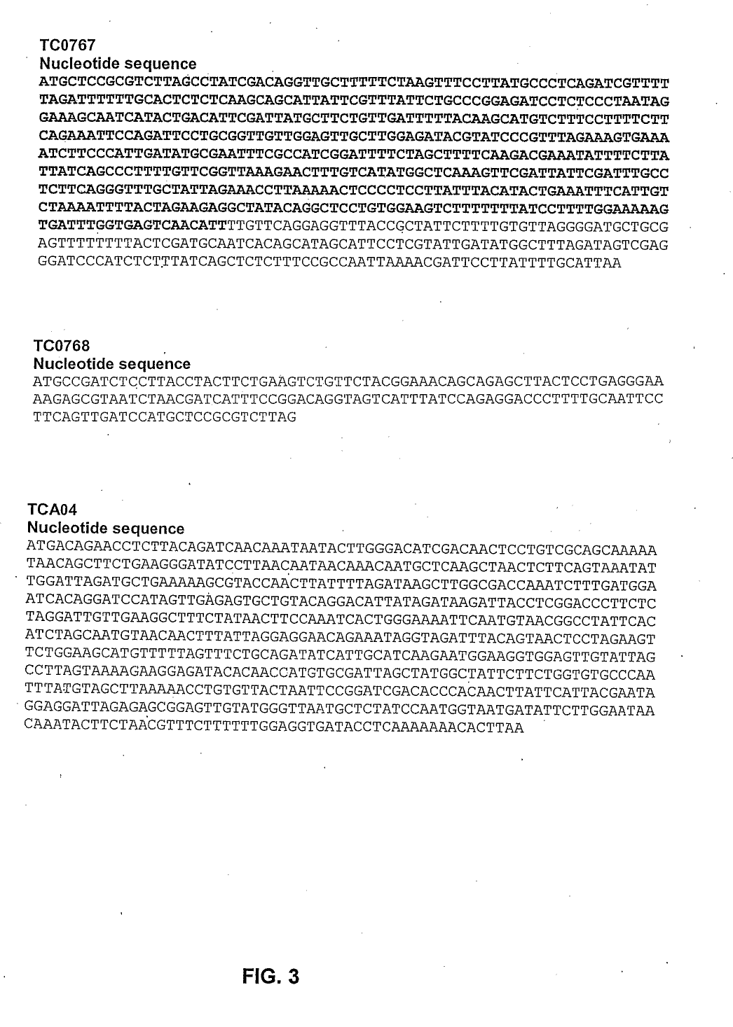

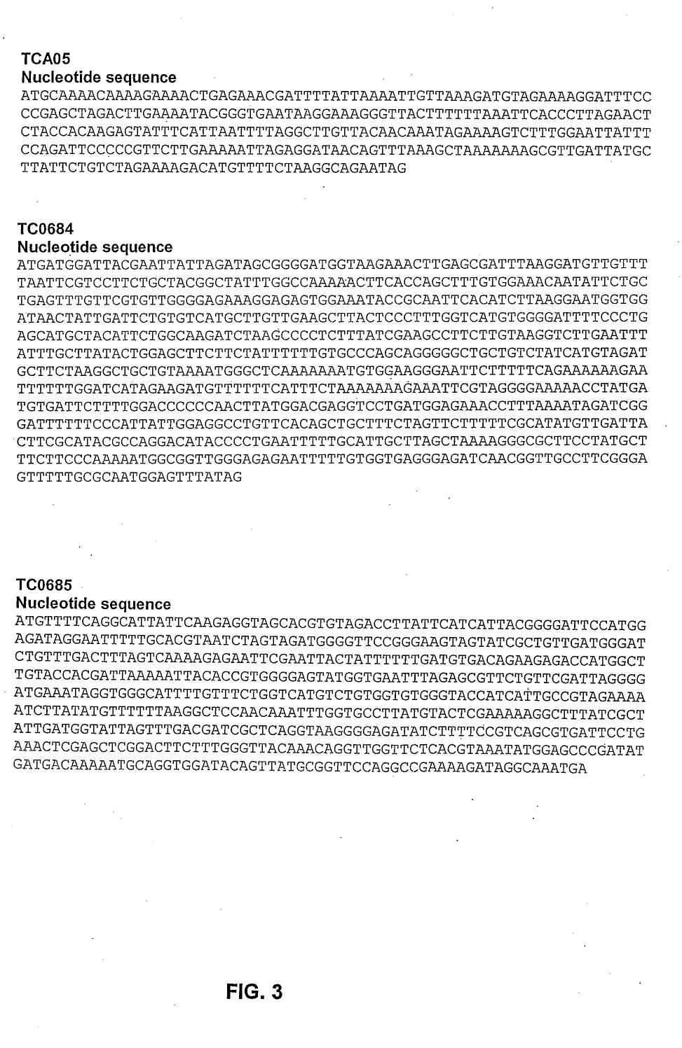

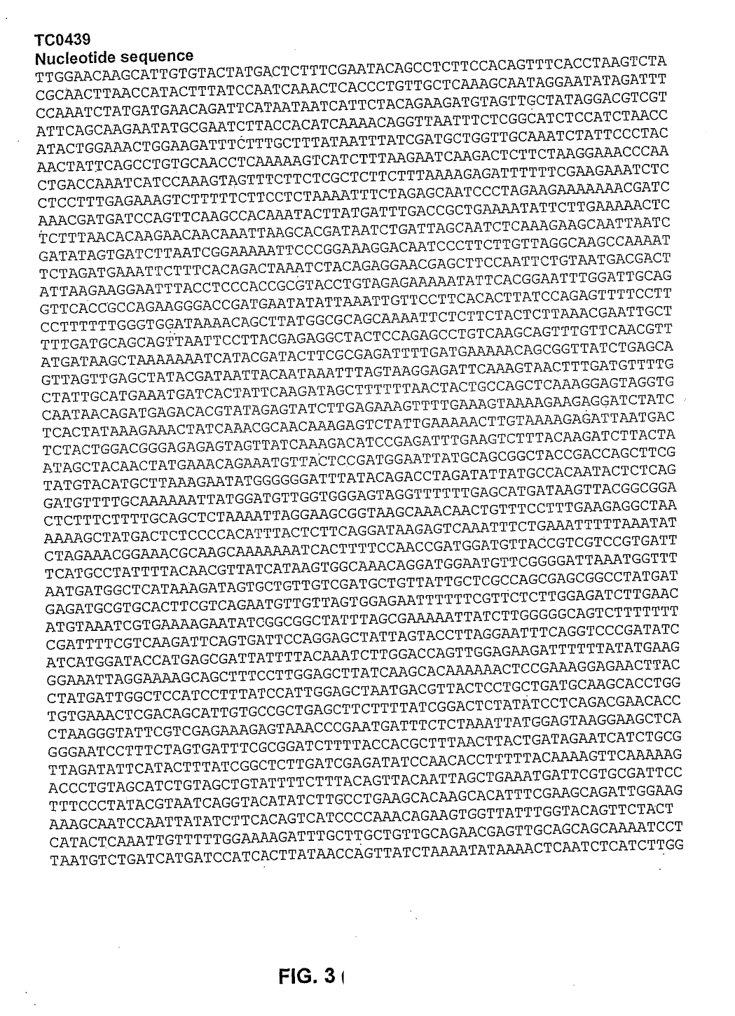

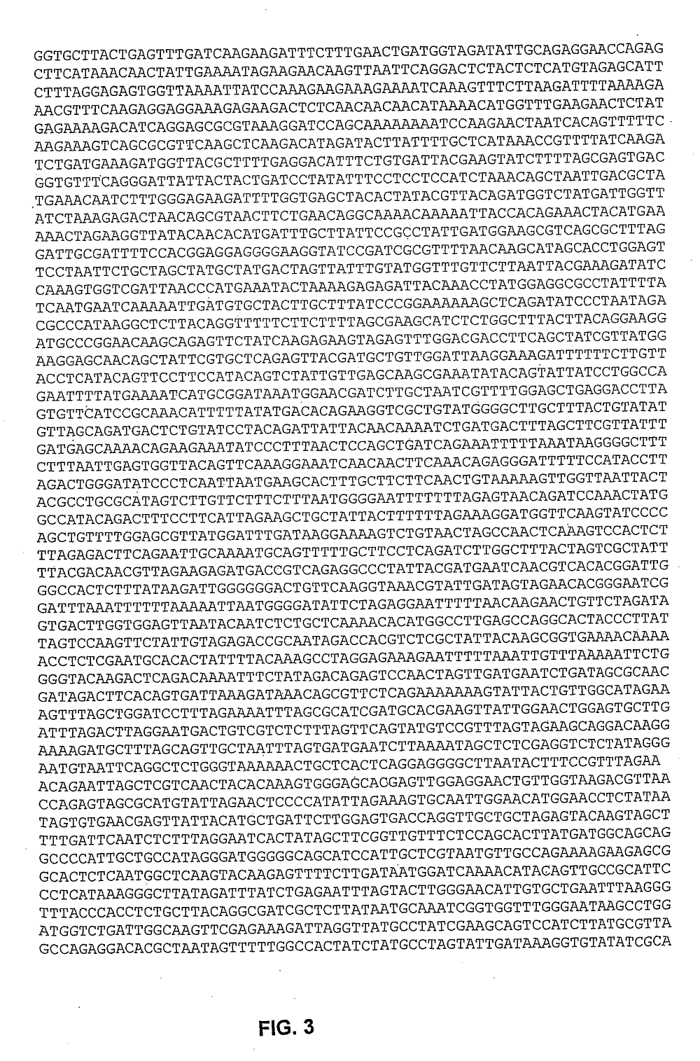

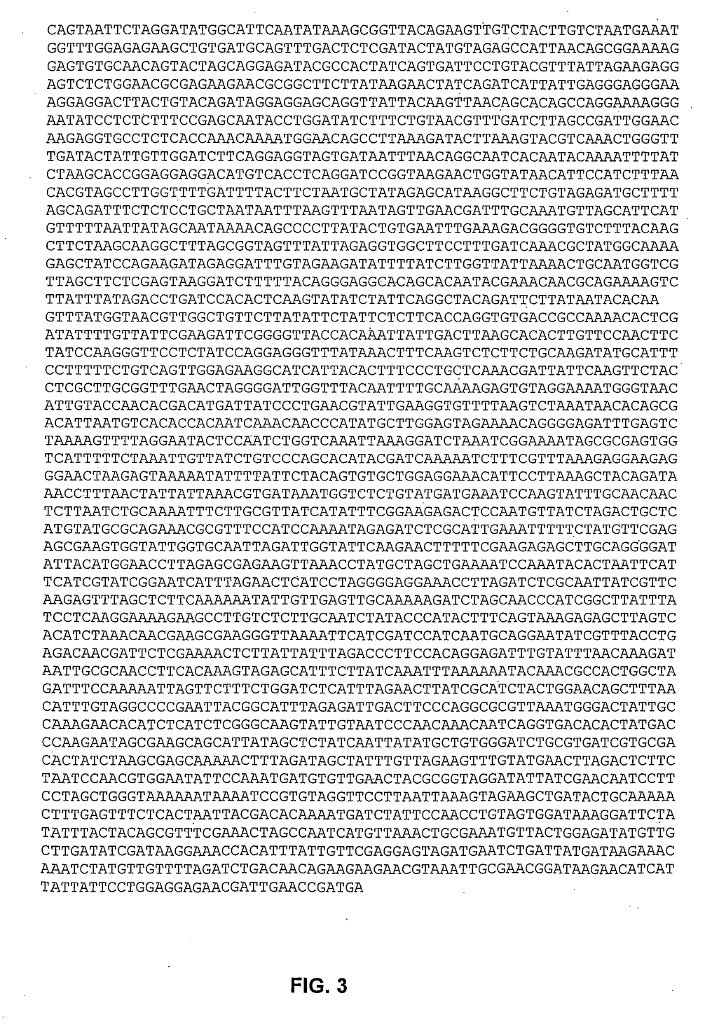

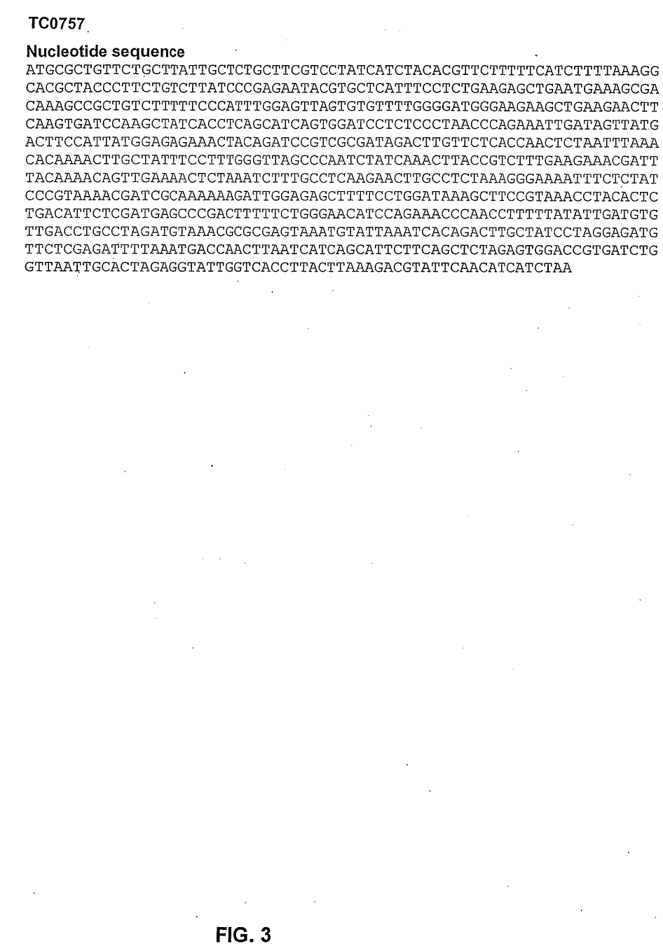

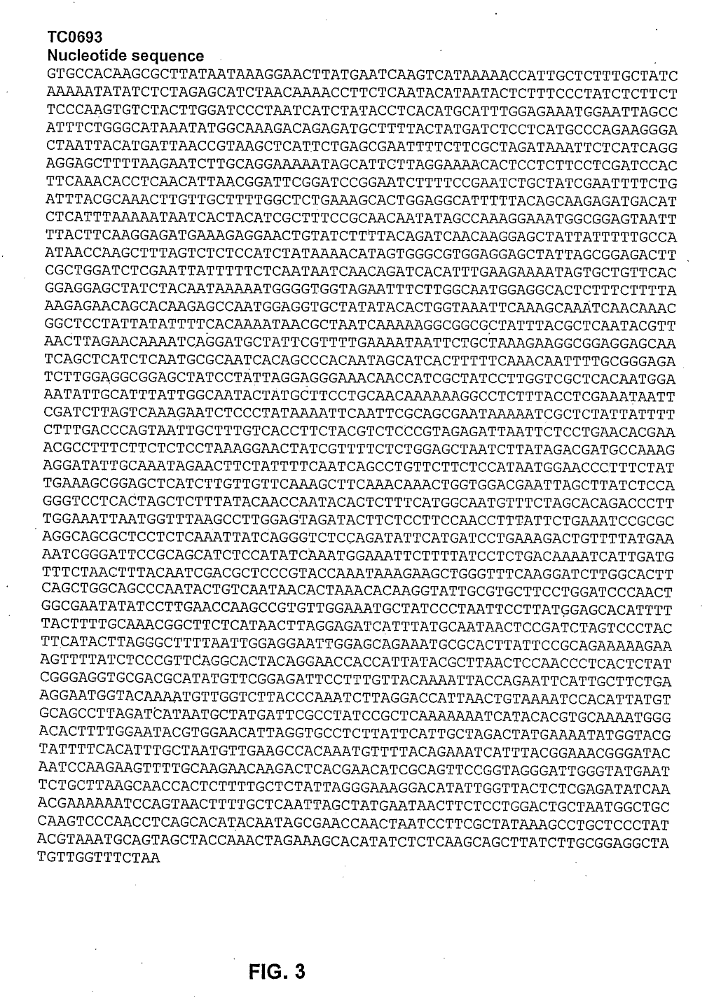

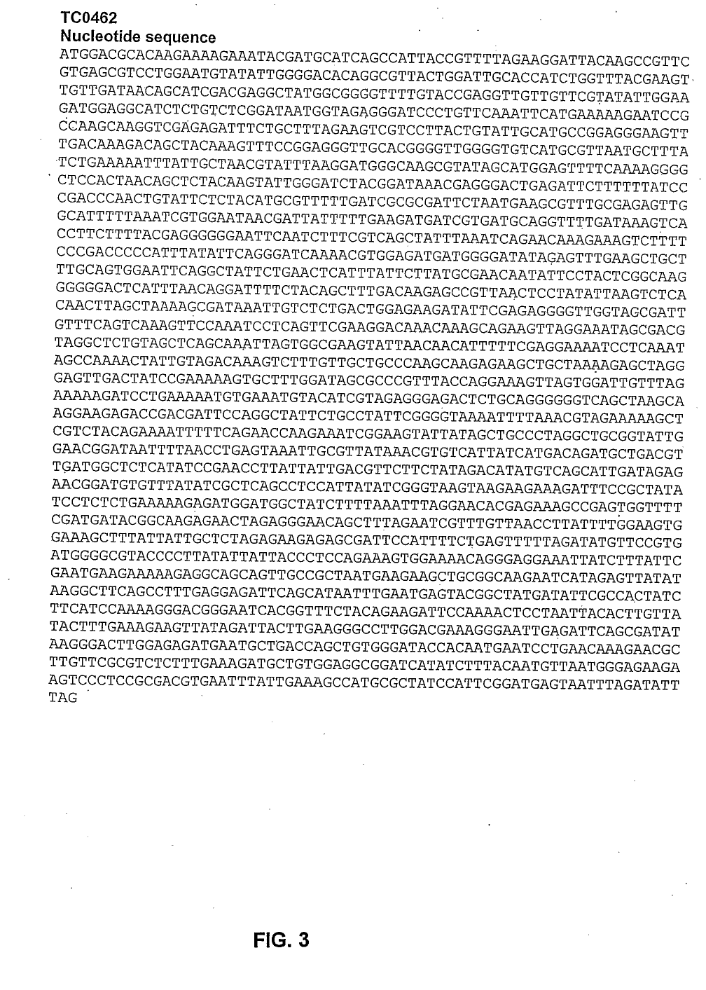

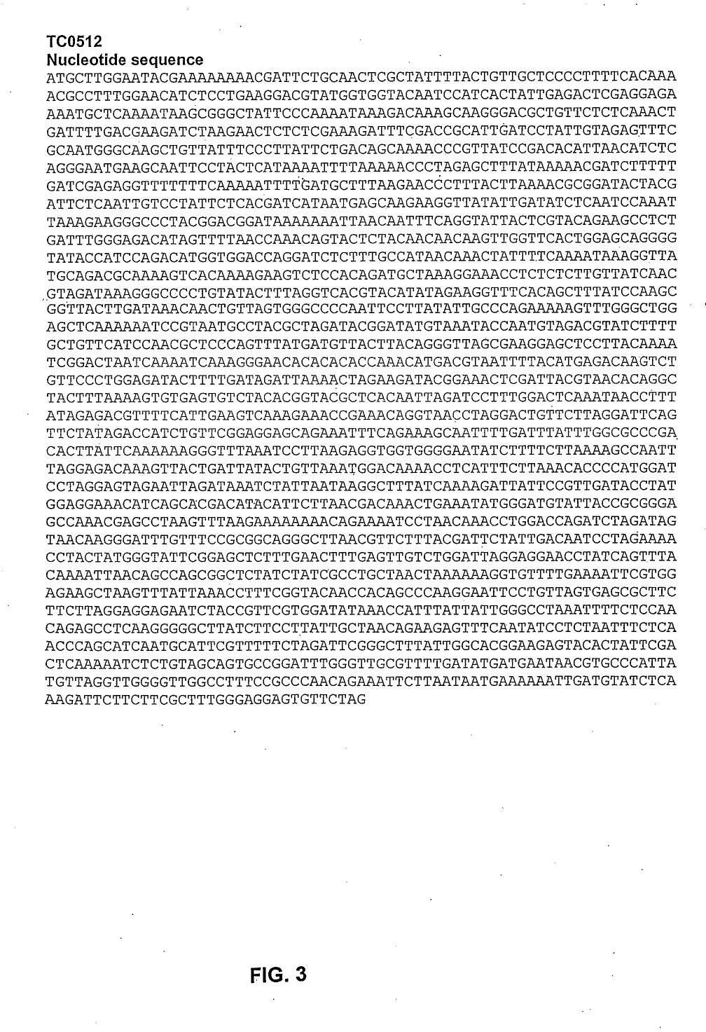

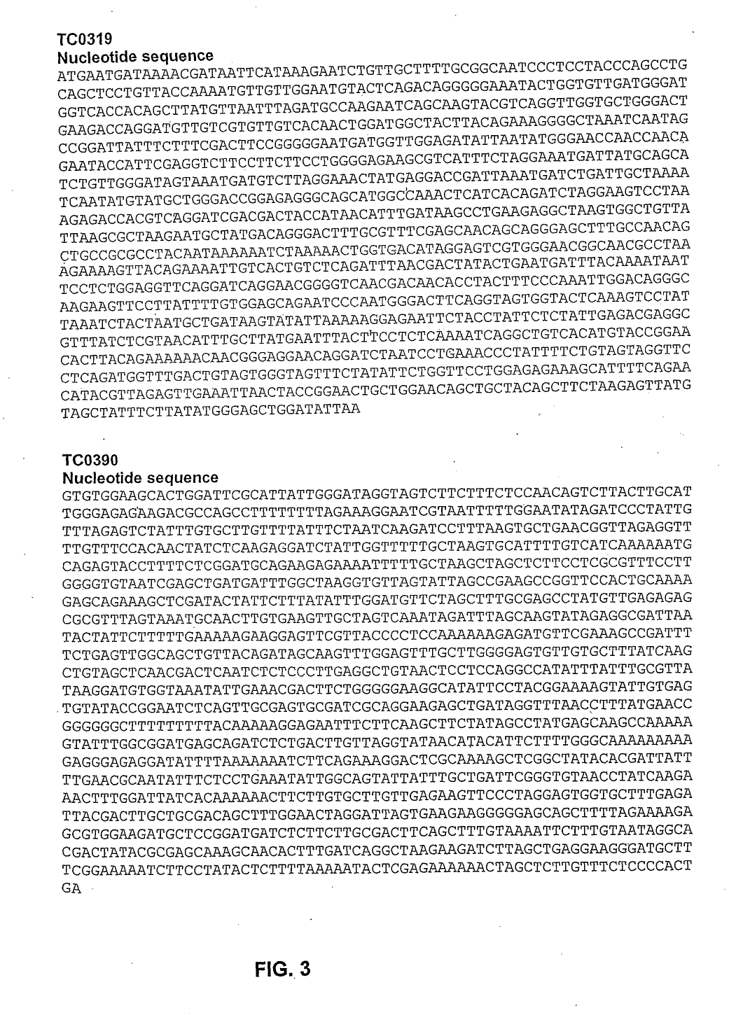

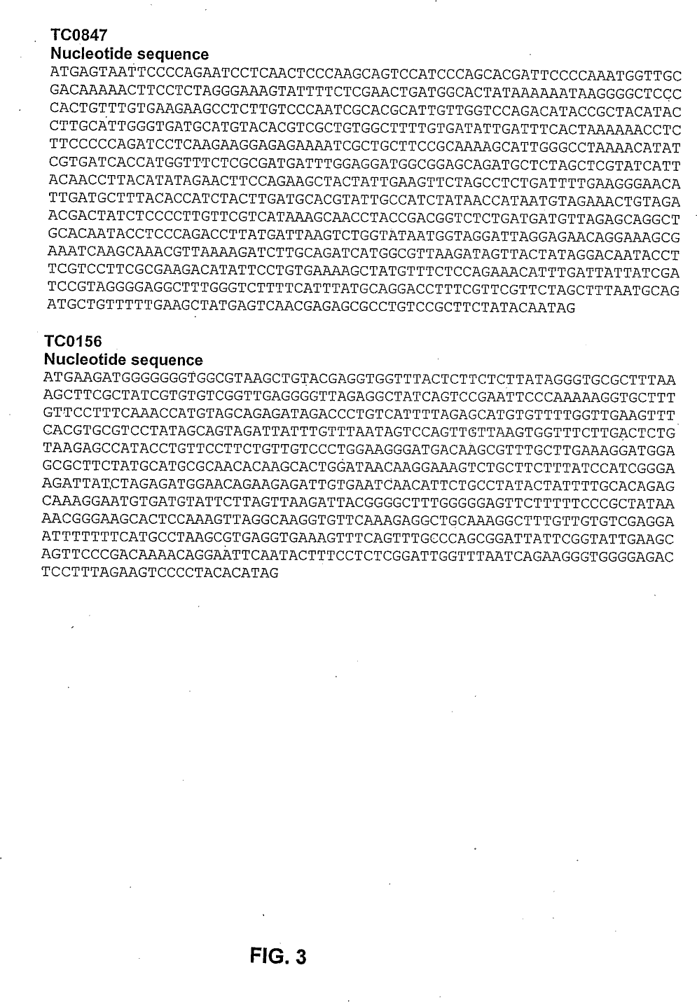

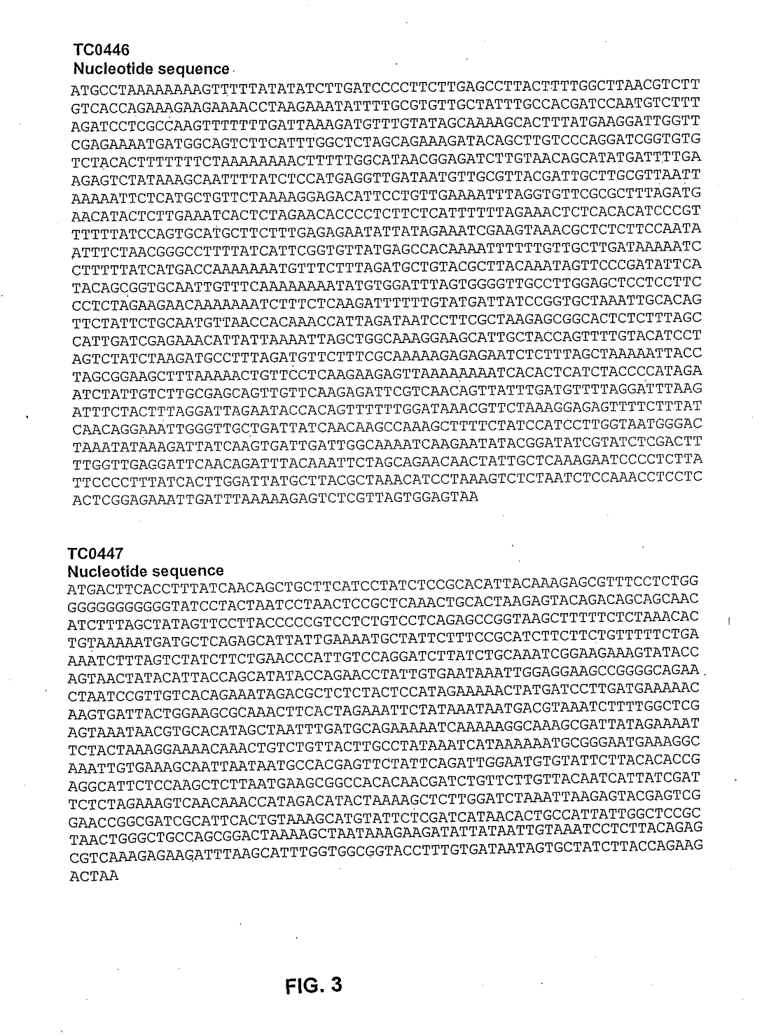

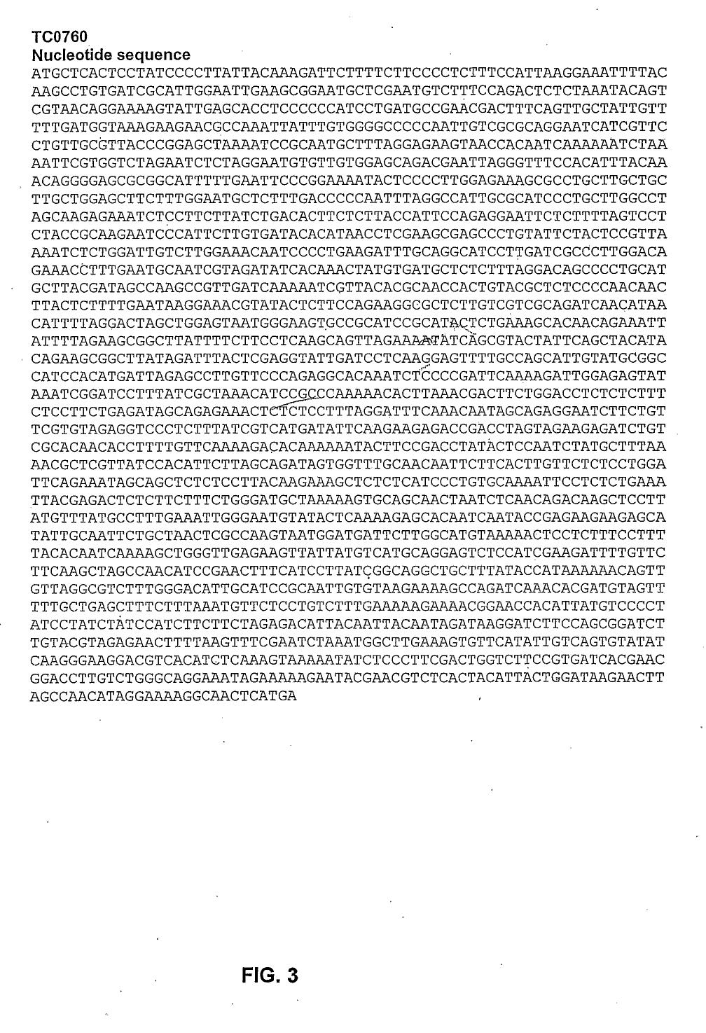

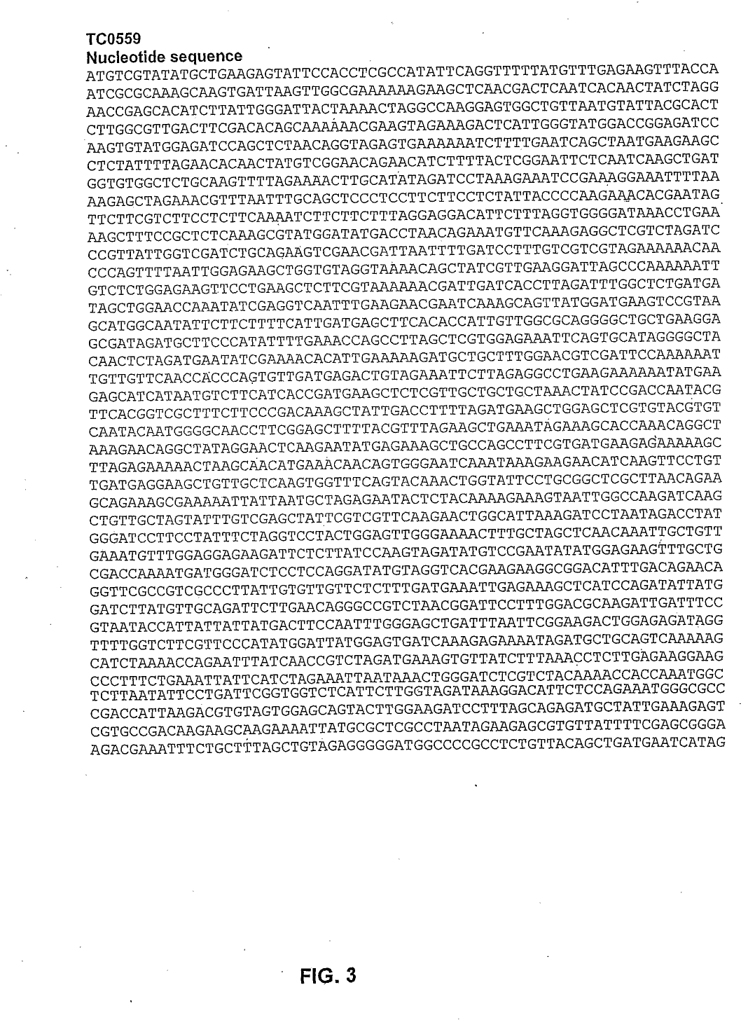

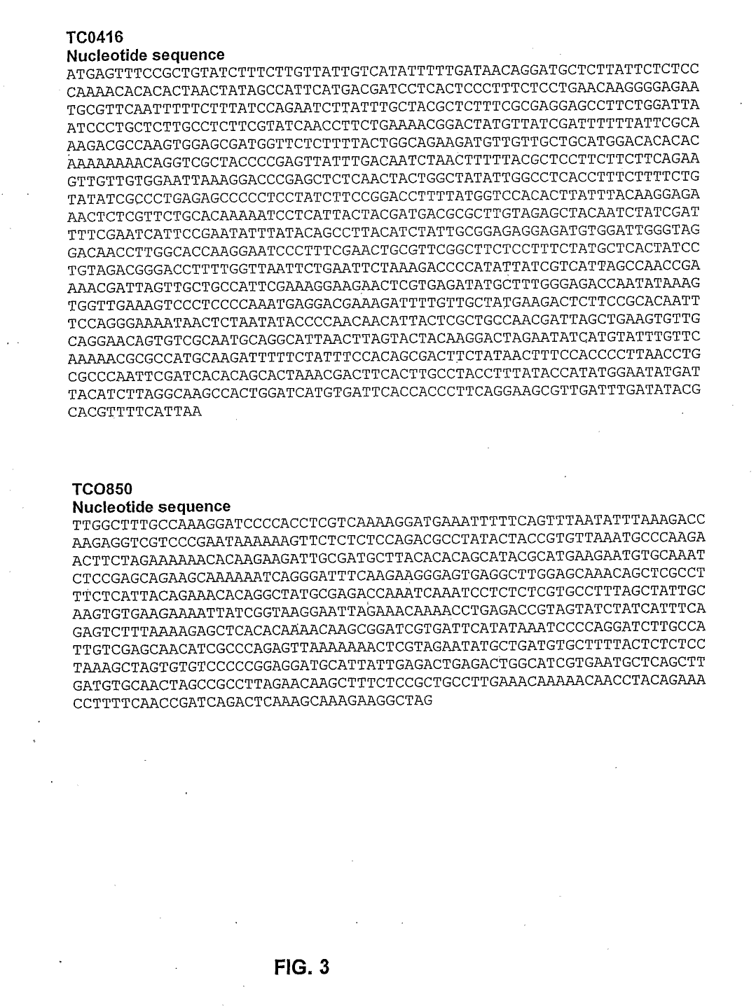

Subsequent studies identified a further 24 chlamydial gene candidates, SEQ ID NOS: 128 to 148 in the mouse and SEQ ID NOS: 85 to 105 and 125 to 127 in human (FIG. 3).

### EXAMPLE 3

### Routes of Immunization

The genes that are identified as being involved in protection against C. trachomatis infection are selected and evaluated for use as vaccine candidates. The selected protein is extracted and used in investigating routes of immunization and delivery methods, for example intranasal and transcutaneous, that elicit the best protection against C. trachomatis infection using the established mouse model. Immunological responses are measured by antibody analysis, T-cell assays and cytokine profiling. The use of adjuvants and interleukins in enhancing the immune response will also be investigated in the mouse model, for example use of cholera toxin and/or CpG-oligonucleotides.

Suitable methods for transcutaneous immunization are exemplified by known methods taught by Berry et al, 2004 Infect Immun 72 1019. Briefly, one or more Chlamydia antigens are prepared and isolated as described above. The Chlamydia antigen(s) may be administered without an adjuvant, however, one or more adjuvants, such as CpG-oligo and/or Cholera toxin, is used. As described in Berry et al, 2004, female BALB/c mice, 6 to 10 weeks old, are anesthetized. The lower back region of each mouse is shaved, with care taken not to break the skin. The shaved area is swabbed with acetone followed by sterile PBS and allowed to dry. A pharmaceutical composition comprising 501 volume of PBS, 200 μg of the isolated Chlamydia protein is mixed with 10 μg of CpG-ODN (an example of a suitable CpG-ODN is that used by Berry et al, 2004, namely 5-TCC ATG ACG TTC CTG ACG TT-3), and 10 μg of Cholera toxin (Sigma-Aldrich) is applied to the shaved area. Mice are maintained under anesthesia for 30 min to prevent grooming and oral immunization. After 30 min the application site is swabbed thoroughly with PBS. Immunizations are administered weekly for three consecutive weeks, followed by a 6-week booster. Non-immunized controls are preferably also included. A similar method may be used for humans, however, a part of the body not covered by hair is preferably selected for administration of the pharmaceutical composition.

For intranasal administration of a Chlamydia protein, the protein may be combined with an adjuvant such as CpG-ODN and/or Cholera toxin as described above and administered to external nasal passages of the subject, for example mouse, human or other animal. Preferably, the pharmaceutical composition is administered using a pipette, such as a micropipette.

As one preferred example, TC0665 (SEQ ID NO: 79) is a 292 amino acid protein obtainable from C. muridarum that produced good immunity in a genital challenge mouse model. It resulted in a reduced peak of infectivity and also cleared the infection faster than control vaccines. The protein has no known homologues outside the Chlamydia genus. The C. trachomatis human homologue, CT386 (SEQ ID NO: 35), has 96% similarity and 91% identity to the mouse C. muridarum strain. The full length nucleic acid, or fragment thereof, is cloned into a suitable expression vector as is well known in the art and described herein. The recombinant protein is preferably expressed as a soluble fusion protein, with for example a His-tag. The recombinant protein is mixed with a suitable adjuvant and applied either intranasally or via transcutaneous route to an animal, preferably a naïve animal, more preferably a human. Immunity is detected by measuring antibodies and/or a T-cell mediated response in the circulating blood of the animal administered the immunogenic composition.

A second preferred example that produced good immunity in a genital challenge mouse model is TC0579 (SEQ ID NO: 78). The C. trachomatis human homologue, CT305 (SEQ ID NO: 34), has 86% similarity to the mouse C. muridarum strain.

A third preferred example that produced good immunity in a genital challenge mouse model is TC0850 (SEQ ID NO: 148). The C. trachomatis human homologue, CT561 (SEQ ID NO: 20), has 89% similarity and 84% identity to the mouse C. muridarum strain.

### EXAMPLE 4

### Method of Protein Discovery and Production

Target proteins were initially identified from a genome wide, in silico analysis, aiming to uncover novel antigens containing promiscuous T cell epitopes.

The investigation utilised the published Chlamydia trachomatis serovar D genome information, along with human leukocyte antigen class II epitope prediction algorithms. It identified proteins containing promiscuous T cell epitopes. Table 3 contains the results of this investigation. The top four ranking targets from this list were cloned successfully into an N terminal hexa-histidine tagged expression vector. Targets were expressed and purified via immobilised metal affinity chromatography for further immunological analysis. It must be noted that the top ranking target CT421 was found to be highly toxic to the Escherichia coli host cell during expression of the target proteins. This protein could not be expressed.

Three candidate C. trachomatis human proteins; CT425, CT562 and CT828 (SEQ ID NOS: 41 to 43, respectively) were identified by this investigation. The C. muridarum homologues are TC0708, TC0851 and TC0215, respectively (SEQ ID NOS: 83 to 85, respectively).

Throughout the specification the aim has been to describe the preferred embodiments of the invention without limiting the invention to any one embodiment or specific collection of features. It will therefore be appreciated by those of skill in the art that, in light of the instant disclosure, various modifications and changes can be made in the particular embodiments exemplified without departing from the scope of the present invention.

The disclosure of each nucleotide and amino acid sequence (including those referred to herein by gene number and/or GenBank Accession number), patent and scientific document, computer program and algorithm referred to in this specification is incorporated by reference in its entirety.

TABLE 1

| | Corresponding C. | | | |
|---|---|---|---|---|
| C. muridarum gene | trachomatis gene | % homology | C. muridarum protein | C. trachomatis protein |
| (mouse) | (human) | (protein) | function | function |
| TC0032 | CT661 | 92 | DNA gyrase subunit B | DNA gyrase subunit B |
| (AAF38923.1) | (AAC68256.1) | | | |
| TC0155 | CT774 | 90 | 3′(2′),5′-bisphosphate | Sulfite synthesis |
| (AAF39031.1) | (AAC68369.1) | | nucleotidase | |
| TC0207 | CT820 | 84 | signal recognition particle | Cell division protein |
| | (AAC68417.1) | | protein FtsY | FtsY |
| TC0300 | CT031 | 91 | Hypothetical protein | Hypothetical protein |
| (AAF39165.1) | | | | |
| TC0301 | CT032 | 95 | methionyl-tRNA | methionyl-tRNA |
| (AAF39166.1) | | | synthetase | synthetase |
| TC0490 | CT608 | 42 | UvrD/REP helicase | DNA helicase (UvrD) |
| (AAF73563.1) | | | family protein | |
| TC0579 | CT305 | 86 | ATP synthase subunit 1 | ATP synthase subunit 1 |
| (AAF39414.1) | | | | |
| TC0665 | CT386 | 96 | Conserved hypothetical | Predicted metal |
| (AAF39487.1) | | | protein | dependent hydrolase |
| TC0247 | CT857 | 84 | Na+/H+ antiporter | |
| | (AAC68454.1) | | | |
| TC0346 | CT047 | 91 | recF protein | |
| (AAF39207.1) | (AAC67638.1) | | | |
| TC0439 | CT166 | 60 | Adherence factor | |
| (AAF39293.1) | (AAC67757.1) | | | |
| TC0444 | CT174 | 53 | Hypothetical protein | |
| (AAF39298.1) | | | | |
| TC0445 | CT621 | 57 | Hypothetical protein | |
| (AAF39299.1) | (AAC68225.1) | | | |
| TC0446 | CT175 | 86 | Peptide ABC transporter | |
| (AAF39300.1) | (AAC67766.1) | | | |
| TC0447 | CT155 | 53 | Phospholipase D family | |
| (AAF39301.1) | (AAC67746.1) | protein | | |
| TC0717 | CT433 | 83 | Conserved hypothetical | |
| (AAF39529.1) | (AAC68032.1) | protein | | |
| TC0718 | CT434 | 93 | 2-C-methyl-D-erythritol | |
| (AAF39530.1) | (AAC68033.1) | | 2,4-cyclodiphosphate | |
| | | | synthase | |
| TC0760 | CT475 | 88 | phenylalanyl-tRNA | |
| (AAF39564.1) | (AAC68075.1) | | synthetase, beta subunit | |

TABLE 2

| | Corresponding | % | | | | C. |
|---|---|---|---|---|---|---|
| C. muridarum | C. trachomatis | homology | C. muridarum protein | C. trachomatis | % homology | muridarum |
| gene | gene | (nucleotide) | function | protein function | (protein) | gene |
| TC0767 | CT481 | 77 | conserved | hypothetical | 76 | TC0767 |
| | (AAC68081.1) | | hypothetical protein | protein | | |
| TC0768 | NT01CT0505 | 79 | hypothetical protein | hypothetical | 69 | TC0768 |
| | (No GenBank | | | protein | | |
| | ID) | | | | | |
| TCA04 | ?? | | virulence protein | | | TCA04 |
| | | | pGP3-D | | | |
| TCA05 | CT344 | 61 | virulence protein | Ion ATP- | 0 | TCA05 |
| | (AAC67939.1) | | pGP4-D | dependent | | |
| | | | | protease (Ion) | | |
| TC0684 | CT404 | 81 | conserved | SAM | 89 | TC0684 |
| | (AAC68001.1) | | hypothetical protein | dependent | | |
| | | | | methyl- | | |
| | | | | transferase | | |
| TC0685 | CT405 | 82 | riboflavin synthase, | Riboflavin | 88 | TC0685 |
| | (AAC68002.1) | | alpha subunit (ribE) | Synthase | | |
| | | | | (ribC) | | |
| TC0439 | CT166 | 63 | adherence factor | hypothetical | 47 | TC0439 |
| | (AAC67757.1) | | | protein | | |
| TC0757 | CT472 | 83 | conserved | hypothetical | 95 | TC0757 |
| | (AAC68072.1) | | hypothetical protein | protein | | |
| TC0693 | CT412 | 77 | Poly-morphic | Putative outer | 76 | TC0693 |
| | (AAC68009.1) | | membrane protein A | membrane | | |
| | | | family (pmpA) | protein A | | |
| | | | | (pmpA) | | |
| TC0462 | CT190 | 81 | DNA gyrase, | DNA Gyrase | 92 | TC0462 |
| | (AAC67782.1) | | subunit B | Subunit B | | |
| | | | | (gyrB_1) | | |
| TC0512 | CT241 | 82 | outer membrane | Omp85 Analog | 92 | TC0512 |
| | (AAC67834.1) | | protein, putative | (yaeT) | | |
| TC0319 | CT049 | 71 | Hypo-thetical | hypothetical | 60 | TC0319 |
| | (AAC67640.1) | | protein | protein | | |
| TC0390 | CT114 | 72 | conserved | hypothetical | 74 | TC0390 |
| | (AAC67705.1) | | hypothetical protein | protein | | |
| TC0847 | CT558 | 80 | lipoic acid | Lipoate | 88 | TC0847 |
| | (AAC68160.1) | | synthetase | Synthetase | | |
| | | | | (lipA) | | |
| TC0156 | CT775 | 85 | conserved hypo- | snGlycerol 3-P | 89 | TC0156 |
| | (AAC68370.1) | | thetical protein | Acyl- | | |
| | | | | transferase | | |
| TC0446 | CT175 | 73 | peptide ABC | Oligo-peptide | 73 | TC0446 |
| | (AAC67766.1) | | transporterperi- | binding protein | | |
| | | | plasmic peptide-bind- | permease | | |
| | | | ing protein | (oppA_2) | | |
| TC0447 | CT155 | 62 | Phosphor-lipase D | Phospho-lipase | 28 | TC0447 |
| | (AAC67746.1) | | family protein | D Endo- | | |
| | | | | nuclease | | |
| | | | | Superfamily | | |
| TC0760 | CT475 | 80 | phenylalanyl-tRNA | phenylalanyl | 84 | TC0760 |
| | (AAC68075.1) | | synthetasebeta | tRNA | | |
| | | | subunit | Synthetase | | |
| | | | | Beta (pheT) | | |
| TC0559 | CT286 | 85 | ATP-dependent Clp | ClpC Protease | 96 | TC0559 |
| | (AAC67879.1) | | protease, ATP- | ATPase | | |
| | | | binding subunit | | | |
| | | | ClpC | | | |
| TC0416 | CT139 | 73 | peptide ABC | Oligopeptide | 84 | TC0416 |
| | (AAC67730.1) | | transporterperi- | Binding Protein | | |
| | | | plasmic peptide-bind- | | | |
| | | | ing protein, putative | | | |
| TCO850 | CT561 | 84 | TYPE 3 | YOP | 89 | TCO850 |
| | (AAC68163.1) | | SECRETION GENE | TRANSLOCATION | | |
| | | | (sctL) | I (yscL) | | |

TABLE 3

| | | | | | | Full length amino acid sequence | | |
|---|---|---|---|---|---|---|---|---|
| | | | | | No of | homology between | | |
| | | | | | Predicted | C. trachomatis & C. pneumoniae | | |
| | C. trachomatis | C. pneumoniae | | Selection | Promiscuous | | Identity | |
| Rank | Gene Number | Gene Number | Description of Gene | Score | Epitopes | Similarity (%) | (%) | E value |
| 1 | CT421 | CPn0506 | CT421 hypothetical protein | 76 | 1 | 82 | 74 | 6.00E- |
| | | | | | | | | 78 |
| 2 | CT425 | CPn0512 | Hypothetical protein | 72 | 1 | 81 | 68 | 3.30E- |
| | | | | | | | | 241 |
| 3 | CT562 | CPn0825 | Yop Translocation R | 66 | 3 | 84 | 77 | 3.00E- |
| | | | | | | | | 92 |
| 4 | CT828 | CPn0985 | Ribonucleoside Reductase, | 64 | 1 | 93 | 86 | 0 |
| | | | Small Chain | | | | | |
| 5 | CT572 | CPn0815 | General Secretion protein D | 62 | 1 | 78 | 63 | 8.90E- |
| | | | | | | | | 251 |
| 6 | CT701 | CPn0841 | Protein Translocase secA 2) | 60 | 1 | 87 | 78 | 0 |
| 7 | CT808 | CPn0959 | Axial filament protein | 57 | 1 | 93 | 87 | 1.10E- |
| | | | | | | | | 241 |
| 8 | CT443 | CPn0557 | 60 kDa Cysteine-Rich OMP | 54 | 1 | 84 | 71 | 3.90E- |
| | | | | | | | | 225 |
| 9 | CY653 | CPn0723 | ABC Transporter ATPase | 53 | 1 | 95 | 88 | 1.00E- |
| | | | | | | | | 114 |

### REFERENCES

Barron, A. L., White, H. J., Rank, R. G., Soloff, B. L. and Moses, E. B. (1981). “A new animal model for the study of Chlamydia trachomatis genital infections: infection of mice with the agent of mouse pneumonitis.” The Journal of Infectious Diseases 143(1): 63-66.
Barry, M. A., Lai, W. C. and Johnston, S. A. (1995). “Protection against mycoplasma infection using expression-library immunization.” Nature 377(6550): 632-635.
Bavoil, P. M., Hsia, R.-c. and Rank, R. G. (1996). “Prospects for a vaccine against Chlamydia genital disease I.—Microbiology and pathogenesis.” Bulletin de l'Institut Pasteur 94(1): 5-54.
Beatty, P. R. and Stephens, R. S. (1994). “CD8+ T lymphocyte-mediated lysis of Chlamydia-infected L cells using an endogenous antigen pathway.” Journal of Immunology (Baltimore, Md.: 1950) 153(10): 4588-4595.
Belshe, R. B., Mendelman, P. M., Treanor, J., King, J., Gruber, W. C., Piedra, P., Bernstein, D. I., Hayden, F. G., Kotloff, K. and Zangwill et, al. (1998). “The efficacy of live attenuated, cold-adapted, trivalent, intranasal influenza virus vaccine in children.” The New England Journal of Medicine 338(20): 1405-1412.
Bouvet, J. P., Belec, L., Pires, R. and Pillot, J. (1994). “Immunoglobulin G antibodies in human vaginal secretions after parenteral vaccination.” Infection and Immunity 62(9): 3957-3961.
Brayton, K. A., Vogel, S. W. and Allsopp, B. A. (1998). “Expression library immunization to identify protective antigens from Cowdria ruminantium.” Annals of the New York Academy of Sciences 849: 369-371.
Brunham, R. C., Kuo, C. C., Cles, L. and Holmes, K. K. (1983). “Correlation of host immune response with quantitative recovery of Chlamydia trachomatis from the human endocervix.” Infection and Immunity 39(3): 1491-1494.
Brunham, R. C., Paavonen, J., Stevens, C. E., Kiviat, N., Kuo, C. C., Critchlow, C. W. and Holmes, K. K. (1984). “Mucopurulent cervicitis—the ignored counterpart in women of urethritis in men.” The New England Journal of Medicine 311(1): 1-6.
Cates, W., Jr and Wasserheit, J. N. (1991). “Genital chlamydial infections: epidemiology and reproductive sequelae.” American Journal of Obstetrics and Gynecology 164(2): 1771-1781.
Cotter, T. W., Ramsey, K. H., Miranpuri, G. S., Poulsen, C. E. and Byrne, G. I. (1997). “Dissemination of Chlamydia trachomatis chronic genital tract infection in gamma interferon gene knockout mice.” Infection and Immunity 65(6): 2145-2152.
Cui, Z. D., Tristram, D., LaScolea, L. J., Kwiatkowski, T., Jr, Kopti, S, and Ogra, P. L. (1991). “Induction of antibody response to Chlamydia trachomatis in the genital tract by oral immunization.” Infection and Immunity 59(4): 1465-1469.
Czerkinsky, C., Svennerholm, A. M. and Holmgren, J. (1993). “Induction and assessment of immunity at enteromucosal surfaces in humans: implications for vaccine development.” Clinical Infectious Diseases: an Official Publication of the Infectious Diseases Society of America 16(2): S106-S116.
Donnelly, J. J., Ulmer, J. B., Shiver, J. W. and Liu, M. A. (1997). “DNA vaccines.” Annual Review of Immunology 15: 617-648.
Everett, K. D., Bush, R. M. and Andersen, A. A. (1999). “Emended description of the order Chlamydiales, proposal of Parachlamydiaceae fam. nov. and Simkaniaceae fam. nov., each containing one monotypic genus, revised taxonomy of the family Chlamydiaceae, including a new genus and five new species, and standards for the identification of organisms.” International Journal of Systematic Bacteriology 49(2): 415-440.
Fynan, E. F., Webster, R. G., Fuller, D. H., Haynes, J. R., Santoro, J. C. and Robinson, H. L. (1993). “DNA vaccines: protective immunizations by parenteral, mucosal, and gene-gun inoculations.” Proceedings of the National Academy of Sciences of the United States of America 90(24): 11478-11482. Gencay, M., Koskiniemi, M., Saikku, P., Puolakkainen, M., Raivio, K., Koskela, P. and Vaheri, A. (1995). “Chlamydia trachomatis seropositivity during pregnancy is associated with perinatal complications.” Clinical Infectious Diseases: an Official Publication of the Infectious Diseases Society of America 21(2): 424-426.
Glenn, G. M., Scharton-Kersten, T., Vassell, R., Mallett, C. P., Hale, T. L. and Alving, C. R. (1998). “Transcutaneous immunization with cholera toxin protects mice against lethal mucosal toxin challenge.” Journal of Immunology (Baltimore, Md.: 1950) 161(7): 3211-3214.
Glenn, G. M., Scharton-Kersten, T., Vassell, R., Matyas, G. R. and Alving, C. R. (1999). “Transcutaneous immunization with bacterial ADP-ribosylating exotoxins as antigens and adjuvants.” Infection and Immunity 67(3): 1100-1106.
Glenn, G. M., Taylor, D. N., L1, X., Frankel, S., Montemarano, A. and Alving, C. R. (2000). “Transcutaneous immunization: a human vaccine delivery strategy using a patch.” Nature Medicine 6(12): 1403-1406.
Gockel, C. M., Bao, S, and Beagley, K. W. (2000). “Transcutaneous immunization induces mucosal and systemic immunity: a potent method for targeting immunity to the female reproductive tract.” Molecular Immunology 37(9): 537-544.
Gravett, M. G., Nelson, H. P., DeRouen, T., Critchlow, C., Eschenbach, D. A. and Holmes, K. K. (1986). “Independent associations of bacterial vaginosis and Chlamydia trachomatis infection with adverse pregnancy outcome.” JAMA: the Journal of the American Medical Association 256(14): 1899-1903. Grayston, J. T. and Wang, S. P. (1978). “The potential for vaccine against infection of the genital tract with Chlamydia trachomatis.” Sexually Transmitted Diseases 5(2): 73-77.
Greendale, G. A., Haas, S. T., Holbrook, K., Walsh, B., Schachter, J. and Phillips, R. S. (1993). “The relationship of Chlamydia trachomatis infection and male infertility.” American Journal of Public Health 83(7): 996-1001.
Hammond, S. A., Guebre-Xabier, M., Yu, J. and Glenn, G. M. (2001). “Transcutaneous immunization: an emerging route of immunization and potent immunostimulation strategy.” Critical Reviews in Therapeutic Drug Carrier Systems 18(5): 503-526.
Haneberg, B., Kendall, D., Amerongen, H. M., Apter, F. M., Kraehenbuhl, J. P. and Neutra, M. R. (1994). “Induction of specific immunoglobulin A in the small intestine, colon-rectum, and vagina measured by a new method for collection of secretions from local mucosal surfaces.” Infection and Immunity 62(1): 15-23.
Igietseme, J. U. (1996). “The molecular mechanism of T-cell control of Chlamydia in mice: role of nitric oxide.” Immunology 87(1): 1-8.
Johnston, S. A. and Barry, M. A. (1997). “Genetic to genomic vaccination.” Vaccine 15(8): 808-809.
Jones, P. P. and Cebra, J. J. (1974). “Restriction of gene expression in B lymphocytes and their progeny. III. Endogenous IgA and IgM on the membranes of different plasma cell precursors.” The Journal of Experimental Medicine 140(4): 966-976.
Jones, R. B., Ardery, B. R., Hui, S. L. and Cleary, R. E. (1982). “Correlation between serum antichlamydial antibodies and tubal factor as a cause of infertility.” Fertility and Sterility 38(5): 553-558.
Kelly, K. A., Robinson, E. A. and Rank, R. G. (1996). “Initial route of antigen administration alters the T-cell cytokine profile produced in response to the mouse pneumonitis biovar of Chlamydia trachomatis following genital infection.” Infection and Immunity 64(12): 4976-4983.
Koskela, P., Anitila, T., Bjorge, T., Brunsvig, A., Dillner, J., Hakama, M., Hakulinen, T., Jellum, E., Lehtinen, M. and Lenner et, a. (2000). “Chlamydia trachomatis infection as a risk factor for invasive cervical cancer.” International Journal of Cancer. Journal International Du Cancer 85(1): 35-39.
Kozlowski, P. A., Cu-Uvin, S., Neutra, M. R. and Flanigan, T. P. (1997). “Comparison of the oral, rectal, and vaginal immunization routes for induction of antibodies in rectal and genital tract secretions of women.” Infection and Immunity 65(4): 1387-1394.
Kuno-Sakai, H., Kimura, M., Ohta, K., Shimojima, R., Oh, Y. and Fukumi, H. (1994). “Developments in mucosal influenza virus vaccines.” Vaccine 12(14): 1303-1310.
Lucero, M. E. and Kuo, C. C. (1985). “Neutralization of Chlamydia trachomatis cell culture infection by serovar-specific monoclonal antibodies.” Infection and Immunity 50(2): 595-597.
Magee, D. M., Williams, D. M., Smith, J. G., Bleicker, C. A., Grubbs, B. G., Schachter, J. and Rank, R. G. (1995). “Role of CD8 T cells in primary Chlamydia infection.” Infection and Immunity 63(2): 516-521.
Marra, C. A., Patrick, D. M., Reynolds, R. and Marra, F. (1998). “Chlamydia trachomatis in adolescents and adults. Clinical and economic implications.” PharmacoEconomics 13(2): 191-222.
Martin, D. H., Koutsky, L., Eschenbach, D. A., Daling, J. R., Alexander, E. R., Benedetti, J. K. and Holmes, K. K. (1982). “Prematurity and perinatal mortality in pregnancies complicated by maternal Chlamydia trachomatis infections.”JAMA: the Journal of the American Medical Association 247(11): 1585-1588.
McGhee, J. R., Mestecky, J., Dertzbaugh, M. T., Eldridge, J. H., Hirasawa, M. and Kiyono, H. (1992). “The mucosal immune system: from fundamental concepts to vaccine development.” Vaccine 10(2): 75-88.
Melby, P. C., Ogden, G. B., Flores, H. A., Zhao, W., Geldmacher, C., Biediger, N. M., Ahuja, S. K., Uranga, J. and Melendez, M. (2000). “Identification of vaccine candidates for experimental visceral leishmaniasis by immunization with sequential fractions of a cDNA expression library.” Infection and Immunity 68(10): 5595-5602.
Mestecky, J. and McGhee, J. R. (1993). Local Immunity in Reproductive Tract Tissues: The secretory IgA system. Oxford, Oxford University Press.
Moore, R. J., Lenghaus, C., Sheedy, S. A. and Doran, T. J. (2001). “Improved vectors for expression library immunization—application to Mycoplasma hyopneumoniae infection in pigs.” Vaccine 20(1-2): 115-120.
Morrison, R. P., Feilzer, K. and Tumas, D. B. (1995). “Gene knockout mice establish a primary protective role for major histocompatibility complex class II-restricted responses in Chlamydia trachomatis genital tract infection.”Infection and Immunity 63(12): 4661-4668.
Morrison, S. G. and Morrison, R. P. (2000). “In situ analysis of the evolution of the primary immune response in murine Chlamydia trachomatis genital tract infection.” Infection and Immunity 68(5): 2870-2879.
Moulder, J. W. (1991). “Interaction of chlamydiae and host cells in vitro.” Microbiological Reviews 55(1): 143-190.
Murray, E. S., Charbonnet, L. T. and MacDonald, A. B. (1973). “Immunity to chlamydial infections of the eye. I. The role of circulatory and secretory antibodies in resistance to reinfection with guinea pig inclusion conjunctivitis.” Journal of Immunology (Baltimore, Md.: 1950) 110(6): 1518-1525.
Murray, H. W., Szuro-Sudol, A., Wellner, D., Oca, M. J., Granger, A. M., Libby, D. M., Rothermel, C. D. and Rubin, B. Y. (1989). “Role of tryptophan degradation in respiratory burst-independent antimicrobial activity of gamma interferon-stimulated human macrophages.” Infection and Immunity 57(3): 845-849.
Paavonen, J. and Eggert-Kruse, W. (1999). “Chlamydia trachomatis: impact on human reproduction.” Human Reproduction Update 5(5): 433-447. Pal, S., Fielder, T. J., Peterson, E. M. and de la Maza, L. M. (1994). “Protection against infertility in a BALB/c mouse salpingitis model by intranasal immunization with the mouse pneumonitis biovar of Chlamydia trachomatis.”Infection and Immunity 62(8): 3354-3362.
Peeling, R., Maclean, I. W. and Brunham, R. C. (1984). “In vitro neutralisation of Chlamydia trachomatis with monoclonal antibody to an epitope on the major outer membrane protein.” Infection and Immunity 46: 484-488.
Peeling, R. W. and Brunham, R. C. (1996). “Chlamydiae as pathogens: new species and new issues.” Emerging Infectious Diseases 2(4): 307-319.
Perry, L. L., Feilzer, K. and Caldwell, H. D. (1997). “Immunity to Chlamydia trachomatis is mediated by T helper 1 cells through IFN-gamma-dependent and -independent pathways.” Journal of Immunology (Baltimore, Md.: 1950) 158(7): 3344-3352.
Peterson, E. M., de la Maza, L. M., Brade, L. and Brade, H. (1998).

“Characterization of a neutralizing monoclonal antibody directed at the lipopolysaccharide of Chlamydia pneumoniae.” Infection and Immunity 66(8): 3848-3855.
Purvis, K. and Christiansen, E. (1993). “Infection in the male reproductive tract. Impact, diagnosis and treatment in relation to male infertility.” International Journal of Andrology 16(1): 1-13.
Rank, R. G. and Barron, A. L. (1983). “Humoral immune response in acquired immunity to chlamydial genital infection of female guinea pigs.” Infection and Immunity 39(1): 463-465.
Rank, R. G. and Bavoil, P. M. (1996). “Prospects for a vaccine against Chlamydia genital disease II.—Immunity and vaccine development.” Bulletin de I'Institut Pasteur 94(1): 55-82.
Rank, R. G., Soderberg, L. S, and Barron, A. L. (1985). “Chronic chlamydial genital infection in congenitally athymic nude mice.” Infection and Immunity 48(3): 847-849.
Rank, R. G., White, H. J. and Barron, A. L. (1979). “Humoral immunity in the resolution of genital infection in female guinea pigs infected with the agent of guinea pig inclusion conjunctivitis.” Infection and Immunity 26: 573-579.
Smooker, P. M., Setiady, Y. Y., Rainczuk, A. and Spithill, T. W. (2000). “Expression library immunization protects mice against a challenge with virulent rodent malaria.” Vaccine 18(23): 2533-2540.
Stamm, W. E. (1999). “Chlamydia trachomatis infections: progress and problems.” The Journal of Infectious Diseases 179(2): S380-S383.
Starnbach, M. N., Bevan, M. J. and Lampe, M. F. (1994). “Protective cytotoxic T lymphocytes are induced during murine infection with Chlamydia trachomatis.” Journal of Immunology (Baltimore, Md.: 1950) 153(11): 5183-5189.
Su, H. and Caldwell, H. D. (1995). “CD4+ T cells play a significant role in adoptive immunity to Chlamydia trachomatis infection of the mouse genital tract.” Infection and Immunity 63(9): 3302-3308.
Su, H., Feilzer, K., Caldwell, H. D. and Morrison, R. P. (1997). “Chlamydia trachomatis genital tract infection of antibody-deficient gene knockout mice.”Infection and Immunity 65(6): 1993-1999.
Svanholm, C., Bandholtz, L., Castanos-Velez, E., Wigzell, H. and Rottenberg, M. E. (2000). “Protective DNA immunization against Chlamydia pneumoniae.”Scandinavian Journal of Immunology 51(4): 345-353.
Tang, D. C., DeVit, M. and Johnston, S. A. (1992). “Genetic immunization is a simple method for eliciting an immune response.” Nature 356(6365): 152-154.
Tomasi, T. B. and Ziegelbaum, S. (1963). “The selective occurrence of gamma 1A globulins in certain body fluids.” Journal of Clinical Investigation 42:1552-1560.
Wang, S. P. and Grayston, J. T. (1970). “Immunologic relationship between genital TRIC, lymphogranuloma venereum, and related organisms in a new microtiter indirect immunofluorescence test.” American Journal of Opthalmology 70(3): 367-374.
Wang, S. P., Grayston, J. T. and Gale, J. L. (1973). “Three new immunologic types of trachoma-inclusion conjunctivitis organisms.” Journal of Immunology (Baltimore, Md.: 1950) 110(3): 873-879.
Ward, M. E. (1995). “The immunobiology and immunopathology of chlamydial infections.” APMIS: Acta Pathologica, Microbiologica, et Immunologica Scandinavica 103(11): 769-796.
Whalen, R. G. and Davis, H. L. (1995). “DNA-mediated immunization and the energetic immune response to hepatitis B surface antigen.” Clinical Immunology and Immunopathology 75(1): 1-12.
Wolff, H., Neubert, U., Zebhauser, M., Bezold, G., Korting, H. C. and Meurer, M. (1991). “Chlamydia trachomatis induces an inflammatory response in the male genital tract and is associated with altered semen quality.” Fertility and Sterility 55(5): 1017-1019.
Wu, H. Y., Nguyen, H. H. and Russell, M. W. (1997). “Nasal lymphoid tissue (NALT) as a mucosal immune inductive site.” Scandinavian Journal of Immunology 46(5): 506-513.
Zelin, J. M., Robinson, A. J., Ridgway, G. L., Allason-Jones, E. and Williams, P. (1995). “Chlamydial urethritis in heterosexual men attending a genitourinary medicine clinic: prevalence, symptoms, condom usage and partner change.” International Journal of STD & AIDS 6(1): 27-30.
Zhang, Y. X., Stewart, S., Joseph, T., Taylor, H. R. and Caldwell, H. D. (1987). “Protective monoclonal antibodies recognize epitopes located on the major outer membrane protein of Chlamydia trachomatis.” Journal of Immunology (Baltimore, Md.: 1950) 138(2): 575-581.

1. A pharmaceutical composition for preventing or ameliorating Chlamydia or Chlamydophila infection comprising
(i) an isolated protein that comprises an amino acid sequence selected from the group consisting of the sequences set forth in SEQ ID NOS 20, 34 and 35, and
(ii) a pharmaceutically-acceptable carrier, diluent or excipient.

2. A pharmaceutical composition comprising a homolog and/or fragment of an isolated protein that comprises an amino acid sequence selected from the group consisting of the sequences set forth in SEQ ID NOS 20, 34, and 35, wherein said homolog and/or fragment has at least 75% sequence identity to any one of said isolated proteins.

3. The pharmaceutical composition of claim 2, wherein said homolog is an ortholog.

4. The pharmaceutical composition of claim 3, wherein the ortholog is obtainable from the group consisting of Chlamydia suis, Chlamydia trachomatis, Chlamydophila abortus, Chlamydophila psittaci, Chlamydophila caviae, Chlamydophila felis Chlamydophila pecorum and C. pneumoniae.

5. A pharmaceutical composition comprising
(i) an isolated nucleic acid encoding a protein that comprises an amino acid sequence selected from the group consisting of the sequences set forth in SEQ ID NOS 20, 34 and 35, and
(ii) a pharmaceutically-acceptable carrier diluent or excipient.

6. The pharmaceutical composition of claim 5, wherein the nucleic acid comprises a nucleotide sequence selected from the group consisting of the sequences set forth in nucleotide residues 37 to 708 of SEQ ID NO: 105, SEQ ID NOS: 120 and 121.

7. A pharmaceutical composition comprising
(i) an isolated nucleic acid encoding a homolog and/or fragment of a protein that comprises an amino acid sequence selected from the group consisting of the sequences set forth in SEQ ID NOS 20, 34 and 35, wherein said homolog and/or fragment has at least 75% sequence identity to any one of said isolated proteins, and
(ii) a pharmaceutically-acceptable carrier diluent or excipient.

8. The pharmaceutical composition of claim 1 is formulated as an immunotherapeutic composition capable of eliciting an immune response in an animal.

9. The pharmaceutical composition of claim 8 is formulated to prevent infection, reduce severity of infection, reduce symptoms caused by infection or ameliorate infection by one or more species, one or more biovar and/or one or more serovar of Chlamydia or Chlamydophila in an animal.

10. The pharmaceutical composition of claim 9, wherein the Chlamydia or Chlamydophila species is selected from the group consisting of Chlamydia trachomatis, Chlamydophila pneumoniae, Chlamydia muridarum, Chlamydia suis, Chlamydophila abortus, Chlamydophila psittaci, Chlamydophila caviae, Chlamydophila felis Chlamydophila pecorum and C. pneumoniae.

11. The pharmaceutical composition of claim 10 wherein the Chlamydia or Chlamydophila species is selected from Chlamydia trachomatis and Chlamydophila pneumoniae.

12. The pharmaceutical composition of claim 9, wherein infection comprises infection of the genital tract, rectum or pharynx.

13. The pharmaceutical composition of claim 9, wherein the pharmaceutical composition is capable of treating or preventing a disorder selected from the group consisting of atherosclerosis, sexually transmitted disease, urethritis, epididymitis, cervicitis, pelvic inflammatory disease, ectopic pregnancy, infertility, tubal factor infertility, epididymitis, proctitis and reactive arthritis, conjunctivitis, including neonatal conjunctivitis, mucopurulent cervicitis, rupture of the membranes, premature delivery, cervical carcinomas, stenosis in an infected organ and inflammation.

14. The pharmaceutical composition of claim 13, wherein the sexually transmitted disease comprises Lymphogranuloma venereum (LGV).

15. The pharmaceutical composition of claim 8, wherein the immunotherapeutic composition is a vaccine.

16. The vaccine of claim 15, wherein the animal is a mammal.

17. The vaccine of claim 16, wherein the mammal is a human.

18. A method for inducing an immune response in an animal, including the step of administering the pharmaceutical composition of claim 1 to an animal.

19. The method of claim 18, wherein the animal is a mammal or avian.

20. The method of claim 19, wherein the mammal is a human, mouse, rat, hamster, swine, cattle, sheep, goat, feline, canine, guinea pig, koala or horse.

21. The method of claim 20, wherein the mammal is a human.

22. The method of claim 18 when used to prevent infection, reduce severity of infection, reduce symptoms caused by infection or ameliorate infection by one or more species, one or more biovar and/or one or more serovar of Chlamydia or Chlamydophila in an animal.

23. The method of claim 22, wherein the Chlamydia or Chlamydophila spp is selected from the group consisting of Chlamydia trachomatis, Chlamydophila pneumoniae, Chlamydia muridarum, Chlamydia suis, Chlamydophila abortus, Chlamydophila psittaci, Chlamydophila caviae, Chlamydophila felis Chlamydophila pecorum and C. pneumoniae.

24. The method of claim 23, wherein the Chlamydia or Chlamydophila spp is selected from Chlamydia trachomatis and Chlamydophila pneumoniae.

25. The method of claim 24, wherein Chlamydia trachomatis comprises a biovar and/or serovar thereof.

26. The method of claim 25, wherein the serovar is selected form the group consisting of serovars A, B, Ba, C, D, Da, E, F, G, H, I, Ia, J, K, L1, L2 and L3.

27. A method for detecting one or more species, one or more biovar and/or one or more serovar of Chlamydia or Chlamydophila in a biological sample including the steps of:—
(I) combining with the biological sample an antibody or antibody fragment capable of binding one or more proteins that comprise an amino acid sequence selected from the group consisting of the sequences set forth in SEQ ID NOS 20, 34 and 35, or a fragment thereof; and
(II) detecting specifically bound antibody or antibody fragment which indicates the presence of said Chlamydia or Chlamydophila.

28. A method of detecting one or more species, one or more biovar and/or one or more serovar of Chlamydia or Chlamydophila in a biological sample including the step of detecting a nucleic acid comprising a nucleotide sequence encoding an amino acid sequence selected from the group consisting of the sequences set forth in SEQ ID NOS 20, 34 and 35, which indicates the presence of said bacteria in the biological sample.

29. The method of claim 28, wherein the nucleotide sequence is selected from the group consisting of the sequences set forth in SEQ ID NOS: 105, 120 and 121.

30. A method of diagnosing infection of an animal by one or more species, one or more biovar and/or one or more serovar of Chlamydia or Chlamydophila, or absence of infection, including the steps of:—
(i) contacting a biological sample from said animal with a protein that comprises an amino acid sequence selected from the group consisting of the sequences set forth in SEQ ID NOS 20, 34 and 35, or a fragment thereof; and
(ii) determining the presence or absence of a complex between said protein and Chlamydia or Chlamydophila-specific antibodies in said sample, wherein the presence of said complex is indicative of said infection.

31. The method of claim 30, wherein the biological sample comprises a cell, tissue, biological fluid, mucus sample, blood sample, serum sample, respiratory wash sample, lavage sample, coronary sample carotid plaque sample, blood vessel or plasma sample.

32. A kit for detecting Chlamydia or Chlamydophila in a biological sample or diagnosing Chlamydia or Chlamydophila infection in an animal, wherein said kit comprises one or more isolated proteins that comprise an amino acid sequence selected from the group consisting of the sequences set forth in SEQ ID NOS 20, 34 and 35.

33. A kit for detecting Chlamydia or Chlamydophila in a biological sample or diagnosing Chlamydia or Chlamydophila infection in an animal, wherein the kit comprises one or more isolated nucleic acids, or fragments thereof, wherein the one or more isolated nucleic acids respectively encode an amino acid sequence selected from the group consisting of the sequences set forth in SEQ ID NOS 20, 34 and 35.

34. The kit of claim 33, wherein the one or more nucleic acids comprise a nucleotide sequence selected from the group consisting of the sequences set forth in nucleotide residues 37 to 708 of SEQ ID NO: 105, SEQ ID NOS: 120 and 121.

35. The kit of claim 32 further comprising a thermostable polymerase.

36. A kit for detecting Chlamydia or Chlamydophila in a biological sample or diagnosing Chlamydia or Chlamydophila infection in an animal, wherein the kit comprises one or more antibody that are capable of binding at least one or more isolated proteins that comprise an amino acid sequence selected from the group consisting of the sequences set forth in SEQ ID NOS 20, 34 and 35.
